(19) United States
(12) Patent Application Publication
(10) Pub. No.: US 2019/0262469 A1
(43) Pub. Date: Aug. 29, 2019

(54) PROTOCELLS TO TREAT MICROBIAL INFECTION AND FOR SYNERGISTIC DELIVERY

(71) Applicants: C. Jeffrey Brinker, Albuquerque, NM (US); Eric C. Carnes, Albuquerque, NM (US); Carlee Erin Ashley, Albuquerque, NM (US); Oscar Negrete, Pleasanton, CA (US); Dan C Wilkinson, Los Angeles, CA (US); Brian S Wilkinson, Albuquerque, NM (US); David Patrick Padilla, Albuquerque, NM (US)

(72) Inventors: C. Jeffrey Brinker, Albuquerque, NM (US); Eric C. Carnes, Albuquerque, NM (US); Carlee Erin Ashley, Albuquerque, NM (US); Oscar Negrete, Pleasanton, CA (US); Dan C Wilkinson, Los Angeles, CA (US); Brian S Wilkinson, Albuquerque, NM (US); David Patrick Padilla, Albuquerque, NM (US)

(21) Appl. No.: 15/757,254

(22) PCT Filed: Sep. 2, 2016
(86) PCT No.: PCT/US2016/050259

§ 371 Date: Mar. 2, 2018

Related U.S. Application Data
(60) Provisional application No. 62/214,381, filed on Sep. 4, 2015.
(60) Provisional application No. 62/214,316, filed on Sep. 4, 2015.
(60) Provisional application No. 62/214,406, filed on Sep. 4, 2015.

(51) Int. Cl. A61K 47/69; A61K 31/337; A61P 31/04

(52) U.S. Cl. CPC A61K 47/6929; A61P 31/04; A61K 31/337; A61K 47/6913

(57) ABSTRACT

The present disclosure relates to protocells that are useful in the treatment and prevention of viral infections, including but not limited to infections caused by a Hendra virus and Nipah virus (NiV). The present disclosure relates to protocells that are useful in the treatment of bacterial infections, including antibiotic-resistant bacterial infections. The protocells are coated with a lipid bi- or multilayer comprising at least one moiety that targets a viral cellular receptor and at least one moiety that ruptures a virally-infected cell membrane. The present disclosure further relates to novel mesoporous metal oxide nanoparticles and related protocells that are useful in the treatment and/or prevention of a wide variety of disorders, including a cancer or a bacterial or viral infection. Such nanoparticles and protocells can be functionalized to allow for synergistic loading of a wide variety of active ingredients.

### CROSS-REFERENCE TO RELATED APPLICATIONS

This application claims the benefit of the filing date of U.S. application Ser. No. 62/214,381, filed on Sep. 4, 2015, U.S. application Ser. No. 62/214,316, filed on Sep. 4, 2015, and U.S. application Ser. No. 62/214,406, filed on Sep. 4, 2015, the disclosures of which are incorporated by reference herein.

### GOVERNMENT SUPPORT

This invention was made with government support under grant nos. 151379 and 166539, under contract no. DE-ACO4-94AL85000 awarded by the U.S. Department of Energy to Sandia Corporation, grant nos. EY016570 and U01 CA151792 awarded by the National Institutes of Health, and grant no. FA9550 10 1 0054 awarded by Air Force Office of Scientific Research. The government has certain rights in the invention.

### FIELD OF THE DISCLOSURE

The present disclosure relates to protocells that are useful in the treatment and prevention of microbial infection, e.g., viral infections, including but not limited to infections caused by a Hendra virus, a Nipah virus (NiV), and a Group A Arbovirus (Alphavirus of the Togavirus family), including Eastern equine encephalitis (EEEV) and Venezuelan equine encephalitis (VEEV), and bacterial infections, e.g., antibiotic-resistant bacterial infection. In certain embodiments, the protocells comprise aminated mesoporous metal oxide nanoparticles which are loaded with small molecules, peptides, silica, nucleic acids, or peptide nucleic acids (PNAs) having antisense RNAs. or antibiotics. The protocells are coated with a lipid bi- or multi-layer optionally comprising at least one moiety that targets a viral cellular receptor and optionally at least one moiety that ruptures a virally-infected cell membrane or that optionally targets a bacterial cellular receptor and optionally at least one moiety that ruptures a bacterially-infected cell membrane. Related methods of treating microbial infections, pharmaceutical compositions and diagnostic and screening assays are also provided.

The present disclosure also relates to mesoporous silica and metal oxide nanoparticles and related protocells that are useful in the treatment and/or prevention of a number of disorders, including cancer or microbial infection. The nanoparticles may be mesoporous silica nanoparticles (MSNPs) that are functionalized with either a polar group (e.g., an amino group) for loading of hydrophilic cargo or a non-polar group (e.g., a methyl or phenyl group) for loading of hydrophobic cargo. By using porous metal oxide nanoparticles that are characterized by a high surface area and well-defined porosity, and through synergistic loading strategies, nanoparticles achieve high-concentration loadings of a wide variety of active ingredients (including hydrophobic, hydrophilic, basic and acidic active ingredients).

### BACKGROUND

New World alphaviruses, including VEEV, EEEV, and WEEV, cause highly pathogenic diseases in humans that exhibit overt encephalitis in a significant percentage of cases (Steele et al., 2010). Because of their high infectivity, their ability to induce devastating disease, the ease with which they can be produced, their high degree of stability, and their potential for aerosolization, VEEV, EEEV, and WEEV are considered potential biological weapons and have been classified as Category B agents by the CDC and NIAID. There are currently no FDA-approved vaccines or drugs to prevent or treat neurotropic infections caused by these and similar encephalitic viruses, and development of effective therapeutics for conditions that affect the CNS is further confounded by the BBB.

Many antiviral compounds, including ribavirin, effectively inhibit a broad range of RNA viruses in vitro but fail to treat infections caused by alphaviruses, flaviviruses or henipaviruses in animal models of viral encephalitis (Diamond, 2009; Rocks et al., 2010; Stephen et al., 1979). Ribavirin, specifically, does not readily cross the BBB when administered intravenously or orally, resulting in subtherapeutic concentrations in the CNS (Georges-Courbot et al., 2006; Salazar et al., 2012; Snell, 2001). Direct administration of ribavirin into the CNS has, however, been show to successfully treat human patients with subacute sclerosing panencephalitis (Hosoya et al., 2004), which suggests that existing antiviral compounds might effectively treat viral encephalitis if they can be delivered to the CNS in a less invasive fashion.

For example, Nipah virus (NiV) is a member of the family Paramyxoviridae, genus Henipavirus.

NiV was initially isolated and identified in 1999 during an outbreak of encephalitis and respiratory illness among pig farmers and people with close contact with pigs in Malaysia and Singapore. Its name originated from Sungai Nipah, a village in the Malaysian Peninsula where pig farmers became ill with encephalitis. Given the relatedness of NiV to Hendra virus (HeV), bat species were quickly singled out for investigation and flying foxes of the genus Pteropus were subsequently identified as the reservoir for NiV.

In the 1999 outbreak, Nipah virus caused a relatively mild disease in pigs, but nearly 300 human cases with over 100 deaths were reported. In order to stop the outbreak, more than a million pigs were euthanized, causing tremendous trade loss for Malaysia. Since this outbreak, no subsequent cases (in neither swine nor human) have been reported in either Malaysia or Singapore. In 2001, NV was again identified as the causative agent in an outbreak of human disease occurring in Bangladesh. Genetic sequencing confirmed this virus as Nipah virus, but a strain different from the one identified in 1999. In the same year, another outbreak was identified retrospectively in Siliguri, India with reports of person-to-person transmission in hospital settings (nosocomial transmission). Unlike the Malaysian NiV outbreak, outbreaks occur almost annually in Bangladesh and have been reported several times in India. CDC Website: Nipah Virus.

Another example is Hendra virus (HeV), also a member of the family Paramyxoviridae and one of two virus species in the genus Henipavirus (the other being Nipah virus). HeV was first isolated in 1994 from specimens obtained during an outbreak of respiratory and neurologic disease in horses and humans in Hendra, a suburb of Brisbane, Australia. The natural reservoir for Hendra virus has since been identified as the flying fox (bats of the genus Pteropus). CDC Website: Hendra Virus.

Treatment of Nipah virus is limited to supportive care. Because Nipah virus encephalitis can be transmitted person-to-person, standard infection control practices and proper barrier nursing techniques are important in preventing hospital-acquired infections (nosocomial transmission). The drug ribavirin has been shown to be effective against Nipah virus in vitro, but human investigations to date have been inconclusive and the clinical usefulness of ribavirin remains uncertain. Similarly, the ribavirin has been shown to be effective against Hendra virus (HeV) in vitro, but its clinical usefulness in the treatment of Hendra virus remains uncertain.

Known anti-viral drugs have caused adverse reactions (e.g., abacavir) or have been associated with drug resistance (e.g., Tamiflu-resistant strains of H1N1). Known antivirals also do not accumulate sufficiently within infected cells (e.g., ribavirin), whereas small interfering RNAs (siRNAs) that target viral mRNA(s) have limited stability in serum and exhibit poor penetration into cells.

Bacteria have developed resistance to all FDA-licensed antibiotics, and their ability to rapidly evolve resistance to new drugs is often demonstrated during the development process (Woodford and Warcham, 2009). More worrisome is the increased prevalence in hospitals of carbapenem-resistant Enterobacteriaceae (CRE). As recently as Mar. 5, 2013, the director of the CDC called CRE ‘nightmare bacteria’ because it is resistant to virtually all antibiotics, including those used as a last resort, it can transfer resistance to other bacteria, and it can result in fatality rate as high as 50%.

Carbapenem-resistant Enterobacteriaceae, vancomycn-resistant Enterococci, multidrug-resistant Pseudomonas aeruginosa and methicillin-resistant Staphylococcus aureus (MRSA) are prominent examples of antibiotic resistant bacteria. See U.S. Patent Application Document No. 20150038705, citing Arias et al., (2012); Jain et al., (2011); Nordmann et al., (2009); Aloush et al., (2006). As noted in the above-cited references, antibiotic-resistant bacteria pose a grave threat to military personnel. Calhoun et al., (2008); Murray et al., (2006); Hujer et al., (2006).

Although nanotechnology promises to revolutionize the diagnosis, prevention, and treatment of infectious disease, existing state-of-the-art nanoparticle delivery vehicles, including many liposomal and polymeric nanoparticle formulations, suffer from limited capacities, uncontrollable release profiles, and complex, specialized synthesis procedures that must be re-adapted for each new cargo molecule, leading to drug- and disease-specific ‘one-off’ approaches (Peer et al., 2007). Furthermore, most nanoparticle delivery vehicles have highly interdependent properties, whereby changing one property, such as loading efficiency, affects numerous other properties, such as size, charge, and stability.

Moreover, despite recent improvements in encapsulation efficiencies and serum stabilities, state-of-the-art liposomes, multilamellar vesicles, and polymeric nanoparticles still suffer from several limitations, including complex processing techniques that are highly sensitive to pH, temperature, ionic strength, presence of organic solvents, lipid or polymer size and composition, and physicochemical properties of the cargo molecule, all of which impact the resulting nanoparticle's size, stability, entrapment efficiency, and release rate (Conley at al., 1997; Couvreur et al., 2006; Morilla et al., 2011; Wong et al., 2003).

### SUMMARY

In one embodiment, the present disclosure provides for flexible, modular platforms for delivery (including but not limited to intranasal (IN) delivery) of a broad-spectrum of small molecules, nucleic acids and antibody-based antivirals to central nervous system (CNS) tissues and cells infected with a wide variety of viruses, including encephalitic New World alphaviruses (e.g., Venezuelan (VEEV), eastern (EEEV), and western (WEEV) equine encephalitis viruses). In certain embodiments, the antiviral platforms are based on protocells that are composed of an aminated mesoporous silica nanoparticlde (MSNP) core that is encapsulated within a supported lipid bilayer (SLB). These protocells possess advantages of both MSNPs and liposomes, including high loading capacities, modifiable release rates, exquisite targeting specificities, long-term colloidal stability and shelf-life, controllable biodistribution, high biocompatibility and biodegradability and low immunogenicity (see, e.g., Ashley et al., 2011; Giri at al., 2007; Lu et al., 2010; Souris et al., 2010; Zhang et al., 2012).

The anti-virus protocells are engineered for. (1) high capacity encapsulation of physicochemically disparate antivirals (2) effective penetration across relevant cellular barriers, including the nasal epithelium and BBB, resulting in rapid CNS accumulation (3) selective internalization by CNS neurons and other potential host cells (4) controlled release of encapsulated antivirals within the cytosol of potential host cells, and (5) optimizable levels of biodegradation and excretion.

In certain aspects, the disclosure provides: (1) an intranasal formulation of antiviral-loaded protocells that protects mammals from fatal challenge with fully-virulent viruses such as VEEV and/or EEEV (2) a cost-effective, scalable synthesis technique amenable to GMP; and (3) a flexible, modular nanoparticle delivery platform that can be easily adapted for additional applications pertinent to chemical and biological defense.

Thus, in one embodiment, an antiviral protocell is provided comprising a mesoporous metal oxide nanoparticle which is loaded with an anti-viral cargo and which is coated with a lipid bi- or multiayer, wherein: (a) the mesoporous metal oxide nanoparticle has a pore size which ranges from about 0.001 to about 100 nm, e.g., from about 0.1 nm to about 100 nm, from about 0.01 nm to about 50 nm, e.g., from about 0.1 nm to about 35 nm, and e.g., from about 2 nm to about 25 nm, and a diameter ranging from about 25 nm to about 500 nm, e.g., from about 100 nm to about 300 nm; and (b) the lipid bi- or multilayer comprises at least one moiety that targets a viral cellular receptor (e.g., a peptide or single chain variable fragment (scFv) that targets ephrin B2 and/or ephrin B3) and at least one moiety that ruptures a viral-infected cell membrane (e.g., octaarginine (R8), H5WYG, Penetratin-HA2, modified HA2-TAT, 4₃E and Histidine 10). In one embodiment, a moiety that targets a viral cellular receptor comprises a peptide or single chain variable fragment (scFv) that targets ephrin B2 and/or ephrin B3 and that comprises one or more amino acid sequences selected from the groups consisting of TGAILHP (SEQ ID NO:52), QGAINHP (SEQ ID NO:53), QHIPKPP (SEQ ID NO:54), QHIRKPP (SEQ ID NO:55), QHRIKPP (SEQ ID NO:56) and QHILNPP (SEQ ID NO:57). In one embodiment, a lipid bi- or multilayer comprises DOPC or DPPC in combination with DOPE, cholesterol and PEG-2000 PE (18:1). In one embodiment, antiviral cargoes include antiviral small molecules, peptides, peptide nucleic acids (PNA's), a mRNA, a siRNA, a shRNA, a micro RNA, an antibody, a protein, a protein toxin (e.g., ricin toxin A-chain or diphtheria toxin A-chain) and/or DNA (including double stranded or linear DNA, minicrcle DNA, plasmid DNA which may be supercoiled and/or packaged (e.g. with histones) and which may be optionally modified with a nuclear localization sequence).

In certain embodiments, the protocels are loaded with ribavirin alone or ribavirin in combination with siRNAs that target conserved regions of viruses (e.g., conserved regions of VEEV and EEEV; Bhomia et al., 2013; O'Brien, 2007) and antibody fragments that have been shown to neutralize viruses (e.g., antibodies that neutralize VEEV and EEEV; O'Brien et al., 2012; Rulker t al., 2012).

In certain embodiments, antiviral protocells are loaded with humanized mAb m102.4 to treat NiV and HeV infections, the anti-EEV humanized mAb HulA3B-7, ZMapp (chimeric monoclonal antibodies c13C6, c2G4 and c4G7) to treat Ebola infections, or pavilizumab or motavizumab (to treat respiratory syncytial virus (RSV), or are loaded with any one of the clinically effective antibodies described in Nasser et al., (2010), or any of the mAb's useful in the treatment of treat Arbovirus infections listed in Table 3 and references cited therein.

Antiviral protocells may be loaded with broadly neutralizing antibodies (bnAbs) that are effective in the treatment of a wide variety of viruses, e.g., the bnAbs AR3A, AR3B, and AR4A to treat Hepatitis C; the bnAbs 2F5, 4E10. M66.6, CAP206-CH12, 10E8 I, PG9, PG16. CHO1-04, PGT 141-145, 2G12, PGT121-123, PGT125-131, PGT135-137, B12. HJ16, CH103-106, VRC01-03, VRC-PG04, 04b, VRC-CH30-34. VRC-CH30-34, 3BNC117, 3BNC60. NIH45-46, 12A12, 12A21, 8ANC131, 134, 1NC9, and 1B2530 to treat HIV: and the bnAbs CR9114, PN-SIA28, CR8033 to treat influenza A and influenza B.

In another embodiment, anti-HCV protocells may be loaded with Hairpin ribozyme or RNAi that targets HCV 5′-UTR, 3′-UTR, and core regions, or such protocells can be load with HCV siRNA 12 (5′-gcccccgauugggggcgacTT-3) (SEQ ID NO:1), siRNA 82 (5′-gcgucuagccauggcguuaTT-3) (SEQ ID NO:2), siRNA 189 (5′-ggacgaccggguccuuucuTT-3) (SEQ ID NO:3), siRNA 286 (5′-ggccuugugguacugccugTT-3′ (SEQ ID NO:4) and 5′-ggccuugugguacugccugTT-3) (SEQ ID NO:5), siRNA 331 (5′-ggucucguagaccgugcacTT-3′ (SEQ ID NO:6) and 3′-TTccggaacaccaugacggac-5) (SEQ ID NO:7).

The protocell antiviral cargos described or exemplified herein are merely illustrative, and those of ordinary skill in the art will readily identify other useful anti-viral cargos that can be loaded into the protocels.

In various embodiments of the antiviral protocells: (a) the protocel is useful in the treatment of HIV and the viral cellular receptor is CD4, CCR5, CXCR4, CD4 glycoprotein or galactosyl ceramide; or (b) the protocell is useful in the treatment of Adenovirus type 2 and the viral cellular receptor is the integrin α₅β₃ or α₅β₅; or (c) the protocell is useful in the treatment of coxsackie virus and the viral cellular receptor is CAR (adenovirus receptor); or (d) the protocell is useful in the treatment of Epstein-Barr virus and the viral cellular receptor is Type 2 complement receptor (CR2) or CD21; or (e) the protocell is useful in the treatment of TGEV and human coronavirus 229E and the viral cellular receptor is Aminopeptidase N; or (f) the protocell is useful in the treatment of Human coronavirus OC43 and bovine coronavirus and the viral cellular receptor is Acetyl-9-0-acetylneuraminic acid; or (g) the protocell is useful in the treatment of Epstein-Barr virus and the viral cellular receptor is Acetyl-9-0-acetylneuraminic acid; or (h) the protocell is useful in the treatment of Epstein-Barr virus and the viral cellular receptor is Acetyl-9-0-acetylneuraminic acid; or (i) the protocell is useful in the treatment of Herpes simplex virus, cytomegalovirus, pseudorabies virus, and bovine herpesvirus and the viral cellular receptor is Heparan sulfate moieties of proteoglycans and partially identified second receptors; or (j) the protocell is useful in the treatment of Influenza A and B viruses and paramyxoviruses and the viral cellular receptor is Neu5Ac (11) on glycoproteins or gangliosides; or (k) the protocell is useful in the treatment of Influenza C virus and the viral cellular receptor is N-Acetyl-9-0-acetylneuraminic acid; or (I) the protocel is useful in the treatment of Measles virus and the viral cellular receptor is Membrane cofactor protein (CD46); or (m) the protocell is useful in the treatment of Poliovirus and the viral cellular receptor is PVR; or (n) the protocell is useful in the treatment of Rhinoviruses (major group) and the viral cellular receptor is ICAM-1; or (o) the protocell is useful in the treatment of Echovirus 1 and the viral cellular receptor is Integrin VLA-2; or (p) the protocell is useful in the treatment of MuLV and the viral cellular receptor is Y*; or (q) the protocell is useful in the treatment of Gibbon ape leukemia virus and the viral cellular receptor is Phosphate permease; or (r) the protocel is useful in the treatment of Sindbis virus and the viral cellular receptor is High-affinity laminin receptor.

In certain embodiments, the protocel is useful in the treatment of Eastern equine encephalitis (EEEV) and Venezuelan equine encephalitis (VEEV) and the viral cellular receptor is heparan sulfate (HS).

In certain embodiments, the antiviral protocell cargo comprises ribavirin or is a siRNA or microRNA which targets conserved regions of EEEV or VEEV RNA-dependent RNA polymerase (RdRp) or nsp1 and E1 glycoprotein genes.

RNA interference (RNAO can be effective when short (about 22 nt), double-stranded fragments of RNA (small interfering RNAs (siRNAs)) are loaded into the RNA-Induced Silencing Complex (RISC), where the strands are separated, and one strand guides cleavage by Argonaute of target mRNAs in a sequence homology-dependent manner. Gavrilov and Saltzman, (2012). As described herein, protocels can be loaded with anti-viral siRNA cargo. In addition to the examples provided herein, anti-viral RNAi techniques and cargo as described in Molaie et al., and other recombinant methods known to those of ordinary skill in the art, can be used in making therapeutic protocells. For example, NiV glycoprotein (G) binds to ephrin B2 and ephrin B3, while NiV fusion protein (F) induces macropinocytosis. Other NiV proteins include RNA polymerase (L), matrix protein (M), nucleocapsid protein (N), phosphoprotein (P). Recombinant techniques including RNAi can be employed to interfere with NiV infection and replication. In certain embodiments, the nanoparticle is an aminated mesoporous silica nanoparticle (MSNP) which has a Zeta (ζ) potential of between about 0 mV to about +40 mV.

In certain embodiments, an antiviral protocell nanoparticle is loaded with: (a) an anti-HIV agent selected from the group consisting of 3TC (Lamivudine), AZT (Zidovudine), (−)-FTC, ddl (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), T20 and fuseon and mixtures thereof; or (b) an anti-HBV agent selected from the group consisting of hepsera (adefovir dipivoxil), lamivudine, entecavir, telbivudine, tenofovir, emtricitabine, clevudine, valtoricitabine, amdoxovir, pradefovir, racivir, BAM 205, nitazoxanide, UT 231-B, Bay 41-4109, EHT899, zadaxin (thymosin alpha-1) and mixtures thereof; or (c) an anti-HCV agent selected from the group consisting of interferon, pegylated interferon, ribavirin, NM 283, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, ACH-1095, GSK625433, TG4040 (MVA-HCV), A-831, F351, NS5A. NS4B, ANA598, A-689, GNI-104, IDX102, ADX184, GL59728, GL60667, PSI-7851, TLR9 Agonist, PHX1766, SP-30 and mixtures thereof.

In certain embodiments: (a) the anti-viral cargo which is effective in the treatment of a Hendra virus (HeV) is ribavirin and/or a Nipah/Hendra neutralizing antibody; and (b) the anti-viral cargo which is effective in the treatment of a Nipah virus (NiV) is ribavirin and/or a human monoclonal antibody which targets the Nipah G glycoprotein.

In other embodiments, antiviral protocells include those in which: (a) the nanoparticle is loaded with the naked siRNA ALN-RSV01 and the protocell is useful in treating RSV; (b) the nanoparticle is loaded with the plasmid DNA NUC B1000 or DPC ARC-520 and the protocell is useful in treating HBV; (c) the nanoparticle is loaded with the Lentivirus pHIV7-shl-TARCCR5RZ and the protocell is useful in treating HIV; and (d) the nanoparticle is loaded with the naked locked nucleic acid (LNA) SPC3649 (LNA) and the protocell is useful in treating HCV.

In certain embodiments, anti-viral protocells include MSNPs that are made by a evaporation-induced self-assembly (EISA) process in which the degree of silica condensation is increased by thermal calcination to maximize the number of Si—O—Si bonds.

As illustrated hereinafter, protocells were loaded with a siRNA cargo that interfered with Nipah protein N translation; the protocells' lipid bilayer coating contained a peptide that targeted ephrin B2 and that comprised one or more amino acid sequences selected from the groups consisting of TGAILHP (SEQ ID NO:52), QGAINHP (SEQ ID NO:53), QHIPKPP (SEQ ID NO:54), QHIRKPP (SEQ ID NO:55), QHRIKPP (SEQ ID NO:56) and QHILNPP (SEQ ID NO:57). These protocells bound host immune cells that expressed the remnant viral protein from prior infection and did not bind to healthy, non-infected host cells. The targeting peptide did not appear to allow the protoceus to be internalized into the host cells, precluding delivery of nucleic acids in proximity of viral production. Another peptide, (R8), was added to the lipid coating and this enabled the protocells to bind infected cells and promote internalization into host cells by macropinocytosis.

Following macropinocytosis, the R8 peptide functioned like an endosomolytic peptide and promoted disruption of the macropinosomes. The reduced pH of the macropinosomes striped the lipid bilayer from the protocells, resulting in cargo release. Once the macropinosomes were disrupted, the remaining silica cores and cargo were delivered into cytosol site of viral assembly. Nucleic acids were released in proximity to reproducing viruses, and were able to effectively stop viral replication and hence limit infection. Each dose of protocells silenced viral replication for approximately five days. Protocells containing plasmids that encoded the same silencing nucleic acid sequence were made, siRNA was substituted for pDNA, and mRNA silencing for around twenty-eight days was achieved. FIG. 12 shows a non-limiting embodiment of use of a nanoparticle (e.g., described herein, such as a protocell) to promote selective delivery of an anti-viral to the host cell.

The aforementioned therapeutic strategy can be used for any virus. Prophylactic delivery of cargo such as anti-viral siRNA, mRNA, or in one embodiment pDNA, enables use of the protocells to prevent viral infections.

Protocells exhibit increased solubility, increased drug circulation half-life, reduced clearance by the kidney, reduced uptake by the reticuloendothelial system (RES) and organ and cell specific targeting. They promote the concentration of ribavirin in the liver as opposed to the kidneys. The protocells achieve enhanced accumulation of drug within target cells. In contrast, ribavirin cannot enter many cell types (e.g., red blood cells or vascular epithelial cells) which are targeted by viruses such as the Nipah virus. In general, drugs with long half-lives do not readily enter cells and therefore show little efficacy against viruses. However, protocells that target a cell type may dramatically increase concentration of drug within cells. This dramatic increase in concentration also aids in overcoming drug resistance mechanisms. In one embodiment, protocells provide for delivery of multiple agents in high concentrations, remain stable in physiologic fluid, penetrate the blood-brain barrier, may target cells, e.g., in the CNS, and/or controllably replace agents.

The antiviral protocells described herein enable the targeted delivery of large dosages (e.g., greater than 10 wt % of the protocell) of antiviral compositions that are effective in the treatment of a wide variety of viral infections. Further, the protocells are highly stable and can retain cargo ex vivo for over three months. Their lipid bi- or multi-layer is engineered to optimize an antiviral delivery profile depending on a variety of parameters, including patient health, targeted site (e.g., systemic drug delivery or delivery at infected cells) and nature of the antibiotic cargo.

High-throughput bioinformatic approaches were employed to identify genes that contribute to antibiotic resistance and to design antisense RNAs that interfere with drug resistance mechanisms. In parallel, mesoporous silica nanoparticle-supported lipid bilayers (‘protocells’) for high capacity delivery of antisense RNA and antibiotics to drug-resistant bacteria were designed.

Anti-bacterial protocels enable high capacity delivery of combinations of numerous antisense RNAs and, when appropriate, FDA-approved antibiotic(s) to target bacteria. To maximize delivery efficacy, the protocells can utilize peptide/nudeic acid hybrids (PNAs), induding cell-penetrating peptide PNA conjugates (CPP-PNAs), which have been shown to readily penetrate Gram-negative and Gram-positive bacteria and highly resistant to nucleases (Rosi et al., 2006). PNAs and antibiotic(s) can be loaded in mesoporous silica nanoparticles (MSNPs) which are encapsulated in a supported lipid bilayer (SLB) to form the protocell construct. In order to promote concentrated release of PNAs and antibiotics at the site of infection, the protocell surface can be modified with ligand(s) that bind to target bacteria, and the SLB can be composed of lipids that degrade in the presence of reactive oxygen species (ROS), which are prevalent at sites of infection.

In one embodiment, an anti-bacterial protocell is provided comprising a mesoporous metal oxide nanoparticle which is loaded with an anti-bacterial cargo and which is coated with a lipid bi- or multilayer, wherein: (a) the mesoporous metal oxide nanoparticle has a pore size which ranges from about 0.001 to about 100 nm. e.g., from about 0.1 nm to about 100 nm, from about 0.01 nm to about 50 nm, e.g., from about 0.1 nm to about 35 nm, and e.g., from about 2 nm to about 25 nm, and a diameter ranging from about 25 nm to about 500 nm, e.g., from about 100 nm to about 300 nm; and (b) the lipid bi- or multilayer comprises at least one moiety that targets a bacterial cellular receptor (e.g., a peptide or single chain variable fragment (scFv) that targets Fcγ from human IgG, human complement C3, ephrin B2 or mannosylated cholesterol) and at least one moiety that ruptures a bacterially-infected cell membrane (e.g., a peptide selected from the group consisting of octaarginine (R8), H5WYG, Penetratin-HA2, modified HA2-TAT, 4₃E and Histidine 10). In one embodiment, lipid bi- or multi-layer comprises DOPC or DPPC in combination with DOPE, cholesterol and PEG-2000 PE (18:1). In one embodiment, anti-bacterial cargos include novel peptide nucleic acids (PNAs) which bind to a ribosomal binding site of one or more genes selected from the group consisting of qnrB9, aac(6′)-Ib, sul1, bla_{SHV-11}, bla_{TX-M-15}, blaNDM-1, the bla gene encoding TEM-1 and equivalents thereof. Novel PNA's include asRNA molecules that inhibit the translation of a wide variety of antibiotic resistance-conferring peptides.

In certain non-limiting examples, the anti-bacterial protocell comprise a nucleic acid molecule capable of inhibiting the translation of hemolysin produced by S. aureus or extracellular toxin complex (ETC) produced by K. pneumoniae, or a peptide nucleic acid (PNA) comprising nucleic acid molecules capable of inhibiting the translation of hemolysin produced by S. aureus or extracellular toxin complex (ETC) produced by K. pneumoniae. In other non-limiting embodiments, the anti-bacterial cargo comprises siRNAs, miRNAs and/or shRNAs. In one embodiment, the antibacterial cargo is a peptide nucleic acid (PNA) comprised of nucleic acid molecules which inhibit the translation of a mRNA selected from the group consisting of a TEM beta-lactamase (class A) mRNA, a SHV beta-lactamase (class A) mRNA, a CTX-M beta-lactamase (class A) mRNA, an OXA beta-lactamase (class D) mRNA, a PER mRNA, a VEB mRNA, a GES mRNA, an IBC beta-lactamase mRNA, an AmpC type β-lactamase mRNA, and a carbapenemase mRNA (including but not limited to KPC (K. pneumoniae carbapenemase) (Class A) mRNA), and the mammalian and non-mammalian orthologs thereof. In one embodiment, the antibacterial cargo is a peptide nucleic acid (PNA) comprised of nucleic acid molecules capable of inhibiting the translation of a mRNA selected from the group consisting of Metallo-beta-lactamase NDM-1 mRNA, SHV and TEM beta-lactamase mRNA, CMY-6 AmpC-type beta-lactamase mRNA, CTX-M-15 extended spectrum beta-lactamase mRNA; TEM-1 beta-lactamase mRNA; OXA-1 beta-lactamase mRNA; Aminoglycoside-(3)(9)-adenyftransferase AADA2 mRNA; Sul1 dihydropteroate synthase mRNA; Undecaprenyl-diphosphatase mRNA; 16S ribosomal RNA methyltransferase mRNA; AAC(6)-Ib aminoglycoside 6-N-acetyl transferase type Ib mRNA; Sul1 dihydropteroate synthase mRNA; 16S rRNA methyltransferase RmtC mRNA; Aminoglycoside 3 phosphotransferase APH(3)-Ib (strA) mRNA; Sul2 mRNA, sulfonamide insensitive dihydropteroate synthetase mRNA; Streptomycin 3-O-adenylyltransferase aadA ANT(3)-Ia mRNA; Dfra14 trimethoprim-resistant dihydrofolate reductase mRNA; QnrB10 mRNA; Aminoglycoside N(3)-acetyltransferase II (ACC(3)-II)mRNA; Tetracycline efflux protein TetA mRNA; and Macrolide 2-phosphotransferase mphA mRNA, and the mammalian and non-mammalian orthologs thereof. In one embodiment, the antibacterial cargo is a nucleic acid molecule capable of inhibiting the translation of one or more efflux pumps of the families MFS (major facilitator superfamily), SMR (small multidrug resistance), ABC (ATP-binding cassette) and MATE (Multidrug and Toxic Compound Extrusion). In one embodiment, the antibacterial cargo is a nucleic acid molecule capable of inhibiting the transcription of one or more genes selected from the group consisting of qnrB9, aac(6′)-Ib, sul1, bla_{SHV-11}, bla_{CTX-M-15}, blaNDM-1, the bla_{TEM-1}, aph-3′-ia (aminoglycoside-3′-phosphotransferase type Ia, APH(3′)-Ia) and equivalents thereof.

Also provided are methods of treating a wide variety of antibiotic resistant bacterial infections, including but not limited to infections caused by a bacterium selected from the group consisting of multidrug-resistant (MDR) Klebsiella pneumoniae (Kpn), methicillin-resistant Staphylococcus aureus (MRSA), F. tularensis and B. pseudomallei. Certain methods of treatment treat a subject who suffers from a methicillin-resistant Staphylococcus aureus (MRSA) skin or soft tissue infection (SSTI). Related pharmaceutical compositions, diagnostic and screening methods and kits are also provided.

In certain embodiments, anti-bacterial protocells include MSNP's that are made by a novel evaporation-induced self-assembly (EISA) process in which the degree of silica condensation is increased by thermal calcination to maximize the number of Si—O—Si bonds and reduced by using acidified ethanol to extract structure-directing surfactants.

In still another embodiment, a plasmid vector is provided comprising a novel asRNA as described herein, the asRNA being operably linked to a promoter. Certain methods of treatment treat a subject who suffers from an antibiotic-resistant bacterial infection by administering therapeutically-effective doses of the novel plasmid vectors to the subject.

Also provided is a screening method which uses quantitative PCR (qPCR) to determine the antibiotic effect of a composition on a sample of bacterially infected cells, as well as a novel microscale test strip compatible with either broth or agar microdilution.

The anti-bacterial protocells described herein enable the targeted delivery of large dosages (e.g., greater than 10 wt % of the protocell) of antibiotic compositions that are effective in the treatment of antibiotic-resistant bacteria. Further, the protocells are highly stable and can retain cargo ex viv for over three months. Their lipid bi- or multi-layer is engineered to optimize an antibiotic delivery profile depending on a variety of parameters, including patient health, targeted site (e.g., systemic drug delivery or delivery at infected cells) and nature of the anti-bacterial cargo.

In another embodiment, using porous metal oxide nanoparticles (such as mesoporous silica characterized by high surface area and well-defined porosity), and/or synergistic loading strategies, high concentration loadings of a variety of compounds (e.g., hydrophobic, hydrophilic, basic and acidic compounds) within a porous nanoparticle core were achieved. Synergistic loading was achieved in part by utilizing hydrophobic-hydrophilic and electrostatic interactions between a cargo and a nanoparticle's porous core (e.g., using positively-charged mesoporous cores to load negatively charged cargo, and using hydrophobic cores to load hydrophobic cargo).

In order to further enhance synergy and improve cargo retention and biocompatibility, a lipid or polymer cap was fused on the surface of the cargo-loaded nanoparticles in some embodiments. Employing synergies between charge and/or hydrophobic-hydrophilic interactions increased cargo loading capacity and retention. The nature of the lipid or polymer was used to control solubility and stability of the cargo-loaded nanoparticle compounds, as well as to provide a surface to attach biofunctional ligands (e.g., targeting ligands).

Optionally, a protocell polymer or lipid coating can also be engineered to destabilize under specific conditions, thereby providing a cargo release trigger mechanism based on a variety of factors, including exposure to altered pH and extemal magnetic-field induced heating. Such triggering is discretionary, as synergistic components in the nanoparticle and surface coatings can be tailored to release compounds without stimulus over desired time profiles (e.g., to effect burst release of all cargo within about twelve hours or sustained release of cargo at a rate of about 10% per day for around ten days).

Thus, in one embodiment, mesoporous metal oxide nanoparticles are provided having a pore size ranging from about 0.001 to about 100 nm, from about 0.01 to about 50 nm, from about 0.1 to about 100 nm, from about 0.1 nm to about 35 nm, and from about 2 nm to about 25 nm, and a diameter ranging from about 25 nm to about 500 nm, e.g., from about 100 nm to about 300 nm. The nanoparticle is functionalized with either a polar group for loading of hydrophilic cargo or a non-polar group for loading of hydrophobic cargo.

In some embodiments, the nanoparticle is a mesoporous silica nanoparticle (MSNP) or a mesoporous aluminum oxide (Al₂O₃) nanoparticle whose pores are functionalized with an amino group polar group or a non-polar methyl or phenyl group. For example, the nanoparticle is aminated or methylated or otherwise functionalized with an organosiloxane. For example, the nanoparticle can be aminated with a primary amine, a secondary amine a tertiary amine, each of which is functionalized with a silicon atom (2) a monoamine or a polyamine (3)N-(2-aminoethyl)-3-aminopropyltrimethoxysilane (AEPTMS) (4) 3-aminopropyltrimethoxysilane (APTMS) (5) 3-aminopropyltriethoxysilane (APTS) (6) an amino-functional trialkoxysilane, and (7) protonated secondary amines, protonated tertiary alkyl amines, protonated amidines, protonated guanidines, protonated pyridines, protonated pyrimidines, protonated pyrazines, protonated purines, protonated imidazoles, protonated pyrroles, and quatemary alkyl amines, or combinations thereof, or methylated with hexamethyldisilazane (HDMS), sodium bis(trimethylsilyl)amide (NaHDMS) or potassium bis(trimethylsilyl)amide (KHDMS), or functionalized with non-polar phenyltriethoxysilane (PTS), or combinations thereof.

In certain embodiments, the nanoparticle has a differential pore volume of between about 0.25 cm³/g to about 10 cm³/g (e.g., from about 0.25 cm³/g to about 1.5 cm³/g) and a nominal BET surface area of between about 50 m²/g to about 1,500 m²/g, e.g., from about 100 m²/g to about 1,300 m²/g.

Examples of therapeutically useful nanoparticles include but are not limited to a mesoporous metal oxide nanoparticle (MSNP) wherein either: (a) the nanoparticle is methylated or functionalized with a phenyl group and is loaded with a cargo having a water solubility of between about less than 0.001 mg/mL to about 0.5 mg/mL; or (b) the nanoparticle is aminated and is loaded with a cargo having a water solubility of between about 0.2 mg/mL to greater than about 3,000 mg/mL. In certain embodiments, the nanoparticle is methylated or functionalized with a phenyl group and is loaded with one or more compositions which have a water solubility of between about less than 0.001 mg/mL to about 0.50 mg/mL. Examples of such compositions include paclitaxel, imatinib, curcumin, ciclopirox and ibuprofen. In other embodiments, the nanoparticle is aminated and is loaded with a cargo having a water solubility of between about 0.2 mg/mL to greater than about 3,000 mg/mL. Such a cargo can include a small molecule, a mRNA, a siRNA, a shRNA, a micro RNA, a PNA, a protein, a protein toxin (e.g., ricin toxin A-chain or diphtheria toxin A-chain) and/or DNA (including double stranded or linear DNA, minicircle DNA, plasmid DNA which may be supercoiled and/or packaged (e.g., with histones) and which may be optionally modified with a nuclear localization sequence). Examples of such cargo include cisplatin, doxorubicn, gemcitabine, carboplatin, ciprofloxacin and ribavirin. The Zeta (ζ) potential of an aminated nanoparticle can vary between about 0 mV to about +40 mV, and the Zeta (ζ) potential of a methylated or phenyl-functionalized nanoparticle can vary between about −40 mV to about 0 mV.

Nanoparticles may comprise a targeting ligand and/or a reporter (as defined hereinafter) and can be loaded with one or more therapeutic cargo components, either by loading exclusively onto the nanoparticle surface or by pore and/or surface loading.

In certain embodiments, the nanoparticle is a mesoporous silica nanoparticle (MSNP) which is self-assembled using a templating surfactant system comprised of at least one cationic surfactant. For example, in one embodiment, mesoporous silica nanoparticles (MSNPs) are made by an evaporation-induced self-assembly (EISA) process which includes the steps of: (a) preparing a precursor solution comprising (1) a surfactant (2) tetraethyl orthosilicate (TEOS), tetramethyl orthosilicate (TMOS) or a mixture thereof (3) a C₁₋₄ alcohol (in one embodiment, ethanol), and (4) water, wherein said surfactant, tetraethyl orthosilicate (TEOS), tetramethyl orthosilicate (TMOS) or a mixture thereof. C₁₋₄ alcohol, and water are combined at a temperature below the surfactant's critical micelle concentration; (b) atomizing the precursor solution to generate droplets; (c) drying the droplets, thereby evaporating solvent and increasing effective surfactant concentration and inducing nanoparticle self-assembly; and (d) heating dried droplets, thereby evaporating residual solvent, inducing silica condensation and forming solid nanoparticles, wherein the degree of silica condensation is increased by thermal calcination to maximize the number of Si—O—Si bonds and reduced by using acidified ethanol to extract structure-directing surfactants.

During the EISA step of heating dried droplets, the degree of silica condensation can be varied by using thermal calcination to maximize the number of Si—O—Si bonds and by using acidified ethanol to reduce the number of Si—O—Si bonds by extracting structure-directing surfactants. Examples of surfactants used in the EISA process include but are not limited to cationic surfactants selected from the group consisting of a dodecylsulfate salt (e.g., sodium dodecylsulfate or lithium dodecylsulfate (SDS)), a tetradecyl-trimethyl-ammonium salt (e.g., tetradecyl-trimethyl-ammonium bromide (C₁₄TAB) or tetradecyl-trimethyl-ammonium chloride), a hexadecyltrimethylammonium salt (e.g., hexadecyltrimethylammonium bromide (C₁₆; CTAB)), an octadecyltrimethylammonium salt (e.g., octadecytrimethylammonium bromide (C₁₈; OTAB)), a dodecylethyldimethylammonium salt (e.g., dodecylethyldimethylammonium bromide), a cetylpyridinium salt (e.g., cetylpyridinium chloride (CPC)), polyethoxylated tallow amine (POEA), hexadecyltrimethylammonium p-toluenesulfonate, a benzalkonium salt (e.g., benzalkonium chloride (BAC)), or a benzethonium salt (e.g., benzethonium chloride (BZT)) and mixtures thereof. In one embodiment, the surfactant is hexadecyltrimethylammonium bromide (C₁₆; CTAB).

In one EISA embodiment, the precursor solution is dispersed within an oil phase to form a multiphase emulsion, and: (a) the precursor solution comprises (1) tetraethyl orthosilicate (TEOS), tetramethyl orthosilicate (TMOS) or a mixture thereof, and (2) at least one cationic surfactant; and (b) the oil phase comprises a C₁₂-C₂₀ alkane and a non-ionic emulsifier soluble in the oil phase.

In one EISA process, the precursor solution is an oi-in-water emulsion that comprises one or more components selected from the group consisting of: (1) hexadecyltrimethylammonium bromide (C₁₆; CTAB) (2) a Brij® surfactant (in one embodiment, Brij®56) (3) a block copolymer based on ethylene oxide and propylene oxide (e.g., Pluronic® F108), optionally in combination with urea and/or polystyrene (PS) or glycerol monooleate (4) a difunctional block copolymer surfactant terminating in a primary hydroxyl group (in one embodiment, Pluronic® P123), optionally in combination with (i) a triblock copolymer of poly(ethylene oxide) (PEO) or poly(propylene oxide) (PPO), and/or (ii) polypropylene glycol acrylate (PPGA). The volumetric ratio of the precursor solution:oil phase is in one embodiment, between about 1:2 to 1:4.

Protocells comprise a lipid bi- or multi-layer-coated nanoparticle. In one embodiment, the lipid bi- or multi-layer comprises DOPC or DPPC in combination with DOPE, cholesterol and PEG-2000 PE (18:1) and includes a cell targeting species (e.g., a peptide that targets cancer cells). Examples of a cell-targeting species include a S94 peptide, a MET binding peptide or mixtures thereof. The lipid bi- or multi-layer can also include a number of other compositions, including cholesterol, SiOH and a reporter.

In some embodiments: (a) the nanoparticle is an aminated silica nanoparticle (MSNP); (b) the lipid bilayer comprises DOPC or DPPC in combination with DOPE, cholesterol and PEG-2000 PE (18:1); (c) the targeting peptide targets cancer cells; (d) the cargo comprises one or more hydrophilic anticancer active ingredients; and (e) at a pH of about 5, the protocell releases between about 30% to about 100% of its cargo at about three hours after delivery, and releases about 60% to about 100% of its cargo at about six hours after delivery.

In other embodiments: (a) the nanoparticle is a methylated or phenyl-functionalized silica nanoparticle (MSNP); (b) the lipid bi-layer comprises DOPC or DPPC in combination with DOPE, cholesterol and PEG-2000 PE (18:1); (c) the targeting peptide targets cancer cells; (d) the cargo comprises one or more hydrophobic anticancer active ingredients; and (e) at a pH of about 5, the protocell releases between about 40% to about 90% of its cargo at about three hours after delivery, and releases about 90% to about 100% of its cargo at about twelve hours after delivery.

In certain embodiments, a protocell is provided comprising a MSNP coated with a lipid bi-layer which: (a) at a pH of about 7 and a period of about 6 hours after delivery, releases no more than about 5 wt % of its cargo; (b) at a pH of about 5 and a period of about 6 hours after delivery, releases no less than about 50 wt % of its cargo; and (c) at a pH of about 5 and a period of about 12 hours after delivery, releases no less than about 90 wt % of its cargo.

In certain embodiments, a protocell is provided comprising a MSNP coated with a lipid bi-layer which: (a) at a pH of about 7 and a period of about 24 hours after delivery, releases no more than about 5 wt % of its cargo; (b) at a pH of about 5 and a period of about 6 hours after delivery, releases no less than about 20 wt % of its cargo; (c) at a pH of about 5 and a period of about 12 hours after delivery, releases no less than about 70 wt % of its cargo; and (d) at a pH of about 5 and a period of about 24 hours after delivery, releases no less than about 90 wt % of its cargo.

In certain embodiments, the nanoparticle is an a mesoporous silica nanoparticle (MSNP) and the protocel is formed by fusing liposomes to the MSNP in the presence of divalent cations, thereby coating the MSNP with a supported lipid multi-layer.

In other embodiments, the nanoparticle is a mesoporous silica nanoparticle (MSNP) and wherein the weight ratio of cargo to silica ranges from about 0.10 to about 0.75.

In certain embodiments, the nanoparticle is a silica nanoparticle (MSNP) having a multimodal pore morphology defined by surface-accessible pores having a diameter of from about 5-50 nm, in one embodiment, from about 5-25 nm, e.g., from about 10-35 nm, e.g., about 20-25 nm interconnected by internal pores ranging in size from about 1-15 nm, in one embodiment, from about 5-15 nm, wherein the surface accessible pores have a larger diameter than the internal pores.

In one embodiment, silica nanoparticles can be generated using an EISA process in which the precursor solution is prepared by combining the surfactant, TEOS, ethanol, and water well below the surfactant's critical micelle concentration. The sol is atomized and the droplet is carried into a drying zone where solvent evaporation begins, increasing the effective surfactant concentration and facilitating self-assembly. The droplet enters the heating zone, which evaporates the remaining solvent and drives silica condensation to form solid particles. This robust process allows for tunable pore size, controllable particle diameter, and dissolution kinetics that can be modulated.

Further, in conjunction with silica core functionalization, a supported-lipid bi- or multilayer (SLB) can be formulated with lipids that are zwitterionic, positively charged, or negatively charged. This allows for synergistic cargo loading upon fusion with the core particle, thereby providing an additional method for controlling the type and amount of cargo that is loaded.

Protocells can have a unique set of biophysical and biochemical properties that can be independently varied in order to encapsulate a variety of disparate cargo types for various drug delivery applications. The ability to independently control each property allows for the physiochemical properties of each cargo to be masked, effectively modulating the cargos aqueous solubility and permeability, which ultimately allows for control over a drug's pharmacokinetics behavior.

Further, the protocells are highly stable and can retain cargo ex vivo for over three months. Their lipid bi- or multilayer is engineered for an active ingredient delivery profile depending on a variety of parameters, including patient health, targeted site (e.g., systemic drug delivery or delivery at cancerous or bacterially or virally infected cells) and nature of the drug cargo.

In one embodiment, an antiviral protocell comprising a mesoporous silica or metal oxide nanoparticle is provided which is loaded with an anti-viral cargo and which is coated with a lipid bi- or multilayer, wherein: (a) the mesoporous metal oxide nanoparticle has a pore size which ranges from about 0.001 to about 100 nm, e.g., from about 0.01 nm to about 50 nm, from about 0.1 to about 100 nm, from about 0.1 nm to about 35 nm, from about 2 nm to about 25 nm, and a diameter ranging from about 25 nm to about 500 nm, e.g., from about 100 nm to about 300 nm; and (b) the lipid bi- or multilayer comprises at least one targeting moiety that targets a virally-infected host cell. In one embodiment, the targeting moiety is a peptide or single chain variable fragment (scFv). In one embodiment, the targeting moiety targets ephrin B2 and/or ephrin B3. In one embodiment, the targeting moiety is a peptide or single chain variable fragment (scFv) that targets ephrin B2 and/or ephrin B3. In one embodiment, the targeting moiety comprises one or more amino acid sequences selected from the groups consisting of TGAILHP, QGAINHP, QHIRKPP, QHRIKPP and QHILNPP. In one embodiment, the anti-viral cargo is effective in the treatment of a Hendra virus (HeV) or a Nipah virus (NiV). In one embodiment, the protocell further comprises an endosomolytic moiety. In one embodiment, the endosomolytic moiety is a peptide. In one embodiment, the endosomolytic moiety ruptures acidic intracellular vesicles of the virally-infected cell. In one embodiment, the endosomolytic moiety is a peptide selected from the group consisting of octaarginine (R8), H5WYG, Penetratin-HA2, modified HA2-TAT, 43E and Histidine 10. In one embodiment, the antiviral cargo is selected from the group consisting of a small molecule, a mRNA, a siRNA, a shRNA, a micro RNA, a PNA, a PNA comprised of RNA's, an antibody, a protein, a protein toxin (e.g., ricin toxin A-chain or diphtheria toxin A-chain) and/or DNA (including double stranded or linear DNA, minicircle DNA, plasmid DNA which may be supercoiled and/or packaged (e.g., with histones) and which may be optionally modified with a nuclear localization sequence). In one embodiment, the lipid bi- or multilayer is comprised of lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)aminop]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures thereof.

In one embodiment, the lipid bi- or multilayer comprises DOPC in combination with DOPE. In one embodiment, the lipid bi- or multi-layer comprises DOTAP, DOPG, DOPC or mixtures thereof. In one embodiment, the lipid bi- or multi-layer comprises DOPG and DOPC. In one embodiment, the lipid bi- or multilayer further comprises cholesterol. In one embodiment, the lipid bi- or multilayer comprises DOPC or DPPC in combination with DOPE, cholesterol and PEG-2000 PE (18:1). In one embodiment, the lipid bi- or multilayer further comprises between about 0.5 wt % to about 5 wt % (e.g., about 1 wt %) of glutathione. In one embodiment, the lipid bi- or multilayer further comprises a RGD (Arg-Gly-Asp) peptide. In one embodiment, the antiviral cargo is a siRNA, an antibody or antibody fragment, an IgG molecule or a fragment thereof, a minicircle DNA vector that encodes antiviral shRNA, or ribavirin. In one embodiment, the nanoparticle is an aminated mesoporous silica nanoparticle (MSNP). In one embodiment, the nanoparticle is aminated with aminopropyltriethoxysilane (APTES) or 3-[2-(2 aminoethylamino)ethylamino]propyltrimethoxysilane (AEPTMS). In one embodiment, the mesoporous silica nanoparticle (MSNP) is made by an evaporation-induced self-assembly (EISA) process.

In one embodiment, the evaporation-induced self-assembly (EISA) process includes the steps of: (a) preparing a precursor solution comprising (1) a surfactant (2) tetraethyl orthosilicate (TEOS), tetramethyl orthosilicate (TMOS) or a mixture thereof (3) a C₁₋₄ alcohol (in one embodiment, ethanol), and (4) water, wherein said surfactant, tetraethyl orthosilicate (TEOS), tetramethyl orthosilicate (TMOS) or a mixture thereof, C₁₋₄ alcohol, and water are combined at a temperature below the surfactant's critical micelle concentration; (b) atomizing the precursor solution to generate droplets; drying the droplets, thereby evaporating solvent and increasing effective surfactant concentration and inducing nanoparticle self-assembly; (d) heating dried droplets, thereby evaporating residual solvent, inducing silica condensation and forming solid nanoparticles. In one embodiment, the evaporation-induced self-assembly (EISA) process further includes the steps of: (a) varying the degree of silica condensation by thermal calcination to maximize the number of Si—O—Si bonds; and (b) adding an amine-containing silane to the precursor solution to replace a controllable fraction of Si—O—Si bonds with Si—R—NH₂ bonds, where R is a C₁₋₁₂ hydrocarbon.

In one embodiment, the nanoparticle has a differential pore volume of between about 0.25 cm³/g to about 10 cm³/g, from about 0.3 cm³/g to about 3 cm³/g, or from about 0.25 cm³/g to about 1.5 cm³/g. In one embodiment, the nanoparticle has a nominal BET surface area of between about 50 m²/g to about 1,500 m²/g, or from about 100 m²/g to about 1,300 m²/g. In one embodiment, the lipid bi- or multilayer is PEGylated. In one embodiment, the nanoparticle is a mesoporous silica nanoparticle (MSNP) and the protocel is formed by fusing liposomes to the MSNP in the presence of divalent cations, thereby coating the MSNP with a supported lipid multilayer. In one embodiment, the nanoparticle is a mesoporous silica nanoparticle (MSNP) and wherein the weight ratio of antiviral cargo to silica ranges from about 0.10 to about 0.75. In one embodiment, the nanoparticle is a silica nanoparticle (MSNP) which is coated with a lipid bi- or multilayer and wherein: (a) at a pH of about 7 and a period of about 12 days after delivery, the protocel will release no more than about 10 wt % of its antiviral cargo; and (b) at a pH of about 5 and a period of about one day after delivery, the protocell will release no less than about 90 wt % of its antiviral cargo.

In one embodiment, the nanoparticle is a mesoporous silica nanoparticle (MSNP) which is coated with a lipid multilayer and wherein: (a) at a pH of about 7 and a period of about 12 days after delivery, the protocel will release no more than about 5 wt % of its antiviral cargo; and (b) at a pH of about 5 and a period of about ten days after delivery, the protocell will release no less than about 10 wt % to about 60 wt % of its antiviral cargo. In one embodiment: (a) the protocell is useful in the treatment of HIV and the viral cellular receptor is CD4, CCR5, CXCR4, CD4 glycoprotein or galactosyl ceramide; or (b) the protocel is useful in the treatment of Adenovirus type 2 and the viral cellular receptor is the integrin asps or as; or (c) the protocell is useful in the treatment of coxsackie virus and the viral cellular receptor is CAR (adenovirus receptor); or (d) the protocell is useful in the treatment of Epstein-Barr virus and the viral cellular receptor is Type 2 complement receptor (CR2) or CD21; or (e) the protocel is useful in the treatment of TGEV and human coronavirus 229E and the viral cellular receptor is Aminopeptidase N; or (f) the protocell is useful in the treatment of Human coronavirus OC43 and bovine coronavirus and the viral cellular receptor is Acetyl-9-0-acetylneuraminic acid; or (g) the protocell is useful in the treatment of Epstein-Barr virus and the viral cellular receptor is Acetyl-9-0-acetylneuraminic acid; or (h) the protocell is useful in the treatment of Epstein-Barr virus and the viral cellular receptor is Acetyl-9-0-acetylneuraminic acid; or (i) the protocell is useful in the treatment of Herpes simplex virus, cytomegalovirus, pseudorabies virus, and bovine herpesvirus and the viral cellular receptor is Heparan sulfate moieties of proteoglycans and partially identified second receptors; or (j) the protocell is useful in the treatment of Influenza A and B viruses and paramyxoviruses and the viral cellular receptor is Neu5Ac (11) on glycoproteins or gangliosides; or (k) the protocell is useful in the treatment of Influenza C virus and the viral cellular receptor is N-Acetyl-9-0-acetylneuraminic acid; or (I) the protocell is useful in the treatment of Measles virus and the viral cellular receptor is Membrane cofactor protein (CD46); or (m) the protocell is useful in the treatment of Poliovirus and the viral cellular receptor is PVR; or (n) the protocel is useful in the treatment of Rhinoviruses (major group) and the viral cellular receptor is ICAM-1; or (o) the protocell is useful in the treatment of Echovirus 1 and the viral cellular receptor is Integrin VLA-2; or (p) the protocell is useful in the treatment of MuLV and the viral cellular receptor is Y⁺; or (q) the protocell is useful in the treatment of Gibbon ape leukemia virus and the viral cellular receptor is Phosphate permease; or (r) the protocell is useful in the treatment of Sindbis virus and the viral cellular receptor is High-affinity laminin receptor.

In one embodiment, the protocell is useful in the treatment of Eastern equine encephalitis (EEEV) and Venezuelan equine encephalitis (VEEV) and the viral cellular receptor is heparan sulfate (HS). In one embodiment, the antiviral cargo comprises ribavirin or a nucleic acid. In one embodiment, the antiviral cargo is a siRNA or microRNA which targets conserved regions of EEEV or VEEV RNA-dependent RNA polymerase (RdRp) or nsp1 and E1 glycoprotein genes. In one embodiment, the nanoparticle is an aminated mesoporous silica nanoparticle (MSNP) which has a Zeta (ζ) potential of between about 0 mV to about +40 mV. In one embodiment, the surfactant is a cationic surfactant selected from the group consisting of a dodecylsulfate salt (e.g., sodium dodecylsulfate or lithium dodecylsulfate (SDS)), a tetradecyl-trimethyl-ammonium salt (e.g., tetradecyl-trimethyl-ammonium bromide (C₁₄TAB) or tetradecyl-trimethyl-ammonium chloride), a hexadecyftrimethylammonium salt (e.g., hexadecyltrimethylammonium bromide (C₁₆; CTAB)), an octadecytrimethylammonium salt (e.g., octadecyltrimethylammonium bromide (C₁₈; OTAB)), a dodecylethyldimethylammonium salt (e.g., dodecylethyldimethylammonium bromide), a cetylpyridinium salt (e.g., cetylpyridinium chloride (CPC)), polyethoxylated tallow amine (POEA), hexadecytrimethylammonium p-toluenesulfonate, a benzalkonium salt (e.g., benzalkonium chloride (BAC)), or a benzethonium salt (e.g., benzethonium chloride (BZT)) and mixtures thereof. In one embodiment, the nanoparticle has a nominal BET surface area of about 1,200 m²/g and the surfactant is hexadecyltrimethylammonium bromide (C₁₆; CTAB). In one embodiment, the nanoparticle is a mesoporous silica nanoparticle (MSNP) which is modified with SiOH. In one embodiment, the nanoparticle is loaded with two or more different anti-viral cargos. In one embodiment, the two or more different anti-viral cargos are of different kinds.

In one embodiment, the nanoparticle is loaded with: (a) an anti-HIV agent selected from the group consisting of 3TC (Lamivudine), AZT (Zidovudine), (−)-FTC, ddl (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir). APV (Amprenavir), LPV (Lopinavir), T20, fuseon, and mixtures thereof; or (b) an anti-HBV agent selected from the group consisting of hepsera (adefovir dipivoxil), lamivudine, entecavir, telbivudine, tenofovir, emtricitabine, clevudine, valtorcitabine, amdoxovir, pradefovir, racivir, BAM 205, nitazoxanide. UT 231-B, Bay 41-4109. EHT899, zadaxin (thymosin alpha-1), and mixtures thereof; or (c) an anti-HCV agent selected from the group consisting of interferon, pegylated interferon, ribavirin, NM 283, VX-950 (telaprevir), SCH 50304, TMC435. VX-500. BX-813, SCH503034, R1626. ITMN-191 (R7227), R7128, PF-868554, TT033, CGH-759, GI 5005, MK-7009, SIRNA-034, MK-0608, A-837093, GS 9190, ACH-1095, GSK625433, TG4040 (MVA-HCV), A-831, F351, NS5A, NS4B, ANA598, A-689, GNI-104, IDX102, ADX184, GL59728, GL60667, PSI-7851, TLR9 Agonist, PHX1766, SP-30, and mixtures thereof. In one embodiment, (a) the anti-viral cargo which is effective in the treatment of a Hendra virus (HeV) is ribavirin and/or a Nipah/Hendra neutralizing antibody; and (b) the anti-viral cargo which is effective in the treatment of a Nipah virus (NiV) is ribavirin and/or a human monoclonal antibody which targets the Nipah G glycoprotein.

A pharmaceutical composition comprising: (a) a therapeutically effective amount of protocells; and (b) optionally, one or more pharmaceutically acceptable excipients. In one embodiment, the composition may be administered intranasally, intradermally, intramuscularly, intraosseously, intraperitoneally, intravenously, subcutaneously or intrathecaly. A method of treating a viral infection, the method comprising administering a therapeutically effective amount of a pharmaceutical composition to a subject in need thereof. A method of inoculating a subject who is at risk of suffering a viral infection, the method comprising administering to the subject a prophylactically effective amount of a pharmaceutical composition. In one embodiment, the pharmaceutical composition is administered intranasally, intradermally, intramuscularly, intraosseously, intraperitoneally, intravenously, subcutaneously or intrathecally. In one embodiment, the subject is infected by, or is at risk of infection from, a Hendra virus (HeV) or a Nipah virus (NiV). In one embodiment, the subject is infected by, or is at risk of infection from, Eastern equine encephalitis (EEEV) or Venezuelan equine encephalitis (VEEV).

In one embodiment, (a) the nanoparticle is loaded with the naked siRNA ALN-RSV01 and the protocel is useful in treating RSV; or (b) the nanoparticle is loaded with the plasmid DNA NUC B1000 or DPC ARC-520 and the protocell is useful in treating HBV; or (c) the nanoparticle is loaded with the Lentivirus pHIV7-shI-TARCCR5RZ and the protocell is useful in treating HIV; or (d) the nanoparticle is loaded with the naked locked nucleic acid (LNA) SPC3649 and the protocell is useful in treating HCV.

In one embodiment, (a) the nanoparticle is an aminated mesoporous silica nanoparticle (MSNP); (b) the lipid bilayer comprises DOPC or DPPC in combination with DOPE, cholesterol and PEG-2000 PE (18:1); (c) the nanoparticle is loaded with between about 30 wt % to about 50 wt % of an antiviral siRNA, plasmid DNA, lentivirus RNA, locked nucleic acid, PNA or miRNA; (d) the endosomolytic moiety is octaarginine (R8); (e) the targeting moiety targets ephrin B2 and/or ephrin B3, and the targeting moiety is a peptide comprising one or more amino acid sequences selected from the groups consisting of TGAILHP, QGAINHP, QHIRKPP, QHRIKPP and QHILNPP; (f) at a pH of about 7 and a period of about 12 days after delivery, the protocell will release no more than about 10 wt % of its antiviral cargo; and (g) at a pH of about 5 and a period of about one day after delivery, the protocell will release no less than about 90 wt % of its antiviral cargo.

A method of diagnosing and/or treating a cancer, a bacterial infection or a viral infection is provided. The method comprising administering to a subject in need thereof a population of protocells, wherein the protocell lipid bi- or multilayer comprises a reporter. A kit comprising a population of protocells and, optionally, instructions for the use of the protocells in the diagnosis and treatment of a viral infection is further provided.

An antibacterial protocell comprising a mesoporous silica or metal oxide nanoparticle is provided which is loaded with an antibacterial cargo and which is coated with a lipid bi- or multilayer, wherein: (a) the mesoporous metal oxide nanoparticle has a pore size which ranges from about 0.001 to about 100 nm, e.g., from about 0.01 nm to about 50 nm, e.g., from about 0.1 to about 100 nm, from about 0.1 nm to about 35 nm, and from about 2 nm to about 25 nm, and a diameter ranging from about 25 nm to about 500 nm, e.g., from about 100 nm to about 300 nm; and wherein (b) the lipid bi- or multilayer comprises at least one targeting moiety that targets a bacterially-infected host cell. In one embodiment, the targeting moiety is a peptide or single chain variable fragment (scFv). In one embodiment, the targeting moiety is a Fcγ from human IgG, human complement C3, ephrin B2 or mannosylated cholesterol. In one embodiment, the antibacterial cargo is effective in the treatment of an infection caused by a bacterium selected from the group consisting of multidrug-resistant (MDR) Klebsiella pneumoniae (Kpn), methicillin-resistant Staphylococcus aureus (MRSA), F. tularensis and B. pseudomallei. In one embodiment, the protocel further comprising an endosomolytic moiety. In one embodiment, the endosomolytic moiety ruptures a bacterially-infected cell membrane ruptures acidic intracellular vesicles of the bacterially-infected cell. In one embodiment, the endosomolytic moiety is a peptide. In one embodiment, the endosomolytic moiety is a peptide selected from the group consisting of octaarginine (R8), H5WYG, Penetratin-HA2, modified HA2-TAT, 4₃E and Histidine 10. In one embodiment, the antibacterial cargo is selected from the group consisting of a smal molecule, an asRNA (anti-sense RNA), a mRNA, a siRNA, a shRNA, a micro RNA, a protein, a protein toxin (e.g. ricin toxin A-chain or diphtheria toxin A-chain), DNA (including double stranded or linear DNA, minicircle DNA, plasmid DNA which may be supercoiled and/or packaged (e.g. with histones) and which may be optionally modified with a nuclear localization sequence), a PNA, a CPP-PNA, or an antibiotic. In one embodiment, the antibacterial cargo is a nucleic acid molecule capable of inhibiting the translation of a mRNA selected from the group consisting of a TEM beta-lactamase (class A) mRNA, a SHV beta-lactamase (class A) mRNA, a CTX-M beta-lactamase (class A) mRNA, an OXA beta-lactamase (class D) mRNA, a PER mRNA, a VEB mRNA, a GES mRNA, an IBC beta-lactamase mRNA, an AmpC type β-lactamase mRNA, and a carbapenemase mRNA (including but not limited to KPC (K. pneumoniae carbapenemase) (Class A) mRNA), and the mammalian and non-mammalian orthologs thereof.

In one embodiment, the antibacterial cargo comprises a nucleic acid molecule capable of inhibiting the translation of a mRNA selected from the group consisting of Metallo-beta-lactamase NDM-1 mRNA, SHV and TEM beta-lactamase mRNA, CMY-6 AmpC-type beta-lactamase mRNA, CTX-M-15 extended spectrum beta-lactamase mRNA: TEM-1 beta-lactamase mRNA: OXA-1 beta-lactamase mRNA; Aminoglycoside-(3)(9)-adenyttransferase AADA2 mRNA; Sul1 dihydropteroate synthase mRNA; Undecaprenyl-diphosphatase mRNA; 16S ribosomal RNA methyltransferase mRNA; AAC(6)-Ib aminoglycoside 6-N-acetyl transferase type Ib mRNA; Sul1 dihydropteroate synthase mRNA; 16S rRNA methyitransferase RmtC mRNA: Aminoglycoside 3 phosphotransferase APH(3)-Ib (strA) mRNA: Sul2 mRNA, sulfonamide insensitive dihydropteroate synthetase mRNA: Streptomycin 3-O-adenylyltransferase aadA ANT(3)-Ia mRNA; Dfra14 trimethoprim-resistant dihydrofolate reductase mRNA; QnrB10 mRNA; Aminoglycoside N(3)-acetyltransferase II (ACC(3)-II)mRNA; Tetracycline efflux protein TetA mRNA; and Macrolide 2-phosphotransferase mphA mRNA, and the mammalian and non-mammalian orthologs thereof. In one embodiment, the nucleic acid molecule is selected from the group comprising siRNA, miRNA, shRNA and/or asRNA. In one embodiment, the nucleic acid molecule has a nucleic acid sequence that targets at least 10 contiguous nucleotides of the mRNA molecules or equivalents and/or fragments of the mRNA molecules.

In one embodiment, the nucleic acid molecule is a siRNA or an asRNA. In one embodiment, the antibacterial cargo is a peptide nucleic acid (PNA) comprising nucleic acid molecules which inhibit the translation of a mRNA selected from the group consisting of a TEM beta-lactamase (class A) mRNA, a SHV beta-lactamase (class A) mRNA, a CTX-M beta-lactamase (class A) mRNA, an OXA beta-lactamase (class D) mRNA, a PER mRNA, a VEB mRNA, a GES mRNA, an IBC beta-lactamase mRNA, an AmpC type lactamase mRNA, and a carbapenemase mRNA (including but not limited to KPC (K. pneumoniae carbapenemase) (Class A) mRNA), and the mammalian and non-mammalian orthologs thereof.

In one embodiment, the antibacterial cargo comprises a peptide nucleic acid (PNA) comprising nucleic acid molecules that inhibit the translation of a mRNA selected from the group consisting of Metallo-beta-lactamase NDM-1 mRNA, SHV and TEM beta-lactamase mRNA, CMY-6 AmpC-type beta-lactamase mRNA, CTX-M-15 extended spectrum beta-lactamase mRNA; TEM-1 beta-lactamase mRNA; OXA-1 beta-lactamase mRNA; Aminoglycoside-(3)(9)-adenyltransferase AADA2 mRNA; Sul1 dihydropteroate synthase mRNA; Undecaprenyl-diphosphatase mRNA; 16S ribosomal RNA methyltransferase mRNA; AAC(6)-Ib aminoglycoside 6-N-acetyl transferase type Ib mRNA; Sul1 dihydropteroate synthase mRNA; 16S rRNA methyltransferase RmtC mRNA; Aminoglycoside 3 phosphotransferase APH(3)-Ib (strA) mRNA; Sul2 mRNA, sulfonamide insensitive dihydropteroate synthetase mRNA; Streptomycin 3-O-adenylyltransferase aadA ANT(3)-Ia mRNA: Dfra14 trimethoprim-resistant dihydrofolate reductase mRNA; QnrB10 mRNA; Aminoglycoside N(3)-acetyttransferase II (ACC(3)-II)mRNA; Tetracycline efflux protein TetA mRNA; and Macrolide 2-phosphotransferase mphA mRNA, and the mammalian and non-mammalian orthologs thereof. In one embodiment, the nucleic acid molecules are siRNA molecules or asRNA molecules. In one embodiment, the nucleic acid molecules are asRNA molecules which comprise one or more nucleotide sequences selected from the group consisting of caagttttc, gaaatcagt, gaaatcagt, gggattcct, actcttcct, ttaatgagg, tcaaaggcc, ggcgtcggc, ccaattaaa, tgggtatta, ttaatgagg, ggcgtoggc, atatggtct, ggaggttc, gggggcttc, gatgtttaa, ggttctcat, atttgtacc, cgcgatatc, gtctggcct and gattcactc and equivalents and fragments thereof.

In one embodiment, the antibacterial cargo is a nucleic acid molecule capable of: (a) inhibiting the translation of an antibiotic resistance-conferring enzyme identified in Table 1 hereof: and/or (b) inhibiting the translation of an antibiotic resistance-conferring reflux pump identified in Table 1 hereof; and/or (c) inhibiting the transcription of an antibiotic resistance-conferring gene identified in Table 1 hereof, mRNA transcribed from those genes, and equivalents thereof. In one embodiment, the antibacterial cargo is a nucleic acid molecule capable of inhibiting the translation of one or more efflux pumps of the families MFS (major facilitator superfamily), SMR (small multidrug resistance), ABC (ATP-binding cassette) and MATE (Multidrug And Toxic Compound Extrusion). In one embodiment, the antibacterial cargo is a nucleic acid molecule capable of inhibiting the transcription of one or more genes selected from the group consisting of qnrB9, aac(6′)-Ib, sul1, bla_{SHV-11}, bla_{CTX-M-15} blaNDM-1, the bla_{TEM-1}, aph-3′-ia (aminoglycoside-3′-phosphotransferase type Ia, APH(3)-Ia) and equivalents thereof. In one embodiment, the antibacterial cargo is a peptide nucleic acid (PNA) which binds to a ribosomal binding site of one or more genes selected from the group consisting of qnrB9, aac(6′)-Ib, sul1, bla_{SHV-1}, bla_{CYX-M-15}, blaNDM-1, the bla gene encoding TEM-1 and equivalents thereof. In one embodiment, the nucleic acid is selected from the group comprising siRNA, miRNA, shRNA and/or asRNA. In one embodiment, the antibacterial cargo is a nucleic acid molecule capable of inhibiting the translation of hemolysin produced by S. aureus or extracellular toxin complex (ETC) produced by K. pneumoniae. In one embodiment, the antibacterial cargo is a peptide nucleic acid (PNA) comprising nucleic acid molecules capable of inhibiting the translation of hemolysin produced by S. aureus or extracellular toxin complex (ETC) produced by K. pneumoniae. In one embodiment, the nucleic acid molecules are selected from the group comprising siRNA, miRNA, shRNA and/or asRNA. In one embodiment, the lipid bi- or multilayer is comprised of lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures thereof. In one embodiment, the lipid bi- or multilayer comprises DOPC in combination with DOPE.

In one embodiment, the lipid bi- or multilayer comprises DOTAP, DOPG, DOPC or mixtures thereof. In one embodiment, the lipid bi- or multilayer comprises DOPG and DOPC. In one embodiment, in the lipid bi- or multilayer further comprises cholesterol. In one embodiment, the lipid bi- or multilayer comprises DOPC or DPPC in combination with DOPE, cholesterol and PEG-2000 PE (18:1). In one embodiment, the lipid bi- or multilayer further comprises between about 0.5 wt % to about 5 wt % (in one embodiment about 1 wt %) of glutathione. In one embodiment, the lipid bi- or multilayer further comprises a RGD (Arg-Gly-Asp) peptide. In one embodiment, the antibacterial cargo comprises one or more anti-bacterial agents selected from the group consisting of davulanic acid, Gentamicin, Kanamycin, Neomycin, Netilmicin, Tobramycin, Paromomycin, Spectinomycin, Geldanamycin, Herbimycin, Rifaximin, Streptomycin, Ertapenem. Doripenem, Imipenem/Cilastatin, Meropenem, Cefadroxil, Cefazolin, Cephalothin, Cephalexin, Cefaclor, Cefamandole, Cefoxitin, Cefprozil, Cefuroxime, Cefixime, Cefdinir, Cefditoren, Cefoperazone Cefotaxime, Cefpodoxime, Ceflazidime, Ceftibuten, Ceftizoxime Cefiriaxone, Cefepime, Ceftaroline fosamil, Ceftobiprole, Teicoplanin, Vancomycin, Telavancin, Daplomycin, Oritavancin, WAP-8294A, Azithromycin, Clarithromycin, Dirithromycin, Erythromycin, Roxithromycin, Telithromycin, Spiramycin, Clindamycin, Lincomycin, Aztreonam, Furazolidone, Nitrofurantoin, Oxazolidinones, Linezolid, Posizolid, Radezolid, Torezolid, Amoxicillin, Ampicillin, Azlocillin, Carbenicillin, Cloxacillin Dicloxacillin, Fludoxacillin, Mezlocilln, Methicillin, Nafcillin, Oxacillin, Penicillin G, Penicillin V, Piperacillin, Temocillin, Ticarcillin, Amoxicillin/clclavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, Mafenide, Sulfacetamide, Sulfadiazine, Sulfadimethoxine, Sulfamethizole, Sulfamethoxazole, Sulfasalazine, Sulfisoxazole, Trimethoprim-Sulfamethoxazole, Sulfonamidochrysoidine, Demedocycline, Doxycycline, Vibramycin Minocydine, Tigecycine, Oxytetracycline, Tetracydine, Clofazimine, Capreomycin, Cycloserine. Ethambutol, Rifampicin, Rifabutin, Rifapentine, Arsphenamine, Chloramphenicol, Fosfomycin, Fusidic acid, Metronidazole, Mupirocin, Platensimycin, Quinupristin/Dalfopristin, Thiamphenicol, Tigecycline and Tinidazole and combinations thereof.

In one embodiment, the nanoparticle is an aminated mesoporous silica nanoparticle (MSNP). In one embodiment, the nanoparticle is aminated with aminopropyltriethoxysilane (APTES) or 3-[2-(2 aminoethylamino)ethylamino]propytrimethoxysilane (AEPTMS). In one embodiment, the mesoporous nanoparticle is made by an evaporation-induced self-assembly (EISA) process. In one embodiment, the evaporation-induced self-assembly (EISA) process includes the steps of: (a) preparing a precursor solution comprising (1) a surfactant (2) tetraethyl orthosilicate (TEOS), tetramethyl orthosilicate (TMOS) or a mixture thereof (3) a C₁₋₄ alcohol (e.g., ethanol), and (4) water, wherein said surfactant, tetraethyl orthosilicate (TEOS), tetramethyl orthosilicate (TMOS) or a mixture thereof, C₁₋₄ alcohol, and water are combined at a temperature below the surfactant's critical micelle concentration; (b) atomizing the precursor solution to generate droplets; (c) drying the droplets, thereby evaporating solvent and increasing effective surfactant concentration and inducing nanoparticle self-assembly; (d) heating dried droplets, thereby evaporating residual solvent, inducing silica condensation and forming solid nanoparticles. In one embodiment, the evaporation-induced self-assembly (EISA) process further includes the steps of: (a) varying the degree of silica condensation by thermal calcination to maximize the number of Si—O—Si bonds; and (b) adding an amine-containing silane to the precursor solution to replace a controllable fraction of Si—O—Si bonds with Si—R—NH₂ bonds, where R is a C₁₋₁₂ hydrocarbon. In one embodiment, the evaporation-induced self-assembly (EISA) process further includes the step(s) of reducing the degree of silica condensation by using acidified ethanol to extract structure-directing surfactants and/or increasing the degree of silica condensation by using thermal calcination.

In one embodiment, the nanoparticle has a differential pore volume of between about 0.25 cm³/g to about 10 cm³/g, from about 0.3 cm³/g to about 3 cm³/g or from about 0.25 cm³/g to about 1.5 cm³/g. In one embodiment, the nanoparticle has a nominal BET surface area of between about 50 mr²/g to about 1,500 m²/g, e.g., from about 100 m²/g to about 1,300 m²/g. In one embodiment, the lipid bi- or multilayer is PEGylated. In one embodiment, the lipid bi- or multilayer contains a reporter. In one embodiment, the nanoparticle is an a mesoporous silica nanoparticle (MSNP) and the protocell is formed by fusing liposomes to the MSNP in the presence of divalent cations, thereby coating the MSNP with a supported lipid multilayer. In one embodiment, the nanoparticle is a mesoporous silica nanoparticle (MSNP) and wherein the weight ratio of antibacterial cargo to silica ranges from about 0.10 to about 0.75.

In one embodiment, the nanoparticle is a silica nanoparticle (MSNP) which is coated with a lipid bilayer and wherein: (a) at a pH of about 7 and a period of about 6 hours after delivery, the protocell will release no more than about 5 wt % of its antiviral cargo; (b) at a pH of about 5 and a period of about 6 hours after delivery, the protocel will release no less than about 50 wt % of its antiviral cargo; and (c) at a pH of about 5 and a period of about 12 hours after delivery, the protocell will release no less than about 90 wt % of its antiviral cargo. In one embodiment, the nanoparticle is a silica nanoparticle (MSNP) which is coated with a lipid bilayer and wherein: (a) at a pH of about 7 and a period of about 24 hours after delivery, the protocell will release no more than about 5 wt % of its antiviral cargo; (b) at a pH of about 5 and a period of about 6 hours after delivery, the protocell will release no less than about 20 wt % of its antiviral cargo; (c) at a pH of about 5 and a period of about 12 hours after delivery, the protocell will release no less than about 70 wt % of its antiviral cargo; and (d) at a pH of about 5 and a period of about 24 hours after delivery, the protocell will release no less than about 90 wt % of its antiviral cargo. In one embodiment, the protocell is useful in the treatment of methicillin-resistant Staphylococcus aureus (MRSA) skin and soft tissue infections (SSTI). In one embodiment, the nanoparticle is a silica nanoparticle (MSNP) having a multimodal pore morphology defined by surface-accessible pores having a diameter of from about 5-50 nm, e.g., from about 5-25 nm, from about 10-35 nm, from about 20-25 nm interconnected by internal pores ranging in size from about 1-15 nm, e.g., about 5-15 nm, wherein the surface accessible pores have a larger diameter than the internal pores. In one embodiment, the nanoparticle is an aminated mesoporous silica nanoparticle (MSNP) which has a Zeta (ζ) potential of between about 0 mV to about +40 mV. In one embodiment, the surfactant is a cationic surfactant selected from the group consisting of a dodecylsulfate salt (e.g., sodium dodecylsulfate or lithium dodecylsulfate (SDS)), a tetradecyl-trimethyl-ammonium salt (e.g., tetradecyl-tnmethyl-ammonium bromide (C₁₄TAB) or tetradecyl-trimethyl-ammonium chloride), a hexadecyltrimethylammonium salt (e.g., hexadecyltrimethylammonium bromide (C₁₆; CTAB)), an octadecyltrimethylammonium salt (e.g., octadecyltrimethylammonium bromide (C₁₈; OTAB)), a dodecylethyldimethylammonium salt (e.g., dodecylethyldimethylammonium bromide), a cetylpyridinium salt (e.g., cetylpyridinium chloride (CPC)), polyethoxylated tallow amine (POEA), hexadecyltrimethylammonium p-toluenesulfonate, a benzalkonium salt (e.g., benzalkonium chloride (BAC)), or a benzethonium salt (e.g., benzethonium chloride (BZT)) and mixtures thereof. In one embodiment, the nanoparticle has a nominal BET surface area of about 1,200 m²/g and the surfactant is hexadecyftrimethylammonium bromide (Cis; CTAB). In one embodiment, the nanoparticle is a mesoporous silica nanoparticle (MSNP). In one embodiment, the nanoparticle is loaded with two or more different antibacterial cargos. In one embodiment, the two or more different antibacterial cargos are of different kinds.

A pharmaceutical composition is provided comprising: (a) a therapeutically effective amount of protocells; and (b) optionally, one or more pharmaceutically acceptable excipients. In one embodiment, the composition may be administered intranasally, intradermally, intramuscularly, intraosseously, intraperitoneally, intravenously, subcutaneously or intrathecally. A method of treating a bacterial infection, the method comprising administering a therapeutically effective amount of a pharmaceutical composition to a subject in need thereof. In one embodiment, the subject suffers from an antibiotic-resistant bacterial infection. In one embodiment, the subject is infected by a bacterium selected from the group consisting of multidrug-resistant (MDR) Klebsiella pneumoniae (Kpn), methicillin-resistant Staphylococcus aureus (MRSA), F. tularensis and B. pseudomallei. In one embodiment, the subject suffers from a methicillin-resistant Staphylococcus aureus (MRSA) skin or soft tissue infection (SSTI).

asRNA molecules which bind to a ribosomal binding site of one or more antibiotic resistance-conferring genes and which comprise one or more nucleotide sequences selected from the group consisting of caagttttc, gaaatcagt, gaaatcagt, gggattcctd, actcttcct, ttaatgagg, tcaaaggcc, ggcgtcggc, ccaattaaa, tgggtatta, ttaatgagg, ggcgtcggc, atatggtct, ggaggttc, gggggcttc, gatgtttaa, ggttctcat, atttgtacc, cgcgatatc, gtctggcct and gattcactc and equivalents thereof. A plasmid vector is also provided comprising an asRNA, said asRNA being operably linked to a promoter. A method of treating a subject who suffers PNA comprising the asRNA. A CPP-PNA comprising the PNA from an antibiotic-resistant bacterial infection is also provided, the method comprising administering the plasmid vector to the subject. A screening method of using quantitative PCR (qPCR) to determine the antibiotic effect of a composition on a sample of bacterially infected cells is further provided, wherein during qPCR each doubling of bacterial levels leads to a decrease of one cycle (delta-Ct=−1) for detection in real-time PCR, the method comprising: (a) contacting the cell sample with the composition and thereafter with a photoreactive intercalating dye that is excluded from cells with intact membranes and that penetrates membranes of dead cells and binds the DNA of the dead cells; (b) exposing the cells to a light source, thereby converting the photoreactive intercalating dye to a species which crosslinks, modifies, or otherwise damages DNA to which the photoreactive intercalating dye is bound, thereby rendering said DNA non-amplifiable by PCR; and (c) continuing to perform PCR on the sample and calculating delta-Ct values, wherein a determination that calculated delta-Ct values increasingly exceed control delta-Ct values determined by performing qPCR on a sample of untreated bacterially infected cells indicates that the composition is effective in the treatment of the bacterial infection.

In one embodiment, the photoreactive intercalating dye is propidium monoazide (PMA) and the light source is a blue light source. A microscale test strip compatible with either broth or agar microdilution, said test strip enabling a determination of a composition's minimum inhibitory concentration (MIC) using only about 5-10 μL of test solution comprising the composition, said test strip comprising a linear or rectangular array of wells configured with up to twenty wells per strip, said wells being spaced from one another at a distance of about 4.5 mm or 3 mm, said spacing being compatible for use of the test strip with a 384-well plate having 4.5 mm well spacing and a 1,536-well plate having a 2.25 mm well spacing, and a 96 well plate. In one embodiment, the strip has a substantially flay bottom with shallow, open chambers to enable efficient gas exchange for microbial growth without shaking or agitation and the accommodation of either liquid or solid growth media.

A method of diagnosing and treating an antibiotic-resistant bacterial infection is provided, the method comprising administering to a subject in need thereof a population of antibacterial protocells. A kit is provided comprising a population of antibacterial protocells and, optionally, instructions for the use of the protocells in the diagnosis and treatment of a bacterial infection. A nanoparticle comprising silica or metal oxide, the nanoparticle functionalized with a hydrophobic group and loaded with a water-insoluble cargo. In one embodiment, wherein the nanoparticle is porous. In one embodiment, the nanoparticle comprises pores with a diameter of about 0.01 nm to about 50 nm. In one embodiment, the nanoparticle has a monomodal pore size distribution. In one embodiment, the nanoparticle has a multimodal pore size distribution. In one embodiment, the nanoparticle has a diameter of about 25 nm to about 500 nm. In one embodiment, the hydrophobic group is a methyl group or a phenyl group. In one embodiment, the nanoparticle is functionalized with a hydrophobic organosiloxane. In one embodiment, the hydrophobic organosiloxane is hexamethyldisilazane (HDMS), sodium bis(trimethylsilyl)amide (NaHDMS), potassium bis(trimethylsilyl)amide (KHDMS), or phenytriethoxysilane (PTS). In one embodiment, the nanoparticle comprises silica. In one embodiment, the nanoparticle has a pore volume fraction of about 25% to about 75%. In one embodiment, the nanoparticle has a surface area of about 100 m²/g to about 1,300 m²/g. In one embodiment, the cargo has a water solubility of less than about 5 mg/ml. In one embodiment, the cargo has a water solubility of less than about 0.5 mg/ml. In one embodiment, the cargo is a small-molecule drug. In one embodiment, the cargo is an anti-cancer agent, an anti-viral agent, or an antibiotic. In one embodiment, the weight ratio of cargo to silica is about 0.10 to about 0.75. In one embodiment, the nanoparticle has a Zeta (ζ) potential of about −40 mV to about 0 mV. In one embodiment, the nanoparticle is spherical or toroidal. In one embodiment, the nanoparticle is condensed by thermal calcination. In one embodiment, the surfactant had been removed from the nanoparticle by an acidified C₁₋₄ alcohol. In one embodiment, the nanoparticle is PEGylated. In one embodiment, the nanoparticle is not PEGylated. A nanoparticle composition comprising a plurality of nanoparticles. In one embodiment, the nanoparticles are monodisperse. In one embodiment, the nanoparticles are polydisperse. In one embodiment, the average diameter of the nanoparticles is about 50 nm to about 150 nm. A protocell comprising the nanoparticle coated with a lipid bilayer or multilayer is further provided. In one embodiment, the lipid bilayer or multilayer comprises a cellular barrier penetrating moiety.

A protocell is provided comprising a silica or metal oxide nanoparticle core coated with a lipid bilayer or multilayer, wherein the lipid bilayer or multilayer comprises a cellular barrier penetrating moiety. In one embodiment, the cellular barrier penetrating moiety is an endothelial cell barrier penetrating moiety. In one embodiment, the cellular barrier penetrating moiety is an epithelial cell barrier penetrating moiety. In one embodiment, the lipid bilayer or multilayer comprises about 0.5 wt % to about 5 wt % cellular barrier penetrating moiety. In one embodiment, the cellular barrier penetrating moiety is glutathione, L-dihydroxyphenylalanine, or a peptide comprising an Arg-Gly-Asp sequence. In one embodiment, the protocell traverses a cellular barrier. In one embodiment, the cellular barrier is an epithelial cell barrier or an endothelial cell barrier. In one embodiment, the cellular barrier is a blood-brain barrier or a nasal epithelium. In one embodiment, the nanoparticle is porous. In one embodiment, the nanoparticle comprises pores with a diameter of about 0.01 nm to about 50 nm. In one embodiment, the nanoparticle has a monomodal pore size distribution. In one embodiment, the nanoparticle has a multimodal pore size distribution. In one embodiment, the nanoparticle has a diameter of about 25 nm to about 500 nm. In one embodiment, the nanoparticle comprises silica. In one embodiment, the nanoparticle has a pore volume fraction of about 25% to about 75%. In one embodiment, the nanoparticle has a surface area of about 100 m²/g to about 1,300 m²/g. In one embodiment, the nanoparticle is loaded with a cargo. In one embodiment, the cargo is a small-molecule drug. In one embodiment, the cargo is an anti-cancer agent, an anti-viral agent, and/or an antibiotic. In one embodiment, the cargo is a polynucleotide.

In one embodiment, the polynucleotide is a DNA or RNA. In one embodiment, the DNA is a plasmid. In one embodiment, the DNA is a minicircle. In one embodiment, the RNA is an mRNA, siRNA, miRNA, or shRNA. In one embodiment, the weight ratio of cargo to silica is about 0.10 to about 0.75. In one embodiment, the nanoparticle core is functionalized with an amine-modified silane. In one embodiment, the nanoparticle core has a Zeta (ζ) potential of about 0 mV to about +50 mV. In one embodiment, the amine-modified silane is a primary amine, a secondary amine a tertiary amine, each of which is functionalized with a silicon atom (2) a monoamine or a polyamine (3)N-(2-aminoethyl)-3-aminopropyltrimethoxysilane (AEPTMS) (4) 3-aminopropytrimethoxysilane (APTMS) (5) 3-aminopropyltriethoxysilane (APTS) (6) an amino-functional trialkoxysilane, and (7) protonated secondary amines, protonated tertiary alkyl amines, protonated amidines, protonated guanidines, protonated pyridines, protonated pyrimidines, protonated pyrazines, protonated purines, protonated imidazoles, protonated pyrroles, and quaternary alkyl amines, or combinations thereof. In one embodiment, the nanoparticle core is spherical or toroidal. In one embodiment, the nanoparticle core is condensed by thermal calcination. In one embodiment, surfactant had been removed from the nanoparticle core by an acidified C₁₋₄ alcohol. In one embodiment, the lipid bilayer or multilayer comprises lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), and cholesterol.

In one embodiment, the lipid bilayer or multilayer comprises DOTAP, DOPG, DPPC, DOPE, or DOPC. In one embodiment, the lipid bilayer or multilayer comprises cholesterol. In one embodiment, the lipid bilayer or multilayer comprises a cell targeting species. In one embodiment, the targeting species is a peptide, an antibody, an antibody fragment, an aptamer, an affibody, a carbohydrate, or a functionalized cholesterol. In one embodiment, the targeting species targets cancer cells. In one embodiment, the targeting species is mannosylated cholesterol. In one embodiment, the lipid bilayer or multilayer comprises a fusogenic peptide. In one embodiment, the lipid bilayer or multilayer comprises PEG. In one embodiment, the lipid bilayer or mukilayer does not comprise PEG. In one embodiment, the protocell has a diameter of about 50 nm to about 150 nm. In one embodiment, the protocell has a zeta potential of about −50 mV to about +50 mV. In one embodiment, the protocell releases about 30% to about 100% of its cargo after about three hours at pH 5. In one embodiment, the protocel releases its cargo through sustained release at a rate of about 7% to about 10% weight cargo per day over a period of about ten days. In one embodiment, the lipid bilayer is conjugated to CD47 or aminopeptidase P antibody. In one embodiment, the protocell does not induce an immune response. In one embodiment, the protocell does not stimulate an IgG or IgM response.

A protocell composition comprising a plurality of protocells is provided. In one embodiment, the protocells are monodisperse. In one embodiment, the protocells are polydisperse. The protocell composition wherein the average diameter of the protocells is about 50 nm to about 300 nm. A pharmaceutical composition comprising the nanoparticle composition and a pharmaceutically acceptable excipient is provided. A pharmaceutical composition comprising the protocel composition and a pharmaceutically acceptable excipient are also provided In one embodiment, the pharmaceutical composition is administered intranasally, intradermaly, intramuscularly, intraosseously, intraperitoneally, intravenously, subcutaneously, or intrathecally. A method of treating a disease comprising administering to a patient a therapeutically effective amount of a pharmaceutical composition. In one embodiment, the disease is cancer. In one embodiment, the nanoparticles are loaded with an anticancer agent. In one embodiment, the disease is hepatocellular carcinoma or acute lymphoblastic leukemia.

A kit comprising a pharmaceutical composition and instructions for the use of the pharmaceutical composition is provided. An article of manufacture comprising a pharmaceutical composition in suitable packaging is also provided.

An evaporation-induced self-assembly (EISA) process for making functionalized silica nanoparticles loaded with a water-insoluble cargo comprising: (a) atomizing a precursor solution to generate droplets; wherein the precursor solution comprises (1) a surfactant, (2) tetraethyl orthosilicate (TEOS) or tetramethyl orthosilicate (TMOS). (3) a C₁₋₄ alcohol, (4) a hydrophobic organosiloxane, and (5) water; (b) drying and heating the droplets, thereby evaporating solvent and increasing effective surfactant concentration; and (c) loading the nanoparticles with a water-insoluble cargo. In one embodiment, the surfactant is below the critical micelle concentration of the surfactant. In one embodiment, the surfactant comprises a cationic surfactant. In one embodiment, the surfactant is selected from the group consisting of a dodecylsulfate salt, a tetradecyl-trimethyl-ammonium salt, a hexadecyltrimethylammonium salt, an octadecyltrimethylammonium salt, a dodecylethyldimethylammonium salt, a cetylpyridinium salt, polyethoxylated tallow amine (POEA), hexadecyltrimethylammonium p-toluenesulfonate, a benzalkonium salt, a Brij® surfactant, a poloxamer, and a benzethonium salt.

In one embodiment, the surfactant is selected from the group consisting of benzethonium chloride, benzalkonium chloride, cetylpyridinium chloride, dodecylethyldimethylammonium bromide, octadecyltrimethylammonium bromide, hexadecyltrimethylammonium bromide, tetradecyl-trimethyl-ammonium bromide, tetradecyl-trimethyl-ammonium chloride, sodium dodecylsulfate, lithium dodecylsulfate, BrijS-56, Pluronic® F108, and Pluronic® P123. In one embodiment, the precursor solution comprises urea, poly(propylene oxide) (PPO), poly(ethylene oxide) (PEO), polypropylene glycol acrylate (PPGA), or glycerol. In one embodiment, the C14 alcohol is ethanol. An evaporation-induced self-assembly (EISA) process for making fundionalized silica nanoparticles loaded with a water-insoluble cargo comprising: (a) combining an aqueous phase precursor solution and an oil phase precursor solution, thereby forming an emulsion, wherein the aqueous phase precursor solution comprises (1) a hydrophobic organosiloxane. (2) a first surfactant. (3) tetraethyl orthosilicate (TEOS) or tetramethyl orthosilicate (TMOS), (4) an acid, and (5) water, and the oil phase precursor solution comprises a second surfactant and an oil; (b) heating the emulsion, thereby generating nanoparticles; (c) separating the nanoparticles from the remaining emulsion; (d) loading the nanoparticles with a water-insoluble cargo. In one embodiment, the concentration of the first surfactant is below the critical micelle concentration of the surfactant in the aqueous phase precursor solution. In one embodiment, the first surfactant is a cationic surfactant.

In one embodiment, the first surfactant is selected from the group consisting of sodium dodecylsulfate, lithium dodecylsulfate, a tetradecyl-trimethyl-ammonium salt, a hexadecyltrimethylammonium salt, an octadecyltrimethylammonium salt, a dodecylethyldimethylammonium salt, a cetylpyridinium salt, polyethoxylated tallow amine (POEA), hexadecyltrimethylammonium p-toluenesulfonate, a benzalkonium salt, and a benzethonium salt. In one embodiment, the first surfactant is selected from the group consisting of tetradecyl-trimethyl-ammonium bromide (C₁₄TAB), tetradecyl-trimethyl-ammonium chloride, hexadecyltrimethylammonium bromide (C₁₆TAB), octadecyltrimethylammonium bromide (C₁₈TAB), dodecylethyldimethylammonium bromide (C₁₂TAB), cetylpyridinium chloride (CPC), benzalkonium chloride (BAC), and benzethonium chloride (BZT). In one embodiment, the second surfactant is a nonionic surfactant. In one embodiment, the second surfactant is a poloxamer or a nonionic silicon-based surfactant. In one embodiment, the second surfactant is selected from the group consisting of a Brij® surfactant, Pluronic® F108, Pluronic® P123, or ABIL EM 90.

In one embodiment, the oil is a C₁₂-C₂₀ alkane. In one embodiment, the volumetric ratio of the aqueous phase:oil phase is about 1:2 to about 1:4. In one embodiment, the method further comprises thermally calcining the nanoparticles to induce silica condensation prior to the loading step. In one embodiment, the method further comprising extracting the surfactant from the nanoparticles using an acidified C14 alcohol to reduce silica condensation prior to the loading step. In one embodiment, the hydrophobic organosiloxane is a methyl-containing organosiloxane or a phenyl-containing organosiloxane. In one embodiment, the hydrophobic organosiloxane is hexamethyldisilazane (HDMS), sodium bis(trimethylsilyl)amide (NaHDMS), potassium bis(trimethylsilyl)amide (KHDMS), or phenyltnethoxysilane (PTS). A functionalized silica nanoparticle formed by the process is also provided. A method of forming a cellular barrier penetrating protocell is provided comprising surrounding a nanoparticle core with a lipid bilayer, wherein the core is loaded with a cargo; and attaching a cellular barrier penetrating moiety to the lipid bilayer. In one embodiment, the cellular barrier penetrating moiety is glutathione. In one embodiment, the surrounding step is performed in the presence of divalent cations. A method of forming a protocell is provided comprising surrounding a nanoparticle core with a lipid bilayer, wherein the core is functionalized with a hydrophobic group and is loaded with a water-insoluble cargo In one embodiment, the surrounding step is performed in the presence of divalent cations. A protocell is provided comprising a nanoparticle core surrounded by a lipid bilayer, wherein the lipid bilayer comprises CD47, aminopeptidase P antibody, or Fcγ.

In one embodiment, (a) the nanoparticle is an aminated mesoporous silica nanoparticle (MSNP); (b) the lipid bilayer comprises DOPC or DPPC in combination with DOPE, cholesterol and PEG-2000 PE (18:1); (c) the nanoparticle is loaded with between about 30 wt % to about 50 wt % of an antiviral siRNA, plasmid DNA, lentivirus RNA, locked nucleic acid, PNA or miRNA; (d) the endosomolytic moiety is octaarginine (R8); (e) the targeting moiety targets ephrin B2 and/or ephrin B3, and the targeting moiety is a peptide comprising one or more amino acid sequences selected from the groups consisting of TGAILHP, QGAINHP, QHIRKPP, QHRIKPP and QHILNPP; (f) at a pH of about 7 and a period of about 12 days after delivery, the protocell will release no more than about 10 wt % of its antiviral cargo; and (g) at a pH of about 5 and a period of about one day after delivery, the protocell will release no less than about 90 wt % of its antiviral cargo.

### BRIEF DESCRIPTION OF THE FIGURES

FIGS. 1A-B. Pore size and charge can be tailored to maximize the loading capacities of protocells. A) Loading capacities of 150-nm protocells with 2.5-nm pores, 4.4-nm pores, 7.9-nm pores, and 18-25 nm pores for different classes of antivirals; loading capacities of 150-nm liposomes are provided for comparison. B) Loading capacities of 150-nm protocells with unmodified MSNP cores ((=−34 mV) and APTES-modified MSNP cores ((=+22 mV) for different classes of antivirals. Lipid bilayers were composed of DOPC with 30 wt % cholesterol and 10 wt % PEG-2000 in all experiments. Data represent the mean+std. dev. for n=3. Molecular weights (MW), mean hydrodynamic sizes in 1×PBS, and pKa values or isoelectric (pl) points are given for each cargo molecule.

FIGS. 2A-B. The thickness of the lipid shell can be controlled to tailor release rates under physiological and intracellular conditions, as shown by comparing results from a protocell having a bilayer (two lipid layers) or a thicker multilayer (e.g., three lipid layers). A) Rates of ribavirin release from protocels with DOPC supported lipid bilayers (SLBs) when incubated in a simulated body fluid (pH 7.4) or a simulated endolysosomal fluid (pH 5.0) at 37° C. for 7 days; the rate of ribavirin release from DSPC liposomes upon incubation in a simulated body fluid is given for comparison. B) Rates of ribavirin release from protocells with DOPC supported lipid multilayers (SLMs) three layers thick upon continuous incubation in a simulated body fluid (pH 7.4), continuous incubation in a simulated endolysosomal fluid (pH 5.0), or iterative incubation in simulated body and endolysosomal fluids; arrows indicate the time periods during which the latter sample was incubated in the simulated endolysosomal fluid. MSNPs with a low degree of silica condensation (i.e., burst release kinetics) were used in all experiments. Data represent the mean±std. dev. for n=3.

FIGS. 3A-C. Modification of the SLB with targeting peptides or scFvs enables selective binding, uptake, and cargo delivery. A) Confocal fluorescence microscopy images of CHO-K1, CHO-K1 transfected to express ephrin B1 (EB1), CHO-K1 transfected to express ephrin B2 (EB2), and HEK 293 cells after being incubated with a 10⁴-fold excess of Alexa Fluor 647-labeled protocells (white) for 1 hour at 37° C.; protocells were targeted with an EB2-binding peptide identified via phage display. Cells were stained using an Alexa Fluor 555-labeled monoclonal antibody against EB2 (red), Alexa Fluor 488-labeled phalloidin (green), and DAPI (blue). B) Mean fluorescence intensities of EB2-negative (CHO-K1, CHO-K1/EB1) and EB2-positive (CHO-K1/EB2, HEK 293, Vero) cells upon incubation with a 10⁴-fold excess of Alexa Fluor 647-labeled protocells for 1 hour at 37° C. Protocells with zwitterionic (DOPC), anionic (DOPS), and cationic (DOTAP) SLBs were tested, along with DOPC protocells modified with an EB2-binding peptide or scFv, both of which were identified using phage display. C) In vitro efficacy of free, protocel-encapsulated, and liposome-encapsulated ribavirin, siRNA specific for Renilla luciferase (Rluc), and a minicircle DNA vector (mcDNA) that expresses smal hairpin RNA (shRNA) specific for Rluc in Vero cells infected with a Nipah pseudovirus (NiVpp) that encodes Rluc; protocells and liposomes were modified with an EB2-binding peptide. ‘Carrier’ refers to empty protocells and liposomes; liposomes are unable to simultaneously encapsulate ribavirin and nucleic acids, so these samples were omitted from the plot. Concentrations of ribavirin, siRNA, and mcDNA were maintained at 5 μM, 10 pM, and 1 pM, respectively. Relative light units (RLUs) were measured 18-hours post-infection and normalized based on the RLUs of Vero cells incubated with NiVpp alone. Data represent the mean±std. dev. for n=3.

FIGS. 4A-B. Modification of the protocel SLB with glutathione enhances penetration across the BBB in vitro and in vivo. A) Mass of silica (SiO₂) that passed through a hCMEC/D3 cell monolayer upon incubation with 1 mg of protocells for 2 hours at 37PC. Protocells were 50-nm, 100-nm, 150-nm, or 250-nm in diameter. SLBs were composed of DOPC (zwitterionic), DOTAP (cationic), DOPS (anionic), or DOPC modified with 1 wt % of glutathione (GSH). Silica was quantified using atomic adsorption spectrometry. Data represent the mean±std. dev. for n=3. B) Relative fluorescence units (RFUs) of the liver, spleen, kidneys, bladder, lungs, heart, and brain harvested from Balb/c mice 1 hour, 12 hours, or 24 hours after IV injection with 200 mg/kg of DyLight® 633-labeled DOPC protocells or DyLight® 633-labeled DOPC protocels with 1 wt % of GSH. Organs were imaged using an IVIS Lumina II, and RFUs were normalized according to the total weight of each organ. Data represent the mean±std. dev. for n=2 mice.

FIGS. 5A-B. MSNPs with controllable overall sizes, pore sizes, and pore geometries can be generated in scalable, cost-effective fashion using aerosol-assisted EISA. Schematic A) and photograph B) of the reactor we use to generate MSNPs via aerosol-assisted EISA. Numbers indicate corresponding portions of the reactor.

FIGS. 6A-F. Aerosol-assisted EISA can be used to generate MSNPs with different pore geometries. TEM images of MSNPs with hexagonal A), cubic B), lamellar C), and cellular D)-F) pore geometries. See Lu et al. (1999).

FIGS. 7A-D. Aerosol-assisted EISA can be used to generate MSNPs with different pore sizes. TEM images of MSNPs with hexagonal 2.5-nm (A), 4.4-nm (B), 7.9-nm (C), and 18-25 nm (D) pores that were templated by CTAB, F68, F127, and F127+FC-4, respectively. The inset in (D) is a SEM micrograph that shows the presence of surface-accessible pores.

FIGS. 8A-D. Burst and sustained release rates can be achieved by controlling the MSNP's degree of condensation. Rates of ribavirin release from MSNPS with a low (A) and high (B) degree of silica condensation. Silica forms via a condensation reaction (C) and dissolves via a hydrolysis reaction (D); the degree of silica condensation dictates that number of Si—O—Si bonds that must be broken for the particle to dissolve and can, therefore, be used to control release rates. Data represent the mean±std. dev. for n=3.

FIGS. 9A-B. SLBs formed of zwitterionic lipids prevent adsorption of serum proteins to the protocel surface upon dispersion in blood. Mean hydrodynamic size (A) and zeta potential (B) of DOPC protocels, DOPC protocells modified with 10 wt % of PEG-2000, bare MSNPs, and MSNPs coated with the cationic polymer, PEI, upon incubation in whole blood for 7 days at 37PC. Data represent the mean±std. dev. for n=3.

FIG. 10. Rates of ribavirin release from protocells and liposomes with lipid bilayers composed of DOPC (Tₘ=−1PC; fluid at physiological temperatures) or DSPC (Tₘ=55° C.; non-fluid at physiological temperatures) upon incubation in whole blood at 37° C. Data represent the mean±std. dev. for n=3.

FIG. 11. Rates of ribavirin release from protocells with supported lipid multilayers (SLMs) three or six layers thick upon incubation in a simulated body fluid (pH 7.4) or a simulated endolysosomal fluid (pH 5.0) at 37° C. Data represent the mean±std. dev. for n=3.

FIG. 12. Modification of the SLB with peptides that promote selective binding, internalization, and endo/lyso/macropinosomal escape enables selective delivery of antivirals to the cytosol of potential host cells. Schematic depicting one embodiment: (1) binding of ephrin B2-targeted protocells to an ephrin B2-positive host cell; (2) internalization of protocells (via octaarginine (R8)-mediated macropinocytosis, in this case); (3) destabilization of the SLB in acidified intracellular vesicles (macropinosomes, in this case) and release of encapsulated antivirals (siRNA, in this case); (4) rupture of intracellular vesicles caused by the ‘proton sponge’ effect of endosomolytic peptides (R8, in this case); and (5) cytosolic dispersion of antivirals within potential host cells.

FIG. 13. Protocells modified with the R8 peptide are internalized by mammalian cells via macropinocytosis, the mechanism we use to trigger uptake of protocells if the targeting ligand does not stimulate receptor-mediated endocytosis. The number of ephrin B2-targeted protocells internalized by each HEK 293 cell upon incubation of cells with a 10⁴-fold excess of protocells for 1 hour at 37° C. HEK 293 cells were pretreated with wortmannin, blebbistatin, and latrunculin A to inhibit macropinocytosis, the mechanism of uptake of ephrin B2-targeted protocells by ephrin B2-positive host cells. Data represent the mean+std. dev. for n=3.

FIGS. 14A-B. Protocells can simultaneously encapsulate physicochemicaly disparate cargos and deliver them to the cytosol of target cells. Eight-color confocal fluorescence microscopy images of cells incubated with a 10′-fold excess of protocells for 1 hour (A) or 24 hours (B) at 3PC. Protocells were simultaneously loaded with a fluorescently-labeled drug (green), siRNA mimic (a dsDNA, magenta), and protein (orange), as well as quantum dot (QD)-conjugated minicircie DNA (cyan). Cells were stained with CellTracker™ Violet BMQC (purple) and DAPI (blue). Protocells were modified with a targeting peptide to trigger internalization into intracellular vesicles, which causes the punctuate appearance of protocell components in (A), and an endosomolytic peptide (‘H5WYG’) to enable endosomal escape and cytosolic dispersion of protocell components, which is apparent in (B). The drug and siRNA mimic were modified with a nuclear localization sequence (NLS) (figure adapted from Ashley et al., 2012) to promote their accumulation in cell nuclei, which is evident in (B).

FIGS. 15A-B. Protocells targeted to potential host cells and loaded with minicircle DNA vectors that encode shRNA are able to silence expression of a viral gene for >1 month. Time-dependent concentrations of NiV nucleocapsid (N) mRNA in CHO-K1 and HEK 293 cells upon incubation with ephrin B2-targeted protocells loaded with siRNA (A) or a minicircle DNA vector that encodes shRNA (B) specific for NiV-N. CHO-K1 and HEK 293 cells were stably transfected with NiV-N prior to these experiments; NiV-N mRNA concentrations were determined via qPCR and normalized based on the mRNA concentration in untreated cells. The concentrations of siRNA and minicircle DNA were maintained at 10 pM and 1 pM, respectively. Data represent the mean±std. dev. for n=3.

FIG. 16. Neither empty nor ribavirin-loaded protocells substantially affected the viability of mammalian host cells, even at ribavirin concentrations that exceed the average IC₅₀ value by 1000-fold. The percentage of 1×10⁶ Vero cells that remain viable upon continuous incubation with increasing concentrations of free ribavirin (RBV), empty ephrin B2 (EB2)-targeted protocels, or EB2-targeted, RBV-loaded protocells for 48 hours at 37° C. Cell viability was determined using propidium iodide and was normalized against untreated cells. Data represent the mean±std. dev. for n=3.

FIGS. 17A-B. Protocells are biocompatible. Gross weights (A) and blood chemistry (B) for Balb/c mice injected IP with 200 mg/kg doses of empty DOPC, DOPS, or DOTAP protocells on days 1, 3, 5, 8, 10, 12, 15, 17, 19, 22, 24, and 26. Blood chemistry was measured on day 28. In addition, the liver, spleen, lymph nodes, adrenal glands, kidneys, bladder, bone marrow, lungs, heart, and brain were collected from mice on day 28, sectioned, stained with hematoxylin and eosin, and analyzed by a veterinary pathologist; no evidence of tissue damage was found. KEY for (B): ALB=albumin. ALT=alanine aminotransferase, AST=aspartate aminotransferase, BUN=blood urea nitrogen, CAL=calcium, CHOL=cholesterol, CRE=creatinine, GLU=glucose, PHOS=inorganic phosphorus, TBILI=total bilirubin, TPR=total protein, and TRG=triglycerides. NOTE: IACUC regulations prevented us from injecting mice IV three times a week for a month; protocells injected IV vs. IP have nearly identical biodistributions, however, so we anticipate the safety profiles of protocells injected IV to be similar to data collected using IP injections. Data represent the mean±std. dev. for n=5 mice.

FIG. 18. Protocells are biodegradable. Mass of silica in the urine and feces of Balb/c mice 1 hour, 24 hours, 48 hours, 72 hours, 7 days, and 14 days after being injected IP with a 200 mg/kg dose of empty DOPC protocells; 93.8% of the 5 mg dose was accounted for in the urine and feces after 14 days. Silica was quantified using atomic adsorption spectrometry. Data represent the mean+std. dev. for n=5 mice. ND=none detected.

FIG. 19. Protocels mitigate IgG and IgM responses against encapsulated proteins and surface-displayed targeting peptides. Serum IgG and IgM titers induced upon SC immunization of C5781/6 mice with three doses of protocells or albumin nanoparticles that were loaded with a proprietary enzyme and targeted to hepatocytes with about 5000 copies of a peptide (‘SP94’²) identified via phage display. Mice were immunized on days 0, 14, 28, 56, and 84 with 200 mg/kg of enzyme-loaded protocells or albumin nanoparticles; serum was collected on day 112, and peptide- and enzyme-specific IgG and IgM titers were determined via end-point dilution ELISA. NOTE: SC (versus, e.g., intramuscular) immunization was selected to maximize potential immune responses. Data represent the mean+std. dev. for 3 mice.

FIGS. 20A-D. Protocell size dramatically impacts bulk biodistribution. Total mass of silica (SiO₂) in the blood, liver, spleen, lymph nodes, kidneys, bladder, lungs, heart, brain, urine, and feces of Balb/c mice 1 hour (A), 1 day (B), 1 week (C), and 1 month (D) after being injected IV with 200 mg/kg (˜5 mg of SiO_{z} per mouse) of 50±4 nm, 150±9 nm, or 250±17 nm DOPC protocells. Two mice were sacrificed at the indicated time points, whole blood and organs were homogenized, and SiO₂ was quantified using atomic adsorption spectrometry. Each bar represents the mean+std. dev. for 2 mice. ND=none detected.

FIGS. 21A-B. 150-nm protocells modified with CD47 remain in circulation for up to 3 weeks. (A) Concentrations of silica (SiO₂) and organophosphorus hydrolase (OPH) in the blood of Balb/c mice that were injected IV with free OPH or OPH-loaded protocells; the doses of SiO₂ and OPH were 200 mg/kg and about 100 mg/kg, respectively. Two mice from the ‘free OPH’ group were sacrificed immediately after injection and 1 day post-irrnjection (PI); two mice from the ‘OPH-loaded protocell’ group were sacrificed immediately after injection and at 1, 2, 3, 5, 7, 14, and 21, and 28 days PI. SiO₂ was quantified using atomic adsorption spectrometry, and OPH was quantified using ELISA; concentrations are expressed as μg per mL of blood (about 1.5 mL/mouse). Each data point represents the mean±std. dev. for 2 mice. (B) Total mass of silica (SiO₂) in the blood, liver, spleen, lymph nodes, kidneys, bladder, lungs, heart, brain, urine, and feces of Balb/c mice that were injected IV with 200 mg/kg (about 5 mg of SiO₂ per mouse) of OPH-loaded protocells. Two mice were sacrificed 1, 7, 14, 21, and 28 days PI, whole blood and organs were homogenized, and SiO₂ was quantified using atomic adsorption spectrometry. Each bar represents the mean+std. dev. for 2 mice. ND=none detected. In all experiments, protocells were modified with CD47, a molecule expressed by erythrocytes that innate immune cells recognize as ‘self’ (Giri et al., 2007).

FIGS. 22A-B. 150-nm protocells modified with an aminopeptidase P antibody rapidly accumulate in the lungs. A) Concentrations of silica (SiO₂) and levofloxacin (LEVO) in the lungs of Balb/c mice that were injected IV with free LEVO or LEVO-loaded protocells; the doses of SiO₂ and LEVO were 200 mg/kg and about 160 mg/kg, respectively. Two mice from the ‘free LEVO’ group were sacrificed immediately after injection and one and two days post-injection (PI); two mice from the ‘LEVO-loaded protocel’ group were sacrificed immediately after injection and at 1, 2, 3, 5, 7, 14, and 21 days PI. SiO₂ was quantified using atomic adsorption spectrometry, and LEVO was quantified using a fluorescence-based HPLC method; concentrations are expressed as μg per mg of lung tissue (about 240 mg/mouse). Each data point represents the mean±std. dev. for 2 mice. B) Total mass of silica (SiO₂) in the blood, liver, spleen, lymph nodes, kidneys, bladder, lungs, heart, brain, urine, and feces of Balb/c mice that were injected IV with 200 mg/kg (about 5 mg of SiO₂ per mouse) of levofloxacin-loaded protocells. Two mice were sacrificed 1, 7, and 14 days PI, whole blood and organs were homogenized, and SiO₂ was quantified using atomic adsorption spectrometry. Each bar represents the mean+std. dev. for 2 mice. ND=none detected. In all experiments, protocells were targeted to the lung via surface-modification with an antibody against aminopeptidase P.

FIGS. 23A-C. SPECT provides quantitative information about protocell biodistribution. SPECT images of Balb/c mice injected IV with 200 mg/kg of DOPC protocells labeled with indium-111. Protocells were 250-nm in diameter and untargeted in (A), 150-nm in diameter and targeted to the lungs via surface modification with an antibody against aminopeptidase P in (B), and 150-nm in diameter and targeted to innate immune cells via surface-modification with Fcγ in (C). Mice were imaged 24 hours post-injection. KEY: Lv=liver, K=kidney, B=bladder, Ln=lung, and S=spleen.

FIG. 24A-C. EB2-targeted protocells deliver siRNA that silences expression of NiV protein(s) in EB2-positive cells. EB2-targeted protocells silence 90% of NiV-N mRNA at a siRNA concentration of about 5 pM. mRNA concentrations begin to increase after 5 days.

FIG. 25A-G. Delivery of histone-packaged pDNA that encodes shRNA specific for NiV-N promotes long-term silencing of NiV mRNA (more than 4 weeks). A plasmid that encodes shRNA specific for NiV-N is prepackaged with histones into highly condensed, e.g., about 18-nm, nanoparticles. EB2-targeted protocells silence 90% of NiV-N mRNA at a cell:protocell ratio of about 1:20. mRNA levels ramin low for >4 weeks.

FIG. 26A-C. SLB fluidity promotes a high differential affinity for target cells at low ligand densities.

FIG. 27. Protocells are biocompatible and can be engineered for persistence and systemic distribution.

FIG. 28A-B. Filamentous phage display enables identification of peptides that bind to human ephrin B2 (EB2). ELISA reveals that the differential affinity of pooled phage for CHO-K1/EB2 increases after each round of selection and that five of the fifth round clones have a high affinity for CHO-K1/EB2.

FIG. 29. Delivery of histone-packaged pDNA promotes long-term silencing of NiV mRNA. Protocells are about 100- to 1000-fold more effective than comparable lipoplexes.

FIG. 30. Protocells synergistically combine the anti-viral therapeutic advantages of MSNPs and liposomes.

FIGS. 31A-B. A) Peptides that resulted from four rounds of positive selection against CHO-K1 cells that express ephrin B2 and three rounds of affinity selection against parental CHO-K1 cells (SEQ ID NOs:52-55 and 57-59). An M13 phage-based 7-mer library was used. B) Immunofluorescence data showing the relative binding affinity of peptides in (A) for CHO-K1 cells that express ephrin B2. CHO-K1/ephrin B2 cells were incubated with 1000-fold excess of phage displaying each of the peptides for 1 hour at 37° C. before being extensively washed to remove unbound phage. Bound phage were detected using a fluorescently-labeled antibody against M13 pVIII coat protein. Higher fluorescence intensities indicate higher affinities. For this reason, TGAILHP was selected for use in targeted delivery experiments.

FIG. 32. Schematic of the nanoparticle system allowing targeted, programmable delivery of antibiotics and/or cell penetrating peptide-peptide nucleic acid (CPP-PNA) conjugates to simultaneously silence multiple resistance genes.

FIGS. 33A-B. A) bla_{TEM-1} in E. coli ER2420/pACYC177 is a good model for bla_{TEM-1} in K. pneumoniae BAA-2146 with >99% nucleotide identity and 100% amino acid sequence identity. B) design of antisense PNA targeting ribosomal binding site (RBS) and start codon of bla_{TEM-1} gene (SEQ ID NOs: 60 and 61).

FIG. 34. Test array filled with solid agar pads filled with different drug or drug/inhibitor combinations. The image at left was taken with a color camera, while the image at right was a fluorescence image recorded using green illumination and red emission. Similar fluorescence images could be obtained with a UV transilluminator set up for imaging DNA gels stained with ethidium bromide. The example above shows that the E. coli is fully resistant to amoxicillin (MIC>128 μg/mL), but addition of the beta-lactamase inhibitor davulanic acid drops the MIC to 4 μg/mL (the 2/1 mixture of amoxicillin/davulanic acid is commonly prescribed in the United States under the trade name “augmentin”). The E. coli is also susceptible to rifampicin (MIC=8 μg/mL). The large rectangular well at the right side of the array was intended as a sterility control, but was inadvertently inoculated when spreading cells across the agar pads.

FIG. 35A-B. 20-well linear test strips for MIC determination. Left panel: testing MIC of E. coli to orthogonal antibiotics. Result: high level resistance to kanamycin (MIC>256 μg/mL), susceptible to ciprofloxacin (MIC<0.031 μg/mL). Right panel: testing response of E co/iwith TEM-1 b-lactamase to b-lactamase inhibitor (davulanic acid, CLV). Result: CLV reduces MIC for AMX from >256 μg/mL depending on dose. The top row (constant ratio AMX/CLV) and bottom row (constant concentration CLV) represent American (CLSI) and European (EUCAST) recommendations for testing beta-lactam+inhibitor combinations. The discrepancy between the two methods also reflects the ±2-fold uncertainty typical for MIC testing.

FIG. 36A-B. E. coli ER2420/pACYC177 (1/2000 dilution of stationary phase culture) was inoculated into wells with dehydrated amoxicilin (AMX) at 256 or 64 μg/mL, with either a specific blaTEM-1 silencer PNA probe or nonsense (control) probe, at concentrations from 0-40 μM, and incubated for 4.5 hours. The color change (blue to pink, A) or fluorescence (B) indicates growth.

FIGS. 37A-B. PMA-PCR for rapid determination of drug sensitivity for E. coli. A) Real-time PCR curves targeting the Enterobacteriaceae 16S rRNA gene for samples treated with PMA and photolysed for 15 minutes, vs untreated controls. The heat-treated control represents the initial inoculum, heat killed prior to incubation (the large shift in Ct indicates that PMA successfully “destroys” most DNA in this sample). The other samples were incubated with varying concentrations of cefotaxime for 2 hours, prior to PMA treatment. B) Change in cycle time upon incubation for 2 hours (red bars), or upon PMA treatment and photolysis of samples incubated for 2 hours (blue bars). Interpretation: In theory |delta-Ct_{growth}| is equal the number of cell divisions that occurred during the 2 hour incubation. Dela-Ct_{PMA} reflects the relative proportion of DNA from dead vs. live cells in the sample, such that -delta-Ct_{PMA}=log₂ (DNA from live cells/total DNA, live+dead). In practice, the efficiency of PMA inactivation is less than 100%, so some DNA from dead cells is still amplified.

FIGS. 38A-D. MSNPs generated via aerosol-assisted EISA have a high capacity for physicochemically disparate antibiotics, the release rates of which can be tailored by altering the degree of condensation of the MSNP framework. A) The loading capacities of MSNPs with 2.5-nm pores for several physicochemically disparate antibiotics. Approximate weight percentages of individual antibiotics when MSNPs are loaded with cocktails of levofloxacin (LEV), doxycycline (DOX), and gentamicin (GEN) or ceflazidime (CEF), sulfamethoxazole (SMX), and trimethoprim (TMP) are included at the far right. B) The loading capacities of MSNPs for acidic (doxycycline, pK, =4.7), basic (gentamicin, pKa=13.2), and hydrophobic (levofloxacin, log P=2.1) drugs can be enhanced by altering the charge or degree of hydrophobicity of the MSNP framework. MSNPs are naturally negatively-charged (ζ=−20 mV in 0.5×PBS, pH 7.4) but were modified with (3-aminopropyl)triethoxysilane (APTES) to make pores positively-charged (ζ=+25 mV in 0.5×PBS, pH 7.4) and with hexamethyldisilazane (HMDS) to make pores more hydrophobic. C) Time-dependent release of levofloxacin from MSNPs with a low or high degree of silica (SiO₂) framework condensation upon incubation in a simulated body fluid (10% serum, pH 7.4) at 37° C. A low degree of silica condensation was achieved using acidified ethanol to extract structure-directing surfactants, while a high degree of silica condensation was promoted via thermal calcination. D) The percentage of levofloxacin released from MSNPs with a high degree of silica framework condensation upon incubation in a simulated body fluid (10% serum, pH 7.4) at 37° C. for the indicated periods of time. Data represent the mean±std. dev. for n=3.

FIGS. 39A-C. Encapsulation of levofloxacin-loaded MSNPs in a PEGylated SLB enables long-term colloidal stability and drug retention in simulated body fluids, and SLB stability can be modulated to control release of levofloxacin. A) The mean hydrodynamic diameters, as determined by DLS, of DOPC protoceHs and PEI-coated MSNPs upon incubation in a simulated body fluid (10% FBS, pH 7.4) at 37° C. for the indicated periods of time. B) The percentage of levofloxacin released from DOPC protocells and DSPC liposomes upon incubation in a simulated body fluid (10% serum, pH 7.4) at 37° C. for the indicated periods of time. C) The percentage of levofloxacin released from protocells with SLBs composed of DOPC (‘DOPC SLB’), SLBs composed of 70 wt % DOPC and 30 wt % of photopolymerized 16:0-23:2 Diyene PC (‘Crosslinked SLB’), and SLMs composed of 70% DOPC with 30 wt % 18:1 MPB PE (‘Crosslinked SLM’) upon incubation in a simulated body fluid (10% serum, pH 7.4), 50 mM sodium citrate at pH 5.0, or 1×PBS with 500 ng/mL of phospholipase A at 37° C. for the indicated periods of time. The levofloxacin release profile for MSNPs without a SLB is included for comparison. MSNPs with a low degree of silica condensation were used in all experiments. Protocell SLBs were composed of DOPC with 30 wt % of cholesterol and 10 wt % of PEG unless otherwise noted. Liposomes in (B) were composed of DSPC with 30 wt % cholesterol and 10 wt % of PEG. Data represent the mean±std. dev. for n=3.

FIGS. 40A-B. Protocells have a high capacity for physicochemically disparate therapeutic agents, tailorable release rates, and exquisite targeting specificities, all of which enable levofloxacin-loaded, Fcγ-targeted protocells to effectively kill intracellular Ft in a cell-specific fashion. A) Mean fluorescence intensities of THP-1, A549, and HepG2 after incubation with DOPC protocells modified with 5 wt % of human Fcγ, 5 wt % of human complement C3, 30 wt % of mannosylated cholesterol, 5 wt % of human ephrin B2, or 5 wt % of SP94. MSNPs were labeled with pHrodo® Red, the fluorescence intensity of which dramatically increases under acidic (i.e., phagosomal or endosomal) conditions. Protocells coated with just DOPC (electrically neutral) or with a cationic lipid (DOTAP) were included to demonstrate that positively-charged nanoparticles are indiscriminately internalized by most cell types. B) Number of colony-forming units (CFUs) of Ft LVS that remain upon treatment of LVS-infected THP-1 or A549 cells with empty protocells, free levofloxacin (Levo), or levofloxacin loaded in DOPC protocells or DPPC liposomes modified with 5 wt % of Fcγ and 5 wt % of H5WYG. The viability of THP-1 cells treated with empty protocells and A549 cells treated with levofloxacin-loaded, Fcγ-targeted protocells dramatically declined between 24 and 36 hours due to LVS-mediated apoptosis. Data represent the mean±std. dev. for n=3.

FIGS. 41A-D. Characterization of the nanoporous silica particles that form the protocell core. A) Transmission electron microscopy (TEM) image of multimodal silica particles formed via the emulsion processing technique described by Carroll at al. Scale bar=100 nm. The inset shows a scanning electron microscopy (SEM) image of a 5-μm multimodal silica particle, in which surface-accessible pores are visible; large particles were used to enhance resolution. Inset scale bar=200 nm. B) Dynamic light scattering (DLS) of multimodal silica particles after size-based separation. Resulting particles had an average diameter of about 165 nm. C) Nitrogen sorption isotherm for size-separated multimodal particles. The presence of hysteresis is consistent with a network of larger pores interconnected by smaller pores. D) A cumulative pore volume plot, calculated from the adsorption branch of the isotherm in C) using the Barrett-Joyner-Halenda (BJH) model, demonstrates the presence of large (23-30 nm) pores and small (3-13 nm) pores.

FIGS. 42A-C. Protocells have a high capacity for nucleic acids, the release of which is triggered by acidic pH. A) The concentrations of nucleic acids that can be loaded within 10¹⁰ protocells and lipid nanoparticles (LNPs). Zeta potential values for unmodified and AEPTMS-modified silica cores in 0.5×PBS (pH 7.4) are −32 mV and +12 mV, respectively. B) and C) The rates at which nucleic acid is released from DOPC protocells with AEPTMS-modified cores, DOPC protocells, and DOTAP protocells upon exposure to a pH 7.4 simulated body fluid B) or a pH 5.0 buffer C) at 37° C. The average diameters of nucleic acid loaded protocells, DOPC protocells, and DOTAP protocells were 178-nm (±24.3-nm), 135-nm (+19.1-nm), and 144-nm (+14.8-nm), respectively. Error bars represent 95% confidence intervals (1.96 σ) for n=3.

FIG. 43. Air-pouch model of invasive S. aureus skin and soft tissue infection. Air (5 cc) is injected subcutaneously six days prior to infection. On day 3 before infection, air (2.5 cc) is again injected into the preformed pouch. Mice are infected by directly injecting S. aureus into the air-pouch. The pouch is lavaged post-infection to assess bacterial burden.

FIG. 44. Recovery and relative quantification of protocells from the air-pouch of MRSA infected mice. Dylight® 633-labeled protocells were quantified by flow cytometry from pouch lavage taken 4 and 24 hours after mouse air-pouches were infected with MRSA.

FIG. 45. Biodistribution of Dylight® 633-labeled protocells during MRSA SSTI. In vivo imaging of Dylight® 633-labeled protocells in mouse kidneys, liver, spleen and lavaged/extracted air-pouch at four hours post-infection. Tissues from mice infected but not injected with protocells are shown as a fluorescent control.

FIG. 46A-B. Tissue specific distribution of Dylight® 633-labeled protocells during MRSA SSTI. Comparison of protocell concentrations (based on fluorescence intensity) in mouse kidneys, liver, spleen and lavaged/extracted air-pouch at four and 24 hours post MRSA infection. Tissues from mice infected but not injected with protocells are shown as control. Yellow=high intensity, dark red=low intensity.

FIG. 47. Quantification of Dylight® 633-labeled protocells in pouch and kidneys at four and 24 hours post-infection. IVIS in vivo imaging of lavaged and extracted air-pouches and kidneys from mice after MRSA infection.

FIG. 48. Protocels colocalize with S. aureus during SSTI. Confocal imaging of air-pouches from mice after MRSA infection.

FIG. 49. Protocels bind MRSA in vitro. Flow cytometry analysis of Dylight® 633-labeled protocels binding to MRSA.

FIGS. 50A-C. Vancomycn-loaded protocells reduce morbidity and bacterial burden in MRSA infected mice. 5×10⁷ CFU MRSA isolate LAC injected into air-pouch (BALB/c mice) along with 2.5 mg of vancomycin-loaded protocels or empty controls. Twenty-four hours post-infection, mice were sacrificed and bacterial burden in (A) the pouch lavage and (B) dissemination to the kidney was determined by plating serial dilutions on sheep blood agar. (C) Morbidity scoring was based grooming, natural behavior, provoked behavior and weight loss

FIG. 51A-D. MSNP (MSN) EISA synthesis and characterization in accordance with certain compositional and process conditions as described herein. Silcia nanoparticles are generated using an aerosol-assisted evaporation induced self-assembly method. A precursor sol is prepared by combining a surfactant, TEOS, ethanol and water, well below the surfactant's critical micelle concentration. The sol is atomized and the droplet is carried into a drying zome where solvent evaporation begins, increasing the effective surfactant concentration, allowing self-assembly to occur. The droplet enters the heating zome, which evaporates the remaining solvent and drives silica condensation to form solid particles. This method allows for tunable pore size, controllable particle diameter and modulation of dissolution kinetics.

FIG. 52A-G. MSNP biochemical and biophysical properties, e.g., particle diameter, pore size, pore volume, dissolution rate and charge, properties which can be independently controlled, e.g., in accordance with certain compositional and process conditions described herein, can accommodate various types of cargo and allow for control of a cargo's pharmacokinetics.

FIG. 53A-G. Core functionalization enables high-capacity MSNP loading of disparate cargos. Exemplary cargo includes but is not limited to paclitaxel, carboplatin, gemcitabine, ibuprofen, imatinib, doxorubicin, camptothecin, and ciclopirox.

FIG. 54. Modification of the supported lipid bilayer formulation achieve synergistic protocell cargo loading. Biochemical and biophysical properties can be varied to encapsulate disparate cargo types for various delivery applications. For example, the physicochemical properties of each cargo can be masked, efficiently modulating the cargo's aqueous solubility and permeability, which allows for control over pharmacokinetic behavior.

### DETAILED DESCRIPTION

### Definitions

The following terms shall be used throughout the specification. Where a term is not specifically defined herein, that term shall be understood to be used in a manner consistent with its use by those of ordinary skill in the art.

Where a range of values is provided, it is understood that each intervening value, to the tenth of the unit of the lower limit unless the context dearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention. In instances where a substituent is a possibility in one or more Markush groups, it is understood that only those substituents which form stable bonds are to be used.

Unless defined otherwise, all technical and scientific terms used herein have the same meaning as commonly understood by one of ordinary skill in the art to which this invention belongs. Although any methods and materials similar or equivalent to those described herein can also be used in the practice or testing, in one embodiment methods and materials are now described.

It must be noted that as used herein and in the appended claims, the singular forms “a,” “and” and “the” include plural references unless the context clearly dictates otherwise.

The term “broadly neutralizing Ab” means an Ab that can recognize and neutralize multiple variants of highly variable antigens, which is particularly important in protection against fast mutating viruses such as the influenza virus. Artificial passive immunization relies on administration of pathogen-specific and neutralizing Abs present in the serum of an immunized individual or purified either from such a serum or from any protein expression system. This process leads to curing of the existing infection (therapeutic immunization) and/or to a short-term protection against subsequent infections (protective immunization).

The term “protocell” is used to describe a porous nanoparticle which is made of a material comprising silica, polystyrene, alumina, titania, zirconia, or generally metal oxides, organometallates, organosilicates or mixtures thereof. A porous (typically spherical) silica nanoparticle is used for the in one embodiment protocells and is surrounded by a supported lipid or polymer bilayer or multilayer. Various embodiments provide nanostructures and methods for constructing and using the nanostructures and providing protocells.

“Multiphase pore-surface structure” means that a nanoparticulate's pores and surface exhibit two (biphasic) or more distinct morphologies (e.g., crystalline or amorphous structures), as determined through well-known techniques such as X-ray absorption spectroscopy (XAS), X-ray diffraction (XRD) and inductively coupled plasma (ICP). See e.g., Moreau, et al., (2013).

“Cationic surfactant and the poloxamer have different phases” means that the cationic surfactant and poloxamer exhibit apparent absolute immiscibility at all ratios of the cationic surfactant and poloxamer.

“Self-assembly using a templating surfactant system”, e.g., as employed in aerosol-assisted evaporation-induced self-assembly (EISA), is described in Lu, Y. F. and Brinker J. C. et al. Aerosol-assisted self-assembly of mesostructured spherical nanoparticles”, Nature 398, 223-226 (1999)), the complete contents of which are hereby incorporated by reference. As explained in U.S. Pat. No. 8,334,014, “[t]emplating of oxide materials with surfactant micelles is a powerful method to obtain mesoporous oxide structures with controlled morphology. In this method, an oxide precursor solution is mixed with a templating surfactant and evaporation of the solvent leads to an increase in the surfactant concentration. The surfactant forms supra-molecular structures according to the solution phase diagram. This is known as evaporative induced self-assembly (EISA) and has been used to obtain bulk porous materials or microparticles using high-temperature aerosol methods. Alternatively, mesoporous particle synthesis via EISA can be performed in water in oil emulsion droplets under milder temperature stresses (citations omitted).”

A “multi-modal pore size distribution” means that there are two or more nanoparticle pore size distributions, as opposed to a monomodal pore size distribution which exhibits a Gaussian or log normal form.

“Differential pore volume distributions” can be considered in the broadest sense to be logarithmic differential pore volume distributions defined by plots of (dV/dlog(D) vs. D (or [dV/dr]/[d(log (r)/dr] vs. r, where V is nanoparticle volume, D is nanoparticle diameter and r is nanoparticle radius. Differential pore volume distributions may be determined in a number of ways, including through use of the Barret-Joyner-Halenda (BJH) model, the Horvath-Kawazoe (HK) model and the Density Functional Theory (DFT) model, as illustrated in Muhammad Afiq Aizuddin Musa, Chun-Yang Yin and Robert Mikhail Savory, 2011, Analysis of the Textural Characteristics and Pore Size Distribution of a Commercial Zeolite using Various Adsorption Models, Journal of Applied Sciences, 11: 3650-3654. The theoretical bases of differential pore size distribution are presented in Meyer, et al., Comparison between different presentations of pore size distribution in porous materials, Fresenius' Journal of Analytical Chemistry, Vol. 363, Issue 2, pp. 174-178.

The term “prophylactic administration” refers to any action in advance of the occurrence of disease to reduce the likelihood of that disease or any action to reduce the likelihood of the subsequent occurrence of disease in the subject.

The term “patient” or “subject” is used throughout the specification within context to describe an animal, generally a mammal, especially including a domesticated animal and, e.g., a human, to whom treatment, including prophylactic treatment (prophylaxis), with the compounds or compositions is provided. For treatment of those infections, conditions or disease states which are specific for a specific animal such as a human patient, the term patient refers to that specific animal. In most instances, the patient or subject is a human patient of either or both genders.

The term “effective” is used herein, unless otherwise indicated, to describe an amount of a compound or component which, when used within the context of its use, produces or effects an intended result, whether that result relates to the prophylaxis and/or therapy of an infection and/or disease state or as otherwise described herein. The term effective subsumes all other effective amount or effective concentration terms (including the term therapeutically effective) which are otherwise described or used in the present application.

The term “compound” is used herein to describe any specific compound or bioactive agent disclosed herein, including any and all stereoisomers (including diastereomers), individual optical isomers (enantiomers) or racemic mixtures, pharmaceutically acceptable salts and prodrug forms. The term compound herein refers to stable compounds. Within its use in context, the term compound may refer to a single compound or a mixture of compounds as otherwise described herein.

The term “cargo” is used herein to describe any molecule or compound, whether a small molecule or macromolecule having an activity relevant to its use in MSNPs (MSNs), especially including biological activity, which can be included in MSNPs. The cargo may be included within the pores and/or on the surface of the MSNP. Representative cargo may include, for example, a small molecule bioactive agent, a nucleic acid (e.g., RNA or DNA), a polypeptide, including a protein or a carbohydrate. Particular examples of such cargo include RNA, such PNA's, PNA's comprising asRNA, mRNA, siRNA, shRNA micro RNA, a polypeptide or protein and/or DNA (including double stranded or linear DNA, complementary DNA (cDNA), minidrcle DNA, naked DNA and plasmid DNA (including CRISPR plasmids) which optionally may be supercoiled and/or packaged (e.g., with histones) and which may be optionally modified with a nuclear localization sequence). The nanoparticles may also be loaded with Locked Nucleic Acids (LNA™), which are nucleic acid analogues in which the ribose ring is “locked” by a methylene bridge connecting the 2′-O atom and the 4′-C atom. Cargo may also include a reporter as described herein. Cargos may also include antibiotics or CPP-PNAs.

The term “PEGylated” in its principal use refers to an MSNP which has been produced using PEG-containing silanes or zwitterionic group-containing silanes to form the MSNP. In general, the amount of the PEG-containing silanes and/or zwitterionic-containing silanes which optionally are used to produce MSNPs represent about 0.05% to about 50% (about 0.1% to about 35%, about 0.5% to about 25%, about 1% to about 20%, about 2.5% to about 30%, about 0.25% to about 10%, about 0.75% to about 15%) by weight of these monomers in combination with the silane monomers which are typically used to form MSNPs. A PEG-containing silane is any silane which contains a PEG as one of the substituents and the remaining groups can facilitate the silane reacting with other silanes to produce MSNPs. For example, PEG-containing silanes and/or zwitterionic-containing silanes which are used in the present invention to create PEGylated MSNPs include 2-[methoxy(polyethyleneoxy)propyl]trimethoxysilane (containing varying molecular weights of PEG ranging from about 100 to 10,000 average molecule weight, often about 200 to 5,000 average molecular weight, about 1,000-2,500 average molecular weight, about 1500-2000 average molecular weight) and 3-{[Dimethoxyl(3-tnmethoxysilyl)propyl]ammonio)propane-1-sulfonate and mixtures thereof, among others. The term “PEGylated” may also refer to lipid bilayers which contain a portion of lipids which are PEGylated (from about 0.02% up to about 50%, about 0.1% to about 35%, about 0.5% to about 25%, about 1% to about 15%, about 0.5% to about 7.5%, about 1% to about 12.5% by weight of the lipids used to form the lipid bilayer or multilayer). These lipids often are amine-containing lipids (e.g., DOPE and DPPE) which are conjugated or derivatized to contain a PEG group (having an average molecule weight ranging from about 100 to 10,000, about 200 to 5,000, about 1,000-5,000, including 1,000, 2000, 3000 and 3400) and combined with other lipids to form the bilayer/multilayer which encapsulates the MSNP.

The terms “targeting ligand,” “targeting active species,” and “targeting moiety” are used interchangeably herein to describe a compound or moiety (for example, an antigen, antibody, or peptide) which is complexed or for example covalently bonded to the surface of a MSNPs and/or protocells which binds to a moiety on the surface of a cell to be targeted so that the MSNPs and/or protocells may selectively bind to the surface of the targeted cell and deposit their contents into the cell. The targeting active species for use in the present invention is, for example, a targeting peptide as otherwise described herein, a polypeptide including an antibody or antibody fragment, an aptamer, or a carbohydrate, among other species which bind to a targeted cell.

The terms “treat”, “treating”, and “treatment”, are used synonymously to refer to any action providing a benefit to a patient at risk for or afflicted with a disease, including improvement in the condition through lessening, inhibition, suppression or elimination of at least one symptom, delay in progression of the disease, prevention, delay in or inhibition of the likelihood of the onset of the disease, etc. In the case of microbial (e.g., viral and/or bacterial) infections, these terms, for example, include, in certain particularly favorable embodiments the eradication or elimination (as provided by limits of diagnostics) of the microbe (e.g., virus and/or bacterium) which is the causative agent of the infection. In one embodiment, the symptom is due to tetanus, anthrax, haemophilus, pertussis, diphtheria, cholera, lyme disease, bacterial meningitis, Streptococcus pneumoniae, or typhoid, or an infection by a fungus, protest, archaea, or virus.

Treatment, as used herein, encompasses both prophylactic and therapeutic treatment. Pharmaceutical formulations can, for example, be administered prophylactically to a mammal in advance of the occurrence of disease to reduce the likelihood of that disease. Prophylactic administration is effective to reduce or decrease the likelihood of the subsequent occurrence of disease in the mammal, or decrease the severity of disease (inhibition) that subsequently occurs. Alternatively, compounds can, for example, be administered therapeutically to a mammal that is already afflicted by disease. In one embodiment of therapeutic administration, administration of the present compounds is effective to eliminate the disease and produce a remission or substantially eliminate the likelihood of a recurrence. Administration of the protocels and pharmaceutical formulations is effective to decrease the severity of the disease or lengthen the lifespan of the mammal so afflicted, or inhibit or even eliminate the causative agent of the disease. In another embodiment of therapeutic administration, administration of the present compounds is effective to decrease the likelihood of infection or re-infection by a microbe and/or to decrease the symptom(s) or severity of an infection.

The term “pharmaceutically acceptable” as used herein means that the compound or composition is suitable for administration to a subject, including a human patient, to achieve the treatments described herein, without unduly deleterious side effects in light of the severity of the disease and necessity of the treatment.

The term “lipid” is used to describe the components which are used to form lipid bi- or multilayers on the surface of the nanoparticles which are used in the present invention and may indude a PEGylated lipid. Various embodiments provide nanostructures which are constructed from nanoparticles which support a lipid bilayer(s). In embodiments, the nanostructures, for instance, include, for example, a core-shell structure including a porous particle core surrounded by a shell of lipid bilayer(s). The nanostructure, for instance, a porous silica or alum nanostructure as described above, supports the lipid bilayer membrane structure.

The term “reporter” is used to describe an imaging agent or moiety which is incorporated into the phospholipid bilayer or cargo of MSNPs according to an embodiment and provides a signal which can be measured. The moiety may provide a fluorescent signal or may be a radioisotope which allows radiation detection, among others. Exemplary fluorescent labels for use in MSNPs and protocells (for example, via conjugation or adsorption to the lipid bi- or multilayer or silica core, although these labels may also be incorporated into cargo elements such as DNA, RNA, polypeptides and small molecules which are delivered to cells by the protocells) include Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa FluoPr 488 conjugate of annexin V (495/519), Alexa Fluor® 488 goat anti-mouse IgG (H+L) (495/519), Click-iT® AHA Alexa Fluor® 488 Protein Synthesis HCS Assay (495/519), LIVEOEAD® Fixable Green Dead Cell Stain Kit (495/519), SYTOX® Green nudeic acid stain (504/523), MitoSOX™ Red mitochondrial superoxide indicator (510/580). Alexa Fluor® 532 carboxylic acid, succinimidyl ester(532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608). Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670) and Alexa Fluor® 647 conjugate of annexin V (650/665). Moieties which enhance the fluorescent signal or slow the fluorescent fading may also be incorporated and include SlowFade® Gold antifade reagent (with and without DAPI) and Image-iT® FX signal enhancer. All of these are well known in the art. Additional reporters include polypeptide reporters which may be expressed by plasmids (such as histone-packaged supercoiled DNA plasmids) and include polypeptide reporters such as fluorescent green protein and fluorescent red protein. Reporters pursuant to the present invention are utilized principally in diagnostic applications including diagnosing the existence or progression of a viral infection in a patient and or the progress of therapy in a patient or subject.

The term “histone-packaged supercoiled plasmid DNA” is used to describe one embodiment of protocells which utilize in one embodiment plasmid DNA which has been “supercoiled” (i.e., folded in on itself using a supersaturated salt solution or other ionic solution which causes the plasmid to fold in on itself and “supercoil” in order to become more dense for efficient packaging into the protocells). The plasmid may be virtually any plasmid which expresses any number of polypeptides or encode RNA, including small hairpin RNA/shRNA or small interfering RNAl/siRNA, as otherwise described herein. Once supercoiled (using the concentrated salt or other anionic solution), the supercoiled plasmid DNA is then complexed with histone proteins to produce a histone-packaged “complexed” supercoiled plasmid DNA.

“Packaged” DNA herein refers to DNA that is loaded into protocells (either adsorbed into the pores or confined directly within the nanoporous silica core itself). To minimize the DNA spatially, it is often packaged, which can be accomplished in several different ways, from adjusting the charge of the surrounding medium to creation of small complexes of the DNA with, for example, lipids, proteins, or other nanoparticles (usually, although not exclusively cationic). Packaged DNA is often achieved via lipoplexes (i.e., complexing DNA with cationic lipid mixtures). In addition, DNA has also been packaged with cationic proteins (including proteins other than histones), as well as gold nanoparticles (e.g., NanoFlares- an engineered DNA and metal complex in which the core of the nanoparticle is gold).

The term “nuclear localization sequence” (NLS) refers to a peptide sequence incorporated or otherwise crosslinked into histone proteins which comprise the histone-packaged supercoiled plasmid DNA. In certain embodiments, protocels may further comprise a plasmid (often a histone-packaged supercoiled plasmid DNA) which is modified (crosslinked) with a nuclear localization sequence (note that the histone proteins may be crosslinked with the nuclear localization sequence or the plasmid itself can be modified to express a nuclear localization sequence) which enhances the ability of the histone-packaged plasmid to penetrate the nucleus of a cell and deposit its contents there (to facilitate expression and ultimately cell death. These peptide sequences assist in carrying the histone-packaged plasmid DNA and the associated histones into the nucleus of a targeted cell whereupon the plasmid will express peptides and/or nucleotides as desired to deliver therapeutic and/or diagnostic molecules (polypeptide and/or nucleotide) into the nucleus of the targeted cell. Any number of crosslinking agents, well known in the art, may be used to covalently link a nuclear localization sequence to a histone protein (often at a lysine group or other group which has a nucleophilic or electrophilic group in the side chain of the amino acid exposed pendant to the polypeptide) which can be used to introduce the histone packaged plasmid into the nucleus of a cell. Alternatively, a nucleotide sequence which expresses the nuclear localization sequence can be positioned in a plasmid in proximity to that which expresses histone protein such that the expression of the histone protein conjugated to the nuclear localization sequence will occur thus facilitating transfer of a plasmid into the nucleus of a targeted cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation, as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgamo sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence. Transcriptional and translational control sequences are DNA regulatory sequences, such as promoters, enhancers, polyadenylation signals, terminators, and the like, that provide for the expression of a coding sequence in a host cell.

A “signal sequence” can be included before the coding sequence. This sequence encodes a signal peptide. N-terminal to the polypeptide, that communicates to the host cell to direct the polypeptide to the cell surface or secrete the polypeptide into the media, and this signal peptide is dipped off by the host cell before the protein leaves the cell. Signal sequences can be found associated with a variety of proteins native to prokaryotes and eukaryotes.

A nucleic acid molecule is “operatively linked” to, or “operably associated with”, an expression control sequence when the expression control sequence controls and regulates the transcription and translation of nucleic acid sequence. The term “operatively linked” includes having an appropriate start signal (e.g., ATG) in front of the nucleic acid sequence to be expressed and maintaining the correct reading frame to permit expression of the nucleic acid sequence under the control of the expression control sequence and production of the desired product encoded by the nucleic acid sequence. If a gene that one desires to insert into a recombinant DNA molecule does not contain an appropriate start signal, such a start signal can be inserted in front of the gene.

A “peptide nucleic acid (PNA)” is a synthetic nucleic acid mimic in which the sugar-phosphate backbone is replaced by a peptide backbone. PNAs hybridize to complementary DNA and RNA with higher affinity and superior sequence selectivity compared to DNA. PNAs are resistant to nucleases and proteases and have a low affinity for proteins. These properties make PNAs an attractive agent for biological and medical applications. To improve the antisense and antigene properties of PNAs, many backbone modifications of PNAs have been explored under the concept of preorganization. Sugiyama, et al., “Chiral peptide nucleic acids with a substituent in the N-(2-aminoethy)glycne backbone”, Molecules, 2012 Dec. 27:18(1):287-310. doi: 10.3390/moleculesl 8010287.

A “control” as used herein may be a positive or negative control as known in the art and can refer to a control cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. For instance, as can be appreciated by a skilled artisan, a control may comprise data from one or more control subjects that is stored in a reference database. The control may be a subject who is similar to the test subject (for instance, may be of the same gender, same race, same general age and/or same general health) but who is known to not have a fibrotic disease. As can be appreciated by a skilled artisan, the methods can also be modified to compare a test subject to a control subject who is similar to the test subject (for instance, may be of the same gender, same race, same general age and/or same general health) but who is known to express symptoms of a disease. In this embodiment, a diagnosis of a disease or staging of a disease can be made by determining whether protein or gene expression levels as described herein are statistically similar between the test and control subjects.

As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e g., dimers, tetramers). Thus, the terms peptide, oligopeptide, and protein are all included within the definition of polypeptide and these terms are used interchangeably. It should be understood that these terms do not connote a specific length of a polymer of amino acids, nor are they intended to imply or distinguish whether the polypeptide is produced using recombinant techniques, chemical or enzymatic synthesis, or is naturally occurring.

A “ligand” can be any natural or synthetic moiety, including but not limited to a small molecule, an antibody, a nucleic acid, an amino acid, a protein (e.g., an enzyme) or a hormone that binds to a cell, for example, at a receptor (binding site) located on he surface of the cell. The term “ligand” therefore includes any targeting active species (compound or moiety, e.g., antigen) which binds to a moiety (for example, a receptor) on, in or associated with a cell. In some embodiments, a ligand is a peptide, a polypeptide including an antibody or antibody fragment, an aptamer, or a carbohydrate, among other species which bind to a targeted cell.

“Binding site” as used herein is not limited to receptor protein surface areas that interact directly with ligands, but also indudes any atomic sequence, whether or not on the surface of a receptor, that is implicated (by affecting conformation or otherwise) in ligand binding. A purely illustrative list of binding sites include those targeted by detector antibodies which are specific to a viral infection cell receptors, as illustrated by the antibodies described in the Examples herein and as otherwise identifiable by techniques which are well-known to those of ordinary skill in the art.

The phrase “effective average particle size” as used herein to describe a multiparticulate (e.g., a porous nanoparticulate) means that at least 50% of the particles therein are of a specified size. Accordingly, “effective average particle size of less than about 2,000 nm in diameter” means that at least 50% of the particles therein are less than about 2,000 nm in diameter. In certain embodiments, nanoparticulates have an effective average particle size of less than about 2,000 nm (i.e., 2 microns), less than about 1,900 nm, less than about 1,800 nm, less than about 1,700 nm, less than about 1,600 nm, less than about 1,500 nm, less than about 1,400 nm, less than about 1,300 nm, less than about 1,200 nm, less than about 1,100 nm, less than about 1,000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, less than about 50 nm, less than 30 nm, less than 25 nm, less than 20 nm, less than 15 nm, less than 10 nm, as measured by light-scattering methods, microscopy, or other appropriate methods. “D₅₀” refers to the particle size below which 50% of the particles in a multiparticulate fall. Similarly, “D₉₀” is the particle size below which 90% of the particles in a multiparticulate fall.

The term “neoplasia” refers to the uncontrolled and progressive multiplication of tumor cells, under conditions that would not elicit, or would cause cessation of, multiplication of normal cells. Neoplasia results in a “neoplasm”, which is defined herein to mean any new and abnormal growth, particularly a new growth of tissue, in which the growth of cells is uncontrolled and progressive. Thus, neoplasia includes “cancer”, which herein refers to a proliferation of tumor cells having the unique trait of loss of normal controls, resulting in unregulated growth, lack of differentiation, local tissue invasion, and/or metastasis.

As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of anaplasia, or loss of differentiation and orientation of cells, and have the properties of invasion and metastasis. Examples of neoplasms or neoplasias from which the target cell may be derived include, without limitation, carcinomas (e.g., squamous-cel carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas (Beers and Berkow (eds.), The Merck Manual of Diagnosis and Therapy, 17.sup.th ed. (Whitehouse Station, N.J.: Merck Research Laboratories, 1999) 973-74, 976, 986, 988, 991.

The need further exists for clinically effective, widely-applicable nanoparticle-based formulations that will target selected cells or pathogens in vivo and that will administer requisite active ingredient dosages, e.g., combinations of active ingredients over a wide range of therapeutic time periods.

Porous silica particles of varying sizes ranging in size (diameter) from less than 5 nm to 200 nm or 500 nm or more are readily available in the art or can be readily prepared using methods known in the art or alternatively, can be purchased from Melorium Technologies, Rochester, N.Y. Sky Spring Nanomaterials, Inc., Houston, Tex., USA or from Discovery Scientific, Inc., Vancouver, British Columbia. Multimodal silica nanoparticles may be readily prepared using the procedure of Carroll, et al., Langmuir, 25, 13540-13544 (2009).

The examples herein provide various methodologies for obtaining protocells which are useful in the present invention. Useful general techniques include those described in Liu et al., (2009), Liu et al, (2009), Liu et al., (2009); Lu et al., (1999), Ashley et al., (2011), Lu et al., (1999), and Caroll et al, (2009).

Nanostructures include a core-shell structure which comprises a porous particle core surrounded by a shell of lipid for example a bilayer, but possibly a monolayer or mutilayer (see Liu, et al., J. Amer. Chem. Soc., 131, 7567-7569 (2009)). The porous particle core can include, for example, a porous nanoparticle made of an inorganic and/or organic material as set forth above surrounded by a lipid bilayer.

The porous particle core of the protocells can be loaded with various desired species (“cargo”), including small molecules (e.g., antiviral agents as otherwise described herein), large molecules (e.g., including macromolecules such as asRNA, siRNA or shRNA or a polypeptide which may include an antiviral polypeptide or a reporter polypeptide (e.g., fluorescent green protein, among others), semiconductor quantum dots, or metallic nanoparticles, or metal oxide nanoparticles or combinations thereof). Representative cargo may include, for example, a small molecule bioactive agent, a nucleic acid (e.g., RNA or DNA), a polypeptide, including a protein, or a carbohydrate. Particular examples of such cargo include RNA, such PNA's, PNA's comprising asRNA, mRNA, siRNA, shRNA, micro RNA, a polypeptide or protein and/or DNA (including double stranded or linear DNA, complementary DNA (cDNA), minicircle DNA, naked DNA and plasmid DNA (including CRISPR plasmids) which optionally may be supercoiled and/or packaged (e.g., with histones) and which may be optionally modified with a nuclear localization sequence). Cargo may also include a reporter as described herein. For example, protocells can also be loaded with super-coiled plasmid DNA, which can be used to deliver a therapeutic and/or diagnostic peptide(s) or a small hairpin RNA/shRNA or small interfering RNA/siRNA, which can be used to inhibit expression of proteins associated with antibiotic resistance.

In some embodiments, protocells are comprised of a spherical mesoporous silica nanoparticle (MSNP) core encased within a supported lipid bilayer (SLB). MSNPs have an extremely high surface area (>1200 m²/g), which enables high concentrations of various therapeutic and diagnostic agents to be adsorbed within the core by simple immersion in a solution of the cargo(s) of interest. Furthermore, since the aerosol-assisted evaporation-induced self-assembly (EISA) process we pioneered to synthesize MSNPs is compatible with a wide range of structure-directing surfactants and amenable to post-synthesis processing, the overall size can be varied from 20-nm to >10-μm, the pore size can be varied from 2.5-nm to 50-nm, and the naturally negatively-charged pore walls can be modified with a variety of functional moieties, enabling facile encapsulation of physicochemically disparate molecules, including acidic, basic, and hydrophobic drugs, proteins, small interfering RNA, minicircle DNA vectors, plasmids, and diagnostic agents like quantum dots and iron oxide nanoparticles.

Protocells are provided that have a loading capacity of up to 60 wt % for small molecule drugs, which is 10-fold higher than other MSNP-based delivery vehicles and 1000-fold higher than similarly-sized liposomes. Release rates can be tailored by controlling the core's degree of silica condensation and, therefore, its dissolution rate under physiological conditions; thermal calcination maximizes condensation and results in particles with sustained release profiles (7-10% release per day for up to 2 weeks), while use of acidified ethanol to extract surfactants enhances particle solubility and results in burst release of encapsulated drugs (100% release within 12 hours). Liposome fusion to cargo-loaded MSNPs results in the formation of a coherent SLB that provides a stable, fluid, biocompatible interface for display of functional molecules, such as polyethylene glycol (PEG) and targeting ligands.

Protocells stably encapsulate small molecule drugs for up to 4 weeks when dispersed in complex biological fluids (e.g., complete growth medium and blood), regardless of whether the SLB is composed of lipids that are fluid or non-fluid at body temperature: in contrast, liposomes rapidly leak their encapsulated drugs, even when their bilayers are composed of fully saturated lipids, which have a high packing density and should, therefore, limit diffusion of drugs across the bilayer. The fluid, yet stable SLB enables us to achieve exquisitely high targeting specificities at low ligand densities, which, in turn, reduces immunogenicity and non-specific interactions; we have shown that protocells modified with an average of just 6 targeting peptides per particle have a 10,000-fold higher affinity for target cells than for non-target cells when the SLB is composed of the fluid, zwittenonic lipid, 1, 2-dioleoyl-sn-glycero-3-phosphocholine (DOPC).

Protocells are highly biocompatible and can be engineered for both broad distribution and persistence within target tissues. Balb/c mice injected intravenously (i.v.) with 200 mg/kg doses of PEGylated protocells three times each week for three weeks show no signs of gross or histopathological toxicity. Given their high loading capacity, this result indicates that protocells can deliver at least 900 mg/kg of therapeutic molecules with either burst or sustained release kinetics. Furthermore, PEGylated protocells 20-200 nm in diameter remain broadly distributed for 2-7 days when injected i.v., which provides a sufficient period of time for targeted protocells to accumulate within target tissues, where they can persist for up to 4 weeks with no adverse effects. Additionally, we and others have demonstrated that MSNPs are biodegradable and ultimately excreted in the urine and feces as silicic acid. Finally, protocells modified with up to 10 wt % of targeting ligands induce neither IgG nor IgM responses when injected in C57Bl/6 mice at a total dose of 400 mg/kg. Depending upon the biodistribution required for a specific application, the MSNP size and shape (spherical, disk-shaped, and rod-shaped and the SLB charge and surface modification(s), may be designed making the protocell a highly modular, flexible nanoparticle delivery system.

Conventionally, a mesoporous nanoparticle has pores whose diameters range in size from about 2 nm to about 50 nm, a “microporous” nanoparticle has pores whose diameters are less than about 2 nm (often about 0.001 to about 2 nm) and a “macroporous” nanoparticle has pores whose diameters are from about 50 nm to about 100 nm. MSNPs can have both mesoporous, microporous and macroporous pores, but often have pores whose diameters range in size from about 2 nm to about

A nanoparticle may have a variety of shapes and cross-sectional geometries that may depend, in part, upon the process used to produce the particles. In one embodiment, a nanoparticle may have a shape that is a torus (toroidal). A nanoparticle may include particles having two or more of the aforementioned shapes. In one embodiment, a cross-sectional geometry of the particle may be one or more of toroidal, circular, ellipsoidal, triangular, rectangular, or polygonal. In one embodiment, a nanoparticle may consist essentially of non-spherical particles. For example, such particles may have the form of ellipsoids, which may have all three principal axes of differing lengths, or may be oblate or prelate ellipsoids of revolution. Non-spherical nanoparticles alternatively may be laminar in form, wherein laminar refers to particles in which the maximum dimension along one axis is substantially less than the maximum dimension along each of the other two axes. Non-spherical nanoparticles may also have the shape of frusta of pyramids or cones, or of elongated rods. In one embodiment, the nanoparticles may be irregular in shape. In one embodiment, a plurality of nanoparticles may consist essentially of spherical nanoparticles.

Effective average particle size includes a multiparticulate (e.g., a porous nanoparticulate) where at least 50% of the particles therein are of a specified size. Accordingly, effective average particle size of less than about 2,000 nm in diameter” means that at least 50% of the particles therein are less than about 2,000 nm in diameter. In certain embodiments, nanoparticulates have an effective average particle size of less than about 2,000 nm (i.e., 2 microns), less than about 1,900 nm, less than about 1,800 nm, less than about 1,700 nm, less than about 1,600 nm, less than about 1,500 nm, less than about 1,400 nm, less than about 1,300 nm, less than about 1,200 nm, less than about 1.100 nm, less than about 1,000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, less than about 50 nm, less than 30 nm, less than 25 nm, less than 20 nm, less than 15 nm, less than 10 nm, as measured by light-scattering methods, microscopy, or other appropriate methods. “D₅₀” refers to the particle size below which 50% of the particles in a multiparticulate fall. Similarly, “D₉₀” is the particle size below which 90% of the particles in a multiparticulate fall.

The MSNP size distribution, depends on the application, but is principally monodisperse (e.g., a uniform sized population varying no more than about 5-20% in diameter, as otherwise described herein). The term “monodisperse” is used as a standard definition established by the National Institute of Standards and Technology (NIST) (Particle Size Characterization, Special Publication 960-1, January 2001) to describe a distribution of particle size within a population of particles, in this case nanoparticles, which particle distribution may be considered monodisperse if at least 90% of the distribution lies within 5% of the median size. See Takeuchi, et al., Advanced Materials, 2005, 17, No. 8, 1067-1072.

In certain embodiments, mesoporous silica nanoparticles can be range, e.g., from around 5 nm to around 500 nm (for example, about 50 nm to about 500 nm) in size, including all integers and ranges there between. The size is measured as the longest axis of the particle. In various embodiments, the particles are from around 10 nm to around 500 nm and from around 10 nm to around 100 nm in size. The mesoporous silica nanoparticles have a porous structure. The pores can be from around 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

In one embodiment, MSNPs are monodisperse and range in size from about 25 nm to about 300 nm; exhibit stability (colloidal stability); have single cell binding specification to the substantial exclusion of non-targeted cells; are neutral or cationic for specific targeting (for example, cationic); are optionally modified with agents such as PEI, NMe3+, dye, crosslinker, ligands (ligands provide neutral charge); and optionally, are used in combination with a cargo to be delivered to a targeted cell.

In certain embodiments, the MSNPs are monodisperse and range in size from about 25 nm to about 300 nm. The sizes used, for example, include 50 nm (+/−10 nm) and 150 nm (+1-15 nm), within a narrow monodisperse range, but may be more narrow in range. A broad range of particles is not used because such a population is difficult to control and to target specifically.

Illustrative examples of a cationic surfactant include, but are not limited to, cetyl trimethylammonium bromide (CTAB), dodecylethydimethylammonium bromide, cetylpyridinium chloride (CPC), polyethoxylated tallow amine (POEA), hexadecyltrimethylammonium p-toluenesulfonate, benzalkonium chloride (BAC), or benzethonium chloride (BZT).

Poloxamers such as F127 are difunctional block copolymer surfactants terminating in primary hydroxyl groups. They are composed of a central hydrophobic chain of polyoxypropylene (poly(propylene oxide)) flanked by two hydrophilic chains of polyoxyethylene (poly(ethylene oxide)). Because the lengths of the polymer blocks can be customized, many different poloxamers exist having slightly different properties. For the generic term “poloxamer”, these copolymers are commonly named with the letter “P” (for poloxamer) followed by three digits, the first two digits.times.100 give the approximate molecular mass of the polyoxypropylene core, and the last digit.times.10 gives the percentage polyoxyethylene content (e.g., P407=Poloxamer with a polyoxypropylene molecular mass of 4,000 g/mol and a 70% polyoxyethylene content). For the Pluronic® tradename, coding of these copolymers starts with a letter to define it's physical form at room temperature (L=liquid, P=paste, F=flake (solid)) followed by two or three digits, the first digit(s) refer to the molecular mass of the polyoxypropylene core (determined from BASF's PluronicdD grid) and the last digit.times.10 gives the percentage polyoxyethylene content (e.g., F127 is a Pluronic® with a polyoxypropylene molecular mass of 4,000 g/mol and a 70% polyoxyethylene content). In the example given, poloxamer 407 (P407) is Pluronic® F127.

Targeting ligands which may be used to target cells include but are not limited to peptides, affibodies and antibodies (including monoclonal and/or polyclonal antibodies). In certain embodiments, targeting ligands selected from the group consisting of Fcγ from human IgG (which binds to Fcγ receptors on macrophages and dendritic cells), human complement C3 (which binds to CR1 on macrophages and dendritic cells), ephrin B2 (which binds to EphB4 receptors on alveolar type II epithelial cells), and the SP94 peptide (which binds to unknown receptor(s) on hepatocyte-derived cells).

In some embodiments, targeting moieties induce protocell binding to virally-infected host cells. In some embodiments, the targeting moiety targets a virally-infected host cell. In some embodiments, the targeting moiety specifically targets a host cell surface molecule specifically present during a viral infection.

In some embodiments, the protocells described herein further comprise an endosomolytic moiety. After binding to a virally-infected host cell, the protocells described herein are internalized by the host cell. The endosomolytic moiety promotes escape of the antiviral cargo into the host cell, where it can promote death of the intracellular bacteria. In some embodiments, the endosomolytic moiety ruptures a virally-infected cell membrane ruptures acidic intracellular vesicles of the virally-infected host cell. In some embodiments, the endosomolytic moiety is a peptide. In some embodiments, the endosomolytic moiety is octaarginine (R8), H5WYG, Penetratin-HA2, modified HA2-TAT, 43E or Histidine 10.

The charge is controlled based on what is to be accomplished (via PEI, NMe3+, dye, crosslinker, ligands, etc.), but for targeting the charge is, for example, cationic. Charge also changes throughout the process of formation. Initially the targeted particles are cationic and are often delivered as cationically charged nanoparticles, however post modification with ligands they are closer to neutral. The ligands which find use in the present invention include peptides, affibodies and antibodies, among others. These ligands are site specific and are useful for targeting specific cells which express peptides to which the ligand may bind selectively to targeted cells.

MSNPs may be used to deliver cargo to a targeted cell, including, for example, cargo component selected from the group consisting of at least one polynucleotide, such as double stranded linear DNA, minicircle DNA, naked DNA or plasmid DNA, messenger RNA, small interfering RNA, small hairpin RNA, microRNA, a polypeptide, a protein, a drug (in particular, an antibiotic drug), an imaging agent, or a mixture thereof. The MSNPs present are effective for accommodating cargo which are long and thin (e.g., naked) in three-dimensional structure, such as polynucleotides (e.g., various DNA and RNA) and polypeptides.

In protocells, a PEGylated lipid bi- or multilayer encapsulates a population of MSNPs as described herein and comprises (1) a PEGylated lipid which is optionally-thiolated (2) at least one additional lipid and, optionally (3) at least one targeting ligand which is conjugated to the outer surface of the lipid bi- or multilayer and which is specific against one or more receptors of virally-infected cells.

Protocells are highly flexible and modular. High concentrations of physiochemically-disparate molecules can be loaded into the protocells and their therapeutic and/or diagnostic agent release rates can be altered without altering the protocell's size, size distribution, stability, or synthesis strategy. Properties of the supported lipid bi- or multilayer and mesoporous silica nanoparticle core can also be modulated independently, thereby altering properties as surface charge, colloidal stability, and targeting specificity independently from overall size, type of cargo(s), loading capacity, and release rate.

Exemplary Anti-Microbial Protocels

In the anti-viral uses, silencing of a target gene will result in a reduction in “viral titer” in the cell or in the subject. As used herein, “reduction in viral titer” refers to a decrease in the number of viable virus produced by a cell or found in an organism undergoing the silencing of a viral target gene. Reduction in the cellular amount of virus produced may lead to a decrease in the amount of measurable virus produced in the tissues of a subject undergoing treatment and a reduction in the severity of the symptoms of the viral infection.

Nipah virus (NiV), a highly pathogenic member of the Paramyxoviridae family, was first isolated and identified after a 1998-1999 outbreak of fatal encephalitis among pig farmers and abattoir workers in Southeast Asia. NiV and its dose relative, Hendra virus, have been classified as Biosafety Level 4 (BSL-4) select agents due to their broad host range, their numerous routes of transmission, and the high rates of mortality associated with infection. Despite recent advances in understanding the cellular tropism of NiV, there is currently no prophylaxis available for animals or humans, and treatment remains primarily supportive.

The Hendra (Hev) and Nipah Viruses (NiV) are recently emerged zoonotic pathogens of the family Paramyxoviridae. Found naturally in bats, they have a wide host range and can infect humans as well as a number of other animal species. Hendra was discovered in Australia in 1994 when it killed 13 horses and their human trainer near Brisbane, Australia. The closely related virus called Nipah appeared in 1999 as an infection of pigs in Malaysia. It resulted in the culling of about a million pigs, but is known also to have caused 257 human infections, 105 of them resulting in death. The henipaviruses are considered important emerging natural pathogens and potential bioweapons. Both viruses are characterized by a pleomorphic, enveloped virion ranging in size from 40 to 600 nm, and containing a single-stranded negative sense RNA genome about 18.2 kb in length. The lipid envelope is decorated with an attachment protein (called G-protein) and a fusion protein (the F-protein). The literature reports the isolation of a number of monoclonal antibodies capable of neutralizing NiV through binding of the G-protein.

NiV glycoprotein (G) binds to ephrin B2 and ephrin B3, while NiV fusion protein (F) induces macropinocytosis. Other NiV proteins include RNA polymerase (L), matrix protein (M), nucleocapsid protein (N) and phosphoprotein (P).

Non-limiting examples of NiV M siRNA and NiV N siRNA sequences include the following sequences (all sequences presented 5′ to 3):

Niv-N siRNA Sequences:

(SEQ ID NO: 8)
CUG CUC UGC CUU UAG CAG AUC CUC CUU-Antisense
(SEQ ID NO: 9)
GGA GGA UCU GCU AAA GGC AGA GCA G-Sense
(SEQ ID NO: 10)
GCU GGU ACA AAU AUC CUU AUC UUG GUU-Antisense
(SEQ ID NO: 11)
CCA AGA UAA GGA UAU UUG UAC CAG C-Sense
(SEQ ID NO: 12)
GAA UCC UGC CAU ACC AGU UUC CUC GAC-Antisense
(SEQ ID NO: 13)
CGA GGA AAC UGG UAU GGC AGG AUT C-Sense
(SEQ ID NO: 14)
CUU GAG UUC UGU UGC UGA UUG CUG GAU-Antisense
(SEQ ID NO: 15)
CCA GCA AUC AGC AAC AGA ACU CAA G-Sense

Niv-M siRNA Sequences:

(SEQ ID NO: 16)
AAA UAU UCU CAG AGC UUG AUG CUU GUC-Antisense
(SEQ ID NO: 17)
CAA GCA UCA AGC UCU GAG AAU AUT T-Sense
(SEQ ID NO: 18)
CCA GAA UCA UUG AGC UUU GUG AUA CUG-Antisense
(SEQ ID NO: 19)
GUA UCA CAA AGC UCA AUG AUU CUG G-Sense
(SEQ ID NO: 20)
AUC UUC UUG CGU UUC CCU GUC UCU GGG-Antisense
(SEQ ID NO: 21)
CAG AGA CAG GGA AAC GCA AGA AGA T-Sense
(SEQ ID NO: 22)
ACC ACU AGU CAG UAC UUU CUU CCA CGG-Antisense
(SEQ ID NO: 23)
GUG GAA GAA AGU ACU GAC UAG UGG T-Sense

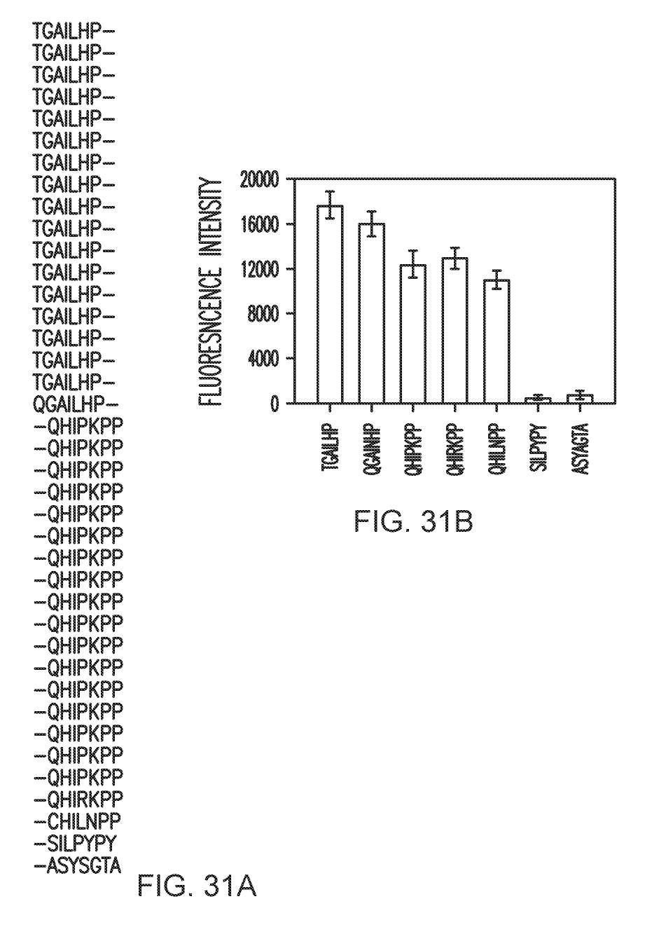

Non-limiting examples of targeting moieties that target ephrin B2 and/or ephrin B3 include the ephrin B2-targeting peptide sequences identified in FIG. 31. TGAILHP is an example of an ephrin B2-targeting peptide sequence. Other targeting moieties include, but are not limited to, antibodies or antibody fragments that bind ephrin B2 or ephrin B3. In some embodiments, the targeting moiety binds any other surface molecule on a virally-infected host cell.

Exemplary Antiviral Protocell Compositions

Ribavirin is a nucleoside-based, anti-metabolite prodrug that exerts a mutagenic effect on RNA viruses by facilitating G-to-A and C-to-U nucleotide transitions (Dietz et al., 2013). It has broad in vitro activity against RNA viruses and is a component of the FDA-approved treatment for chronic hepatitis C infection. Ribavirin has also been shown to have IC₅₀ values in the low micromolar range for several alphaviruses (Huggins et al., 1984; Sindac et al., 2012). We use protocells to improve its circulation half-life (currently <48 hours for a single dose) and to concentrate it in the CNS.

Nucleic acids, including siRNAs and artificial microRNAs, that target highly conserved regions of divergent VEEV strains have demonstrated in vitro efficacy (Steele et al., 2010; Diamond, 2009). Furthermore, since complete genome sequences are readily available for multiple alphaviruses, novel nucleic acid-based antivirals that target viral genes can be designed using web-based tools. Published siRNA sequences that target conserved regions of VEEV RNA-dependent RNA polymerase (RdRp), as well as nsp1 and E1 glycoprotein genes, can be used in our protocells (Bhomia et al., 2013; O'Brien, 2007). Since siRNAs for EEEV have not been reported, computational sequence analysis and siRNA design software can be used to generate novel siRNAs that target similar conserved sequences as described for VEEV.

In some embodiments, protocells have enhanced blood-brain barrier (BBB) penetration, which enables them to deliver antivirals to the cytosol of target cells in the central nervous system (CNS).

Broadly cross-reactive neutralizing monoclonal antibodies and antibody fragments have been reported to protect mouse models of VEEV infection upon post-exposure treatment (Goodchild et al., 2011; O'Brien et al., 2012). The therapeutic window for post-exposure antibody treatment is narrow, however. Therefore, protocells can be used to deliver therapeutic antibody fragments (F(ab′)₂ or scFvs) to the CNS in order to prevent neuronal virus spread and increase the therapeutic window; these antibody fragments have been shown to neutralize several VEEV subtypes (IA/B, IE) and EEEV (O'Brien et al., 2012; Rulker et al., 2012).

VEEV Strains.

The VEEV vaccine strain TC-83 can used in identifying useful antivirals; TC-83 is a live attenuated, licensed veterinary vaccine and is used to immunize horses in regions endemic for IAB and IC strains, as well as laboratory workers and military personnel. TC-83 was generated by 83 serial passages of the Trinidad donkey (TrD) IAB strain in guinea pig heart cells (Berge et al., 1961). Use the fully-virulent 3908 and TrD strains. TrD (subtype IA/B) was isolated in 1943 and can cause severe, often fatal infections in horses and humans (Weaver et al., 1999). 3908 is an epidemic subtype IC strain and was isolated in 1995 from a febrile human during a major epidemic in Venezuela (Weaver et al., 1996).

EEEV Strain.

The North American EEEV strain FL93-939 can be used in identifying useful antivirals. This strain was isolated from a pool of Culiseta melanura mosquitoes collected in Florida during 1993 and was passaged once in Vero cells. Virus stocks were prepared from BHK-21 cell cultures (White et al., 2011).

Disease Progression of Alphaviral Encephalitis in Mouse Models.

New World alphaviruses are naturally transmitted to vertebrate hosts through the bite of infected mosquitoes. In humans, cardinal features of the more severe consequences of VEE include a biphasic febrile illness with CNS manifestations and damage to lymphoid tissues (Steele et al., 2010). Mouse models of VEEV infection mimic both encephalitis and lymphotropism of human disease. Natural mosquito-transmitted infection is modeled by subcutaneous inoculation of mice with virulent strains of VEEV. Using this route of infection, virus first moves to the draining lymph nodes via dermal dendrilic cells and begins to replicate by about 4 hours post-infection (PI). Viremia begins about 12 hours PI and causes systemic infection with a strong tropism for lymphoid tissue, including the spleen. GALT, thymus, and bone marrow. VEEV then enters the CNS through the olfactory bulbs and begins to replicate in the brain by 36-48 hours PI.

More pertinent to biodefense is the aerosol route of inoculation, where VEEV moves directly through olfactory neurons to the CNS without causing viremia. Once in the CNS, neurons are the primary target of viral infection in the brain and spinal cord, resulting in the majority of clinical symptoms of VEE in mice and the near-uniform lethality, which occurs between 6-9 days PI for virulent strains of VEEV. Similar to VEE in mice and mimicking aspects of EEE in humans, young mice infected with EEEV develop a biphasic disease course that manifests as initial virus replication in peripheral tissues followed by viremia, CNS invasion, and encephalitis. Mice typically die 4-6 days after peripheral inoculation with EEEV.

Disease Progression of Alphaviral Encephalitis in Hamster Models.

Intraperitoneal inoculation of VEEV produces different symptoms in hamsters than in mice (Jackson et al., 1991). In the hamster model, VEEV causes acute, fulminant disease typified by massive necrosis of lymphoid tissues (especially the GALT), and animals often die before the onset of CNS disease. Since VEEV does not cause encephalitis in hamsters via peripheral routes of infection, we will challenge hamsters with aerosolized VEEV. Although the pathology of aerosol VEEV infection in hamsters has not been well described in the literature, aerosol inoculation of virulent VEEV and EEEV strains cause neurological disease in mice and NHPs; we, therefore, anticipate a similar progression of neurological disease in the hamster model (Steele et al., 2010). Similar to mice, hamsters inoculated peripherally with EEEV develop an early visceral phase, which is accompanied by viremia and followed by neuroinvasion and death due to encephalitis about 4-6 days PI (Paessler et al., 2004). EEEV enters the brain about 2 days PI and replicates progressively thereafter, reaching titers that grossly exceed those in other tissues. The appearance of virus in multiple regions of the brain at the same time suggests entry from the vasculature. Histopathological features of EEE in the brains of hamsters include neuronal tropism and necrosis, and inflammatory cell infiltration.

Disease Progression of Alphaviral Encephalitis in NHP Models.

Most VEE studies involving NHPs have used Cynomolgus macaques (Cynos) (Steele et al., 2010). Key features of VEE in Cynos include the development of fever, viremia, and lymphopenia within 1-3 days of infection and signs of encephalitis 3-6 days after infection; most animals recover from VEE, however (Reed et al., 2005; Reed et al., 2004). In contrast, Cynos challenged with aerosolized EEEV develop fever, elevated white blood cell counts and liver enzymes, and major CNS changes, including severe meningoencephalomyelitis, widespread neuronal necrosis, and the presence of perivascular cuffs, cellular debris, gliosis, satelitosis, edema, and hemorrhage, causing the majority of animals to succumb 5-9 days after infection (Reed et al., 2007). Taken together, these studies suggest that NHP models develop key features of EEE in humans, such as neuronal tropism and necrosis, meningoencephalitis, and vascular damage.

In exemplary embodiments, pharmaceutical formulations and protocells can also be used in the treatment of an infection caused by dengue virus: yellow fever virus; West Nile virus; Japanese encephalitis virus; HIV; HTLV-I, Bunyaviridae viruses including the hantaviruses, Crimean-Congo hemorrhagic fever, Rift Valley fever virus, and fever and severe fever and thrombocytopenia virus; arenaviruses including al agents of South American hemorrhagic fever, Lassa virus and lymphocytic choriomeningitis virus; filoviruses including Ebola and Marburg viruses; paramyxoviruses including morbilliviruses, henipaviruses, respiroviruses including RSV and metapneumovirus and rubellaviruses: Alphaviruses including Chikungunya, O'nyung-nyung, Semliki Forest, Ross River, Sindbis, eastern, western and Venezuelan equine encephalitis; picornaviruses; papillomaviruses including HPV; herpesviruses including HSV-1/2, EBV, CMV, HHV-6, 7, and 8; polyomaviruses including SV40, JC and BK viruses; poxviruses including variola and vaccinia viruses.

In certain embodiments, the anti-viral pharmaceutical formulations and protocels can be used in the treatment of subject who is infected by a Hendra virus, a Nipah virus (NiV), a Group A arbovirus (Alphavirus of the Togavirus family) including Eastern equine encephalitis (EEEV) or a Venezuelan equine encephalitis (VEEV) and who has not responded successfully to treatment with ribavirin.

In addition to, or as an alternative to, the therapeutic nucleic acid cargo described herein, pharmaceutical formulations and protocells can also contain one or more antiviral agents including, but not limited to anti-HIV agents including, for example, nucleoside reverse transcriptase inhibitors (NRTI), non-nucleoside reverse transcriptase inhibitors (NNRTI), protease inhibitors, fusion inhibitors, among others, exemplary compounds of which may include, for example, 3TC (Lamivudine), AZT (Zidovudine), (−)-FTC, ddl (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), fusion inhibitors such as T20, among others, fuseon and mixtures thereof, including anti-HIV compounds presently in clinical trials or in development, anti-HBV agents including, for example, hepsera (adefovir dipivoxil), lamivudine, entecavir, telbivudine, tenofovir, emtricitabine, clevudine, valtorcitabine, amdoxovir, pradefovir, racivir, BAM 205, nitazoxanide, UT 231-B, Bay 41-4109. EHT899, zadaxin (thymosin alpha-1) and mixtures thereof, and anti-HCV agents including, for example, interferon, pegylated interferon, ribavirin, NM 283, VX-950 (telaprevir). SCH 50304, TMC435, VX-500, BX-813, SCH503034, R1626, ITMN-191 (R7227), R7128, PF-868554, TT033, CGH-759, GI 5005. MK-7009, SIRNA-034, MK-0608, A-837093, GS 9190, ACH-1095, GSK625433, TG4040 (MVA-HCV), A-831, F351, NS5A, NS4B, ANA598, A-689, GNI-104, IDX102, ADX184, GL59728, GL60667, PSI-7851, TLR9 Agonist, PHX1766, SP-30 and mixtures thereof.

Typically pharmaceutical formulations and protocells can be loaded with cargo to a capacity up to about 10, 20, 30, 40, 50, 60, 70, 80 or about 90 weight % or more (or from about 0.01% to about 70%, about 0.02% to about 60%, about 0.2 to about 55%, about 0.5% to about 45%, about 1% to about 35%, about 1.5% to about 25%, about 0.1% to about 10%, about 0.01% to about 5%): defined as (cargo weight/weight of loaded protocell)×100. The optimal loading of cargo is often about 0.01 to 60% but this depends on the drug or drug combination which is incorporated as cargo into the MSNPs. This is generally expressed in μM per 10¹⁰ particles where we have values ranging from 2000-100 μM per 10¹⁰ particles. For example, MSNPs exhibit release of cargo at pH about 5.5, which is that of the endosome, but are stable at physiological pH of 7 or higher (7.4).

The surface area of the internal space for loading is the pore volume whose optimal value ranges from about 1.1 to 0.5 cubic centimeters per gram (cc/g). Note that in the MSNPs according to one embodiment, the surface area is mainly internal as opposed to the external geometric surface area of the nanoparticle.

The lipid bi- or multilayer supported on the porous particle according to one embodiment has a lower melting transition temperature, i.e., is more fluid than a lipid bi- or multilayer supported on a non-porous support or the lipid bi- or multilayer in a liposome. This is sometimes important in achieving high affinity binding of immunogenic peptides or targeting ligands at low peptide densities, as it is the bilayer fluidity that allows lateral diffusion and recruitment of peptides by target cell surface receptors. One embodiment provides for peptides to duster, which facilitates binding to a complementary target.

The lipid bi- or multilayer may vary significantly in composition. Ordinarily, any lipid or polymer which may be used in liposomes may also be used in MSNPs. For example, lipids are as otherwise described herein.

In some embodiments according, the lipid bi- or multilayer of the protocells can provide biocompatibility and can be modified to possess targeting species including, for example, antigens, targeting peptides, fusogenic peptides, antibodies, aptamers, and PEG (polyethylene glycol) to allow, for example, further stability of the protocels and/or a targeted delivery into a cell to maximize an immunogenic response. PEG, when included in lipid bilayers, can vary widely in molecular weight (although PEG ranging from about 10 to about 100 units of ethylene glycol, about 15 to about 50 units, about 15 to about 20 units, about 15 to about 25 units, about 16 to about 18 units, etc., may be used) and the PEG component which is generally conjugated to phospholipid through an amine group comprises about 1% to about 20%, for example, about 5% to about 15%, about 10% by weight of the lipids which are included in the lipid bi- or multilayer. The PEG component is generally conjugated to an amine-containing lipid such as DOPE or DPPE or other lipid, but in alterative embodiments may also be incorporated into the MSNPs, through inclusion of a PEG containing silane.

Numerous lipids which are used in liposome delivery systems may be used to form the lipid bi- or multilayer on nanoparticles. Virtually any lipid which is used to form a liposome may be used in the lipid bi- or multilayer which surrounds the nanoparticles according to an embodiment. For example, lipids for use in the present invention include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]auroyl]-sn-glycero)-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment given the fact that cholesterol may be an important component of the lipid bilayer of protocells according to an embodiment. Often cholesterol is incorporated into lipid bilayers of protocells in order to enhance structural integrity of the bilayer. These lipids are all readily available commercially from Avanti Polar Lipids, Inc. (Alabaster, Ala., USA). DOPE and DPPE are particularly useful for conjugating (through an appropriate crosslinker) PEG, peptides, polypeptides, including immunogenic peptides, proteins and antibodies, RNA and DNA through the amine group on the lipid.

MSNPs and protocells can be PEGylated with a variety of polyethylene glycol-containing compositions as described herein. PEG molecules can have a variety of lengths and molecular weights and include, but are not limited to, PEG 200, PEG 1000, PEG 1500, PEG 4600, PEG 10,000, PEG-peptide conjugates or combinations thereof.

Pharmaceutical compositions comprise an effective population of MSNPs and/or protocells as otherwise described herein formulated to effect an intended result (e.g., immunogenic result, therapeutic result and/or diagnostic analysis, including the monitoring of therapy) formulated in combination with a pharmaceutically acceptable carrier, additive or excipient. The MSNPs and/or protocells within the population of the composition may be the same or different depending upon the desired result to be obtained. Pharmaceutical compositions may also comprise an additional bioactive agent or drug, such as an antiviral agent.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g., orally, transdermally, perineurally or parenteraly, that is, by intravenous, subcutaneous, intraperitoneal, intrathecal or intramuscular injection, among others, including buccal, rectal and transdermal administration. Subjects contemplated for treatment according to the method include humans, companion animals, laboratory animals, and the like. The invention contemplates immediate and/or sustained/controlled release compositions, including compositions which comprise both immediate and sustained release formulations. This is particularly true when different populations of MSNPs and/or protocells are used in the pharmaceutical compositions or when additional bioactive agent(s) are used in combination with one or more populations of protocells as otherwise described herein.

Formulations containing the compounds may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like. In one embodiment, in unit dosage forms suitable for simple administration of precise dosages.

Pharmaceutical compositions typically include a conventional pharmaceutical carrier or excipient and may additionally include other medicinal agents, carriers, adjuvants, additives and the like. In one embodiment, the composition is about 0.1% to about 85%, about 0.5% to about 75% by weight of a compound or compounds, with the remainder consisting essentially of suitable pharmaceutical excipients.

An injectable composition for parenteral administration (e.g., intravenous, intramuscular or intrathecal) will typically contain the compound in a suitable i.v. solution, such as sterile physiological salt solution. The composition may also be formulated as a suspension in an aqueous emulsion.

Liquid compositions can be prepared by dissolving or dispersing the population of MSNPs and/or protocels (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g., an excipient such as a saccharide or cellulose preparation, a binder such as starch paste or methyl cellulose, a filler, a disintegrator, and other additives typically used in the manufacture of medical preparations.

Methods for preparing such dosage forms are known or is apparent to those skilled in the art; for example, see Remington's Pharmaceutical Sciences (17th Ed., Mack Pub. Co., 1985). The composition to be administered will contain a quantity of the selected compound in a pharmaceutically effective amount for therapeutic use in a biological system, including a patient or subject.

Methods of treating patients or subjects in need for a particular disease state or infection comprise administration an effective amount of a pharmaceutical composition comprising therapeutic MSNPs and/or protocells and optionally at least one additional bioactive (e.g., antiviral, antibiotic, and/or antimicrobial) agent.

Intranasal (IN) delivery of broad-spectrum small molecule, nucleic acid, and antibody-based antivirals to central nervous system (CNS) tissues and cells infected with encephalitic New World alphaviruses (e.g., Venezuelan (VEEV), eastern (EEEV), and western (WEEV) equine encephalitis viruses) illustrates in one embodiment treatment modality.

Diagnostic methods may comprise administering to a patient in need an effective amount of a population of diagnostic MSNPs and/or protocels (e.g., MSNPs and/or protocells which comprise a target species, such as a targeting peptide which binds selectively to virally-infected cells and a reporter component to indicate the binding of the protocells) whereupon the binding of the MSNPs and/or protocells to cells as evidenced by the reporter component (moiety) will enable a diagnosis of the existence of a disease state in the patient.

An alternative of the diagnostic method may be used to monitor the therapy of a disease state in a patient, the method comprising administering an effective population of diagnostic MSNPs and/or protocels (e.g., MSNPs and/or protocels which comprise a target species, such as a targeting peptide which binds selectively to target cells and a reporter component to indicate the binding of the protocells to virally-infected cells if such cells are present) to a patient or subject prior to treatment, determining the level of binding of diagnostic protocells to target cells in said patient and during and/or after therapy, determining the level of binding of diagnostic protocels to target cells in said patient, whereupon the difference in binding before the start of therapy in the patient and during and/or after therapy will evidence the effectiveness of therapy in the patient, including whether the patient has completed therapy or whether the disease state has been inhibited or eliminated.

In accordance with the present disclosure may be employed conventional molecular biology, microbiology, and recombinant DNA techniques within the skill of the art. Such techniques are explained fully in the literature. See, e.g., Sambrook at al, 2001, “Molecular Cloning: A Laboratory Manual”; Ausubel, ed., 1994, “Current Protocols in Molecular Biology” Volumes I-III: Cells, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL.

Any number of histone proteins, as well as other means to package the DNA into a smaller volume such as normally cationic nanoparticles, lipids, or proteins, may be used to package the supercoiled plasmid DNA “histone-packaged supercoiled plasmid DNA”, but in therapeutic aspects which relate to treating human patients, the use of human histone proteins are for example used. In certain aspects, a combination of human histone proteins H1, H2A, H2B, H3 and H4 in one embodiment ratio of 1:2:2:2:2, although other histone proteins may be used in other, similar ratios, as is known in the art or may be readily practiced pursuant to the teachings. The DNA may also be double stranded linear DNA, instead of plasmid DNA, which also may be optionally supercoiled and/or packaged with histones or other packaging components.

Other histone proteins which may be used in this aspect include, for example, H1F, H1F0, H1FNT, H1FOO, H1FX H1H1 HIST1H1A, HIST1H1B, HIST1H1C, HIST1H1D, HIST1H1E, HIST1H1T, H2AF, H2AFB1, H2AFB2, H2AFB3, H2AFJ, H2AFV, H2AFX, H2AFY, H2AFY2, H2AFZ, H2A1, HIST1H2AA, HIST1H2AB, HIST1H2AC, HIST1H2AD, HIST1H2AE, HIST1H2AG, HIST1H2AI, HIST1H2AJ, HIST1H2AK, HIST1H2AL, HIST1H2AM, H2A2, HIST2H2AA3, HIST2H2AC, H2BF, H2BFM, HSBFS, HSBFWT, H2B1, HIST1H2BA, HIST1HSBB, HIST1HSBC, HIST1HSBD, HIST1H2BE, HIST1H2BF, HIST1H2BG, HIST1H2BH, HIST1H2BI, HIST1H2BJ, HIST1H2BK, HIST1H2BL, HIST1H2BM, HIST1H2BN, HIST1H2BO, H2B2, HIST2H2BE, H3A1, HIST1H3A, HIST1H3B, HIST1H3C, HIST1H3D, HIST1H3E, HIST1H3F, HIST1H3G, HIST1H3H, HIST1H31, HIST1H3J, H3A2, HIST2H3C, H3A3, HIST3H3, H41, HIST1H4A, HIST1H4B, HIST1H4C, HIST1H4D, HIST1H4E, HIST1H4F, HIST1H4G, HIST1H4H, HIST1H41, HIST1H4J, HIST1H4K, HIST1H4L, H44 and HIST4H4.

Proteins gain entry into the nucleus through the nuclear envelope. The nuclear envelope consists of concentric membranes, the outer and the inner membrane. These are the gateways to the nucleus. The envelope consists of pores or large nuclear complexes. A protein translated with a NLS will bind strongly to importin (aka karyopherin), and together, the complex will move through the nuclear pore. Any number of nuclear localization sequences may be used to introduce histone-packaged plasmid DNA into the nucleus of a cell. Exemplary nuclear localization sequences include H₂N-GNQSSNFGPMKGGNFGGRSSGPYGGGGQYFAKPRNQGGYGGC-COOH, RRMKWKK, PKKKRKV, and KR[PAATKKAGQA]KKKK (SEQ ID NOs: 24-27, the NLS of nucleoplasmin, a prototypical bipartite signal comprising two clusters of basic amino acids, separated by a spacer of about 10 amino acids. Numerous other nuclear localization sequences are well known in the art. See LaCasse et al., (1995); Weis, (1998); and Murat Cokol et al., at the website ubic.bioc.columbia.edulpapers/2000 nls/paper.html#tab2.

A peptide nucleic acid can consist of repeating N-(2-aminoethyl)-glycine units linked by amide bonds. The purine (A, G) and pyimidine (C, T) bases are attached to the backbone through methylene carbonyl linkages. Unlike DNA or DNA analogs, PNAs do not contain any (pentose) sugar moieties or phosphate groups. Surprisingly, PNA's in many respects mimic the behavior of DNA, and in some applications demonstrate superior properties. By convention, PNAs are depicted like peptides, with the N-terminus at the (left) position and the C-terminus at the right. Besides the obvious structural difference, PNA is set apart from DNA in that the backbone of PNA is acyclic, achiral and neutral. PNAs can bind to complementary nucleic acids in both antiparallel and parallel orientation. However, the antiparallel orientation is strongly in one embodiment, and the parallel duplex has been shown to have a different structure. Nielsen, et al., “An Introduction to Peptide Nucleic Acid”, Current Issues Molec. Biol. (1999) 1(2): 89-104.

A level and/or an activity and/or expression of a translation product of a gene and/or of a fragment, or derivative, or variant of said translation product, and/or the level or activity of said translation product, and/or of a fragment, or derivative, or variant thereof, can be detected using an immunoassay, an activity assay, and/or a binding assay. These assays can measure the amount of binding between said protein molecule and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Immunoassays which can be used include, e.g., ELISAs, Western blots and other techniques known to those of ordinary skill in the art (see Harlow and Lane, Antibodies: A Laboratory Manual, Cold Spring Harbor Laboratory Press, Cold Spring Harbor. N.Y., 1999 and Edwards R, Immunodiagnostics: A Practical Approach, Oxford University Press, Oxford; England, 1999). All these detection techniques may also be employed in the format of microarrays, protein-arrays, antibody microarrays, tissue microarrays, electronic biochip or protein-chip based technologies (see Schena M., Microarray Biochip Technology, Eaton Publishing, Natick, Mass., 2000).

Certain diagnostic and screening methods utilize an antibody, for example, a monoclonal antibody, capable of specifically binding to a protein as described herein or active fragments thereof. The method of utilizing an antibody to measure the levels of protein allows for non-invasive diagnosis of the pathological states of kidney diseases. In one embodiment, the antibody is human or is humanized. In one embodiment, antibodies may be used, for example, in standard radioimmunoassays or enzyme-linked immunosorbent assays or other assays which utilize antibodies for measurement of levels of protein in sample. In a particular embodiment, the antibodies are used to detect and to measure the levels of protein present in a sample.

Humanized antibodies are antibodies, or antibody fragments, that have the same binding specificity as a parent antibody, (e.g., typically of mouse origin) and increased human characteristics. Humanized antibodies may be obtained, for example, by chain shuffling or by using phage display technology. For example, a polypeptide comprising a heavy or light chain variable domain of a non-human antibody specific for a disease related protein is combined with a repertoire of human complementary (light or heavy) chain variable domains. Hybrid pairings specific for the antigen of interest are selected. Human chains from the selected pairings may then be combined with a repertoire of human complementary variable domains (heavy or light) and humanized antibody polypeptide dimers can be selected for binding specificity for an antigen. Techniques described for generation of humanized antibodies that can be used in the method are disclosed in, for example, U.S. Pat. Nos. 5,565,332; 5,585,089; 5,694,761; and 5,693,762. Furthermore, techniques described for the production of human antibodies in transgenic mice are described in, for example, U.S. Pat. Nos. 5,545,806 and 5,569,825.

In order to identify small molecules and other agents useful in the present methods for treating a viral infection by modulating the activity and expression of a disease-related protein and biologically active fragments thereof can be used for screening therapeutic compounds in any of a variety of screening techniques. Fragments employed in such screening tests may be free in solution, affixed to a solid support, bore on a cell surface, or located intracellularly. The blocking or reduction of biological activity or the formation of binding complexes between the disease-related protein and the agent being tested can be measured by methods available in the art.

Other techniques for drug screening which provide for a high throughput screening of compounds having suitable binding affinity to a protein, or to another target polypeptide useful in modulating, regulating, or inhibiting the expression and/or activity of a disease, are known in the art. For example, microarrays carrying test compounds can be prepared, used, and analyzed using methods available in the art. See, e.g., Shalon, D. et al., 1995, International Publication No. WO95/35505, Baldeschweiler et al., 1995, International Publication No. WO95/251116; Brennan et al., 1995, U.S. Pat. No. 5,474,796; Heller et al., 1997, U.S. Pat. No. 5,605,662.

To determine specific binding, various immunoassays may be employed for detecting, for example, human or primate antibodies bound to the cells. Thus, one may use labeled anti-hlg, e.g., anti-hlgM, hlgG or combinations thereof to detect specifically bound human antibody. Various labels can be used such as radioisotopes, enzymes, fluorescers, chemiluminescers, particles, etc. There are numerous commercially available kits providing labeled anti-hlg, which may be employed in accordance with the manufacturer's protocol.

In one embodiment, a kit can comprise: (a) at least one reagent which is selected from the group consisting of (i) reagents that detect a transcription product of the gene coding for a protein marker as described herein (ii) reagents that detect a translation product of the gene coding for proteins, and/or reagents that detect a fragment or derivative or variant of said transcription or translation product; (b) instructions for diagnosing, or prognosticating a disease, or determining the propensity or predisposition of a subject to develop such a disease or of monitoring the effect of a treatment by determining a level, or an activity, or both said level and said activity, and/or expression of said transcription product and/or said translation product and/or of fragments, derivatives or variants of the foregoing, in a sample obtained from said subject; and comparing said level and/or said activity and/or expression of said transcription product and/or said translation product and/or fragments, derivatives or variants thereof to a reference value representing a known disease status (patient) and/or to a reference value representing a known health status (control) and/or to a reference value; and analyzing whether said level and/or said activity and/or expression is varied compared to a reference value representing a known health status, and/or is similar or equal to a reference value representing a known disease status or a reference value; and diagnosing or prognosticating a disease, or determining the propensity or predisposition of said subject to develop such a disease, wherein a varied or altered level, expression or activity, or both said level and said activity, of said transcription product and/or said translation product and/or said fragments, derivatives or variants thereof compared to a reference value representing a known health status (control) and/or wherein a level, or activity, or both said level and said activity, of said transcription product and/or said translation product and/or said fragments, derivatives or variants thereof is similar or equal to a reference value and/or to a reference value representing a known disease stage, indicates a diagnosis or prognosis of a disease, or an increased propensity or predisposition of developing such a disease, a high risk of developing signs and symptoms of a disease.

Reagents that selectively detect a transcription product and/or a translation product of the gene coding for proteins can be sequences of various length, fragments of sequences, antibodies, aptamers, siRNA, microRNA, and ribozymes. Such reagents may be used also to detect fragments, derivatives or variants thereof.

Purely by way of example, comparing measured levels of a viral infection biomarker (e.g., viral titer) in a sample to corresponding control levels, or comparing measured viral marker levels to control viral marker levels determined in a healthy control subject, and determining that a subject suffers from a viral infection or that a subject's viral infection is progressing, can include determinations based on comparative level differences of about between about 5-10%, or about 10-15%, or about 15-20%, or about 20-25%, or about 25-30%, or about 30-35%, or about 35-40%, or about 40-45%, or about 45-50%, or about 50-55%, or about 55-60%, or about 60-65%, or about 65-70%, or about 70-75%, or about 75-80%, or about 80-85%, or about 85-90%, or about 90-95%, or about 95-100%, or about 100-110%, or about 110-120%, or about 120-130%, or about 130-140%, or about 140-150%, or about 150-160%, or about 160-170%, or about 170-180%, or about 180-190%, or 190-200%, or 200-210%, or 210-220%, or 220-230%, or 230-240%, or 240-250%, or 250-260%, or about 260-270%, or about 270-280%, or about 280-290%, or about 290-300%, or differences of about between about ±50% to about ±0.5%, or about ±45% to about ±1%, or about ±40% to about ±1.5%, or about ±35% to about ±2.0%, or about ±30% to about ±2.5%, or about ±25% to about ±3.0%, or about ±20% to about ±3.5%, or about ±15% to about ±4.0%, or about ±10% to about ±5.0%, or about ±9% to about ±1.0%, or about ±8% to about ±2%, or about ±7% to about ±3%, or about ±6% to about ±5%, or about ±5%, or about ±4.5%, or about ±4.0%, or about ±3.5%, or about ±3.0%, or about ±2.5%, or about ±2.0%, or about ±1.5%, or about ±1.0%.

Exemplary Anti-Bacterial Protocell Compositions

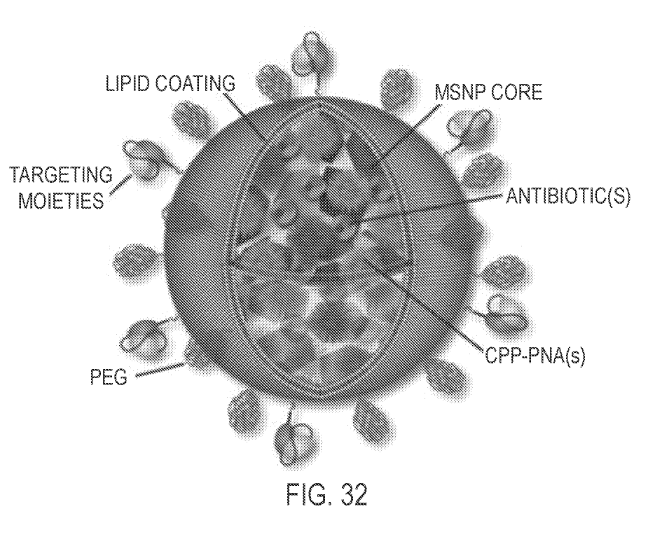

The present disclosure provides protocells comprising a porous nanoparticle core encapsulated by a lipid bilayer or lipid multilayer, a targeting moiety attached to the lipid bilayer, and an antibacterial cargo. In some embodiments, the targeting moiety binds to a bacterial cellular receptor. In some embodiments, the targeting moiety binds to a protein present on the surface of a bacterially-infected host cell. In some embodiments, the antibacterial cargo is an antibiotic. In some embodiments, the antibacterial cargo is a peptide nucleic acid (PNA) or a cell penetrating peptide-peptide nucleic acid (CPP-PNA) conjugate. In some embodiments, the PNA or CPP-PNA inhibits the expression of a bacterial protein that enables antibiotic-resistance to the bacteria. In some embodiments, the antibacterial cargo comprises an antibiotic and a PNA or CPP-PNA that inhibits the expression of a bacterial protein that enables resistance to the antibiotic. A non-limiting, exemplary protocell is depicted in FIG. 32.

The following terms shall be used throughout the specification to describe the present invention. Where a term is not specifically defined herein, that term shall be understood to be used in a manner consistent with its use by those of ordinary skill in the art.

Where a range of values is provided, it is understood that each intervening value, to the tenth of the unit of the lower limit unless the context dearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention. In instances where a substituent is a possibility in one or more Markush groups, it is understood that only those substituents which form stable bonds are to be used.

Unless defined otherwise, all technical and scientific terms used herein have the same meaning as commonly understood by one of ordinary skill in the art to which this invention belongs. Although any methods and materials similar or equivalent to those described herein can also be used in the practice or testing, in one embodiment methods and materials are now described.

It must be noted that as used herein and in the appended claims, the singular forms “a,” “and” and “the” include plural references unless the context dearly dictates otherwise.

Furthermore, the following terms shall have the definitions set out below.

Porous silica particles of varying sizes ranging in size (diameter) from less than 5 nm to 200 nm or 500 nm or more are readily available in the art or can be readily prepared using methods known in the art or alternatively, can be purchased from Melorium Technologies, Rochester, N.Y. Sky Spring Nanomaterials, Inc., Houston, Tex., USA or from Discovery Scientific. Inc., Vancouver, British Columbia. Multimodal silica nanoparticles may be readily prepared using the procedure of Carroll, et al., Langmuir, 25, 13540-13544 (2009).

The examples herein provide various methodologies for obtaining protocells which are useful in the present invention. Useful general techniques include those described in Liu, et al., Chem. Comm., 5100-5102 (2009), Liu, et al., J. Amer. Chem. Soc., 131, 1354-1355 (2009), Liu, et al., J. Amer. Chem. Soc., 131, 7567-7569 (2009) Lu, et al., Nature, 398, 223-226 (1999), Ashley, et al., Nature Materials, 2011. May; 10(5):389-97, Lu, et al., Nature, 398, 223-226 (1999), and Caroll, et al., Langmuir, 25, 13540-13544 (2009).

Nanostructures include a core-shell structure which comprises a porous particle core surrounded by a shell of lipid for example a bilayer, but possibly a monolayer or multiayer (see Liu, et al., J. Amer. Chem. Soc., 131, 7567-7569 (2009)). The porous particle core can include, for example, a porous nanoparticle made of an inorganic and/or organic material as set forth above surrounded by a lipid bilayer.

The porous particle core of the protocells can be loaded with various desired species (“cargo”), including small molecules (e.g., antibacterial agents as otherwise described herein), large molecules (e.g., including macromolecules such as asRNA, siRNA or shRNA or a polypeptide which may include an antibacterial polypeptide or a reporter polypeptide (e.g., fluorescent green protein, among others), semiconductor quantum dots, or metallic nanoparticles, or metal oxide nanoparticles or combinations thereof). Protocells can also be loaded with super-coiled plasmid DNA, which can be used to deliver a therapeutic and/or diagnostic peptide(s) or a small hairpin RNA/shRNA or small interfering RNA/siRNA, which can be used to inhibit expression of proteins associated with antibiotic resistance.

In some embodiments, protocells are comprised of a spherical mesoporous silica nanoparticle (MSNP) core encased within a supported lipid bilayer (SLB) (Ashley et al., 2012; Ashley et al., 2011; Epler et al., 2012). MSNPs have an extremely high surface area (>1200 m²/g), which enables high concentrations of various therapeutic and diagnostic agents to be adsorbed within the core by simple immersion in a solution of the cargo(s) of interest. Furthermore, since the aerosol-assisted evaporation-induced self-assembly (EISA) process (Lu at al., 1999) we pioneered to synthesize MSNPs is compatible with a wide range of structure-directing surfactants and amenable to post-synthesis processing, the overall size can be varied from 20-nm to >10-μm, the pore size can be varied from 2.5-nm to 50-nm, and the naturally negatively-charged pore walls can be modified with a variety of functional moieties, enabling facile encapsulation of physicochemically disparate molecules, including acidic, basic, and hydrophobic drugs, proteins, small interfering RNA, minicircle DNA vectors, plasmids, and diagnostic agents like quantum dots and iron oxide nanoparticles (Ashley et al., 2012; Ashley et al., 2011; Epler et al., 2012).

Protocells have a loading capacity of up to 60 wt % for small molecule drugs, which is 10-fold higher than other MSNP-based delivery vehicles (Meng et al., 2010) and 1000-fold higher than similarly-sized liposomes (Ashley et al., 2011). Release rates can be tailored by controlling the core's degree of silica condensation and, therefore, its dissolution rate under physiological conditions; thermal calcination maximizes condensation and results in particles with sustained release profiles (7-10% release per day for up to 2 weeks), while use of acidified ethanol to extract surfactants enhances particle solubility and results in burst release of encapsulated drugs (100% release within 12 hours). Liposome fusion to cargo-loaded MSNPs results in the formation of a coherent SLB that provides a stable, fluid, biocompatible interface for display of functional molecules, such as polyethylene glycol (PEG) and targeting ligands.

Protocells stably encapsulate small molecule drugs for up to 4 weeks when dispersed in complex biological fluids (e.g., complete growth medium and blood), regardless of whether the SLB is composed of lipids that are fluid or non-fluid at body temperature; in contrast, liposomes rapidly leak their encapsulated drugs, even when their bilayers are composed of fully saturated lipids, which have a high packing density and should, therefore, limit diffusion of drugs across the bilayer. The fluid, yet stable SLB enables us to achieve exquisitely high targeting specificities at low ligand densities, which, in turn, reduces immunogenicity and non-specific interactions; we have shown that protocells modified with an average of just 6 targeting peptides per particle have a 10,000-fold higher affinity for target cells than for non-target cells when the SLB is composed of the fluid, zwitterionic lipid, 1,2-dioleoyl-sn-glycero-3-phosphocholine (DOPC) (Ashley et al., 2011).

Protocells are highly biocompatible and can be engineered for both broad distribution and persistence within target tissues. Balb/c mice injected intravenously (i.v.) with 200 mg/kg doses of PEGylated protocells three times each week for three weeks show no signs of gross or histopathological toxicity. Given their high loading capacity, this result indicates that protocells can deliver at least 900 mg/kg of therapeutic molecules with either burst or sustained release kinetics. Furthermore, PEGylated protocells 20-200 nm in diameter remain broadly distributed for 2-7 days when injected i.v., which provides a sufficient period of time for targeted protocells to accumulate within target tissues, where they can persist for up to 4 weeks with no adverse effects. Additionally, we and others have demonstrated that MSNPs are biodegradable and ultimately excreted in the urine and feces as silicic acid (Lu et al., 2010). Finally, we have shown that protocels modified with up to 10 wt % of targeting ligands induce neither IgG nor IgM responses when injected in C57Bl/6 mice at a total dose of 400 mg/kg. Depending upon the biodistribution required for a specific application, we can control the MSNP size and shape (spherical, disk-shaped, and rod-shaped (Meng at al., 2011) and the SLB charge and surface modification(s), making the protocell a highly modular, flexible nanoparticle delivery system.

A nanoparticle may have a variety of shapes and cross-sectional geometries that may depend, in part, upon the process used to produce the particles. In one embodiment, a nanoparticle may have a shape that is a torus (toroidal). A nanoparticle may include particles having two or more of the aforementioned shapes. In one embodiment, a cross-sectional geometry of the particle may be one or more of toroidal, circular, ellipsoidal, triangular, rectangular, or polygonal. In one embodiment, a nanoparticle may consist essentially of non-spherical particles. For example, such particles may have the form of ellipsoids, which may have all three principal axes of differing lengths, or may be oblate or prelate ellipsoids of revolution. Non-spherical nanoparticles alternatively may be laminar in form, wherein laminar refers to particles in which the maximum dimension along one axis is substantially less than the maximum dimension along each of the other two axes. Non-spherical nanoparticles may also have the shape of frusta of pyramids or cones, or of elongated rods. In one embodiment, the nanoparticles may be irregular in shape. In one embodiment, a plurality of nanoparticles may consist essentially of spherical nanoparticles.

The MSNP size distribution depends on the application, but is principally monodisperse (e.g., a uniform sized population varying no more than about 5-20% in diameter, as otherwise described herein). The term “monodisperse” is used as a standard definition established by the National Institute of Standards and Technology (NIST) (Particle Size Characterization, Special Publication 960-1, January 2001) to describe a distribution of particle size within a population of particles, in this case nanoparticles, which particle distribution may be considered monodisperse if at least 90% of the distribution lies within 5% of the median size. See Takeuchi, et al., Advanced Materials, 2005, 17, No. 8, 1067-1072.

In certain embodiments, mesoporous silica nanoparticles can be in range, e.g., from around 5 nm to around 500 nm (e.g., about 50 nm to about 500 nm) in size, including all integers and ranges there between. The size is measured as the longest axis of the particle. In various embodiments, the particles are from around 10 nm to around 500 nm and from around 10 nm to around 100 nm in size. The mesoporous silica nanoparticles have a porous structure. The pores can be from around 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

In one embodiment, MSNPs are monodisperse and range in size from about 25 nm to about 300 nm; exhibit stability (colloidal stability); have single cell binding specification to the substantial exclusion of non-targeted cells; are neutral or cationic for specific targeting (for example, cationic); are optionally modified with agents such as PEI, NMe3+, dye, crosslinker, ligands (ligands provide neutral charge); and optionally, are used in combination with a cargo to be delivered to a targeted cell.

In certain embodiments, the MSNPs are monodisperse and range in size from about 25 nm to about 300 nm. The sizes used for example include 50 nm (+/−10 nm) and 150 nm (+/−15 nm), within a narrow monodisperse range, but may be more narrow in range. A broad range of particles is not used because such a population is difficult to control and to target specifically.

Illustrative examples of a cationic surfactant include, but are not limited to, cetyl trimethylammonium bromide (CTAB), dodecylethyldimethylammonium bromide, cetylpyridinium chloride (CPC), polyethoxylated tallow amine (POEA), hexadecyltrimethylammonium p-toluenesulfonate, benzalkonium chloride (BAC), or benzethonium chloride (BZT).

Poloxamers such as F127 are difunctional block copolymer surfactants terminating in primary hydroxyl groups. They are composed of a central hydrophobic chain of polyoxypropylene (poly(propylene oxide)) flanked by two hydrophilic chains of polyoxyethylene (poly(ethylene oxide)). Because the lengths of the polymer blocks can be customized, many different poloxamers exist having slightly different properties. For the generic term “poloxamer”, these copolymers are commonly named with the letter “P” (for poloxamer) followed by three digits, the first two digits.times.100 give the approximate molecular mass of the polyoxypropylene core, and the last digit.times.10 gives the percentage polyoxyethylene content (e.g., P407=Poloxamer with a polyoxypropylene molecular mass of 4,000 g/mol and a 70% polyoxyethylene content). For the Pluronic® tradename, coding of these copolymers starts with a letter to define it's physical form at room temperature (L=liquid, P=paste, F=flake (solid)) followed by two or three digits, the first digit(s) refer to the molecular mass of the polyoxypropylene core (determined from BASF's Pluronic® grid) and the last digit.times.10 gives the percentage polyoxyethylene content (e.g., F127 is a PluronicdD with a polyoxypropylene molecular mass of 4,000 g/mol and a 70% polyoxyethylene content). In the example given, poloxamer 407 (P407) is Pluronic® F127.

In some embodiments, targeting moieties induce protocell binding to bacterially-infected host cells. In some embodiments, the targeting moiety targets a bacterially-infected host cell. In some embodiments, the targeting moiety specifically targets a host cell surface molecule specifically present during an intracellular bacterial infection.

Exemplary targeting ligands which may be used to target cells include peptides, affibodies and antibodies (including monoclonal and/or polydonal antibodies). In certain embodiments, targeting ligands selected from the group consisting of Fcγ from human igG (which binds to Fcγ receptors on macrophages and dendritic cells), human complement C3 (which binds to CR1 on macrophages and dendritic cells), ephrin B2 (which binds to EphB4 receptors on alveolar type II epithelial cells), and the SP94 peptide (which binds to unknown receptor(s) on hepatocyte-derived cells).

In some embodiments, the protocells described herein further comprise an endosomolytic moiety. After binding to a bacterially-infected host cell, the protocells described herein are internalized by the host cell. The endosomolytic moiety promotes escape of the antibacterial cargo into the host cell, where it can promote death of the intracellular bacteria. In some embodiments, the endosomolytic moiety ruptures a bacterially-infected cell membrane ruptures acidic intracellular vesicles of the bacterially-infected host cell. In some embodiments, the endosomolytic moiety is a peptide. In some embodiments, the endosomolytic moiety is octaarginine (R8). H5WYG, Penetratin-HA2, modified HA2-TAT, 43E or Histidine 10.

The charge is controlled based on what is to be accomplished (via PEI, NMe3+, dye, crosslinker, ligands, etc.), but for targeting the charge is for example cationic. Charge also changes throughout the process of formation. Initially the targeted particles are cationic and are often delivered as cationically charged nanoparticles, however post modification with ligands they are closer to neutral. The ligands which find use in the present invention include peptides, affibodies and antibodies, among others. These ligands are site specific and are useful for targeting specific cells which express peptides to which the ligand may bind selectively to targeted cells.

MSNPs may be used to deliver cargo to a targeted cell, including, for example, cargo component selected from the group consisting of at least one polynucleotide, such as double stranded linear DNA, minicircle DNA, naked DNA or plasmid DNA, messenger RNA, small interfering RNA, small hairpin RNA, microRNA, a polypeptide, a protein, a drug (in particular, an antibiotic drug), an imaging agent, or a mixture thereof. The MSNPs pursuant to the present invention are effective for accommodating cargo which are long and thin (e.g., naked) in three-dimensional structure, such as polynucleotides (e.g., various DNA and RNA) and polypeptides. In some embodiments, the cargo is an antibiotic (e.g., an antibiotic) or a CPP-PNA.

In protocells, a PEGylated lipid bi- or multilayer encapsulates a population of MSNPs as described herein and comprises (1) a PEGylated lipid which is optionally-thiolated (2) at least one additional lipid and, optionally (3) at least one targeting ligand which is conjugated to the outer surface of the lipid bi- or multilayer and which is specific against one or more receptors of bacterially-infected cells.

Protocells are highly flexible and modular. High concentrations of physiochemically-disparate molecules can be loaded into the protocells and their therapeutic and/or diagnostic agent release rates can be optimized without altering the protocell's size, size distribution, stability, or synthesis strategy. Properties of the supported lipid bi- or multilayer and mesoporous silica nanoparticle core can also be modulated independently, thereby optimizing properties as surface charge, colloidal stability, and targeting specificity independently from overall size, type of cargo(s), loading capacity, and release rate.

Pharmaceutical formulations and protocells can be used in the treatment of an infection caused by a bacterium selected from the group consisting of multidrug-resistant (MDR) Klebsiella pneumoniae (Kpn), methicillin-resistant Staphylococcus aureus (MRSA), F. tularensis and B. pseudomallei. Infections associated with S. aureus or extracellular toxin complex (ETC) produced by K. pneumoniae can also be treated effectively. The pharmaceutical formulations and protocels are particularly useful in the treatment of methicillin-resistant Staphylococcus aureus (MRSA) skin and soft tissue infections (SSTI).

In certain embodiments, the pharmaceutical formulations and protocells can be used in the treatment of subject who is infected by a bacterium selected from the group consisting of multidrug-resistant (MDR) Klebsiella pneumoniae (Kpn), methicillin-resistant Staphylococcus aureus (MRSA), F. tularensis and B. pseudomallei.

Thus, pharmaceutical formulations and protocells can be used to treat a wide variety of bacterial infections including, but not limited to, infections caused by bacteria selected from the group consisting of F. tularensis, B. pseudomallei, Mycobacterium, staphylococcus, streptococcaceae, neisseriaceae, cocci, enterobacteriaceae, pseudomonadaceae, vibrionaceae, campylobacter, pasteurellaceae, bordetella, francisella, brucella, legionellaceae, bacteroidaceae, gram-negative bacilli, clostridium, corynebacterium, propionibacterium, gram-positive bacilli, anthrax, actinomyces, nocardia, mycobacterium, treponema, b rrelia, leptospira, mycoplasma, ureaplasma, rickettsia, chlamydiae and P. aeruginosa.

In addition to, or as an alternative to, the therapeutic nucleic acid cargo described herein, pharmaceutical formulations and protocells can also contain one or more antibiotics, e.g., antibiotics selected from the group consisting of Gentamicin, Kanamycin, Neomycin, Netilmicin, Tobramycin, Paromomycin, Spectinomycin, Geldanamycin, Herbimycin, Rifaximin, Streptomycin, Ertapenem, Doripenem, Imipenem/Cilastatin, Meropenem, Cefadroxil, Cefazolin, Cephalothin, Cephalexin, Cefaclor, Cefamandole, Cefoxitin, Cefprozil, Cefuroxime, Cefixime, Cefdinir, Cefditoren, Cefoperazone Cefotaxime, Cefpodoxime, Ceftazidime, Ceflibuten, Ceflizoxime Ceftriaxone, Cefepime. Ceftaroline fosamil, Ceftobiprole, Teicoplanin, Vancomycin, Telavancin, Daptomycin. Oritavancin, WAP-8294A, Azithromycin, Clarithromycin, Dirithromycin, Erythromycin, Roxithromycin, Telithromycin. Spiramycin. Clindamycin, Lincomycin, Aztreonam, Furazolidone, Nitrofurantoin, Oxazolidinones, Unezolid, Posizolid, Radezolid, Torezolid, Amoxicillin, Ampicilin, Azlocillin, Carbenicillin, Cloxacilin Dicloxacilin, Fludoxacillin, Mezlocillin, Methicilln, Nafcillin, Oxacillin, Penicillin G, Penicillin V, Piperacillin, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, Mafenide, Sulfacetamide, Sulfadiazine, Sulfadimethoxine, Sulfamethizole, Sulfamethoxazole, Sulfasalazine, Sulfisoxazole, Trimethoprim-Sulfamethoxazole, Sulfonamidochrysoidine, Demedocydine, Doxycydine, Vibramycin Minocycline, Tigecydine, Oxytetracycline, Tetracycline, Clofazimine, Capreomycin, Cycloserine, Ethambutol, Rifampicin, Rifabutin, Rifapentine, Arsphenamine, Chloramphenicol, Fosfomycin, Fusidic acid, Metronidazole, Mupirocin, Platensimycin, Quinupristin/Dalfopristin, Thiamphenicol, Tigecycline and Tinidazole and combinations thereof.

Typically pharmaceutical formulations and protocells can be loaded with cargo to a capacity up to about 10, 20, 30, 40, 50, 60, 70, 80 or about 90 weight % or more (or from about 0.01% to about 70%, about 0.02% to about 60%, about 0.2 to about 55%, about 0.5% to about 45%, about 1% to about 35%, about 1.5% to about 25%, about 0.1% to about 10%, about 0.01% to about 5%): defined as (cargo weight/weight of loaded protocel)×100. The optimal loading of cargo is often about 0.01 to 60% but this depends on the drug or drug combination which is incorporated as cargo into the MSNPs. This is generally expressed in μM per 10¹⁰ particles where we have values ranging from 2000-100 μM per 10¹⁰ particles. For example, MSNPs exhibit release of cargo at pH about 5.5, which is that of the endosome, but are stable at physiological pH of 7 or higher (7.4).

The surface area of the internal space for loading is the pore volume whose optimal value ranges from about 1.1 to 0.5 cubic centimeters per gram (cc/g). Note that in the MSNPs according to one embodiment, the surface area is mainly internal as opposed to the external geometric surface area of the nanoparticle.

The lipid bi- or multilayer supported on the porous particle according to one embodiment has a lower melting transition temperature, i.e. is more fluid than a lipid bi- or multilayer supported on a non-porous support or the lipid bi- or multilayer in a liposome. This is sometimes important in achieving high affinity binding of immunogenic peptides or targeting ligands at low peptide densities, as it is the bilayer fluidity that allows lateral diffusion and recruitment of peptides by target cell surface receptors. One embodiment provides for peptides to cluster, which facilitates binding to a complementary target.

The lipid bi- or multilayer may vary significantly in composition. Ordinarily, any lipid or polymer which may be used in liposomes may also be used in MSNPs. Exemplary lipids are as otherwise described herein.

In some embodiments, the lipid bi- or multilayer of the protocells can provide biocompatibility and can be modified to possess targeting species including, for example, antigens, targeting peptides, fusogenic peptides, antibodies, aptamers, and PEG (polyethylene glycol) to allow, for example, further stability of the protocells and/or a targeted delivery into a cell to maximize an immunogenic response. PEG, when included in lipid bilayers, can vary widely in molecular weight (although PEG ranging from about 10 to about 100 units of ethylene glycol, about 15 to about 50 units, about 15 to about 20 units, about 15 to about 25 units, about 16 to about 18 units, etc., may be used) and the PEG component which is generally conjugated to phospholipid through an amine group comprises about 1% to about 20%, for example about 5% to about 15%, about 10% by weight of the lipids which are included in the lipid bi- or multilayer. The PEG component is generally conjugated to an amine-containing lipid such as DOPE or DPPE or other lipid, but in alternative embodiments may also be incorporated into the MSNPs, through inclusion of a PEG containing silane.

Numerous lipids which are used in liposome delivery systems may be used to form the lipid bi- or multilayer on nanoparticles. Virtually any lipid which is used to form a liposome may be used in the lipid bi- or multilayer which surrounds the nanoparticles according to an embodiment. For example, lipids for use in the present invention include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment given the fact that cholesterol may be an important component of the lipid bilayer of protocels according to an embodiment. Often cholesterol is incorporated into lipid bilayers of protocells in order to enhance structural integrity of the bilayer. These lipids are all readily available commercially from Avanti Polar Lipids, Inc. (Alabaster, Ala. USA). DOPE and DPPE are particularly useful for conjugating (through an appropriate crosslinker) PEG, peptides, polypeptides, including immunogenic peptides, proteins and antibodies, RNA and DNA through the amine group on the lipid.

MSNPs and protocells can be PEGylated with a variety of polyethylene glycol-containing compositions as described herein. PEG molecules can have a variety of lengths and molecular weights and include, but are not limited to, PEG 200, PEG 1000, PEG 1500, PEG 4600, PEG 10,000, PEG-peptide conjugates or combinations thereof.

Exemplary fluorescent labels for use in MSNPs and protocells (for example via conjugation or adsorption to the lipid bi- or multilayer or silica core, although these labels may also be incorporated into cargo elements such as DNA. RNA, polypeptides and small molecules which are delivered to cells by the protocells) include Hoechst 33342 (350/461), 4′,6-diamidino-2-phenylindole (DAPI, 356/451), Alexa Fluor® 405 carboxylic acid, succinimidyl ester (401/421). CellTracker™ Violet BMQC (415/516), CellTracker™ Green CMFDA (492/517), calcein (495/515), Alexa Fluor® 488 conjugate of annexin V (495/519), Alexa FluoP 488 goat anti-mouse IgG (H+L) (495/519), Click-iT® AHA Alexa Fluor® 488 Protein Synthesis HCS Assay (495/519), LIV5tOEAD^{D} Fixable Green Dead Cell Stain Kit (495/519), SYTOX® Green nucleic acid stain (504/523), MitoSOX™ Red mitochondrial superoxide indicator (510/580). Alexa Fluor® 532 carboxylic acid, succinimidyl ester(532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670) and Alexa Fluor® 647 conjugate of annexin V (650/665). Moieties which enhance the fluorescent signal or slow the fluorescent fading may also be incorporated and include SlowFade® Gold antifade reagent (with and without DAPI) and Image-iT® FX signal enhancer. All of these are well known in the art.

Additional reporters include polypeptide reporters which may be expressed by plasmids (such as histone-packaged supercoiled DNA plasmids) and include polypeptide reporters such as fluorescent green protein and fluorescent red protein. Reporters pursuant to the present invention are utilized principally in diagnostic applications including diagnosing the existence or progression of a bacterial infection in a patient and or the progress of therapy in a patient or subject. Using the above-described protocells which comprise at least one reporter, this approach can include a method of first diagnosing and then treating an antibiotic-resistant bacterial infection, the method comprising administering to a subject in need thereof a population of antibacterial protocells after diagnosing the existence of the disease state in the patient by comparing infected tissue from the patient to a standard. In alternative embodiments, the diagnostic method can be used at various times during therapy of an infected patient to measure the impact of the treatment on the progression of disease.

Pharmaceutical compositions comprise an effective population of MSNPs and/or protocells as otherwise described herein formulated to effect an intended result (e.g., immunogenic result, therapeutic result and/or diagnostic analysis, including the monitoring of therapy) formulated in combination with a pharmaceutically acceptable carrier, additive or excipient. The MSNPs and/or protocells within the population of the composition may be the same or different depending upon the desired result to be obtained. Pharmaceutical compositions may also comprise an additional bioactive agent or drug, such as an antiviral agent.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g., orally, transdermally, perineurally or parenterally, that is, by intravenous, subcutaneous, intraperitoneal, intrathecal or intramuscular injection, among others, including buccal, rectal and transdermal administration. Subjects contemplated for treatment according to the method include humans, companion animals, laboratory animals, and the like. The invention contemplates immediate and/or sustained/controlled release compositions, including compositions which comprise both immediate and sustained release formulations. This is particularly true when different populations of MSNPs and/or protocells are used in the pharmaceutical compositions or when additional bioactive agent(s) are used in combination with one or more populations of protocens as otherwise described herein.

Formulations containing the compounds may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, for example in unit dosage forms suitable for simple administration of precise dosages.

Pharmaceutical compositions typically include a conventional pharmaceutical carrier or excipient and may additionally include other medicinal agents, carriers, adjuvants, additives and the like. For example, the composition is about 0.1% to about 85%, about 0.5% to about 75% by weight of a compound or compounds, with the remainder consisting essentially of suitable pharmaceutical excipients.

An injectable composition for parenteral administration (e.g., intravenous, intramuscular or intrathecal) will typically contain the compound in a suitable i.v. solution, such as sterile physiological salt solution. The composition may also be formulated as a suspension in an aqueous emulsion.

Liquid compositions can be prepared by dissolving or dispersing the population of MSNPs and/or protocells (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g., an excipient such as a saccharide or cellulose preparation, a binder such as starch paste or methyl cellulose, a filer, a disintegrator, and other additives typically used in the manufacture of medical preparations.

Methods for preparing such dosage forms are known or is apparent to those skilled in the art; for example, see Remington's Pharmaceutical Sciences (17th Ed., Mack Pub. Co., 1985). The composition to be administered will contain a quantity of the selected compound in a pharmaceutically effective amount for therapeutic use in a biological system, including a patient or subject.

Methods of treating patients or subjects in need for a particular disease state or infection comprise administration an effective amount of a pharmaceutical composition comprising therapeutic MSNPs and/or protocells and optionally at least one additional bioactive (e.g., antibacterial or antiviral) agent.

Diagnostic methods may comprise administering to a patient in need an effective amount of a population of diagnostic MSNPs and/or protocels (e.g., MSNPs and/or protocells which comprise a target species, such as a targeting peptide which binds selectively to bacterially-infected cells and a reporter component to indicate the binding of the protocells) whereupon the binding of the MSNPs and/or protocels to cells as evidenced by the reporter component (moiety) will enable a diagnosis of the existence of a disease state in the patient.

An alternative of the diagnostic method may be used to monitor the therapy of a disease state in a patient, the method comprising administering an effective population of diagnostic MSNPs and/or protocels (e.g., MSNPs and/or protocels which comprise a target species, such as a targeting peptide which binds selectively to target cells and a reporter component to indicate the binding of the protocells to bacterially-infected cells if such cells are present) to a patient or subject prior to treatment, determining the level of binding of diagnostic protocells to target cells in said patient and during and/or after therapy, determining the level of binding of diagnostic protocels to target cells in said patient, whereupon the difference in binding before the start of therapy in the patient and during and/or after therapy will evidence the effectiveness of therapy in the patient, including whether the patient has completed therapy or whether the disease state has been inhibited or eliminated.

There may be employed conventional molecular biology, microbiology, and recombinant DNA techniques within the skill of the art. Such techniques are explained fully in the literature. See, e.g., Sambrook et al, 2001, Ausubel, ed., 1994, Coligan, ed., 1994, Hames & Higgins eds., 1985, Hames & Higgins, eds., 1984, Freshney, ed., 1986.

Any number of histone proteins, as well as other means to package the DNA into a smaller volume such as normally cationic nanoparticles, lipids, or proteins, may be used to package the supercoiled plasmid DNA “histone-packaged supercoiled plasmid DNA”, but in therapeutic aspects which relate to treating human patients, the use of human histone proteins are for example used. In certain aspects, a combination of human histone proteins H1, H2A, H2B, H3 and H4 in one embodiment ratio of 1:2:2:2:2, although other histone proteins may be used in other, similar ratios, as is known in the art or may be readily practiced pursuant to the teachings. The DNA may also be double stranded linear DNA, instead of plasmid DNA, which also may be optionally supercoiled and/or packaged with histones or other packaging components.

Other histone proteins which may be used in this aspect include, for example, H1F, H1FO, H1FNT, H1FOO, H1FX H1H1 HIST1H1A, HIST1H1B, HIST1H1C, HIST1H1D, HIST1H1E, HIST1H1T, H2AF, H2AFB1, H2AFB2, H2AFB3, H2AFJ, H2AFV, H2AFX, H2AFY, H2AFY2, H2AFZ, H2A1, HIST1H2AA, HIST1H2AB, HIST1H2AC, HIST1H2AD, HIST1H2AE, HIST1H2AG, HIST1H2AI, HIST1H2AJ, HIST1H2AK, HIST1H2AL, HIST1H2AM, H2A2, HIST2H2AA3, HIST2H2AC, H2BF, H2BFM, HSBFS, HSBFWT, H2B1, HIST1H2BA, HIST1HSBB, HIST1HSBC, HIST1HSBD, HIST1H2BE, HIST1H2BF, HIST1H2BG, HIST1H2BH, HIST1H2BI, HIST1H2BJ, HIST1H2BK, HIST1H2BL, HIST1H2BM, HIST1H2BN, HIST1H2BO, H2B2, HIST2H2BE, H3A1, HIST1H3A, HIST1H3B, HIST1H3C, HIST1H3D, HIST1H3E, HIST1H3F, HIST1H3G, HIST1H3H, HIST1H31, HIST1H3J, H3A2, HIST2H3C, H3A3, HIST3H3, H41, HIST1H4A, HIST1H4B, HIST1H4C, HIST1H4D, HIST1H4E, HIST1H4F, HIST1H4G, HIST1H4H, HIST1H41, HIST1H4J, HIST1H4K, HIST1H4L, H44 and HIST4H4.

Proteins gain entry into the nucleus through the nuclear envelope. The nuclear envelope consists of concentric membranes, the outer and the inner membrane. These are the gateways to the nucleus. The envelope consists of pores or large nuclear complexes. A protein translated with a NLS will bind strongly to importin (aka karyopherin), and together, the complex will move through the nuclear pore. Any number of nuclear localization sequences may be used to introduce histone-packaged plasmid DNA into the nucleus of a cell. Exemplary nuclear localization sequences include H2N-GNQSSNFGPMKGGNFGGRSSGPYGGGGQYFAKPRNQGGYGGC-COOH, RRMKWKK, PKKKRKV, and KR[PAATKKAGQA]KKKK (SEQ ID NOs:24-27), the NLS of nucleoplasmin, a prototypical bipartite signal comprising two clusters of basic amino acids, separated by a spacer of about 10 amino acids. Numerous other nuclear localization sequences are well known in the art. See, for example, LaCasse et al., (1995); Weis, (1998); and Murat Cokol et al., at the website ubic.bioc.columbia.edu/papers/2000 nls/paper.html#tab2.

A peptide nucleic acid can consist of repeating N-(2-aminoethyl)-glycine units linked by amide bonds. The purine (A, G) and pyrimidine (C, T) bases are attached to the backbone through methylene carbonyl linkages. Unlike DNA or DNA analogs. PNAs do not contain any (pentose) sugar moieties or phosphate groups. Surprisingly, PNA's in many respects mimic the behavior of DNA, and in some applications demonstrate superior properties. By convention, PNAs are depicted like peptides, with the N-terminus at the (left) position and the C-terminus at the right. Besides the obvious structural difference, PNA is set apart from DNA in that the backbone of PNA is acyclic, achiral and neutral. PNAs can bind to complementary nucleic acids in both antiparallel and parallel orientation. However, the antiparallel orientation is strongly in one embodiment, and the parallel duplex has been shown to have a different structure. Nielsen, et al., “An Introduction to Peptide Nucleic Acid”, Current Issues Molec. Biol. (1999) 1(2): 89-104.

A level and/or an activity and/or expression of a translation product of a gene and/or of a fragment, or derivative, or variant of said translation product, and/or the level or activity of said translation product, and/or of a fragment, or derivative, or variant thereof, can be detected using an immunoassay, an activity assay, and/or a binding assay. These assays can measure the amount of binding between said protein molecule and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Immunoassays which can be used include e.g. ELISAs, Western blots and other techniques known to those of ordinary skill in the art (see Harlow and Lane, Antibodies: A Laboratory Manual, Cold Spring Harbor Laboratory Press, Cold Spring Harbor, N.Y., 1999 and Edwards R, Immunodiagnostics: A Practical Approach, Oxford University Press, Oxford; England, 1999). All these detection techniques may also be employed in the format of microarrays, protein-arrays, antibody microarrays, tissue microarrays, electronic biochip or protein-chip based technologies (see Schena M., Microarray Biochip Technology, Eaton Publishing. Natick, Mass., 2000).

Certain diagnostic and screening methods utilize an antibody, for example, a monoclonal antibody, capable of specifically binding to a protein as described herein or active fragments thereof. The method of utilizing an antibody to measure the levels of protein allows for non-invasive diagnosis of the pathological states of kidney diseases. In one embodiment, the antibody is human or is humanized. In one embodiment, antibodies may be used, for example, in standard radioimmunoassays or enzyme-linked immunosorbent assays or other assays which utilize antibodies for measurement of levels of protein in sample. In a particular embodiment, the antibodies are used to detect and to measure the levels of protein present in a sample.

Humanized antibodies are antibodies, or antibody fragments, that have the same binding specificity as a parent antibody, (i.e., typically of mouse origin) and increased human characteristics. Humanized antibodies may be obtained, for example, by chain shuffling or by using phage display technology. For example, a polypeptide comprising a heavy or light chain variable domain of a non-human antibody specific for a disease related protein is combined with a repertoire of human complementary (light or heavy) chain variable domains. Hybrid pairings specific for the antigen of interest are selected. Human chains from the selected pairings may then be combined with a repertoire of human complementary variable domains (heavy or light) and humanized antibody polypeptide dimers can be selected for binding specificity for an antigen. Techniques described for generation of humanized antibodies that can be used in the method are disclosed in, for example, U.S. Pat. Nos. 5,565,332; 5,585,089; 5,694,761; and 5,693,762. Furthermore, techniques described for the production of human antibodies in transgenic mice are described in, for example, U.S. Pat. Nos. 5,545,806 and 5,569,825.

In order to identify small molecules and other agents useful in the present methods for treating an antibiotic resistant bacterial infection by modulating the activity and expression of a disease-related protein and biologically active fragments thereof can be used for screening therapeutic compounds in any of a variety of screening techniques. Fragments employed in such screening tests may be free in solution, affixed to a solid support, borne on a cell surface, or located intracellularly. The blocking or reduction of biological activity or the formation of binding complexes between the disease-related protein and the agent being tested can be measured by methods available in the art.

Other techniques for drug screening which provide for a high throughput screening of compounds having suitable binding affinity to a protein, or to another target polypeptide useful in modulating, regulating, or inhibiting the expression and/or activity of a disease, are known in the art. For example, microarrays carrying test compounds can be prepared, used, and analyzed using methods available in the art. See, e.g., Shalon, D. et al., 1995, International Publication No. WO95/35505, Baldeschweiler et al., 1995. International Publication No. WO95/251116; Brennan et al., 1995. U.S. Pat. No. 5,474,796; Heller et al., 1997, U.S. Pat. No. 5,605,662.

To determine specific binding, various immunoassays may be employed for detecting, for example, human or primate antibodies bound to the cells. Thus, one may use labeled anti-hlg, e.g., anti-hlgM, hlgG or combinations thereof to detect specifically bound human antibody. Various labels can be used such as radioisotopes, enzymes, fluorescers, chemiluminescers, particles, etc. There are numerous commercially available kits providing labeled anti-hlg, which may be employed in accordance with the manufacturers protocol.

In one embodiment, a kit can comprise: (a) at least one reagent which is selected from the group consisting of (i) reagents that detect a transcription product of the gene coding for a protein marker as described herein (ii) reagents that detect a translation product of the gene coding for proteins, and/or reagents that detect a fragment or derivative or variant of said transcription or translation product; (b) instructions for diagnosing, or prognosticating a disease, or determining the propensity or predisposition of a subject to develop such a disease or of monitoring the effect of a treatment by determining a level, or an activity, or both said level and said activity, and/or expression of said transcription product and/or said translation product and/or of fragments, derivatives or variants of the foregoing, in a sample obtained from said subject; and comparing said level and/or said activity and/or expression of said transcription product and/or said translation product and/or fragments, derivatives or variants thereof to a reference value representing a known disease status (patient) and/or to a reference value representing a known health status (control) and/or to a reference value; and analyzing whether said level and/or said activity and/or expression is varied compared to a reference value representing a known health status, and/or is similar or equal to a reference value representing a known disease status or a reference value; and diagnosing or prognosticating a disease, or determining the propensity or predisposition of said subject to develop such a disease, wherein a varied or altered level, expression or activity, or both said level and said activity, of said transcription product and/or said translation product and/or said fragments, derivatives or variants thereof compared to a reference value representing a known health status (control) and/or wherein a level, or activity, or both said level and said activity, of said transcription product and/or said translation product and/or said fragments, derivatives or variants thereof is similar or equal to a reference value and/or to a reference value representing a known disease stage, indicates a diagnosis or prognosis of a disease, or an increased propensity or predisposition of developing such a disease, a high risk of developing signs and symptoms of a disease.

Reagents that selectively detect a transcription product and/or a translation product of the gene coding for proteins can be sequences of various length, fragments of sequences, antibodies, aptamers, siRNA, microRNA, and ribozymes. Such reagents may be used also to detect fragments, derivatives or variants thereof.

Purely by way of example, comparing measured levels of an antibiotic-resistant bacterial infection biomarker in a sample to corresponding control levels, or comparing measured bacterial levels to control bacterial levels determined in a healthy control subject, and determining that a subject suffers from an antibiotic-resistant bacterial infection or that a subject's an antibiotic-resistant bacterial infection is progressing, can include determinations based on comparative level differences of about between about 5-10%, or about 10-15%, or about 15-20%, or about 20-25%, or about 25-30%, or about 30-35%, or about 35-40%, or about 40-45%, or about 45-50%, or about 50-55%, or about 55-60%, or about 60-65%, or about 65-70%, or about 70-75%, or about 75-80%, or about 80-85%, or about 85-90%, or about 90-95%, or about 95-100%, or about 100-110%, or about 110-120%, or about 120-130%, or about 130-140%, or about 140-150%, or about 150-160%, or about 160-170%, or about 170-180%, or about 180-190%, or 190-200%, or 200-210%, or 210-220%, or 220-230%, or 230-240%, or 240-250%, or 250-260%, or about 260-270%, or about 270-280%, or about 280-290%, or about 290-300%, or differences of about between about ±50% to about ±0.5%, or about ±45% to about ±1%, or about ±40% to about ±1.5%, or about ±35% to about ±2.0%, or about ±30% to about ±2.5%, or about ±25% to about ±3.0%, or about ±20% to about ±3.5%, or about ±15% to about ±4.0%, or about ±10% to about ±5.0%, or about ±9% to about ±1.0%, or about ±8% to about ±2%, or about ±7% to about ±3%, or about ±6% to about ±5%, or about ±5%, or about ±4.5%, or about ±4.0%, or about ±3.5%, or about ±3.0%, or about ±2.5%, or about ±2.0%, or about ±1.5%, or about ±1.0%.

Non-limiting examples of moieties that target ephrin B2 and/or ephrin B3 include the ephrin B2-targeting peptide sequences identified in FIG. 31. TGAILHP (SEQ ID NO: 52) is an example of an ephrin B2-targeting peptide sequence.

Synergistic, High Concentration Drug Loaded Protocell Compositions

The present disclosure provides nanoparticles functionalized with a hydrophobic group and loaded with a water-insoluble cargo and protocells comprising such nanoparticles encapsulated by a lipid bilayer. The hydrophobic groups on and within the nanoparticles provide a favorable electrostatic environment for high-capacity loading of water-insoluble cargos, such as small-molecule drugs. In some embodiments, the nanoparticles or protocells further comprise a cell targeting species, which allows targeted delivery of the water-insoluble cargo.

Further provided herein are protocells comprising a cellular barrier penetrating moiety. Cellular barriers, such as the blood-brain barrier or nasal epithelium, limit the ability of orally, intranasally, or intravenously administered therapeutic agents from reaching targeted cells, resulting in subtherapeutic concentrations. By loading therapeutic agents into a protocell comprising a cellular barrier penetrating moiety, as described herein, therapeutic agents penetrate cellular barriers to deliver the cargo to the desired targeted cells. One example of a cellular barrier penetrating moiety is glutathione. For example, in some embodiments, a protocell comprising a cellular barrier penetrating moiety penetrates the blood-brain barrier for delivery to a cell within the central nervous system (CNS).

The following terms shall be used throughout the specification to describe the present invention. Where a term is not specifically defined herein, that term shall be understood to be used in a manner consistent with its use by those of ordinary skill in the art.

The examples herein provide various methodologies for obtaining protocells which are useful in the present invention. Useful general techniques include those described in Liu, et al., Chem. Comm., 5100-5102 (2009), Liu, et al., J. Amer. Chem. Soc., 131, 1354-1355 (2009), Liu, et al., J. Amer. Chem. Soc., 131, 7567-7569 (2009) Lu, et al., Nature, 398, 223-226 (1999), Ashley, et al., Nature Materials, 2011. May; 10(5):389-97, Lu, et al., Nature, 398, 223-226 (1999), and Caroll, et al., Langmuir, 25, 13540-13544 (2009).

Nanostructures include a core-shell structure which comprises a porous particle core surrounded by a shell of lipid for example a bilayer, but possibly a monolayer or multilayer (see Liu, et al., J. Amer. Chem. Soc., 131, 7567-7569 (2009)). The porous particle core can include, for example, a porous nanoparticle made of an inorganic and/or organic material as set forth above surrounded by a lipid bilayer.

The porous particle core of the protocells can be loaded with various desired species (“cargo”), including small molecules (e.g., anti-cancer, antibacterial and antiviral agents), large molecules (e.g., including macromolecules such as miRNA, siRNA or shRNA or a polypeptide which may include an anti-cancer, antibacterial and antiviral agent polypeptide or a reporter polypeptide (e.g., fluorescent green protein, among others), semiconductor quantum dots, or metallic nanoparticles, or metal oxide nanoparticles or combinations thereof). Protocefs can also be loaded with super-coiled plasmid DNA, which can be used to deliver a therapeutic and/or diagnostic peptide(s) or a small hairpin RNAlshRNA or small interfering RNA/siRNA, which can be used to inhibit expression of proteins associated with antibiotic resistance.

In some embodiments, protocells are comprised of a spherical mesoporous silica nanoparticlde (MSNP) core encased within a supported lipid bilayer (SLB). MSNPs have an extremely high surface area (>1200 m²/g), which enables high concentrations of various therapeutic and diagnostic agents to be adsorbed within the core by simple immersion in a solution of the cargo(s) of interest. Furthermore, since the aerosol-assisted evaporation-induced self-assembly (EISA) process we pioneered to synthesize MSNPs is compatible with a wide range of structure-directing surfactants and amenable to post-synthesis processing, the overall size can be varied from 20-nm to >10-μm, the pore size can be varied from 2.5-nm to 50-nm, and the naturally negatively-charged pore walls can be modified with a variety of functional moieties, enabling facile encapsulation of physicochemically disparate molecules, including acidic, basic, and hydrophobic drugs, proteins, small interfering RNA, minicircle DNA vectors, plasmids, and diagnostic agents like quantum dots and iron oxide nanoparticles.

Protocells have a loading capacity of up to 60 wt % for small molecule drugs, which is 10-fold higher than other MSNP-based delivery vehicles and 1000-fold higher than similarly-sized liposomes. Release rates can be tailored by controlling the core's degree of silica condensation and, therefore, its dissolution rate under physiological conditions; thermal calcination maximizes condensation and results in particles with sustained release profiles (7-10% release per day for up to 2 weeks), while use of acidified ethanol to extract surfactants enhances particle solubility and results in burst release of encapsulated drugs (100% release within 12 hours). Liposome fusion to cargo-loaded MSNPs results in the formation of a coherent SLB that provides a stable, fluid, biocompatible interface for display of functional molecules, such as polyethylene glycol (PEG) and targeting ligands.

Protocells stably encapsulate small molecule drugs for up to 4 weeks when dispersed in complex biological fluids (e.g., complete growth medium and blood), regardless of whether the SLB is composed of lipids that are fluid or non-fluid at body temperature; in contrast, liposomes rapidly leak their encapsulated drugs, even when their bilayers are composed of fully saturated lipids, which have a high packing density and should, therefore, limit diffusion of drugs across the bilayer. The fluid, yet stable SLB enables us to achieve exquisitely high targeting specificities at low ligand densities, which, in turn, reduces immunogenicity and non-specific interactions; we have shown that protocells modified with an average of just 6 targeting peptides per particle have a 10,000-fold higher affinity for target cells than for non-target cells when the SLB is composed of the fluid, zwitterionic lipid, 1, 2-dioleoyl-sn-glycero-3-phosphocholine (DOPC).

Protocells are highly biocompatible and can be engineered for both broad distribution and persistence within target tissues. Balb/c mice injected intravenously (i.v.) with 200 mg/kg doses of PEGylated protocells three times each week for three weeks show no signs of gross or histopathological toxicity. Given their high loading capacity, this result indicates that protocells can deliver at least 900 mg/kg of therapeutic molecules with either burst or sustained release kinetics. Furthermore, PEGylated protocells 20-200 nm in diameter remain broadly distributed for 2-7 days when injected i.v., which provides a sufficient period of time for targeted protocells to accumulate within target tissues, where they can persist for up to 4 weeks with no adverse effects. Additionally, we and others have demonstrated that MSNPs are biodegradable and ultimately excreted in the urine and feces as silicic acid. Finally, we have shown that protocells modified with up to 10 wt % of targeting ligands induce neither IgG nor IgM responses when injected in C57Bl/6 mice at a total dose of 400 mg/kg. Depending upon the biodistribution required for a specific application, we can control the MSNP size and shape (spherical, disk-shaped, and rod-shaped(9) and the SLB charge and surface modification(s), making the protocell a highly modular, flexible nanoparticle delivery system.

Conventionally, a mesoporous nanoparticle has pores whose diameters range in size from about 2 nm to about 50 nm, a “microporous” nanoparticle has pores whose diameters are less than about 2 nm (often about 0.001 to about 2 nm) and a “macroporous” nanoparticle has pores whose diameters are from about 50 nm to about 100 nm. MSNPs can have both mesoporous, microporous and macroporous pores, but often have pores whose diameters range in size from about 2 nm to about 50 nm.

A nanoparticle may have a variety of shapes and cross-sectional geometries that may depend, in part, upon the process used to produce the particles. In one embodiment, a nanoparticle may have a shape that is a torus (toroidal). A nanoparticle may include particles having two or more of the aforementioned shapes. In one embodiment, a cross-sectional geometry of the particle may be one or more of toroidal, circular, ellipsoidal, triangular, rectangular, or polygonal. In one embodiment, a nanoparticle may consist essentially of non-spherical particles. For example, such particles may have the form of ellipsoids, which may have all three principal axes of differing lengths, or may be oblate or prelate ellipsoids of revolution. Non-spherical nanoparticles alternatively may be laminar in form, wherein laminar refers to particles in which the maximum dimension along one axis is substantially less than the maximum dimension along each of the other two axes. Non-spherical nanoparticles may also have the shape of frusta of pyramids or cones, or of elongated rods. In one embodiment, the nanoparticles may be irregular in shape. In one embodiment, a plurality of nanoparticles may consist essentially of spherical nanoparticles.

The MSNP size distribution depends on the application, but is principally monodisperse (e.g., a uniform sized population varying no more than about 5-20% in diameter, as otherwise described herein). The term “monodisperse” is used as a standard definition established by the National Institute of Standards and Technology (NIST) (Particle Size Characterization, Special Publication 960-1, January 2001) to describe a distribution of particle size within a population of particles, in this case nanoparticles, which particle distribution may be considered monodisperse if at least 90% of the distribution lies within 5% of the median size. See Takeuchi, et al., Advanced Materials, 2005, 17, No. 8, 1067-1072.

In certain embodiments, mesoporous silica nanoparticles can be range, e.g., from around 5 nm to around 500 nm (for example, about 50 nm to about 500 nm) in size, including all integers and ranges there between. The size is measured as the longest axis of the particle. In various embodiments, the particles are from around 10 nm to around 500 nm and from around 10 nm to around 100 nm in size. The mesoporous silica nanoparticles have a porous structure. The pores can be from around 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

Exemplary MSNPs: are monodisperse and range in size from about 25 nm to about 300 nm; exhibit stability (colloidal stability); have single cell binding specification to the substantial exclusion of non-targeted cells; are neutral or cationic for specific targeting (for example, cationic); are optionally modified with agents such as PEI, NMe₃⁺, dye, crosslinker, ligands (ligands provide neutral charge); and optionally, are used in combination with a cargo to be delivered to a targeted cell.

In certain embodiments, the MSNPs are monodisperse and range in size from about 25 nm to about 300 nm. The sizes used for example include 50 nm (+/−10 nm) and 150 nm (+/−15 nm), within a narrow monodisperse range, but may be more narrow in range. A broad range of particles is not used because such a population is difficult to control and to target specifically.

Illustrative examples of a cationic surfactant include, but are not limited to, cetyl trimethylammonium bromide (CTAB), dodecylethyldimethylammonium bromide, cetylpyridinium chloride (CPC), polyethoxylated tallow amine (POEA), hexadecyitrimethylammonium p-toluenesulfonate, benzalkonium chloride (BAC), or benzethonium chloride (BZT).

Poloxamers such as F127 are difunctional block copolymer surfactants terminating in primary hydroxyl groups. They are composed of a central hydrophobic chain of polyoxypropylene (poly(propylene oxide)) flanked by two hydrophilic chains of polyoxyethylene (poly(ethylene oxide)). Because the lengths of the polymer blocks can be customized, many different poloxamers exist having slightly different properties. For the generic term “poloxamer”, these copolymers are commonly named with the letter “P” (for poloxamer) followed by three digits, the first two digits.times.100 give the approximate molecular mass of the polyoxypropylene core, and the last digit.times.10 gives the percentage polyoxyethylene content (e.g., P407=Poloxamer with a polyoxypropylene molecular mass of 4,000 g/mol and a 70% polyoxyethylene content). For the Pluronic® tradename, coding of these copolymers starts with a letter to define it's physical form at room temperature (L=liquid, P=paste, F=flake (solid)) followed by two or three digits, the first digit(s) refer to the molecular mass of the polyoxypropylene core (determined from BASF's Pluronic® grid) and the last digit.times.10 gives the percentage polyoxyethylene content (e.g., F127 is a Pluronic® with a polyoxypropylene molecular mass of 4,000 g/mol and a 70% polyoxyethylene content). In the example given, poloxamer 407 (P407) is Pluronic® F127.

Exemplary ligands which may be used to target cells include peptides, affibodies and antibodies (including monoclonal and/or polyclonal antibodies). In certain embodiments, targeting ligands selected from the group consisting of Fcγ from human IgG (which binds to Fcγ receptors on macrophages and dendritic cells), human complement C3 (which binds to CR1 on macrophages and dendritic cells), ephrin B2 (which binds to EphB4 receptors on alveolar type II epithelial cells), and the SP94 peptide (which binds to unknown receptor(s) on hepatocyte-derived cells). Targeting ligands in certain aspects target T-Cell for therapy.

The charge is controlled based on what is to be accomplished (via PEI, NMe₃⁺, dye, crosslinker, ligands, etc.), but for targeting the charge is for example cationic. Charge also changes throughout the process of formation. Initially the targeted particles are cationic and are often delivered as cationically charged nanoparticles, however post modification with ligands they are closer to neutral. The ligands which find use in the present invention include peptides, affibodies and antibodies, among others. These ligands are site specific and are useful for targeting specific cells which express peptides to which the ligand may bind selectively to targeted cells.

MSNPs may be used to deliver cargo to a targeted cell, including, for example, cargo component selected from the group consisting of at least one polynucleotide, such as double stranded linear DNA, minicircle DNA, naked DNA or plasmid DNA, messenger RNA, small interfering RNA, small hairpin RNA, microRNA, a polypeptide, a protein, a drug (in particular, an antibiotic drug), an imaging agent, or a mixture thereof. The MSNPs pursuant to the present invention are effective for accommodating cargo which are long and thin (e.g., naked) in three-dimensional structure, such as polynucleotides (e.g., various DNA and RNA) and polypeptides.

In protocells, a PEGylated lipid bi- or multilayer encapsulates a population of MSNPs as described herein and comprises (1) a PEGylated lipid which is optionally-thiolated (2) at least one additional lipid and, optionally (3) at least one targeting ligand which is conjugated to the outer surface of the lipid bi- or multilayer and which is specific against one or more receptors of a cell, e.g., a cancer cell.

Protocells are highly flexible and modular. High concentrations of physiochemically-disparate molecules can be loaded into the protocells and their therapeutic and/or diagnostic agent release rates can be optimized without altering the protocell's size, size distribution, stability, or synthesis strategy. Properties of the supported lipid bi- or multilayer and mesoporous silica nanoparticle core can also be modulated independently, thereby optimizing properties as surface charge, colloidal stability, and targeting specificity independently from overall size, type of cargo(s), loading capacity, and release rate.

Prophylactic administration is effective to reduce or decrease the likelihood of the subsequent occurrence of disease in the mammal, or decrease the severity of disease (inhibition) that subsequently occurs, especially including metastasis of cancer. Alternatively, compounds can, for example, be administered therapeutically to a mammal that is already afflicted by disease. In one embodiment of therapeutic administration, administration of the present compounds is effective to eliminate the disease and produce a remission or substantially eliminate the likelihood of metastasis of a cancer. Administration of the compounds is effective to decrease the severity of the disease or lengthen the lifespan of the mammal so afflicted, as in the case of cancer, or inhibit or even eliminate the causative agent of the disease, as in the case of hepatitis B virus (HBV) and/or hepatitis C virus infections (HCV) infections.

MSNPs and protocells can also be used to treat a wide variety of bacterial infections including, but not limited to, infections caused by bacteria selected from the group consisting of F. tularensis, B. pseudomallei, Mycobacterium, Staphylococcus, streptococcaceae, neisseriaceae, cocci, enterobacteriaceae, pseudomonadaceae, vibrionaceae, campylobacter, pasterellaceae, bordetella, francisella, brucella, legionellaceae, bacteroidaceae, gram-negative bacilli, clostridium, corynebacterium, propionibacterium, gram-positive bacilli, anthrax, actinomyces, nocardia, mycobacterium, treponema, borrelia, leptospira, mycoplasma, ureaplasma, rickettsia, chlamydiae and P. aeruginosa.

Antibiotic MSNPs and protocels can contain one or more antibiotics, e.g., “Antibiotics” include, but are not limited to, compositions selected from the group consisting of Gentamian, Kanamycin, Neomycin, Netilmicin, Tobramycin, Paromomycin, Spectinomycin, Geldanamycin, Herbimycin, Rifaximin, Streptomycin, Ertapenem, Doripenem, Imipenem/Cilastatin, Meropenem, Cefadroxil, Cefazolin, Cephalothin, Cephalexin, Cefador, Cefamandole, Cefoxitin, Cefprozil, Cefuroxime, Cefixime, Cefdinir, Cefditoren, Cefoperazone Cefotaxime, Cefpodoxime, Ceflazidime, Ceftibuten, Ceftizoxime Ceftriaxone, Cefepime, Ceftaroline fosamil, Ceflobiprole, Teicoplanin, Vancomyan, Telavancin, Daptomycin, Oritavancin, WAP-8294A, Azithromycin, Clarithromycin, Dirithromycin, Erythromycin, Roxithromyan, Telithromycin, Spiramycin, Clindamycin, Lincomycin, Aztreonam, Furazolidone, Nitrofurantoin, Oxazolidinones, Linezolid, Posizolid, Radezolid, Torezolid, Amoxicillin, Ampicillin, Azlocillin, Carbenicillin, Cloxacillin Dicloxacillin, Fludoxacillin, Mezlocillin, Methicillin, Nafcillin, Oxacillin, Penicillin G, Penicillin V, Piperacillin, Temocillin, Ticarcillin, Amoxicillin/clavulanate, Ampicillin/sulbactam, Piperacillin/tazobactam, Ticarcilin/lclavulanate, Bacitracin, Colistin, Polymyxin B, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, Mafenide, Sulfacetamide, Sulfadiazine, Sulfadimethoxine, Sulfamethizole, Sulfamethoxazole, Sulfasalazine, Sulfisoxazole, Trimethoprim-Sulfamethoxazole, Sulfonamidochrysoidine, Demeclocycline, Doxycydine, Vibramycin Minocycline, Tigecycline, Oxytetracydine, Tetracycline, Clofazimine, Capreomycin, Cycloserine, Ethambutol, Rifampicin, Rifabutin, Rifapentine, Arsphenamine, Chloramphenicol, Fosfomycin, Fusidic acid, Metronidazole, Mupirocin, Platensimycin, Quinupristin/Dalfopristin, Thiamphenicol, Tigecycline and Tinidazole and combinations thereof.

The term “anticancer agent” shall include any chemotherapeutic agent. In some embodiments, such an agent is selected from the group consisting of microtubule-stabilizing agents, microtubule-disruptor agents, alkylating agents, antimetabolites, epipodophyllotoxins, antineoplastic enzymes, topoisomerase inhibitors, inhibitors of cell cycle progression, and platinum coordination complexes. In some embodiments, the anticancer agent is selected from the group consisting of everolimus, trabectedin, abraxane, TLK 286, AV-299, DN-101, pazopanib, GSK690693. RTA 744, ON 0910.Na, AZD 6244 (ARRY-142886), AMN-107. TKI-258, GSK461364, AZD 1152, enzastaurin, vandetanib. ARQ-197, MK-0457. MLN8054. PHA-739358, R-763, AT-9263, a FLT-3 inhibitor, a VEGFR inhibitor, an EGFR TK inhibitor, an aurora kinase inhibitor, a PIK-1 modulator, a BcI-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespen, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib, PD0325901, AZD-6244, capecitabine, L-glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrozole, exemestane, letrozole, DES(diethylstilbestrol), estradiol, estrogen, conjugated estrogen, bevacizumab, IMC-1C11, CHIR-258, 3-[5-(methylsulfonylpiperadinemethyl)-indolyl]-quinolone, vatalanib, AG-013736, AVE-0005, the acetate salt of [D-Ser(But)6, Azgly10] (pyro-Glu-His-Trp-Ser-Tyr-D-Ser(But)-Leu-Arg-Pro-Azgly-NH₂ acetate [C₅₉H₈₄N₁₈O₁₄—(C₂H₄O₂)x where x=1 to 2.4]), goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatinib, canertinib, ABX-EGF antibody, erbitux, EKB-569, PKI-166, GW-572016, lonafamib, BMS-214662, tipifamib, amifostine. NVP-LAQ824, suberoyl anilide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, dodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deoxyuridine, cytosine arabinoside, 6-mercaptopurine, deoxycoformyan, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxifene, spironolactone, finasteride, cimetidine, trastuzumab, denileukin diftitox, gefitinib, bortezomib, paditaxel, cremophor-free paclitaxel, docetaxel, epithilone B, BMS-247550, BMS-310705, 4-hydroxytamoxifen, pipendoxifene, ERA-923, arzoxifene, fulvestrant, acolbifene, lasofoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696. LY293684. LY293646, wortmannin, ZM336372, L-779,450, PEGfilgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, al-transretinoic acid, ketoconazole, interleukin-2, megestrol, immune globulin, nitrogen mustard, methylprednisolone, ibritumomab tiuxetan, androgens, decitabine, hexamethylmelamine, bexarotene, tositumomab, arsenic trioxide, cortisone, etidronate, mitotane, cydosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, among others.

MSNPs and protocells can comprise anti-cancer agents selected from the group consisting of antimetabolites, inhibitors of topoisomerase I and II, alkylating agents and microtubule inhibitors, adriamycin; aldesleukin; alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; dadribine; cyclophosphamide; cytarabine; dacarbazine; dactinomycin; actinomycn D; Darbepoetin alfa: daunorubicin liposomal; daunorubicin, daunomycin; Denileukin diftitox, dexrazoxane; docetaxel; doxorubicin; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemcitabine, gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); mechlorethamine (nitrogen mustard); megestrol acetate: melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab: Sargramostim; streptozocin; talbuvidine (LDT); talc; tamoxifen; temozolomide; teniposide (VM-26); testolactone: thioguanine (6-TG); thiotepa; topotecan; toremifene; Tositumomab; Trastuzumab; tretinoin (ATRA); uracil mustard; valrubicin; valtorcitabine (monovalyl-LDC); vinblastine; vinorelbine; zoledronate; and mixtures thereof.

In certain embodiments, MSNPs and protocells comprise anti-cancer drugs selected from the group consisting of doxorubicin, melphalan, bevacizumab, dactinomycin, cyclophosphamide, doxorubicin liposomal, amifostine, etoposide, gemcitabine, altretamine, topotecan, cyclophosphamide, paclitaxel, carboplatin, cisplatin, and taxol.

MSNPs and protocells may include one or more antiviral agents to treat viral infections, especially including HIV infections, HBV infections and/or HCV infections. Exemplary anti-HIV agents include, for example, nucleoside reverse transcriptase inhibitors (NRTI), non-nucleoside reverse transcriptase inhibitors (NNRTI), protease inhibitors, fusion inhibitors, among others, exemplary compounds of which may include, for example, 3TC (Lamivudine), AZT (Zidovudine), (−)-FTC, ddl (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate). RTV (Ritonavir), IDV (Indinavir), SQV (Saquinavir), NFV (Nelfinavir), APV (Amprenavir), LPV (Lopinavir), fusion inhibitors such as T20, among others, fuseon and mixtures thereof, including anti-HIV compounds presently in clinical trials or in development. Exemplary anti-HBV agents include, for example, hepsera (adefovir dipivoxil), lamivudine, entecavir, telbivudine, tenofovir, emtricitabine, clevudine, valtorcitabine, amdoxovir, pradefovir, racivir, BAM 205, nitazoxanide, UT 231-B, Bay 41-4109, EHT899, zadaxin (thymosin alpha-1) and mixtures thereof. Anti-HCV agents include, for example, interferon, pegylated interferon, ribavirin, NM 283, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, ACH-1095, GSK625433, TG4040 (MVA-HCV), A-831. F351, NS5A, NS4B, ANA598, A-689, GNI-104, IDX102, ADX184, GL59728, GL60667, PSI-7851, TLR9 Agonist, PHX1766, SP-30 and mixtures thereof.

Other illustrative therapeutic uses of the nanoparticles and protocells include embodiments in which: (a) the nanoparticle is loaded with the naked siRNA TD101 and the nanoparticle or protocell comprising the nanoparticle is useful in the treatment of Pachyonychia Congenita; or (b) the nanoparticle is loaded with the naked siRNA 15NP and the nanoparticle or protocell comprising the nanoparticle is useful in the treatment of delayed graft function associated with kidney transplant; or (c) the nanoparticle is loaded with the naked siRNA SYL040012 and the nanoparticle or protocel comprising the nanoparticle is useful in the treatment of glaucoma and/or ocular hypertension; or (d) the nanoparticle is loaded with the naked siRNA SYL1001 and the nanoparticle or protocell comprising the nanoparticle is useful in the treatment of dry eye syndrome; or (e) the nanoparticle is loaded with the naked siRNA Bevasiranib and the nanoparticle or protocell comprising the nanoparticle is useful in the treatment of Wet AMD or Diabetic AMD; or (f) the nanoparticle is loaded with the naked siRNA QPI-1007 and the nanoparticle or protocell comprising the nanoparticle is useful in the treatment of chronic optic nerve atrophy; or (g) the nanoparticle is loaded with the naked siRNA Sima-027/AGN211745 and the nanoparticle or protocell comprising the nanoparticle is useful in the treatment of AMD and CNV; or (h) the nanoparticle is loaded with the naked siRNA PF-655 and the nanoparticle or protocell comprising the nanoparticle is useful in the treatment of AMD/DME.

Other illustrative therapeutic uses of the nanoparticles and protocells include embodiments in which the nanoparticle is loaded with the siRNA siG12D and the protocell is useful in the treatment of pancreatic cancer. Other illustrative therapeutic uses of the nanoparticles and protocells include embodiments in which the nanoparticle is loaded with the siRNA TKM-PLK1 (PLK1 SNALP, TKM-080301) and the protocell is useful in the treatment of a solid tumor and primary and secondary liver cancer. Other illustrative therapeutic uses of the nanoparticles and protocells include embodiments in which the nanoparticle is loaded with siRNA (e.g., siRNA-EphA2-DOPC), and the protocell is adapted for the treatment of a solid tumor.

Other illustrative therapeutic uses of the nanoparticles and protocells include embodiments in which: (a) the nanoparticle is loaded with the aptamer C2 (2′F RNA) and the protocell is useful in the treatment of leukemia cancer or a skin cancer; (b) the nanoparticle is loaded with the aptamer EpDT3 (2′F RNA) and the protocell is useful in the treatment of colon cancer or breast cancer; (c) the nanoparticle is loaded with the aptamer PSM-A10 (2′F RNA) and the protocell is useful in the treatment of prostate cancer: (d) the nanoparticle is loaded with the aptamer S6 (2′F RNA) and the protocell is useful in the treatment of breast cancer, (e) the nanoparticle is loaded with the aptamer C1 (2′F RNA) and the protocell is useful in the treatment of breast cancer; (f) the nanoparticle is loaded with the aptamer CL4 (2′F RNA) and the protocell is useful in the treatment of breast cancer; (g) the nanoparticle is loaded with the aptamer YJ1 (2′F RNA) and the protocell is useful in the treatment of metastatic colon cancer; (h) the nanoparticle is loaded with the aptamer Aptamer 14 (2′F RNA) and the protocell is useful in the treatment of leukemia; (i) the nanoparticle is loaded with the aptamer C10 (DNA) and the protocell is useful in the treatment of Burkitt like lymphoma; (j) the nanoparticle is loaded with the aptamer Sgc8 (DNA) and the protocel is useful in the treatment of acute lymphoblastic leukemia; or (k) the nanoparticle is loaded with the aptamer TA6 (DNA) and the protocel is useful in the treatment of breast cancer, lymphoma and melanoma.

Typically pharmaceutical formulations and protocells can be loaded with cargo to a capacity up to about 10, 20, 30, 40, 50, 60, 70, 80 or about 90 weight % or more (or from about 0.01% to about 70%, about 0.02% to about 60%, about 0.2 to about 55%, about 0.5% to about 45%, about 1% to about 35%, about 1.5% to about 25%, about 0.1% to about 10%, about 0.01% to about 5%): defined as (cargo weight/weight of loaded protocell)×100. The optimal loading of cargo is often about 0.01 to 60% but this depends on the drug or drug combination which is incorporated as cargo into the MSNPs. This is generally expressed in μM per 10¹⁰ particles where we have values ranging from 2000-100 μM per 10¹⁰ particles. For example, MSNPs exhibit release of cargo at pH about 5.5, which is that of the endosome, but are stable at physiological pH of 7 or higher (e.g., 7.4).

The surface area of the internal space for loading is the pore volume whose optimal value ranges from about 1.1 to 0.5 cubic centimeters per gram (cclg). Note that in the MSNPs according to one embodiment, the surface area is mainly internal as opposed to the external geometric surface area of the nanoparticle.

The lipid bi- or multilayer supported on the porous particle according to one embodiment has a lower melting transition temperature, i.e. is more fluid than a lipid bi- or multilayer supported on a non-porous support or the lipid bi- or multilayer in a liposome. This is sometimes important in achieving high affinity binding of immunogenic peptides or targeting ligands at low peptide densities, as it is the bilayer fluidity that allows lateral diffusion and recruitment of peptides by target cel surface receptors. One embodiment provides for peptides to cluster, which facilitates binding to a complementary target.

The lipid bi- or multilayer may vary significantly in composition. Ordinarily, any lipid or polymer which may be used in liposomes may also be used in MSNPs. For example, lipids are as otherwise described herein.

In some embodiments, the lipid bi- or multilayer of the protocells can provide biocompatibility and can be modified to possess targeting species including, for example, antigens, targeting peptides, fusogenic peptides, antibodies, aptamers, and PEG (polyethylene glycol) to allow, for example, further stability of the protocells and/or a targeted delivery into a cell to maximize an immunogenic response. PEG, when included in lipid bilayers, can vary widely in molecular weight (although PEG ranging from about 10 to about 100 units of ethylene glycol, about 15 to about 50 units, about 15 to about 20 units, about 15 to about 25 units, about 16 to about 18 units, etc., may be used) and the PEG component which is generally conjugated to phospholipid through an amine group comprises about 1% to about 20%, for example about 5% to about 15%, about 10% by weight of the lipids which are included in the lipid bi- or multilayer. The PEG component is generally conjugated to an amine-containing lipid such as DOPE or DPPE or other lipid, but in alternative embodiments may also be incorporated into the MSNPs, through inclusion of a PEG containing silane.

Numerous lipids which are used in liposome delivery systems may be used to form the lipid bi- or multilayer on nanoparticles. Virtually any lipid which is used to form a liposome may be used in the lipid bi- or multilayer which surrounds the nanoparticles according to an embodiment. For example, lipids for use in the present invention include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment given the fact that cholesterol may be an important component of the lipid bilayer of protocels according to an embodiment. Often cholesterol is incorporated into lipid bilayers of protocells in order to enhance structural integrity of the bilayer. These lipids are all readily available commercially from Avanti Polar Lipids, Inc. (Alabaster, Ala., USA). DOPE and DPPE are particularly useful for conjugating (through an appropriate crosslinker) PEG, peptides, polypeptides, including immunogenic peptides, proteins and antibodies, RNA and DNA through the amine group on the lipid.

MSNPs and protocells can be PEGylated with a variety of polyethylene glycol-containing compositions as described herein. PEG molecules can have a variety of lengths and molecular weights and include, but are not limited to, PEG 200, PEG 1000, PEG 1500, PEG 4600, PEG 10,000, PEG-peptide conjugates or combinations thereof.

Additional reporters include polypeptide reporters which may be expressed by plasmids (such as histone-packaged supercoiled DNA plasmids) and include polypeptide reporters such as fluorescent green protein and fluorescent red protein. Reporters pursuant to the present invention are utilized principally in diagnostic applications including diagnosing the existence or progression of a viral infection in a patient and or the progress of therapy in a patient or subject.

Pharmaceutical compositions comprise an effective population of MSNPs and/or protocells as otherwise described herein formulated to effect an intended result (e.g., immunogenic result, therapeutic result and/or diagnostic analysis, including the monitoring of therapy) formulated in combination with a pharmaceutically acceptable carrier, additive, or excipient. The MSNPs and/or protocells within the population of the composition may be the same or different depending upon the desired result to be obtained. Pharmaceutical compositions may also comprise an additional bioactive agent or drug, such as an antiviral agent.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g., orally, transdermally, perineurally or parenterally, that is, by intravenous, subcutaneous, intraperitoneal, intrathecal or intramuscular injection, among others, including buccal, rectal and transdermal administration. Subjects contemplated for treatment according to the method include humans, companion animals, laboratory animals, and the like. The invention contemplates immediate and/or sustained/controlled release compositions, including compositions which comprise both immediate and sustained release formulations. This is particularly true when different populations of MSNPs and/or protocells are used in the pharmaceutical compositions or when additional bioactive agent(s) are used in combination with one or more populations of protocels as otherwise described herein.

Formulations containing the compounds may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, for example in unit dosage forms suitable for simple administration of precise dosages.

Pharmaceutical compositions typically include a conventional pharmaceutical carrier or excipient and may additionally include other medicinal agents, carriers, adjuvants, additives and the like. For example, the composition is about 0.1% to about 85%, about 0.5% to about 75% by weight of a compound or compounds, with the remainder consisting essentially of suitable pharmaceutical excipients.

An injectable composition for parenteral administration (e.g., intravenous, intramuscular or intrathecal) will typically contain the compound in a suitable i.v. solution, such as sterile physiological salt solution. The composition may also be formulated as a suspension in an aqueous emulsion.

Liquid compositions can be prepared by dissolving or dispersing the population of MSNPs and/or protocells (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g., an exapient such as a saccharide or cellulose preparation, a binder such as starch paste or methyl cellulose, a filler, a disintegrator, and other additives typically used in the manufacture of medical preparations.

Methods for preparing such dosage forms are known or is apparent to those skilled in the art; for example, see Remington's Pharmaceutical Sciences (17th Ed., Mack Pub. Co., 1985). The composition to be administered will contain a quantity of the selected compound in a pharmaceutically effective amount for therapeutic use in a biological system, including a patient or subject.

Methods of treating patients or subjects in need for a particular disease state or infection comprise administration an effective amount of a pharmaceutical composition comprising therapeutic MSNPs and/or protocells and optionally at least one additional bioactive (e.g., antiviral) agent.

Intranasal (IN) delivery of broad-spectrum small molecule, nucleic acid, and antibody-based antivirals to central nervous system (CNS) tissues and cells infected with encephalitic New World alphaviruses (e.g., Venezuelan (VEEV), eastern (EEEV), and western (WEEV) equine encephalitis viruses) illustrates in one embodiment treatment modality.

Diagnostic methods comprise administering to a patient in need an effective amount of a population of diagnostic MSNPs and/or protocells (e.g., MSNPs and/or protocells which comprise a target species, such as a targeting peptide which binds selectively to viraly-infected cells and a reporter component to indicate the binding of the protocells) whereupon the binding of the MSNPs and/or protocells to cells as evidenced by the reporter component (moiety) will enable a diagnosis of the existence of a disease state in the patient.

An alternative of the diagnostic method may be used to monitor the therapy of a disease state in a patient, the method comprising administering to a patient an effective population of diagnostic MSNPs and/or protocells (e.g., MSNPs and/or protocells which comprise a target species, such as a targeting peptide which binds selectively to target cells and a reporter component to indicate the binding of the protocels to virally-infected cells if such cells are present) to a patient or subject prior to treatment, determining the level of binding of diagnostic protocells to target cells in said patient and during and/or after therapy, determining the level of binding of diagnostic protocells to target cells in said patient, whereupon the difference in binding before the start of therapy in the patient and during and/or after therapy wil evidence the effectiveness of therapy in the patient, including whether the patient has completed therapy or whether the disease state has been inhibited or eliminated.

There may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994. “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL.

Any number of histone proteins, as well as other means to package the DNA into a smaller volume such as normally cationic nanoparticles, lipids, or proteins, may be used to package the supercoiled plasmid DNA “histone-packaged supercoiled plasmid DNA”, but in therapeutic aspects which relate to treating human patients, the use of human histone proteins are for example used. In certain aspects, a combination of human histone proteins H1. H2A, H2B, H3 and H4 in one embodiment ratio of 1:2:2:2:2, although other histone proteins may be used in other, similar ratios, as is known in the art or may be readily practiced pursuant to the teachings. The DNA may also be double stranded linear DNA, instead of plasmid DNA, which also may be optionally supercoiled and/or packaged with histones or other packaging components.

Other histone proteins which may be used in this aspect include, for example, H1F, H1F0, H1FNT, H1FOO, H1FX H1H1 HIST1H1A, HIST1H1B, HIST1H1C, HIST1H1D, HIST1H1E, HIST1H1T, H2AF, H2AFB1, H2AFB2, H2AFB3, H2AFJ, H2AFV, H2AFX, H2AFY, H2AFY2, H2AFZ, H2A1, HIST1H2AA, HIST1H2AB, HIST1H2AC, HIST1H2AD, HIST1H2AE, HIST1H2AG, HIST1H2AI, HIST1H2AJ, HIST1H2AK, HIST1H2AL, HIST1H2AM, H2A2, HIST2H2AA3, HIST2H2AC, H2BF, H2BFM, HSBFS, HSBFWT, H2B1, HIST1H2BA, HIST1HSBB, HIST1HSBC, HIST1HSBD, HIST1H2BE, HIST1H2BF, HIST1H2BG, HIST1H2BH, HIST1H2BI, HIST1H2BJ, HIST1H2BK, HIST1H2BL, HIST1H2BM, HIST1H2BN, HIST1H2BO, H2B2, HIST2H2BE, H3A1, HIST1H3A, HIST1H3B, HIST1H3C, HIST1H3D, HIST1H3E, HIST1H3F, HIST1H3G, HIST1H3H, HIST1H31, HIST1H3J, H3A2, HIST2H3C, H3A3, HIST3H3, H41, HIST1H4A, HIST1H4B, HIST1H4C, HIST1H4D, HIST1H4E, HIST1H4F, HIST1H4G, HIST1H4H, HIST1H41, HIST1H4J, HIST1H4K, HIST1H4L, H44 and HIST4H4.

Proteins gain entry into the nucleus through the nuclear envelope. The nuclear envelope consists of concentric membranes, the outer and the inner membrane. These are the gateways to the nucleus. The envelope consists of pores or large nuclear complexes. A protein translated with a NLS will bind strongly to importin (aka karyopherin), and together, the complex will move through the nuclear pore. Any number of nuclear localization sequences may be used to introduce histone-packaged plasmid DNA into the nucleus of a cell. Exemplary nuclear localization sequences include H2N-GNQSSNFGPMKGGNFGGRSSGPYGGGGQYFAKPRNQGGYGGC-COOH, RRMKWKK, PKKKRKV, and KR[PAATKKAGQA]KKKK (SEQ ID NOs:24-27), the NLS of nucleoplasmin, a prototypical bipartite signal comprising two clusters of basic amino acids, separated by a spacer of about 10 amino acids. Numerous other nuclear localization sequences are well known in the art. See LaCasse at al., (1995); Weis, (1998); and Murat Cokol at al. et al., (2000).

A peptide nucleic acid can consist of repeating N-(2-aminoethyl)-glycine units linked by amide bonds. The purine (A, G) and pyrimidine (C, T) bases are attached to the backbone through methylene carbonyl linkages. Unlike DNA or DNA analogs. PNAs do not contain any (pentose) sugar moieties or phosphate groups. Surprisingly, PNA's in many respects mimic the behavior of DNA, and in some applications demonstrate superior properties. By convention, PNAs are depicted like peptides, with the N-terminus at the (left) position and the C-terminus at the right. Besides the obvious structural difference, PNA is set apart from DNA in that the backbone of PNA is acyclic, achiral and neutral. PNAs can bind to complementary nucleic acids in both antiparallel and parallel orientation. However, the antiparallel orientation is strongly in one embodiment, and the parallel duplex has been shown to have a different structure. Nielsen, et al., “An Introduction to Peptide Nucleic Acid”, Current Issues Molec. Biol. (1999) 1(2): 89-104.

A level and/or an activity and/or expression of a translation product of a gene and/or of a fragment, or derivative, or variant of said translation product, and/or the level or activity of said translation product, and/or of a fragment, or derivative, or variant thereof, can be detected using an immunoassay, an activity assay, and/or a binding assay. These assays can measure the amount of binding between said protein molecule and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Immunoassays which can be used include e.g., ELISAs, Western blots and other techniques known to those of ordinary skill in the art (see Harlow and Lane, Antibodies: A Laboratory Manual, Cold Spring Harbor Laboratory Press, Cold Spring Harbor, N.Y., 1999 and Edwards R, Immunodiagnostics: A Practical Approach, Oxford University Press, Oxford; England, 1999). All these detection techniques may also be employed in the format of microarrays, protein-arrays, antibody microarrays, tissue microarrays, electronic biochip or protein-chip based technologies (see Schena M., Microarray Biochip Technology, Eaton Publishing. Natick, Mass., 2000).

Certain diagnostic and screening methods utilize an antibody, for example, a monoclonal antibody, capable of specifically binding to a protein as described herein or active fragments thereof. The method of utilizing an antibody to measure the levels of protein allows for non-invasive diagnosis of the pathological states of kidney diseases. In one embodiment, the antibody is human or is humanized. In one embodiment, antibodies may be used, for example, in standard radioimmunoassays or enzyme-linked immunosorbent assays or other assays which utilize antibodies for measurement of levels of protein in sample. In a particular embodiment, the antibodies are used to detect and to measure the levels of protein present in a sample.

Humanized antibodies are antibodies, or antibody fragments, that have the same binding specificity as a parent antibody, (i.e., typically of mouse origin) and increased human characteristics. Humanized antibodies may be obtained, for example, by chain shuffling or by using phage display technology. For example, a polypeptide comprising a heavy or light chain variable domain of a non-human antibody specific for a disease related protein is combined with a repertoire of human complementary (light or heavy) chain variable domains. Hybrid pairings specific for the antigen of interest are selected. Human chains from the selected pairings may then be combined with a repertoire of human complementary variable domains (heavy or light) and humanized antibody polypeptide dimers can be selected for binding specificity for an antigen. Techniques described for generation of humanized antibodies that can be used in the method are disclosed in, for example, U.S. Pat. Nos. 5,565,332; 5,585,089; 5,694,761; and 5.693,762. Furthermore, techniques described for the production of human antibodies in transgenic mice are described in, for example, U.S. Pat. Nos. 5,545,806 and 5,569,825.

In order to identify small molecules and other agents useful in the methods for treating a viral infection by modulating the activity and expression of a disease-related protein and biologically active fragments thereof can be used for screening therapeutic compounds in any of a variety of screening techniques. Fragments employed in such screening tests may be free in solution, affixed to a solid support, borne on a cell surface, or located intracellularly. The blocking or reduction of biological activity or the formation of binding complexes between the disease-related protein and the agent being tested can be measured by methods available in the art.

Other techniques for drug screening which provide for a high throughput screening of compounds having suitable binding affinity to a protein, or to another target polypeptide useful in modulating, regulating, or inhibiting the expression and/or activity of a disease, are known in the art. For example, microarrays carrying test compounds can be prepared, used, and analyzed using methods available in the art. See, e.g., Shalon, D. et al., 1995, International Publication No. WO95/35505, Baldeschweiler et al., 1995. International Publication No. WO95/251116; Brennan et al., 1995. U.S. Pat. No. 5,474,796; Heller et al., 1997, U.S. Pat. No. 5,605,662.

To determine specific binding, various immunoassays may be employed for detecting, for example, human or primate antibodies bound to the cells. Thus, one may use labeled anti-hlg, e.g., anti-hlgM, hlgG or combinations thereof to detect specifically bound human antibody. Various labels can be used such as radioisotopes, enzymes, fluorescers, chemiluminescers, particles, etc. There are numerous commercially available kits providing labeled anti-hlg, which may be employed in accordance with the manufacturers protocol.

In one embodiment, a kit can comprise: (a) at least one reagent which is selected from the group consisting of (i) reagents that detect a transcription product of the gene coding for a protein marker as described herein (ii) reagents that detect a translation product of the gene coding for proteins, and/or reagents that detect a fragment or derivative or variant of said transcription or translation product; (b) instructions for diagnosing, or prognosticating a disease, or determining the propensity or predisposition of a subject to develop such a disease or of monitoring the effect of a treatment by determining a level, or an activity, or both said level and said activity, and/or expression of said transcription product and/or said translation product and/or of fragments, derivatives or variants of the foregoing, in a sample obtained from said subject; and comparing said level and/or said activity and/or expression of said transcription product and/or said translation product and/or fragments, derivatives or variants thereof to a reference value representing a known disease status (patient) and/or to a reference value representing a known health status (control) and/or to a reference value; and analyzing whether said level and/or said activity and/or expression is varied compared to a reference value representing a known health status, and/or is similar or equal to a reference value representing a known disease status or a reference value; and diagnosing or prognosticating a disease, or determining the propensity or predisposition of said subject to develop such a disease, wherein a varied or altered level, expression or activity, or both said level and said activity, of said transcription product and/or said translation product and/or said fragments, derivatives or variants thereof compared to a reference value representing a known health status (control) and/or wherein a level, or activity, or both said level and said activity, of said transcription product and/or said translation product and/or said fragments, derivatives or variants thereof is similar or equal to a reference value and/or to a reference value representing a known disease stage, indicates a diagnosis or prognosis of a disease, or an increased propensity or predisposition of developing such a disease, a high risk of developing signs and symptoms of a disease.

Reagents that selectively detect a transcription product and/or a translation product of the gene coding for proteins can be sequences of various length, fragments of sequences, antibodies, aptamers, siRNA, microRNA, and ribozymes. Such reagents may be used also to detect fragments, derivatives or variants thereof.

Purely by way of example, comparing measured levels of a cancer, bacterial infection or viral infection biomarker (e.g., viral titer) in a sample to corresponding control levels, or comparing measured viral marker levels to control cancer, bacterial infection or viral marker levels determined in a healthy control subject, and determining that a subject suffers from a cancer, bacterial infection or viral infection or that a subject's cancer, bacterial infection or viral infection is progressing, can include determinations based on comparative level differences of about between about 5-10%, or about 10-15%, or about 15-20%, or about 20-25%, or about 25-30%, or about 30-35%, or about 35-40%, or about 40-45%, or about 45-50%, or about 50-55%, or about 55-60%, or about 60-65%, or about 65-70%, or about 70-75%, or about 75-80%, or about 80-85%, or about 85-90%, or about 90-95%, or about 95-100%, or about 100-110%, or about 110-120%, or about 120-130%, or about 130-140%, or about 140-150%, or about 150-160%, or about 160-170%, or about 170-180%, or about 180-190%, or 190-200%, or 200-210%, or 210-220%, or 220-230%, or 230-240%, or 240-250%, or 250-260%, or about 260-270%, or about 270-280%, or about 280-290%, or about 290-300%, or differences of about between about ±50% to about ±0.5%, or about ±45% to about ±1%, or about ±40% to about ±1.5%, or about ±35% to about ±2.0%, or about ±30% to about ±2.5%, or about ±25% to about ±3.0%, or about ±20% to about ±3.5%, or about ±15% to about ±4.0%, or about ±10% to about ±5.0%, or about ±9% to about ±1.0%, or about ±8% to about ±2%, or about ±7% to about ±3%, or about ±6% to about ±5%, or about ±5%, or about ±4.5%, or about ±4.0%, or about ±3.5%, or about ±3.0%, or about ±2.5%, or about ±2.0%, or about ±1.5%, or about ±1.0%.

Protocells described herein are efficiently internalized by host cells, escape intracellular vesicles, and release encapsulated cargos in the cytosol of host cells. A number of factors govern cellular uptake and processing of nanoparticles, including their size, shape, surface charge, and degree of hydrophobicity. Additionally, a variety of molecules, including peptides, proteins, aptamers, and antibodies, can be employed to trigger active uptake by a plethora of specific cells. Incorporation of targeting and endosomolytic peptides that trigger endocytosis and endosomal escape on the protocell SLB enables cell-specific delivery and cytosolic dispersion of encapsulated cargos. SLB fluidity can be tuned to enable exquisite specific affinities for target cells at extremely low targeting ligand densities. In some embodiments, protocells have a sub-nanomolar specific affinity for a target cell. In some embodiments, the sub-nanomolar specific affinity requires about 6 targeting peptides per protocell. The SLB charge can be modulated to reduce non-specific interactions, resulting in protocells that are internalized by target cells 10,000-times more efficiently than non-target cells.

Endosomal escape of protocell-encapsulated cargo maximizes efficacy of the protocell delivery vehicles. Therefore, in some embodiments, the SLBs of protocell are modified with fusogenic peptides (e.g., R8 and H5WYG) that rupture the membranes of acidic intracellular vesicles via the ‘proton sponge’ mechanism.

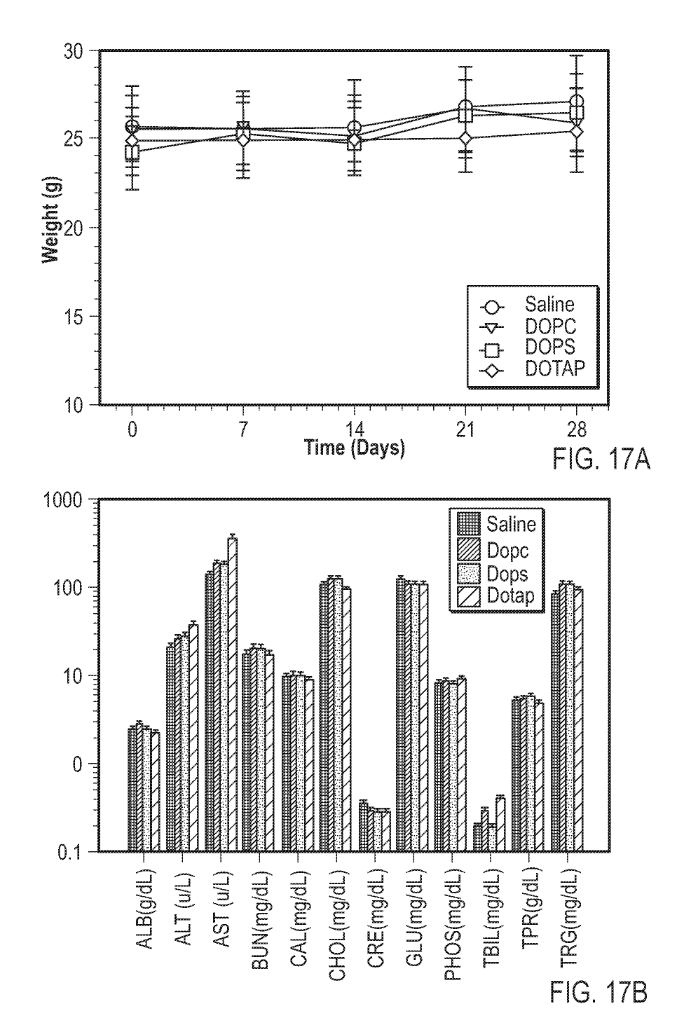

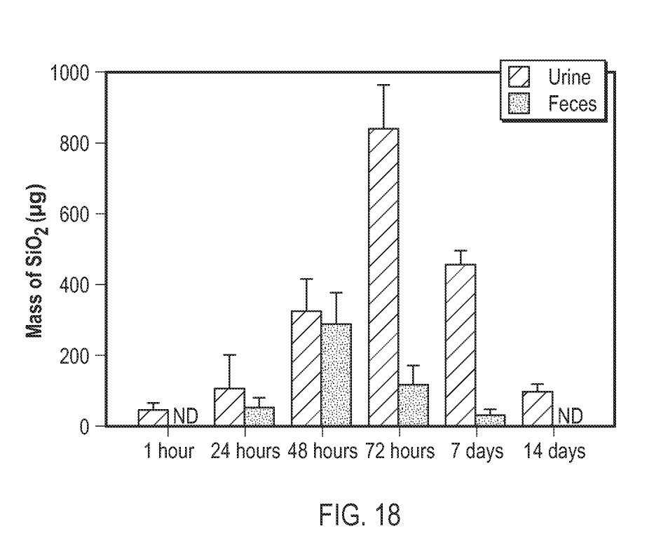

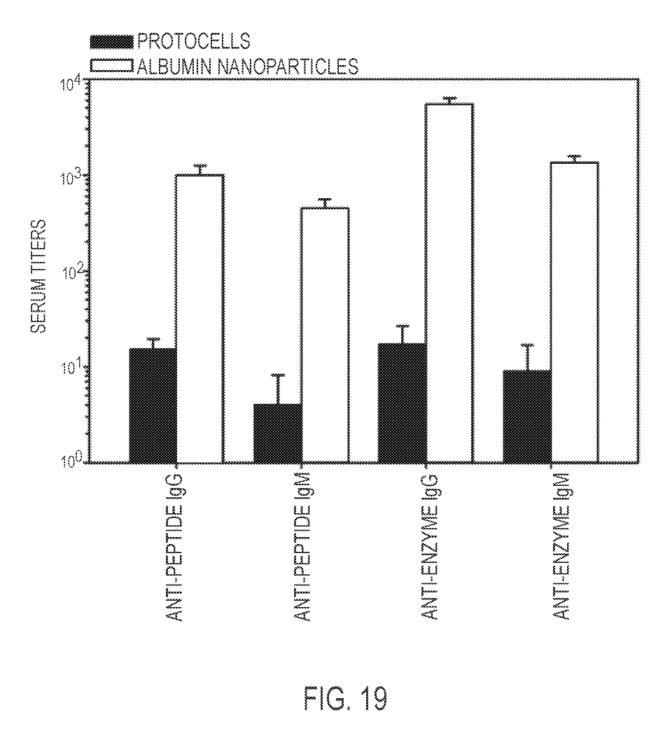

To confirm the safety of the protocells, the biocompatibility, biodegradability, and immunogenicity of the protocells after repeat intraperitoneal (IP) or subcutaneous (SC) injections in Balb/c and C57Bl/6 mice was evaluated. Balb/c mice injected IP with 200 mg/kg doses of DOPC protocells three times each week for 4 weeks showed no signs of gross or histopathological toxicity (see FIG. 17). Furthermore, intact and partially-degraded MSNPs, as well as silicic acid and other byproducts of silica hydrolysis, are excreted in the urine and feces of mice at rates that are determined by the dose, route of administration, and biodistribution (see FIGS. 18 and 20-22). Finally, we have shown that protocells loaded with a therapeutic protein and modified with a high density (about 10 wt % or about 5000 peptides/protocell) of a targeting peptide induce neither IgG nor IgM responses upon SC immunization of C57Bl/6 mice at a total dose of 1000 mg/kg (see FIG. 19).

The Biodistribution of Protocells can be Controlled by Tuning Their Hydrodynamic Size and Surface Modification with Targeting Ligands.

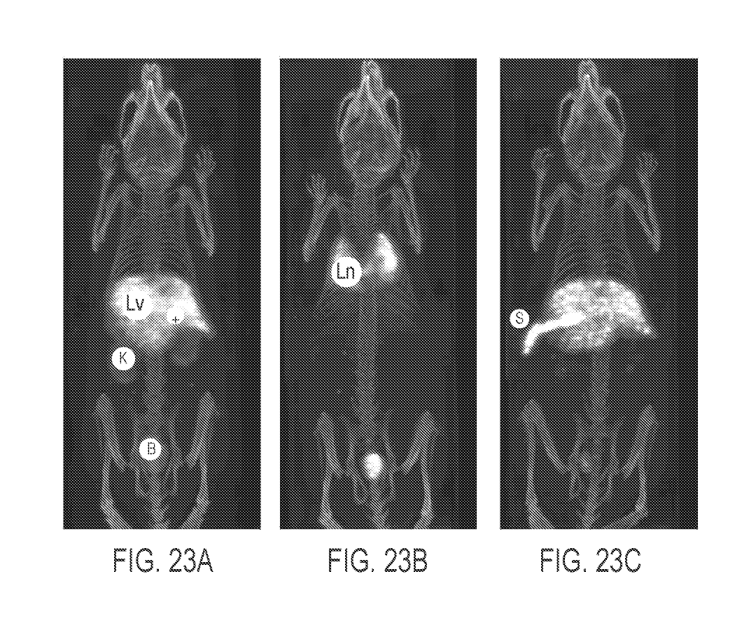

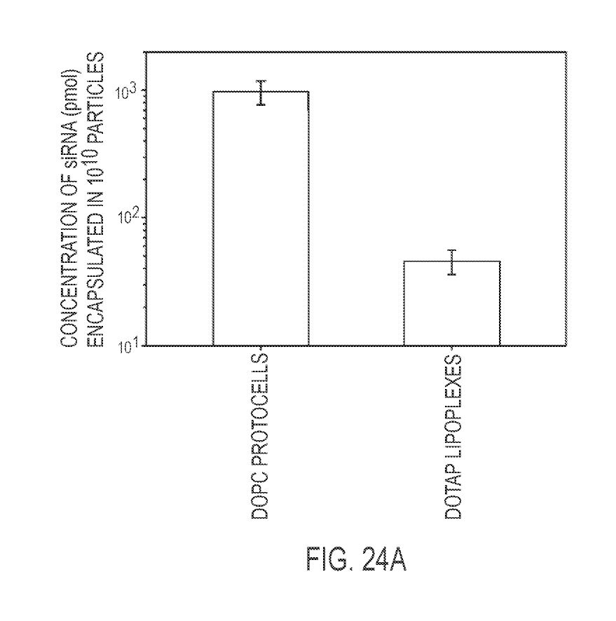

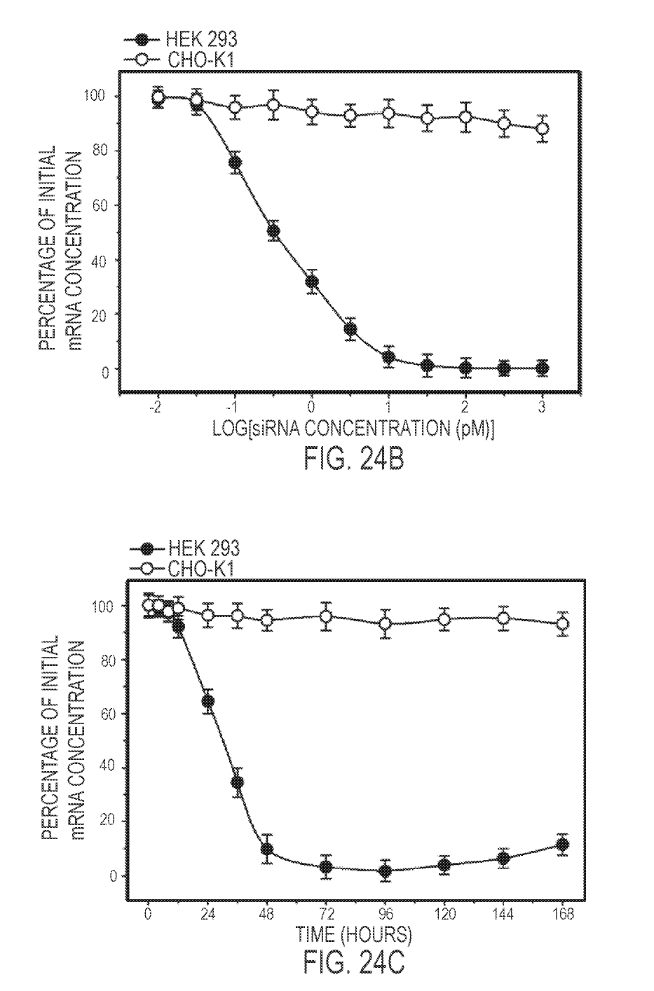

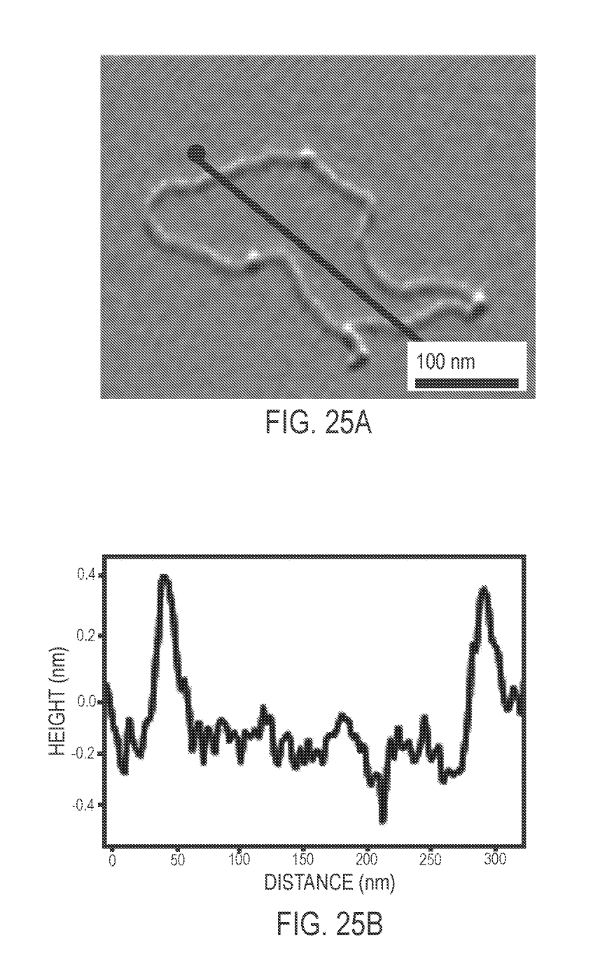

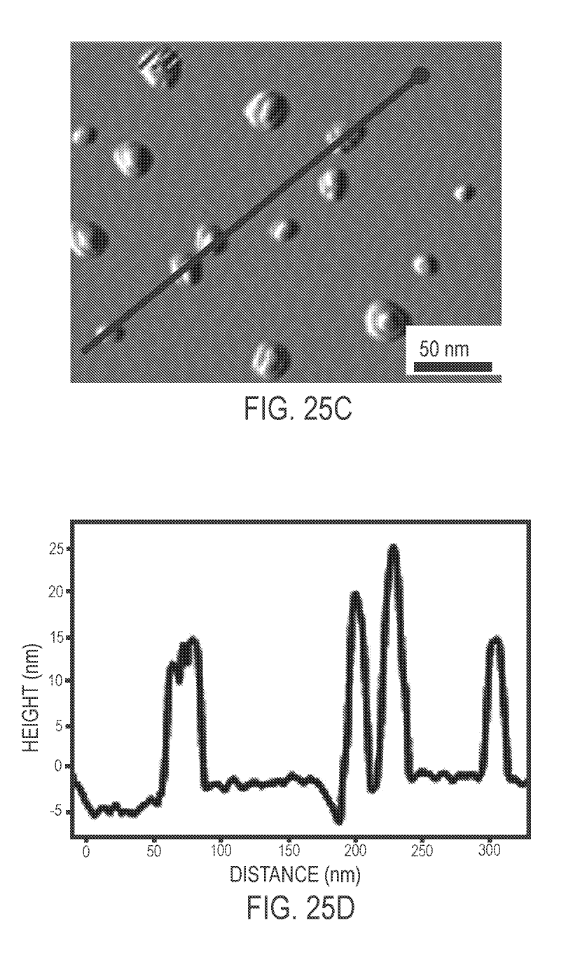

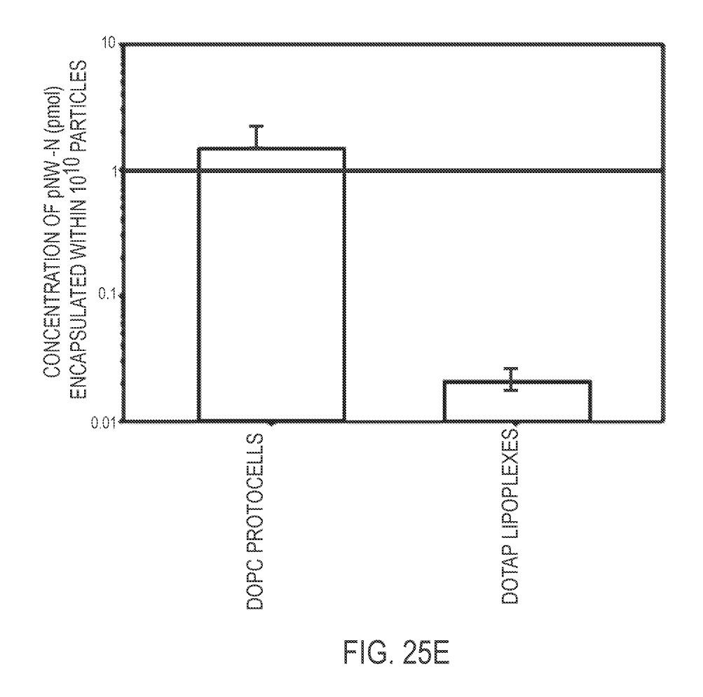

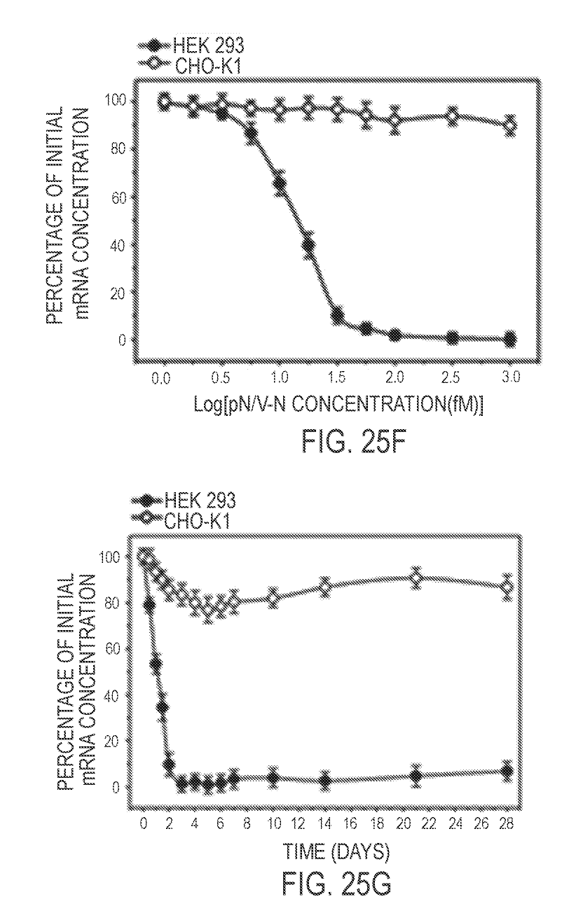

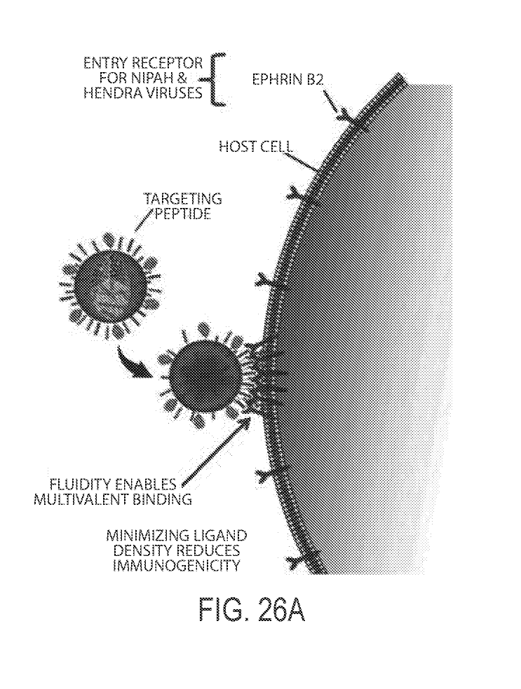

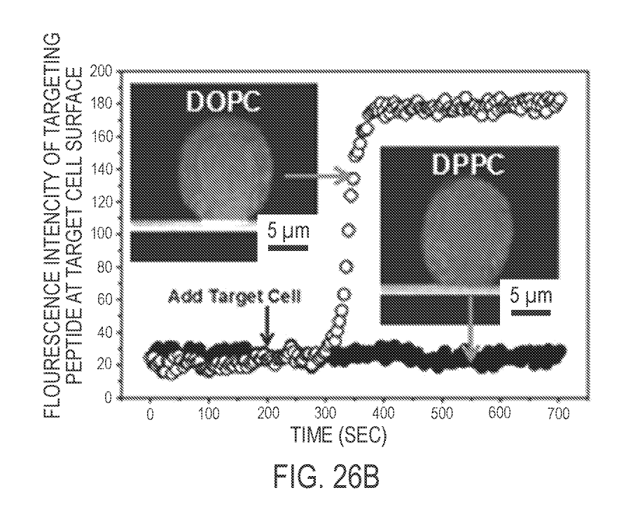

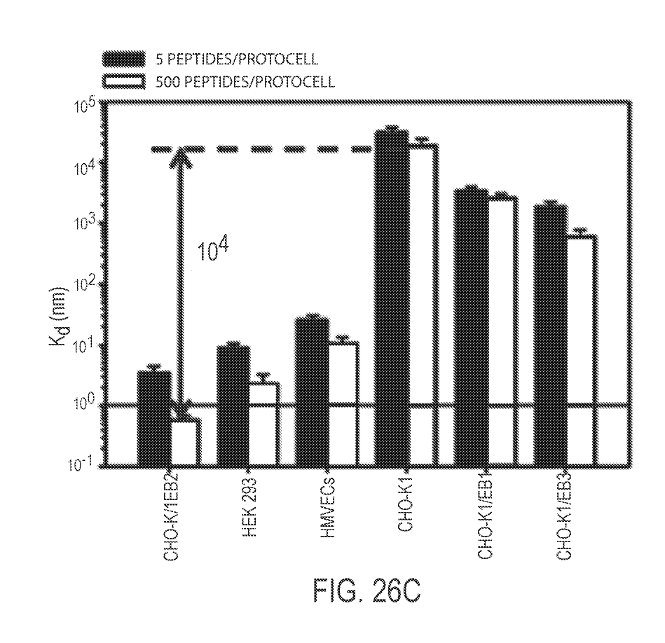

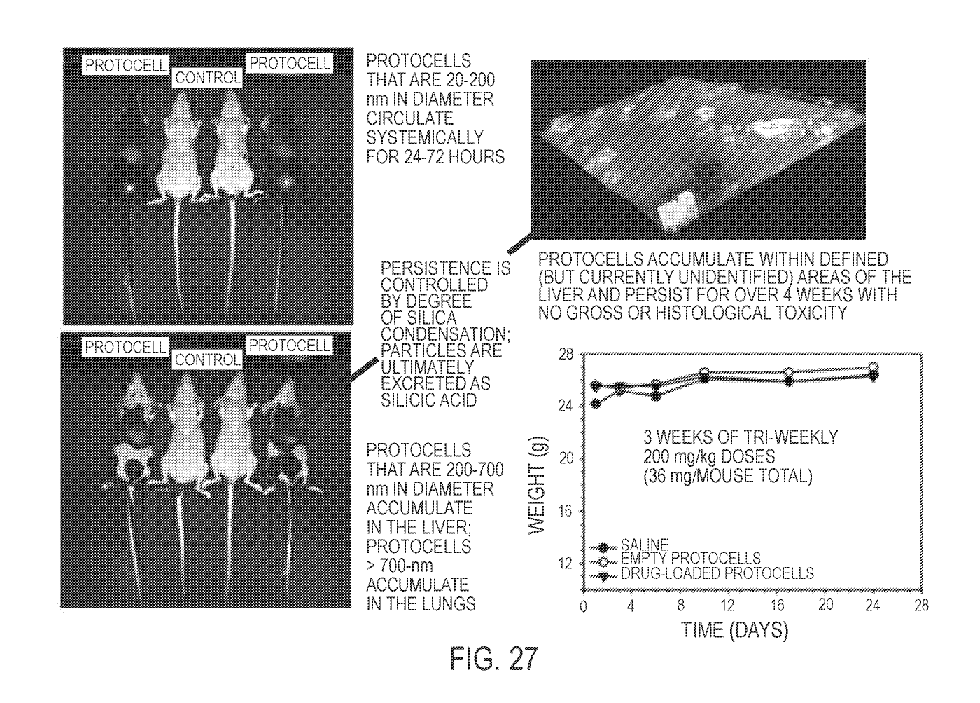

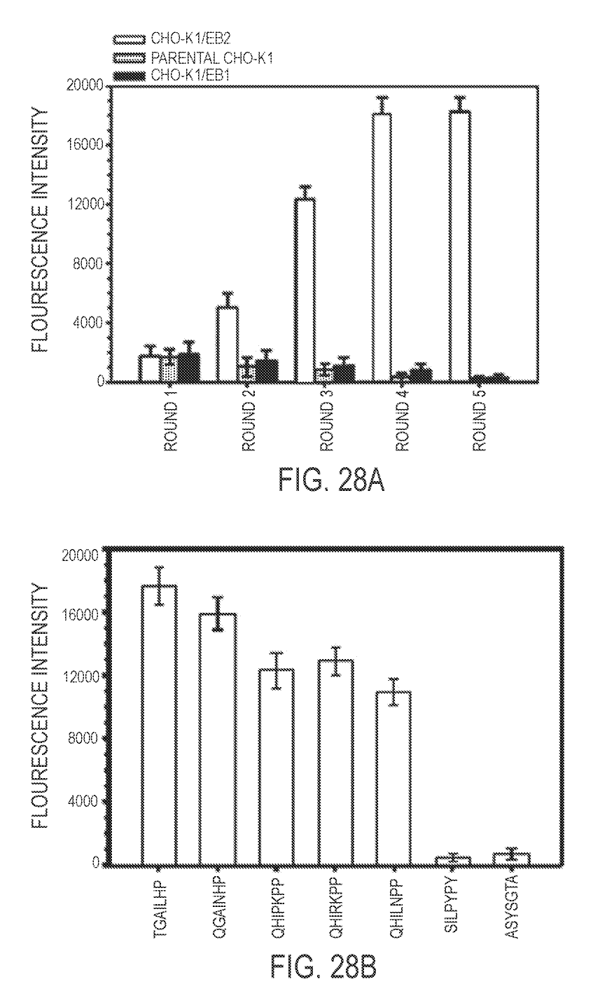

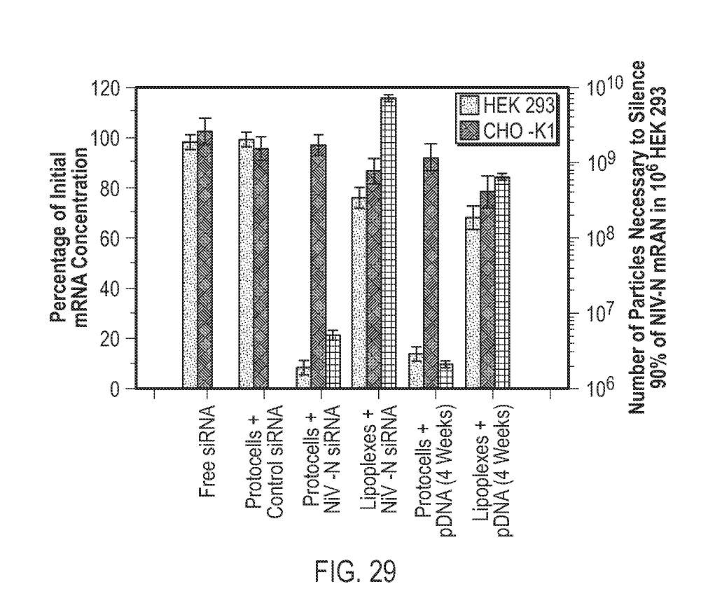

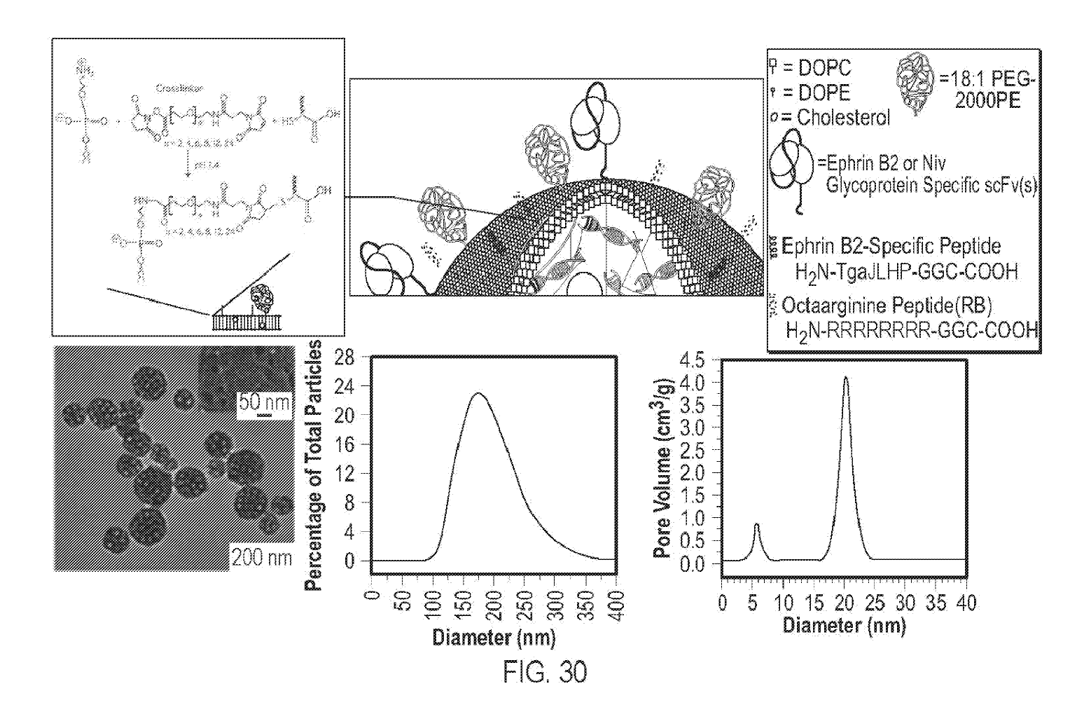

Liposomes and multilamellar vesicles, despite being more elastic that protocells, have biodistributions that are largely governed by their overall size and size distributions, an observation that holds true for protocells as well. The sizes of liposomes and multilamellar vesicles are difficult to control and subject to slight variations in lipid content, buffer pH and ionic strength, and chemical properties of cargo molecules, however. In contrast, the diameter of protocells is governed by the size of the MSNP, which, as we have previously described, is easy to precisely modulate. As demonstrated by FIG. 23, the hydrodynamic size of protocells dramatically affects their bulk biodistributions: protocells 250-nm in diameter accumulate in the liver within 1 hour of injection, while protocells 50-nm in diameter remain in circulation for >1 month. Size-dependent biodistribution can be altered, however, by modifying the surface of DOPC protocells with various types of targeting ligands.

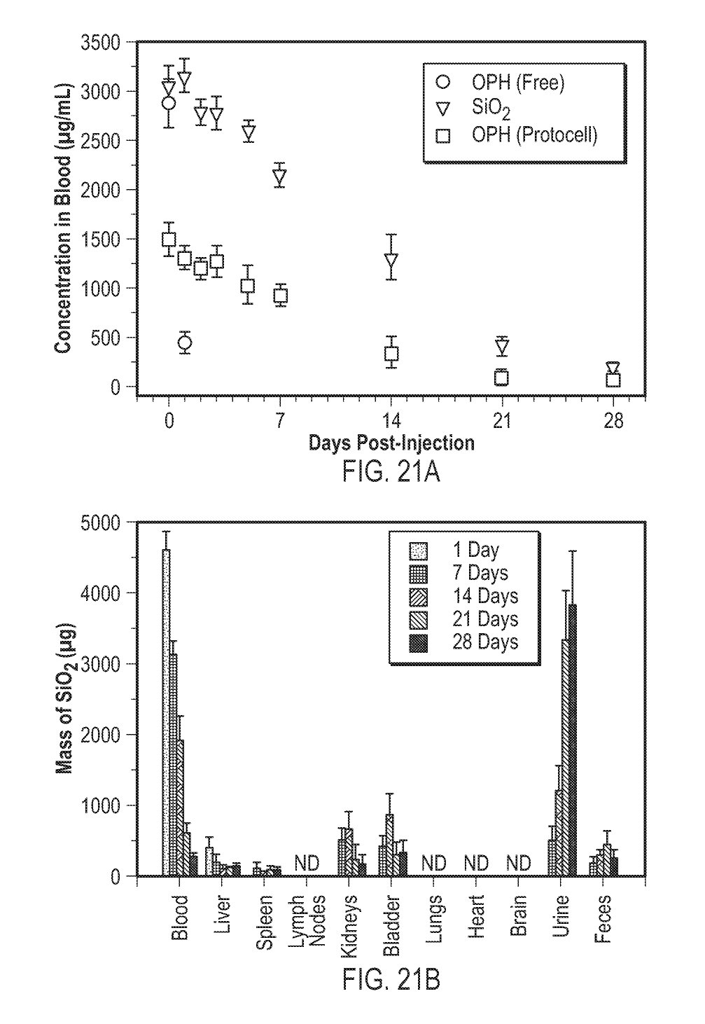

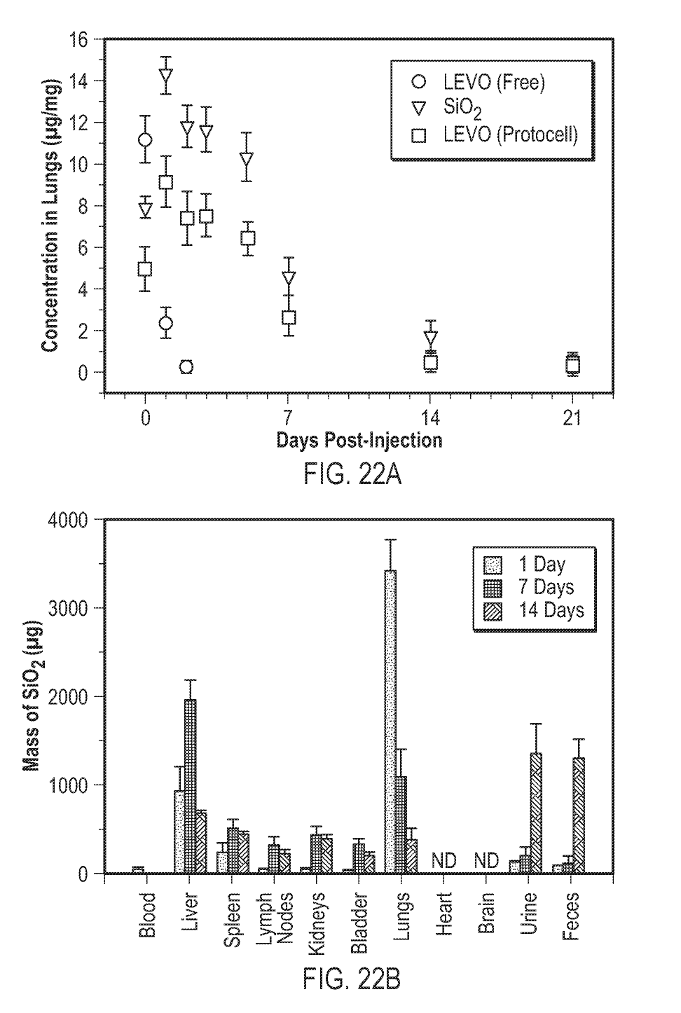

For example, modifying protocels with CD47, a molecule expressed by erythrocytes that innate immune cells recognize as ‘self’, substantially enhances their circulation half-life (see FIG. 21). In contrast, modifying protocells with an aminopeptidase P antibody causes them to rapidly amass in the lung (see FIG. 22). The ability to engineer protocells for both systemic circulation and targeted accumulation within specific organs indicates that we will be able to find a combination of sizes and targeting moieties that enable efficient delivery into the CNS.

A protocell comprises a nanoparticle core surrounded by a lipid bilayer. In some embodiments, the protocell comprises CD47 conjugated to the lipid bilayer. In some embodiments, the protocell comprises aminopeptidase P antibody conjugated to the lipid bilayer. In some embodiments, the protocell comprises Fcγ form conjugated to the lipid bilayer. In some embodiments, the Fcγ is Fcγ from IgG.

Protocels and nanoparticles Functionalized with Hydrophobic Groups and Loaded with Water-Insoluble Cargos.

In some embodiments, the nanoparticles described herein have an enhanced ability to bind water-insoluble cargos. In some embodiments, the water-insoluble cargo has a water solubility of less than about 5 mg/mL. In some embodiments, the water-insoluble cargo has a water solubility of less than about 0.5 mg/mL. In some embodiments, the water-insoluble cargo is a drug, for example an anticancer drug, an antiviral drug, or an antibiotic. In some embodiments, the weight ratio of cargo to silica is about 0.10 to about 0.75. In some embodiments, the nanoparticle comprises silica or metal oxide and is functionalized with a hydrophobic group and loaded with a water-insoluble cargo. In some embodiments, the nanoparticle is porous.

In some embodiments, the nanoparticles functionalized with a hydrophobic group as described herein comprise pores with a diameter of about 0.001 nm to about 100 nm, about 0.01 nm to about 50 nm, about 0.1 to about 100 nm, about 0.1 nm to about 35 nm, or about 0.2 nm to about 25 nm. In some embodiments, the nanoparticles functionalized with a hydrophobic group have a multimodal pore size distribution. In some embodiments, the nanoparticles functionalized with a hydrophobic group have a monomodal pore size distribution.

In some embodiments, the nanoparticles functionalized with a hydrophobic group as described herein have a diameter of about 5 nm to about 500 nm, about 10 nm to about 500 nm, about 25 nm to about 500 nm, about 50 nm to about 500 nm, about 50 nm to about 300 nm, or about 50 nm to about 150 nm. In some embodiments of the nanoparticles functionalized with a hydrophobic group, the nanoparticles have a pore volume fraction of about 25% to about 75%.

In some embodiments of the nanoparticles functionalized with a hydrophobic group as described herein, the hydrophobic group is a phenyl group or a methyl group. In some embodiments of the nanoparticles functionalized with a hydrophobic group, the nanoparticles are functionalized with a hydrophobic organosiloxane, for example hexamethyldisilazane (HDMS), sodium bis(trimethylsilyl)amide (NaHDMS), potassium bis(trimethylsilyl)amide(KHDMS), or phenyltriethoxysilane (PTS).

In some embodiments of the nanoparticles functionalized with a hydrophobic group as described herein, the nanoparticles have a surface area of about 50 m²/g to about 1500 m²/g, or about 100 m²/g to about 1300 m²/g.

In some embodiments of the nanoparticles functionalized with a hydrophobic group as described herein, the nanoparticle has a Zeta (ζ) potential of about −40 mV to about 0 mV. In some embodiments of the nanoparticles functionalized with a hydrophobic group, the nanoparticle is spherical or toroidal.

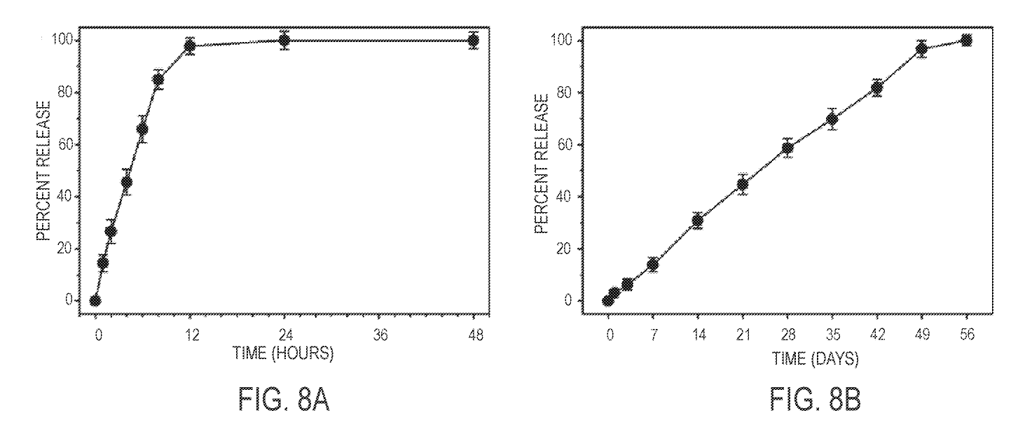

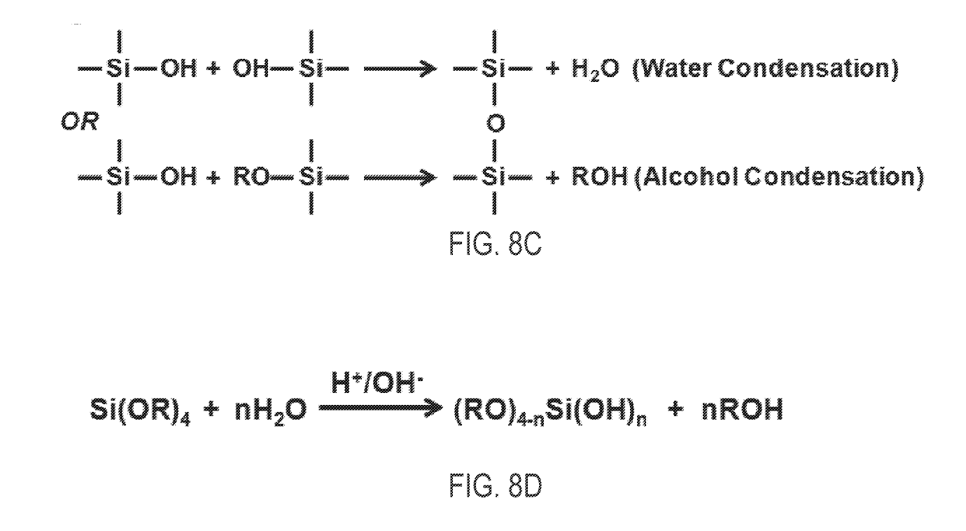

As described in further detail below and illustrated in FIG. 8, the rate at which an encapsulated cargo is released from the nanoparticle can be modulated by varying the degree of silica framework condensation and, therefore, the rate of its dissolution via hydrolysis under physiological conditions. As shown in FIG. 8, silica (SiO₂) forms via condensation and dissolves via hydrolysis. In some embodiments of the nanoparticles functionalized with a hydrophobic group, the nanoparticle is condensed by thermal calcination. In some embodiments of the nanoparticles functionalized with a hydrophobic group, the surfactant has been removed from the nanoparticle core by an acidified C₁₋₄ alcohol to reduce silica condensation.

In some embodiments of the nanoparticles functionalized with a hydrophobic group as described herein, the nanoparticle is PEGylated by covalently attaching a PEG molecule to the surface of the nanoparticle. In some embodiments of the nanoparticles functionalized with a hydrophobic group as described herein, the nanoparticle is not PEGylated.

In some embodiments, a nanoparticle composition comprises a plurality of nanoparticles as described herein. In some embodiments, a nanoparticle composition comprises a plurality of nanoparticles functionalized with a hydrophobic group. In some embodiments of the nanoparticle composition, the nanoparticles are monodisperse. In some embodiments of the nanoparticle composition, the nanoparticles are polydisperse. In some embodiments of the nanoparticle composition, the average diameter of the nanoparticles about 25 nm to about 300 nm, or about 50 nm to about 150 nm.

In any embodiment of the nanoparticles functionalized with a hydrophobic group as described herein, the nanoparticles are coated with a lipid bilayer or a lipid multilayer. In some embodiments of a protocel as described herein, the protocell comprises a nanoparticle functionalized with a hydrophobic group as described herein coated with a lipid bilayer or multilayer.

In some embodiments of a protocell comprising a core functionalized with a hydrophobic group as described herein, the lipid bilayer or multilayer comprises 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]auroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), or cholesterol. In some embodiments of a protocell comprising a core functionalized with a hydrophobic group as described herein, the lipid bilayer or multilayer comprises DOTAP, DOPG, DPPC, DOPE, or DOPC. In some embodiments of a protocell comprising a core functionalized with a hydrophobic group, the lipid bilayer or multilayer comprises DOPC and DOPE. In some embodiments of a protocell comprising a core functionalized with a hydrophobic group, the lipid bilayer or multilayer comprises DOPC and DOPG. In some embodiments of a protocell comprising a core functionalized with a hydrophobic group, the lipid bilayer or multilayer comprises cholesterol. In some embodiments of a protocell comprising a core functionalized with a hydrophobic group, the lipid bilayer or multilayer comprises DOPE, cholesterol, PEG-2000 PE (18:1), and an additional lipid selected from the group consisting of DOPC and DPPC.

In some embodiments of a protocell comprising a core functionalized with a hydrophobic group as described herein, the lipid bilayer or multilayer comprises a cell targeting species. The targeting species allows a protocell to be specifically directed to a targeted cell. Upon binding the targeted cell via the targeting species, the protocell is internalized by the cell. In some embodiments, the cell targeting species is a peptide, an antibody, an antibody fragment, an aptamer, an affibody, a carbohydrate, or functionalized cholesterol. In some embodiments, the targeting species is mannosylated cholesterol. In some embodiments of a protocell comprising a core functionalized with a hydrophobic group and a targeting species, the targeting species targets cancer cells.

In some embodiments of a protocell comprising a core functionalized with a hydrophobic group as described herein, the lipid bilayer or multilayer comprises a fusogenic peptide. Upon binding of the protocell to a targeted cell, the protocell is internalized by the targeted cell by endocytosis. Immediately after being internalized by a targeted cell, the protocell is located in the cellular endosome. The fusogenic peptide promotes endosomal escape, allowing the cargo to be released into the targeted cell.

In some embodiments of a protocell comprising a core functionalized with a hydrophobic group as described herein, the lipid bilayer or multilayer comprises PEG. In some embodiments, the targeting species or the fusogenic peptide is conjugated to the lipid bilayer via a PEG linkage. In some embodiments of a protocell comprising a core functionalized with a hydrophobic group, the lipid bilayer or multilayer does not comprise PEG.

In some embodiments of a protocell comprising a core functionalized with a hydrophobic group as described herein, the protocell has a diameter of about 50 nm to about 300 nm, or about 50 nm to about 150 nm. In some embodiments of a protocell comprising a core functionalized with a hydrophobic group, the protocell has a zeta potential of about −50 mV to about +50 mV.

In some embodiments of a protocell comprising a core functionalized with a hydrophobic group as described herein, the lipid bilayer is modified with glutathione. In some embodiments of a protocell comprising a core functionalized with a hydrophobic group as described herein, the protocell traverses a cellular barrier, such as the blood-brain barrier (BBB) or a nasal epithelium.

In some embodiments of a protocell comprising a core functionalized with a hydrophobic group as described herein, the lipid bilayer is conjugated to CD47. In some embodiments of a protocel comprising a core functionalized with a hydrophobic group, the lipid bilayer is conjugated to aminopeptidase P antibody. In some embodiments of a protocell comprising a core functionalized with a hydrophobic group, the protocell releases about 30% to about 100% of its cargo after three hours at pH 5. In some embodiments of a protocell comprising a core functionalized with a hydrophobic group, the protocell releases about 60% to about 100% of its cargo after six hours at pH 5. In some embodiments of a protocel comprising a core functionalized with a hydrophobic group, the protocel releases substantially all of its cargo after about twelve hours at about physiological pH. In some embodiments of a protocell comprising a core functionalized with a hydrophobic group, the protocell releases its cargo through sustained release at a rate of about 7 wt % to about 10 wt % cargo per day over a period of about ten days.

In some embodiments of a protocell comprising a core functionalized with a hydrophobic group as described herein, the lipid bilayer is conjugated to CD47. In some embodiments of a protocell comprising a core functionalized with a hydrophobic group, the lipid bilayer is conjugated to aminopeptidase P antibody.

In some embodiments of a protocell comprising a core functionalized with a hydrophobic group, the protocells do not stimulate an immune response after administration. In some embodiments of a protocell comprising a core functionalized with a hydrophobic group, the protocells do not stimulate an IgG or IgM response after administration.

Protocels that Traverse a Cellular Barrier

Many drug compounds demonstrate high potential in in vitro studies, but ultimately fail in vivo because of an inability to traverse a cellular barrier, such as the blood-brain barrier. For example, ribavirin does not readily cross the blood brain barrier when administered intravenously or orally, resulting in subtherapeutic concentration in the central nervous system (CNS). Therefore, there is a great need for nanoparticle delivery vehicles that are able to encapsulate multiple types of physicochemicaly disparate drugs in high concentrations, remain stable in blood and other complex biological fluids, and effectively penetrate the blood-brain barrier to target select cells in the CNS, and controllably release the encapsulated drugs into the cytosol of the targeted cells.

In some embodiments, protocells described herein traverse a cellular barrier, for example an endothelial cell barrier (such as the blood-brain barrier) or an epithelial cell barrier (such as the nasal epithelium). By traversing these cellular barriers, protocells accumulate in the central nervous system (CNS) in an increased concentration, thereby facilitating delivery of the protocel cargo to neurons or other CNS cells.

In some embodiments, the protocells comprise a silica or metal oxide nanoparticle core coated with a lipid bilayer or multilayer, the lipid bilayer or multilayer comprises a cellular barrier penetrating moiety. In some embodiments, the cellular barrier penetrating moiety is conjugated to the lipid bilayer or multilayer, for example via a PEG linkage. In some embodiments, the cellular barrier penetrating moiety is an endothelial cell barrier penetrating moiety. In some embodiments, the cellular barrier penetrating moiety is an epithelial cell barrier penetrating moiety. In some embodiments, the cellular barrier penetrating moiety is glutathione. In some embodiments, the cellular barrier penetrating moiety is L-dihydroxyphenylalanine (L-DOPA). In some embodiments, the cellular barrier penetrating moiety is an RGD (Arg-Gly-Asp) peptide or a peptide comprising an RGD sequence. The cellular barrier penetrating moieties attached to the lipid bilayer of the protocells enhance cellular transcytosis, increasing CNS penetration of the protocells.

In some embodiments of a protocell comprising a cellular barrier penetrating moiety as described herein, the nanoparticle core of the protocell is porous. In some embodiments of a protocell comprising a cellular barrier penetrating moiety, the nanoparticle core comprises pores with a diameter of about 1 nm to about 100 nm, about 1 nm to about 50 nm, about 1 nm to about 35 nm, or about 2 nm to about 25 nm. In some embodiments of a protocell comprising a cellular barrier penetrating moiety, the nanoparticle core has a monomodal pore size distribution. In some embodiments of a protocel comprising a cellular barrier penetrating moiety, the nanoparticle core has a multimodal pore size distribution. In some embodiments of a protocell comprising a cellular barrier penetrating moiety, the nanoparticle core has a pore volume fraction of about 25% to about 75%. In some embodiments of a protocell comprising a cellular barrier penetrating moiety, the nanoparticle core has a surface area of about 50 m²/g to about 1500 m²/g, or about 100 m²/g to about 1300 m²/g.

In some embodiments of a protocell comprising a cellular barrier penetrating moiety as described herein, the nanoparticle core has a diameter of about 5 nm to about 500 nm, about 25 nm to about 500 nm, about 50 nm to about 500 nm, about 50 nm to about 300 nm, or about 50 nm to about 150 nm.

In some embodiments of a protocell comprising a cellular barrier penetrating moiety as described herein, the nanoparticle core has a zeta (ζ) potential of about −50 mV to about +50 mV. In some embodiments of a protocell comprising a cellular barrier penetrating moiety as described herein, the nanoparticle core has a zeta (ζ) potential of about −40 mV to about 0 mV. In some embodiments of a protocell comprising a cellular barrier penetrating moiety, the nanoparticle core has a zeta (Q potential of about 0 mV to about +50 mV. In some embodiments of a protocell comprising a cellular barrier penetrating moiety, the protocell core has a zeta (0 potential of about −50 mV to about +50 mV.

In some embodiments of a protocell comprising a cellular barrier penetrating moiety as described herein, the nanoparticle core is loaded with a cargo. In some embodiments of a protocel comprising a cellular barrier penetrating moiety, the weight ratio of cargo to silica is about 0.10 to about 0.75. In some embodiments, the cargo is a small-molecule drug, such as an anticancer agent, an antiviral agent, or an antibiotic. In some embodiments, the cargo is a polynucleotide, such as DNA (for example, a plasmid or minicircle) or RNA (for example, mRNA, siRNA, or miRNA). In some embodiments, the cargo is a hydrophobic cargo. In some embodiments, the hydrophobic cargo has a water solubility of less than about 5 mg/ml, or less than about 0.5 mg/ml.

In some embodiments of a protocell comprising a cellular barrier penetrating moiety as described herein, the nanoparticle core is functionalized with a compound to adjust the zeta potential of the nanoparticle core. In some embodiments of a protocell comprising a cellular barrier penetrating moiety, the nanoparticle core is functionalized with a hydrophobic group. In some embodiments of a protocell comprising a cellular barrier penetrating moiety, the nanoparticle core is functionalized with a hydrophobic group and has a zeta potential of about −40 mV to about 0 mV. In some embodiments, the nanoparticle core is functionalized with a hydrophobic organosiloxane, such as hexamethyldisilazane (HDMS), sodium bis(trimethylsilyl)amide (NaHDMS), potassium bis(trimethylsilyl)amide (KHDMS), or phenyltriethoxysilane (PTS). In some embodiments of a protocel comprising a cellular barrier penetrating moiety as described herein, the nanoparticle core is functionalized with an amine-modified silane, such as a primary amine, a secondary amine a tertiary amine, each of which is functionalized with a silicon atom (2) a monoamine or a polyamine (3)N-(2-aminoethyl)-3-aminopropyltrimethoxysiane (AEPTMS) (4) 3-aminopropyltrimethoxysilane (APTMS) (5) 3-aminopropyltriethoxysilane (APTS) (6) an amino-functional trialkoxysilane, and (7) protonated secondary amines, protonated tertiary alkyl amines, protonated amidines, protonated guanidines, protonated pyridines, protonated pyrimidines, protonated pyrazines, protonated purines, protonated imidazoles, protonated pyrroles, and quatemary alkyl amines, or combinations thereof. In some embodiments of a protocell comprising a cellular barrier penetrating moiety as described herein, the nanoparticle core is functionalized with an amine-modified silane and has a zeta potential of about 0 mV to about +50 mV.

In some embodiments of a protocell comprising a cellular barrier penetrating moiety as described herein, the nanoparticle core is spherical or toroidal. In some embodiments of a protocell comprising a cellular barrier penetrating moiety, the nanoparticle is condensed by thermal calcination. In some embodiments of a protocell comprising a cellular barrier penetrating moiety, the surfactant has been removed from the nanoparticle core by an acidified C₁₋₄ alcohol to reduce silica condensation.

In some embodiments of a protocell comprising a cellular barrier penetrating moiety as described herein, the nanoparticle is coated with a lipid bilayer. In some embodiments of a protocell comprising a cellular barrier penetrating moiety, the nanoparticle is coated with a lipid multilayer. In some embodiments of a protocell comprising a cellular barrier penetrating moiety as described herein, the lipid bilayer or lipid multilayer comprises lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]auroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), and cholesterol. In some embodiments of a protocell comprising a cellular barrier penetrating moiety as described herein, the lipid bilayer or multilayer comprises DOTAP, DOPG, DPPC, DOPE, or DOPC. In some embodiments of a protocel comprising a cellular barrier penetrating moiety, the lipid bilayer or multilayer comprises DOPC and DOPE. In some embodiments of a protocell comprising a cellular barrier penetrating moiety, the lipid bilayer or multilayer comprises DOPC and DOPG. In some embodiments of a protocell comprising a cellular barrier penetrating moiety, the lipid bilayer or multilayer comprises cholesterol. In some embodiments of a protocell comprising a cellular barrier penetrating moiety, the lipid bilayer or multilayer comprises DOPE, cholesterol, PEG-2000 PE (18:1), and an additional lipid selected from the group consisting of DOPC and DPPC.

In some embodiments of a protocell comprising a cellular barrier penetrating moiety as described herein, the lipid bilayer or multilayer comprises a cell targeting species. The targeting species allows a protocell to be specifically directed to a targeted cell. Upon binding the targeted cell via the targeting species, the protocell is internalized by the cell. In some embodiments, the cell targeting species is a peptide, an antibody, an antibody fragment, an aptamer, an affibody, a carbohydrate, or functionalzed cholesterol. In some embodiments, the targeting species is mannosylated cholesterol. In some embodiments of a protocell comprising a cellular barrier penetrating moiety as described herein, the targeting species targets cancer cells. In some embodiments, protocells that are able to traverse a cellular barrier comprise a targeting species to target neurons. For example, in some embodiments, the targeting species comprises apolipoprotein E (ApoE), dihydrolipoic acid (DHLA), or a scFv against neural cell adhesion molecule 1 (NCAM1).

In some embodiments of a protocell comprising a cellular barrier penetrating moiety as described herein, the lipid bilayer or multilayer comprises a fusogenic peptide. Upon binding of the protocell to a targeted cell, the protocel is internalized by the targeted cell by endocytosis. Immediately after being internalized by a targeted cell, the protocell is located in the cellular endosome. The fusogenic peptide promotes endosomal escape, allowing the cargo to be released into the targeted cell.

In some embodiments of a protocell comprising a cellular barrier penetrating moiety as described herein, the lipid bilayer or multilayer comprises PEG. In some embodiments, the targeting species or the fusogenic peptide is conjugated to the lipid bilayer via a PEG linkage. In some embodiments of a protocell comprising a cellular barrier penetrating moiety as described herein, the lipid bilayer or multilayer does not comprise PEG.

In some embodiments of a protocell comprising a cellular barrier penetrating moiety as described herein, the protocell has a diameter of about 50 nm to about 300 nm, or about 50 nm to about 150 nm.

In some embodiments of a protocell comprising a cellular barrier penetrating moiety as described herein, the lipid bilayer is conjugated to CD47. In some embodiments of a protocell comprising a cellular barrier penetrating moiety as described herein, the lipid bilayer is conjugated to aminopeptidase P antibody. In some embodiments of a protocell comprising a cellular barrier penetrating moiety, the protocell releases about 30% to about 100% of its cargo after three hours at pH 5. In some embodiments of a protocell comprising a cellular barrier penetrating moiety, the protocell releases about 60% to about 100% of its cargo after six hours at pH 5. In some embodiments of a protocell comprising a cellular barrier penetrating moiety, the protocell releases substantially all of its cargo after about twelve hours at about physiological pH. In some embodiments of a protocell comprising a cellular barrier penetrating moiety, the protocell releases its cargo through sustained release at a rate of about 7 wt % to about 10 wt % cargo per day over a period of about ten days.

In some embodiments of a protocell comprising a cellular barrier penetrating moiety as described herein, the protocells do not stimulate an immune response after administration. In some embodiments of a protocel comprising a cellular barrier penetrating moiety, the protocells do not stimulate an IgG or IgM response after administration.

Exemplary Methods of Forming Protocells and Nanoparticles.

The nanoparticles as described herein are generally produced using aerosol-assisted evaporation-induced self-assembly (EISA) methods. In some embodiments, the EISA method comprises atomizing a nanoparticle precursor solution to produce droplets, and drying and heating the droplets to produce the nanoparticles. In some embodiments, the EISA method comprises forming an emulsion by combining an aqueous phase precursor solution and an oil phase precursor solution and heating the emulsion to produce the nanoparticles. To produce nanoparticles functionalized with hydrophobic groups, a hydrophobic siloxane is included in the any of the precursor solutions.

Cargo release rates can be controlled by altering the degree of silica condensation in the nanoparticle core. The cargo generally unloads during the dissolution of the nanoparticle core under physiological conditions, and the dissolution rate is determined by the degree of silica condensation. Thermal calcination of the nanoparticles maximizes condensation and results in particles with sustained release profiles (7-10% release per day for up to 2 weeks). In contrast, use of acidified ethanol to extract surfactants results in burst release of encapsulated cargos (100% release within 12 hours). Therefore, following the production of the nanoparticles, in some embodiments, the nanoparticles are thermally calcined. In some embodiments, surfactant is extracted from the nanoparticles using an acidified C₁₋₄ alcohol.

In some embodiments, the EISA process for making nanoparticles comprises atomizing a nanoparticle precursor solution to generate droplets; and drying and heating the droplets, thereby evaporating solvent and increasing effective surfactant concentration. In some embodiments, the nanoparticle precursor solution comprises (1) a surfactant, (2) tetraethyl orthosilicate (TEOS) or tetramethyl orthosilicate (TMOS), (3) a C₁₋₄ alcohol, such as ethanol, and (4) water. Optionally, a hydrophobic organosiloxane is included in the nanoparticle precursor solution. In some embodiments, following the formation of the nanoparticles, a cargo, such as a water-insoluble cargo, is added. In some embodiments, the surfactant in the nanoparticle precursor solution is below the critical micelle concentration of the surfactant.

In some embodiments, the surfactant comprises a cationic surfactant, such as a dodecylsulfate salt, a tetradecyl-trimethyl-ammonium salt, a hexadecyltrimethylammonium salt, an octadecyltrimethylammonium salt, a dodecylethyldimethylammonium salt, a cetylpyridinium salt, polyethoxylated tallow amine (POEA), hexadecyltrimethylammonium p-toluenesulfonate, a benzalkonium salt, a Brij® surfactant, a poloxamer, and a benzethonium salt. Exemplary cationic surfactants includes benzethonium chloride, benzalkonium chloride, cetylpyndinium chloride, dodecylethyldimethylammonium bromide, octdadecyltrimethylammonium bromide, hexadecyltrimethylammonium bromide, tetradecyl-trimethyl-ammonium bromide, tetradecyl-trimethyl-ammonium chloride, sodium dodecylsulfate, lithium dodecylsulfate. Brij®-56, Pluronic® F108, and Pluronic® P123. In some embodiments, the nanoparticle precursor solution further comprises urea, poly(propylene oxide) (PPO), poly(ethylene oxide) (PEO), polypropylene glycol acrylate (PPGA), or glycerol.

In another example of an EISA process, an emulsion is formed by combining a aqueous phase precursor solution and an oil phase precursor solution. Generally the oil phase and aqueous phase precursor solutions are combined at a volumetric ratio of about 1:2 to about 1:4 aqueous phase:oil phase. The aqueous phase precursor solution comprises (1) a first surfactant, (2) tetraethyl orthosilicate (TEOS) or tetramethyl orthosilicate (TMOS), (3) an acid, and (4) water. The oil phase precursor solution comprises a second surfactant and an oil, such as a C₁₂-C₂₀ alkane. Optionally, a hydrophobic organosiloxane is included in either the aqueous phase precursor solution or the oil phase precursor solution. The emulsion is then heated to generate the nanoparticles. Generally, the nanoparticles are separated from the remaining emulsion before being loaded with a cargo, such as a water-insoluble cargo. In some embodiments, the concentration of the first surfactant is above the critical micelle concentration of the surfactant in the aqueous phase precursor solution.

In some embodiments, the first surfactant is a cationic surfactant, such as sodium dodecylsulfate, lithium dodecylsulfate, a tetradecyl-trimethyl-ammonium salt, a hexadecyltrimethylammonium salt, an odadecyltrimethylammonium salt, a dodecylethyldimethylammonium salt, a cetylpyridinium salt, polyethoxylated tallow amine (POEA), hexadecyltrimethylammonium p-toluenesulfonate, a benzalkonium salt, or a benzethonium salt. Examples of the first surfactant include tetradecyl-trimethyl-ammonium bromide (C₁₄TAB), tetradecyl-trimethyl-ammonium chloride, hexadecyltrimethylammonium bromide (C₁₆TAB), octadecyitrimethylammonium bromide (C₁₈TAB), dodecylethyldimethylammonium bromide (C₁₂TAB), cetylpyridinium chloride (CPC), benzalkonium chloride (BAC), and benzethonium chloride (BZT). Generally, the second surfactant is a nonionic surfactant such as a poloxamer or a nonionic silicon-based surfactant. Exemplary second surfactants include a Brij® surfactant, Pluronic® F108, Pluronic® P123, or ABIL EM 90.

In some embodiments, the optional hydrophobic organosiloxane is a methyl-containing organosiloxane or a phenyl-containing organosiloxane. Exemplary hydrophobic organosiloxanes include hexamethyldisilazane (HDMS), sodium bis(trimethylsilyl)amide (NaHDMS), potassium bis(trimethylsilyl)amide (KHDMS), or phenyltriethoxysilane (PTS).

After the nanoparticles are formed, for example by using the EISA methods described herein, the nanoparticles are loaded with a cargo before being surrounded by a lipid bilayer to form a protocell. Alternatively, the nanoparticle is simultaneously loaded with a cargo and surrounded by the lipid bilayer to form the protocell.

A nanoparticle composition as described herein comprises a plurality of nanoparticles. In some embodiments, the nanoparticle composition comprises a plurality of nanoparticle, wherein the nanoparticle comprise a silica or metal oxide, the nanoparticle being functionalized with a hydrophobic group and loaded with a water-insoluble cargo.

In some embodiments of a nanoparticle composition as described herein, the nanoparticle are monodisperse. In some embodiments of a nanoparticle composition as described herein, the nanoparticle are polydisperse. In some embodiments of a nanoparticle composition as described herein, the average diameter of the nanoparticle is about 25 nm to about 300 nm, or about 50 nm to about 150 nm.

A protocell composition as described herein comprises a plurality of protocells. In some embodiments, the protocell composition comprises a plurality of protocells, wherein the protocells comprise a silica or metal oxide nanoparticle core coated with a lipid bilayer or multilayer, wherein the lipid bilayer or multilayer comprises a cellular barrier penetrating moiety. In some embodiments, the protocell composition comprises a plurality of protocells, wherein the protocells comprise a silica or metal oxide nanoparticle core, the nanoparticle being functionalized with a hydrophobic group and loaded with a water-insoluble cargo, and a lipid bilayer surrounding the core.

In some embodiments of a protocell composition as described herein, the protocells are monodisperse. In some embodiments of a protocell composition as described herein, the protocells are polydisperse. In some embodiments of a protocell composition as described herein, the average diameter of the protocells is about 50 nm to about 300 nm, or about 50 nm to about 150 nm.

A pharmaceutical composition as described herein comprises a protocell composition as described herein or a nanoparticle composition as described herein. In some embodiments, the pharmaceutical composition comprises a pharmaceutically acceptable carrier, additive, or excipient. In some embodiments, the pharmaceutical composition is administered intranasally, intradermally, intramuscularly, intraosseously, intraperitoneally, intravenously, subcutaneously, or intrathecally.

In some embodiments, the pharmaceutical compositions described herein are used to treat a disease, such as cancer. In some embodiments, a method of treating a disease comprises administering to a patient a therapeutically effective amount of a pharmaceutical composition as described herein. In some embodiments, the disease is cancer. In some embodiments, the nanoparticles or protocells are loaded with an anticancer agent.

In some embodiments a kit comprises the pharmaceutical composition as described herein and instructions for use of the pharmaceutical composition. In some embodiments, an article of manufacture comprises a pharmaceutical composition as described herein in suitable packaging.

The invention is illustrated further in the following non-limiting Examples.

### Example 1

### Antiviral Protocells

TABLE 1
Established design rules for the protocell platform. The MSNP and SLB properties that
can be precisely controlled to tailor various protocell parameters are listed, along with
the resulting biological effect(s). See Ashley et al. (2011) for further details.

| MSNP or SLB Property | Protocell Parameter(s) | Biological Effect(s) |
|---|---|---|
| Size and Size Distribution | Biodistribution, internalization | Tailor the concentration of |
| | efficiency | drugs(s) in specific organs, |
| | | tissues, and/or cells |
| MSNP Charge | MSNP-SLB interactons | Balance extracellular drug |
| | | retention and intracellular drug |
| | | release by optimizing SLB |
| | | stability |
| Pore Size | Loading capacity, type(s) of | Reduce dose by decreasing the |
| | cargo molecules that can be | number of nanoparticles that |
| | loaded, release rates, SLB fluidity | have to reach target site(s) in |
| | | order to see an effect and/or by |
| | | delivering drug cocktails |
| Pore Chemistry | Loading capacity, types(s) of | Same as above |
| | cargo molecules that can be | |
| | loaded | |
| Degree of Silica Framework | Release rates, biodegradability | Reduce the frequency and |
| Condensation | | duration of treatment through |
| | | optimized release profiles, |
| | | enhance biocompatibility by |
| | | ensuring nanoparticles and/or |
| | | byproducts are benignly excreted |
| SLB Charge | Non-specific (vs. specific) uptake | Maximize the concentration of |
| | | drug(s) in target site(s) by |
| | | decreasing non-specific |
| | | interactions |
| SLB Fluidity | Mobility of targeting ligands, | Maximize the concentration of |
| | specific binding affinities | drug(s) in target site(s) by |
| | | increasing specific interactions |
| Thickness of Lipid Coating, | Tailorable release rates under | Balance extracellular drug |
| Presence/Number of Intra- or | various intracellular conditions | retention and intracellular drug |
| Interbilayer Bonds | | release by optimizing SLB |
| | | stability |
| Degree of PEGylation | SLB stability, colloidal stability | Maximize the concentrateion of |
| | | drug(s) in target site(s) by |
| | | minimizing unwanted cargo |
| | | release; enhance biocompatibility |
| | | by minimizing serum-induced |
| | | aggregation |
| Type and Density of Targeting | Specific binding and uptake | Maximize the concentration of |
| Ligand(s) on SLB Surface | | drug(s) in target cell(s) to |
| | | decrease dose and minimize off- |
| | | target effects |
| Incorporation of | Cytosolic cargo delivery | Tailor the concentration of |
| Endo/Lyso/Phagosomolytic | | drug(s) in specific intracellular |
| Peptides on the SLB | | locations |

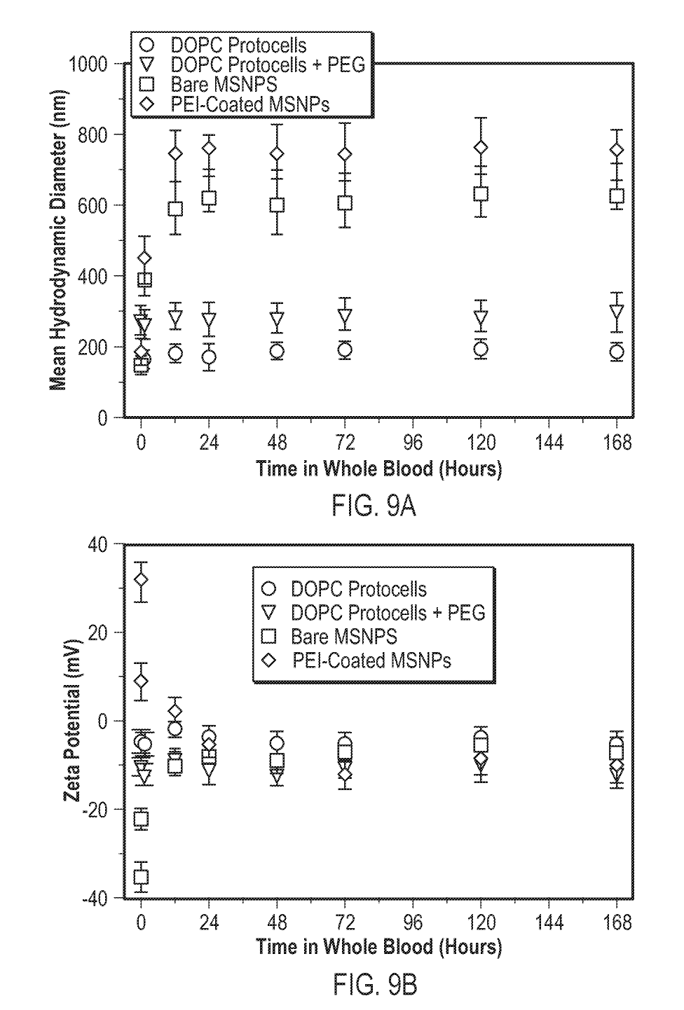

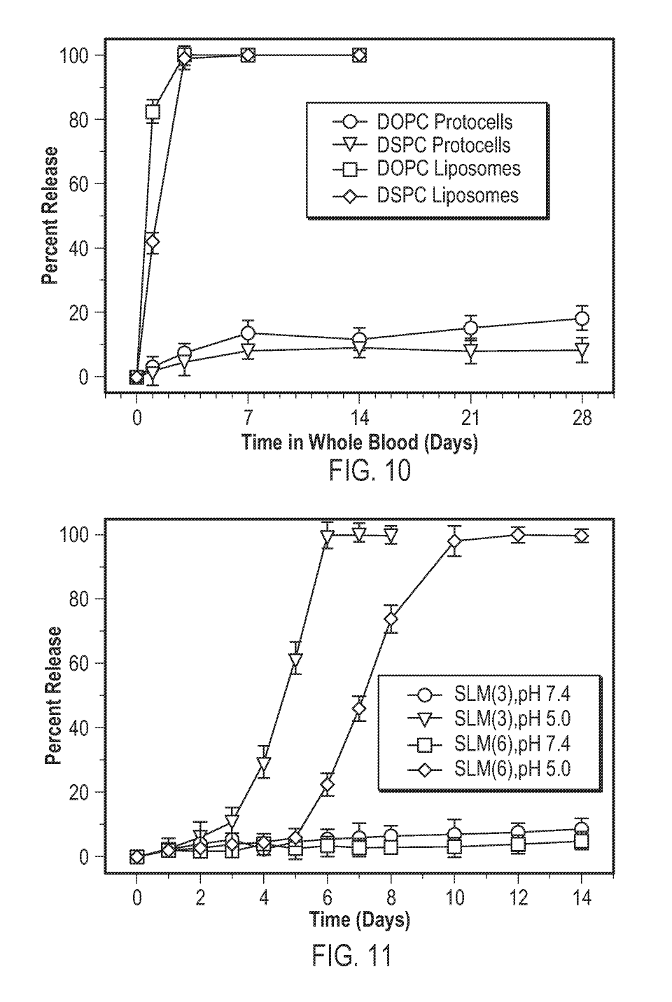

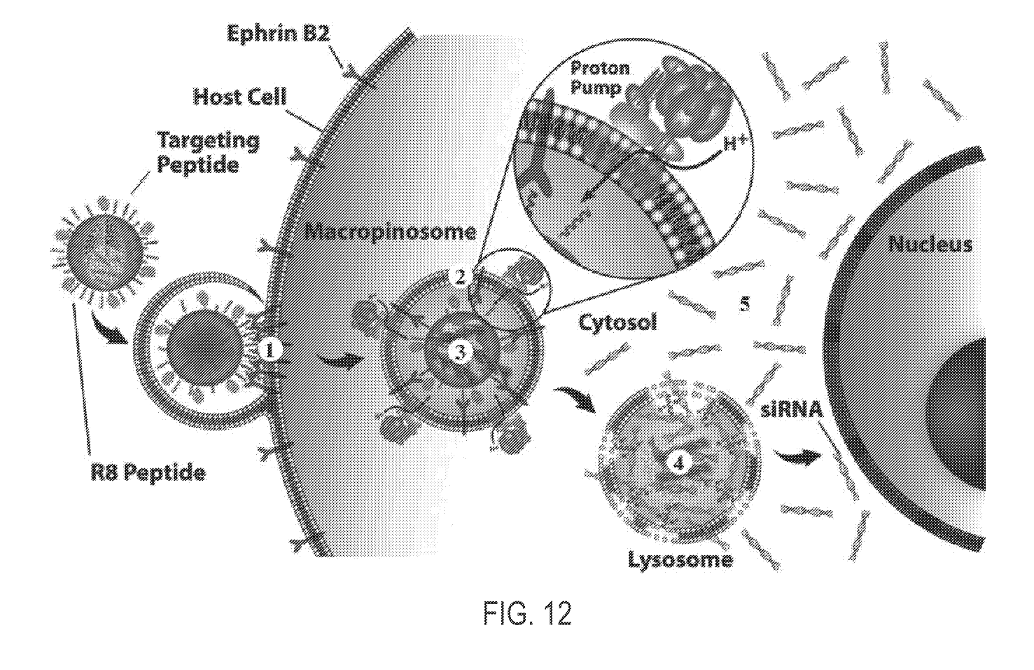

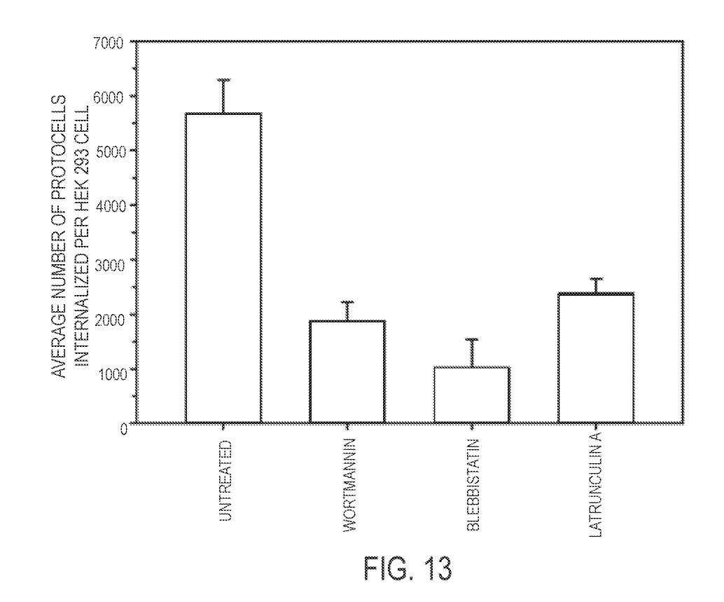

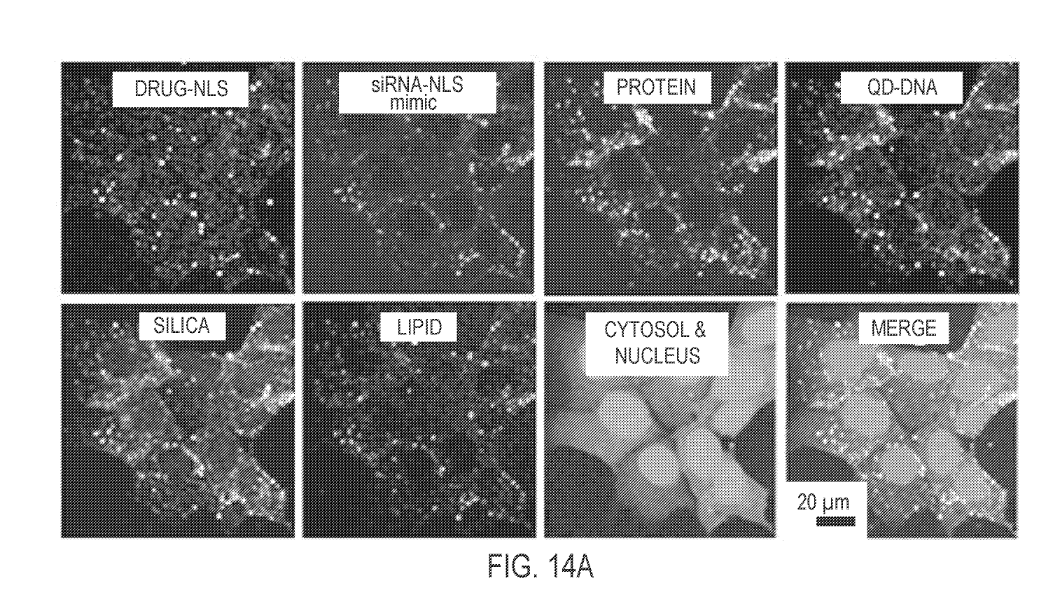

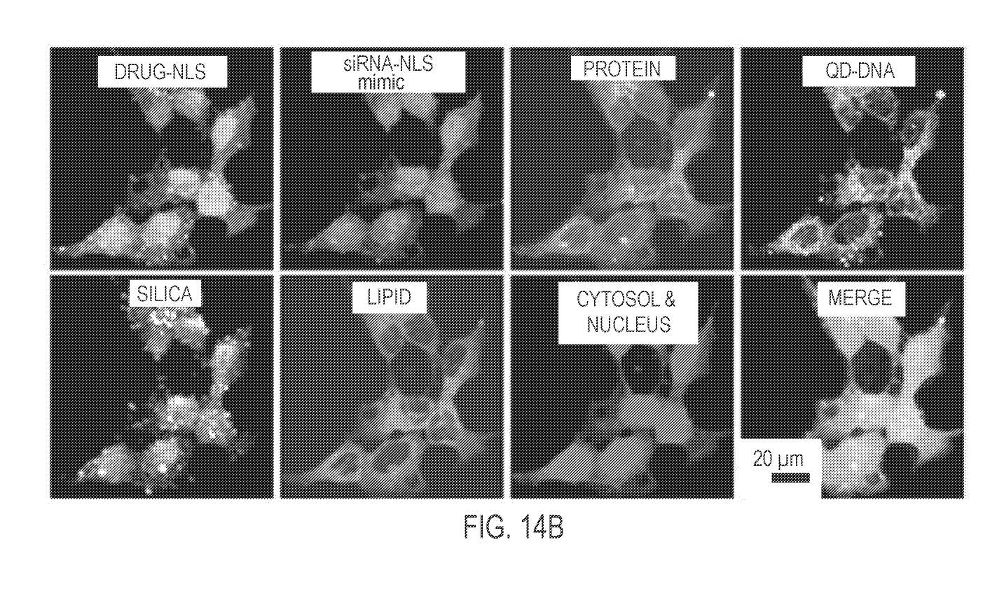

^{‡}As demonstrated by FIG. 9, DOPC protocells remain stable when dispersed in whole blood without being surface-modified with PEG; therefore, given the FDA's increasing concern about hypersensitivity reactions linked to repeat administration of PEG, it is important to note that PEGylation is not required to enhance the in vivo performance of protocells.

Table 1 lists the MSNP and SLB properties that can be controlled and how these properties can be used to tailor the in vitro and in vivo functionality of protocels. Below is a description how the design rules are applied to adapt protocells for high capacity loading and controlled release of various antivirals. In vitro data is provided that show protocells are able to selectively deliver smal molecule and nucleic acid-based antivirals to mammalian cells infected with a BSL-2 pseudotype of Nipah virus. Finally, in vivo data is provided that proves the protocells have tailorable biodistributions, cause neither gross nor histopathological toxicity, are readily degraded and excreted, and induce neither IgG nor IgM responses, even when modified with high densities of targeting peptides.

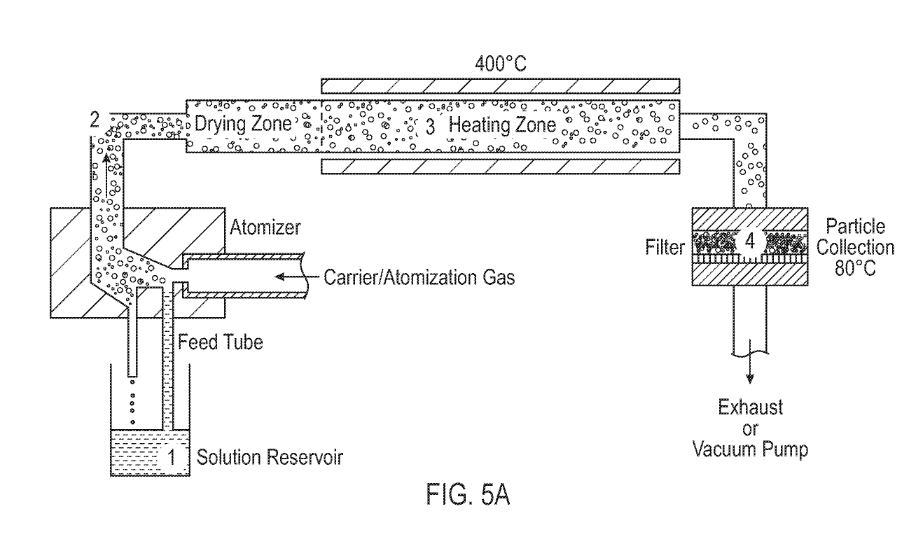

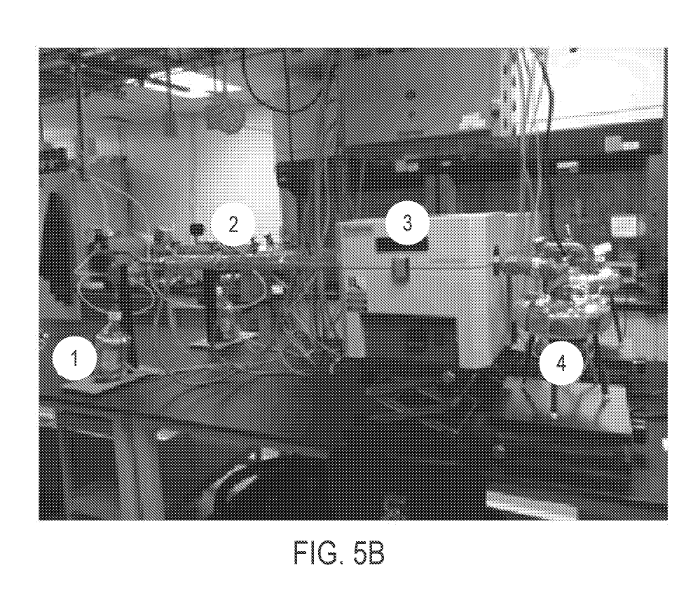

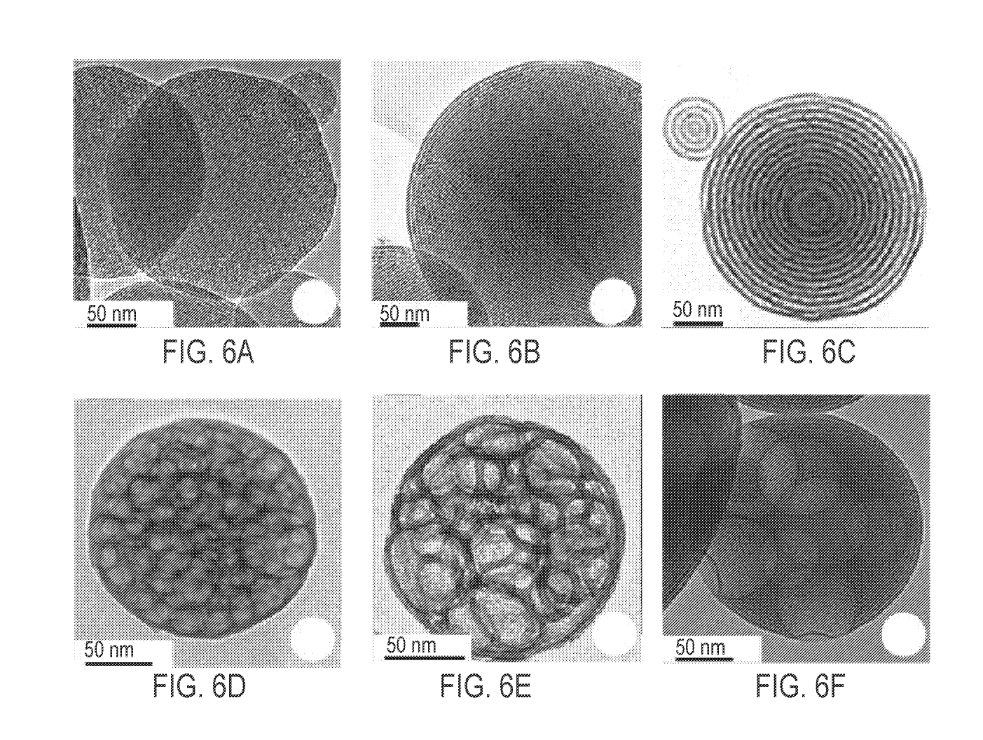

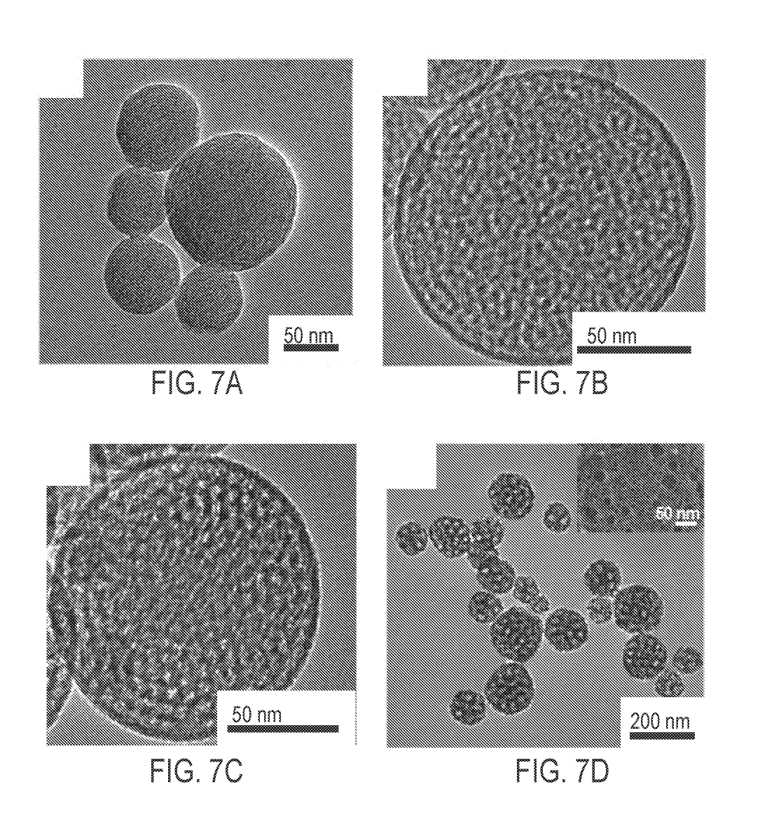

MSNPs with reproducible properties can be synthesized in a scalable fashion via Aerosol-Assisted Evaporation-Induced Self-Assembly (EISA). Aerosol-assisted evaporation-induced self-assembly¹⁶ is a robust, scalable process that we pioneered to synthesize spherical, well-ordered oxide nano- and microparticles with a variety of pore geometries and sizes (see FIGS. 6-7). In the aerosol-assisted EISA process, a dilute solution of a metal salt or metal alkoxide is dissolved in an alcohol/water solvent along with an amphiphilic structure-directing surfactant or block co-polymer; the resulting solution is then aerosolized with a carrier gas and introduced into a laminar flow reactor (see FIG. 5). Solvent evaporation drives a radially-directed self-assembly process to form particles with systematically variable pores sizes (2 to 50-nm), pore geometries (hexagonal, cubic, lamellar, cellular, etc.), and surface areas (100 to >1200 m²/g).

Aerosol-assisted EISA produces particles compatible with a variety of post-synthesis processing procedures, enabling the hydrodynamic size to be varied from 20-nm to >10-μm and the pore walls to be modified with a wide range of functional moieties that facilitate high capacity loading of physicochemically disparate diagnostic and/or therapeutic molecules. Importantly, aerosol-assisted EISA produces MSNPs that can be easily dispersed in a variety of aqueous and organic solvents without any appreciable aggregation, which enables us to load drugs that have high and low solubility in water. Our MSNPs are also easily encapsulated within anionic, cationic, and electrically-neutral SLBs via simple liposome fusion. In contrast, prior MSNPs generated using solution-based techniques tend to aggregate when the pH or ionic strength of their suspension media changes (Liong et al., 2009), typically require complex strategies involving toxic solvents to form SLBs (Cauda et al., 2010; Schloβbauer et al., 2012), and have maximum loading capacities of 1-5 wt % (Clemens et al., 2012), which, our MSNPs exceed by an order of magnitude.

Optimization of pore size and chemistry enables high capacity loading of physicochemically disparate antivirals, while optimization of silica framework condensation results in tailorable release rates.

Despite recent improvements in encapsulation efficiencies and serum stabilities, state-of-the-art liposomes, multilamellar vesicles, and polymeric nanoparticles still suffer from several limitations, including complex processing techniques that are highly sensitive to pH, temperature, ionic strength, presence of organic solvents, lipid or polymer size and composition, and physicochemical properties of the cargo molecule, al of which impact the resulting nanoparticle's size, stability, entrapment efficiency, and release rate. In contrast, MSNPs formed via aerosol-assisted EISA have an extremely high surface area (>1200 m²/g), which enables high concentrations of various therapeutic and diagnostic agents to be adsorbed within the core by simple immersion in a solution of the cargo(s) of interest. Furthermore, since aerosol-assisted EISA yields MSNPs that are compatible with a range of post-synthesis modifications, the naturally negatively-charged pore walls can be modified with a variety of functional moieties, enabling facile encapsulation of physicochemically disparate molecules, including acidic, basic, and hydrophobic drugs, proteins, small interfering RNA (siRNA), minicircle DNA vectors that encode small hairpin RNA (shRNA), plasmids, and diagnostic/contrast agents like quantum dots, iron oxide nanoparticles, gadolinium, and indium-111 (Ashley et al., 2012; Ashley at al., 2011).

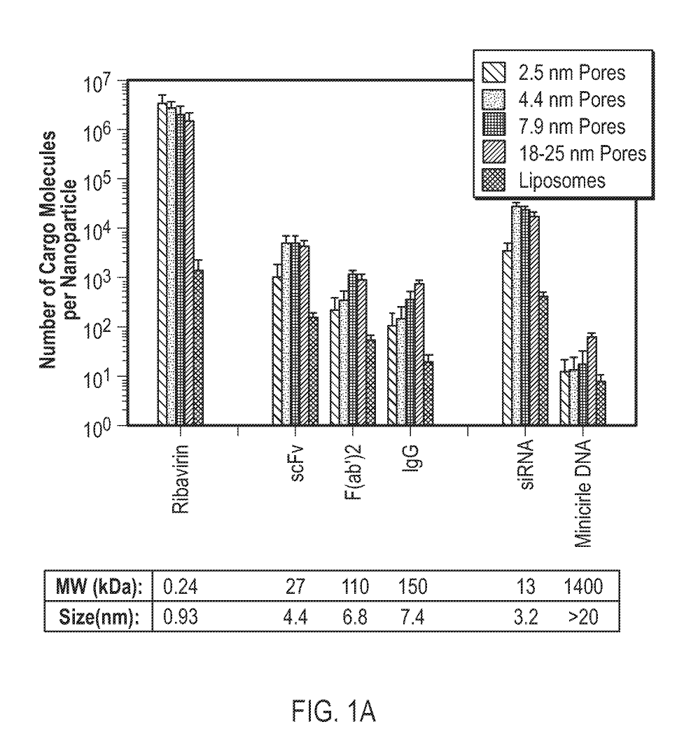

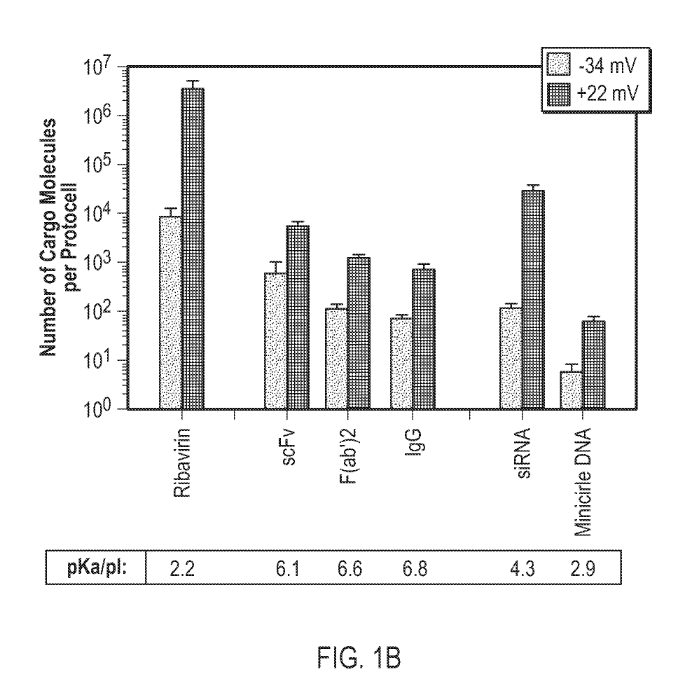

The MSNPs and the protocells comprising such MSNPs described herein demonstrate high-capacity loading of effective antiviral agents. As demonstrated by FIG. 1A, MSNPs formed via aerosol-assisted EISA can be loaded with up to 70 wt % of small-molecule antivirals like ribavirin (>3 million molecules/MSNP), 32 wt % of siRNA-based antivirals (about 30,000 molecules/MSNP), 7.2 wt % of a 2,000-base-pair minicircle DNA vector that encodes shRNA-based antivirals (about 60 vectors/MSNP), and 8.9-12 wt % of various antibody-based antivirals (about 700-5400 molecules/MSNP), including single-chain variable fragments (scFvs), F(ab)₂ fragments, and whole IgGs. It is important to note that these capacities are 10-fold higher than other MSNP-based delivery vehicles (Clemens at al., 2012) and 100 to 1000-fold higher than similarly-sized liposomes and polymeric nanoparticles (Couvreur at al., 2006; Morilla et al., 2011; Wong at al., 2003).

It is also important to note that the MSNPs and protocells comprising such MSNPs can be loaded with complex combinations of physicochemically disparate antivirals (e.g., three small molecule drugs in combination with five separate siRNAs), a capability other nanoparticle delivery vehicles typically do not possess. High loading capacities can be achieved for acidic, basic, and hydrophobic drugs, as well as small molecules and macromolecules by altering the solvent used to dissolve the drug prior to loading and by modulating the pore size and chemistry of the MSNP (see FIG. 1). Unlike MSNPs formed using solution-based techniques, MSNPs formed via aerosol-assisted EISA are compatible with all aqueous and organic solvents, which ensures that the maximum concentration of drug loaded within the pore network is essentially equivalent to the drug's maximum solubility in its ideal solvent. Furthermore, since MSNPs formed via aerosol-assisted EISA remain stable upon post-synthesis processing, the pore chemistry can be precisely altered by, e.g., soaking naturally negatively-charged MSNPs in amine-containing silanes (e.g., (3-aminopropyl)triethoxysilane, or APTES), in order to maximize electrostatic interactions between pore walls and cargo molecules.

Another unique feature of the MSNPs and the protocells comprising such MSNPs is that the rate at which encapsulated drug is released can be precisely modulated by varying the degree of silica framework condensation and, therefore, the rate of its dissolution via hydrolysis under physiological conditions (Ashley et al., 2011). As shown in FIG. 8, silica (SiO₂) forms via condensation and dissolves via hydrolysis. Therefore, MSNPs with a low degree of silica condensation have fewer Si—O—Si bonds, hydrolyze more rapidly at physiological pH, and release 100% of encapsulated ribavirin within 12 hours. In contrast, MSNPs with a high degree of silica condensation hydrolyze slowly at physiological pH and can, therefore, release about 2% of encapsulated ribavirin (about 60,000 molecules/MSNP) per day for 2 months. We can tailor the degree of silica condensation between these extremes by employing different methods to remove structure-directing surfactants from pores (e.g., thermal calcination, which maximizes the number of Si—O—Si bonds vs. extraction via acidified ethanol, which favors the formation of SiOH bonds over Si—O—Si bonds) and by adding various concentrations of amine-containing silanes to the precursor solution in order to replace a controllable fraction of Si—O—Si bonds with Si—R—NH₂ bonds, where R=hydrocarbons of various lengths.

Fusion of Liposomes to Antiviral-Loaded MSNPs Creates a Coherent SLB that Enhances Colloidal Stability and Enables DH-Triggered Release.

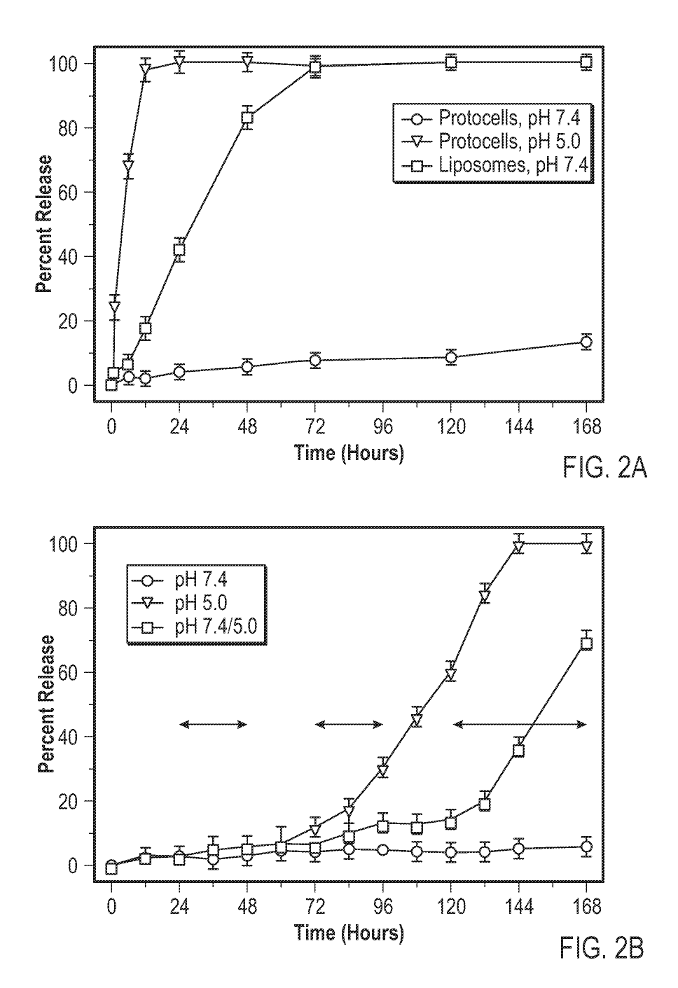

Liposomes and multilamellar vesicles have poor intrinsic chemical stability, especially in the presence of serum, which decreases the effective concentration of drug that reaches target cells and increases the potential for systemic toxicity (Couvreur et al, 2006; Morilla et al., 2011). In contrast, lipid bilayers supported on MSNPs have a high degree of stability in neutral-pH buffers, serum-containing simulated body fluids, and whole blood (FIG. 2), regardless of the melting temperature (Tₘ, which controls whether lipids are in a fluid or non-fluid state at physiological temperature) of lipids used to form the SLB (Ashley et al., 2011). Specifically, protocells with SLBs composed of the zwitterionic, fluid lipid, 1,2-dioleoyl-sn-glycerol-3-phosphocholine (DOPC) have a high degree of colloidal stability (see FIG. 9) and retain small molecule drugs, such as ribavirin, for up to 4 weeks (see FIG. 10) when incubated in whole blood or a serum-containing simulated body fluid at 37° C.; it is important to note that surface-modification with polyethylene glycol (PEG) is not necessary to stabilize DOPC protocells, which is significant given the FDA's increasing concerns about hypersensitivity reactions induced by PEGylated nanoparticles and therapeutic molecules. In dramatic contrast to the behavior of DOPC protocells, serum proteins rapidly adsorb to bare MSNPs and MSNPs coated with cationic polymers, such as polyethyleneimine (PEI), upon dispersion in whole blood or serum-containing simulated body fluids (see FIG. 9), and ribavirin-loaded liposomes rapidly leak their encapsulated drug (see FIG. 2 and FIG. 10), even when composed of the fully-saturated lipid, 1,2-distearoyl-sn-glycero-3-phosphocholine (DSPC), which has a higher packing density than fluid lipids (e.g., DOPC) and would have been expected to limit diffusion of drug across the bilayer (Ashley et al., 2011).

Although protocells are highly stable under neutral pH conditions, the SLB can be selectively destabilized under conditions that simulate the interior volume of intracellular vesicles (e.g., endosomes, lysosomes, macropinosomes), which become acidified via the action of proton pumps. Specifically, DOPC SLBs are destabilized at pH 5.0, which exposes the MSNP core and stimulates its dissolution at a rate dictated by core's degree of silica condensation; DOPC protocells with MSNPs cores that have a low degree of condensation are, therefore, able to retain ribavirin at pH 7.4 but rapidly release it at pH 5.0 (see FIG. 2A). pH-dependent release rates can be further tuned by controlling the thickness of the protocel's lipid shell. Fusing liposomes to MSNPs in the presence of divalent cations (Moon et al., 2011) results in protocells with supported lipid multilayers (SLMs), the thickness of which can be used to control release rates under acidic conditions (see FIG. 11). Protocells with SLMs are also able to withstand iterative exposure to neutral and acidic pH conditions (see FIG. 2B), which indicates that protocells wil be able to retain encapsulated antivirals while transversing cellular barriers, such as the nasal epithelium and the BBB, and release encapsulated antivirals within target CNS cells.

Modifying the SLB with Targeting Ligands Promotes Efficient Uptake of Antiviral-Loaded Protocells by Model Host Cells, which Enables Efficacious In Vitro Inhibition of Viral Replication.

In order to inhibit the intracellular replication of viruses, nanoparticle delivery vehicles must be efficiently internalized by host cells, escape intracellular vesicles, and release encapsulated antivirals in the cytosol of host cells. A number of factors govern cellular uptake and processing of nanoparticles, including their size, shape, surface charge, and degree of hydrophobicity (Peer et al., 2007). Additionally, a variety of molecules, including peptides, proteins, aptamers, and antibodies, can be employed to trigger active uptake by a plethora of specific cells (Peer et al., 2007). We have previously shown that incorporation of targeting peptides and endosomolytic peptides enable cell-specific delivery and cytosolic dispersion of encapsulated cargos (Ashley et al., 2011). As importantly, we have shown that SLB fluidity can be tuned to enable exquisite (sub-nanomolar) specific affinities for target cells at extremely low targeting ligand densities (about 6 targeting peptides per protocell) and that SLB charge can be modulated to reduce non-specific interactions, resulting in protocels that are internalized by target cells 10,000-times more efficiently than non-target cells (Ashley et al., 2011).

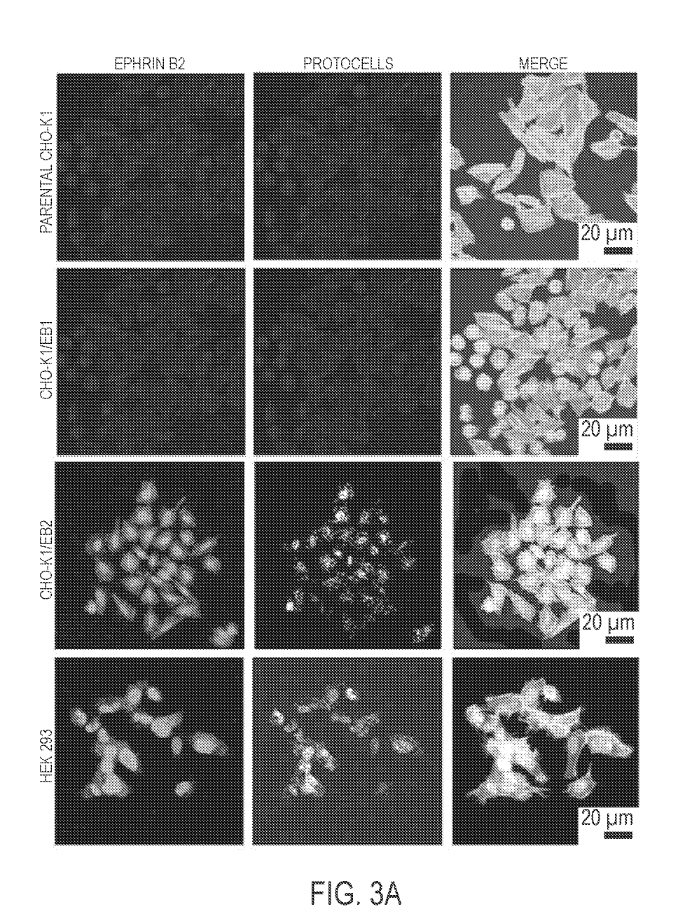

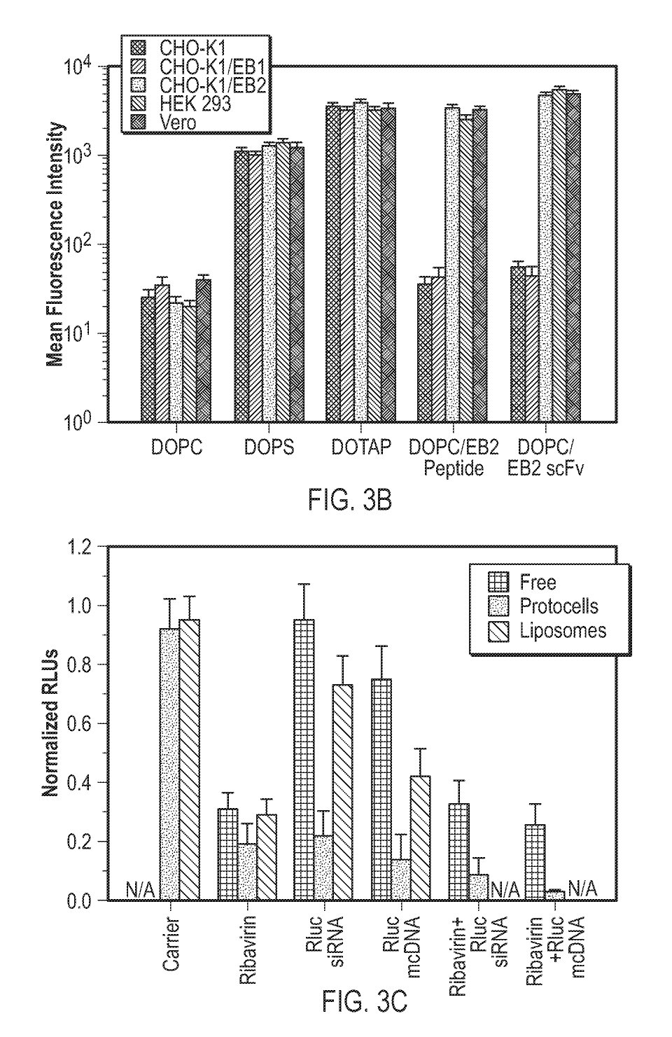

The targeting specificity of protocels was used to deliver various types of antivirals to host cells in which numerous viruses replicate in vitro. For example, modifying DOPC protocells with peptides or scFvs that target ephrin B2, the cellular receptor for Nipah (NiV) and Hendra (HeV) viruses, triggers a 100-fold increase in their selective binding and internalization by ephrin B2-expressing cells (see FIGS. 3A-B, 12 and 13). In contrast, protocells with SLBs composed of the anionic lipid, 1,2-dioleoyl-sn-glycero-3-phospho-L-serine (DOPS) or the cationic lipid, 1,2-dioleoyl-3-trimethylammonium-propane (DOTAP) were non-specifically internalized by both ephrin B2-positive and ephrin B2-negative cells, which demonstrates an important point: although numerous researchers coat MSNPs with cationic lipids and polymers, the resulting non-specific uptake reduces the effective drug concentration that reaches target cells and tissues (Clemens et al., 2012).

As demonstrated by FIG. 3C, protocells loaded with small molecule and nucleic acid-based antivirals and targeted to ephrin B2 are able to more effectively inhibit the in vitro replication of a Nipah pseudovirus in infected Vero cells than unencapsulated or liposome-encapsulated antivirals; it is important to note that, unlike liposomes, protocells are able to simultaneously encapsulate and deliver small molecule and nucleic acid-based antivirals, which virtually eliminates expression of the reporter protein encoded by the Nipah pseudovirus without affecting the viability of host cells (see FIG. 16).

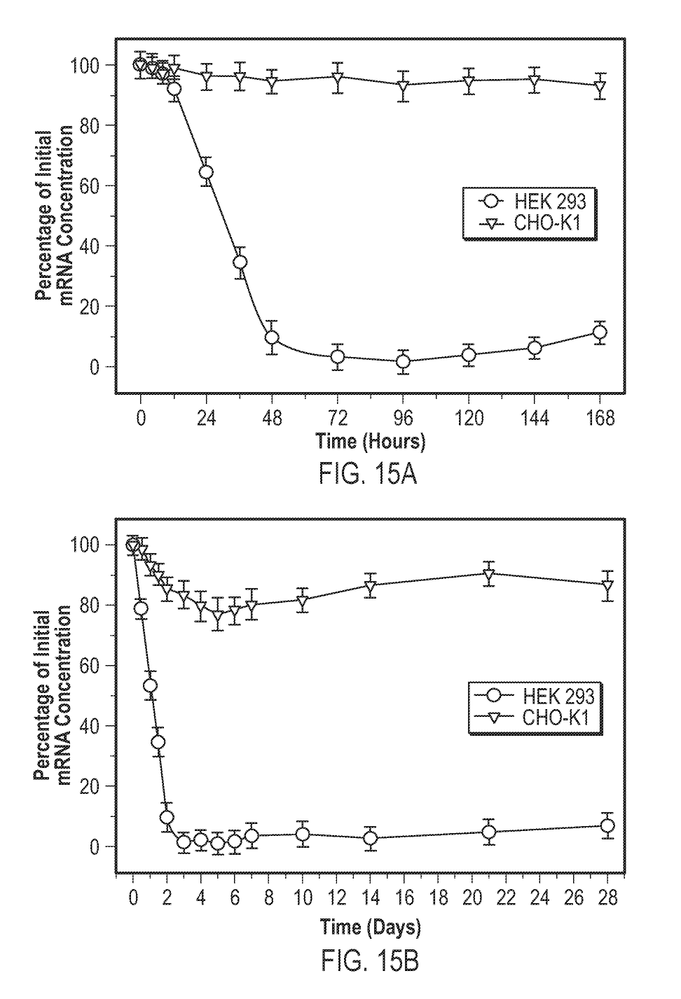

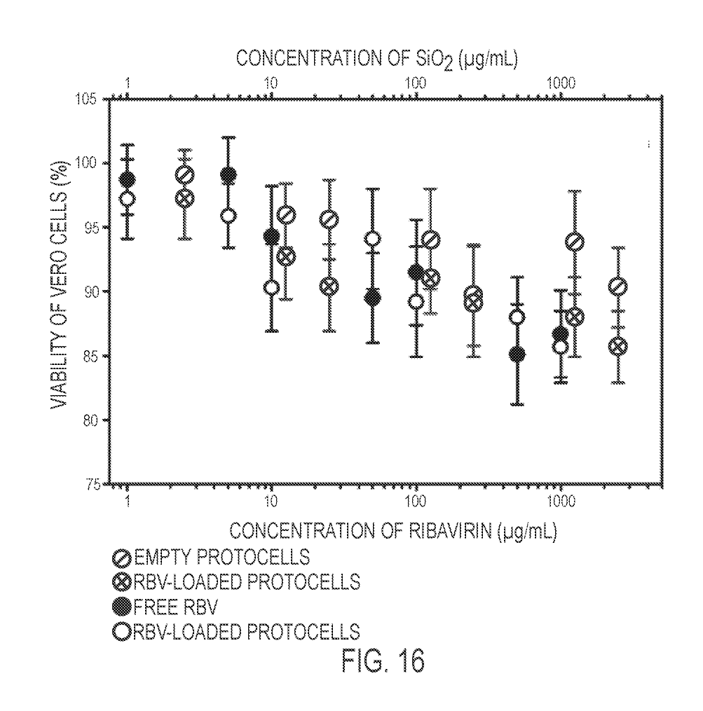

Since endosomal escape of protocell-encapsulated antivirals is critical to maximize efficacy, the SLBs of protocels used in these experiments were further modified with peptides (e.g., R8 and H5WYG) that rupture the membranes of acidic intracellular vesicles via the ‘proton sponge’ mechanism; as shown in FIG. 14, the H5WYG peptide promotes cytosolic dispersion of various fluorescently-labeled cargo molecules, as well as the lipid and silica components of the protocell. Finally, since small molecule and siRNA-based antivirals can only transiently inhibit viral replication, we have demonstrated that protocells loaded with minicircle vectors that encode shRNAs specific for a viral gene are able to silence the target gene for >1 month (see FIG. 15), which indicates that protocells can be adapted for pre- and post-exposure treatment of viral infections.

Protocells are Biocompatible, Biodegradable, and Non-Immunogenic.

Several reasons support the observation that amorphous MSNPs have low toxicity profiles in vivo: (1) amorphous (i.e., non-crystalline) silica is accepted as ‘Generally Recognized As Safe’ (GRAS) by the U.S. FDA; (2) recently, solid, dye-doped silica nanoparticles received approval from the FDA for targeted molecular imaging of cancer (Chen et al., 2011; He et al., 2009); (3) compared to solid silica nanoparticles, MSNPs exhibit reduced toxicity and hemolytic activity since their surface porosity decreases the contact area between surface silanol moieties and cell membranes (Zhang et al., 2012; Tam et al., 2013; Zhao et al., 2011); (4) the high internal surface area (>1000 m²/g) and ultra-thinness of the pore walls (<2-nm) enable MSNPs to dissolve, and soluble silica (e.g., silicic acid, Si(OH)₄) has extremely low toxicity (He et al., 2009; Lin et al., 2010); and (5) in the case of protocells, the SLB further reduces interactions between surface silanol moieties and cell membranes and confers immunological behavior comparable to liposomes (Ashley et al., 2011).

To confirm these predictions, the biocompatibility, biodegradability, and immunogenicity of protocells was evaluated after repeat intraperitoneal (IP) or subcutaneous (SC) injections in Balb/c and C57Bl/6 mice. Balb/c mice injected IP with 200 mg/kg doses of DOPC protocells three times each week for 4 weeks showed no signs of gross or histopathological toxicity (see FIG. 17). Furthermore, we have demonstrated that intact and partially-degraded MSNPs, as well as silcic acid and other byproducts of silica hydrolysis are excreted in the urine and feces of mice at rates that are determined by the dose, route of administration, and biodistribution (see FIGS. 18 and 20-22), observations that are supported by studies performed at the UCLA Center for Environmental Implications of Nanotechnology (CEIN) (Lu et al., 2010). Finally, we have shown that protocells loaded with a therapeutic protein and modified with a high density (about 10 wt % or about 5000 peptides/protocell) of a targeting peptide induce neither IgG nor IgM responses upon SC immunization of C57B/6 mice at a total dose of 1000 mg/kg (see FIG. 19).

The Biodistribution of Protocells can be Controlled by Tuning their Hydrodynamic Size and Surface Modification with Targeting Ligands.

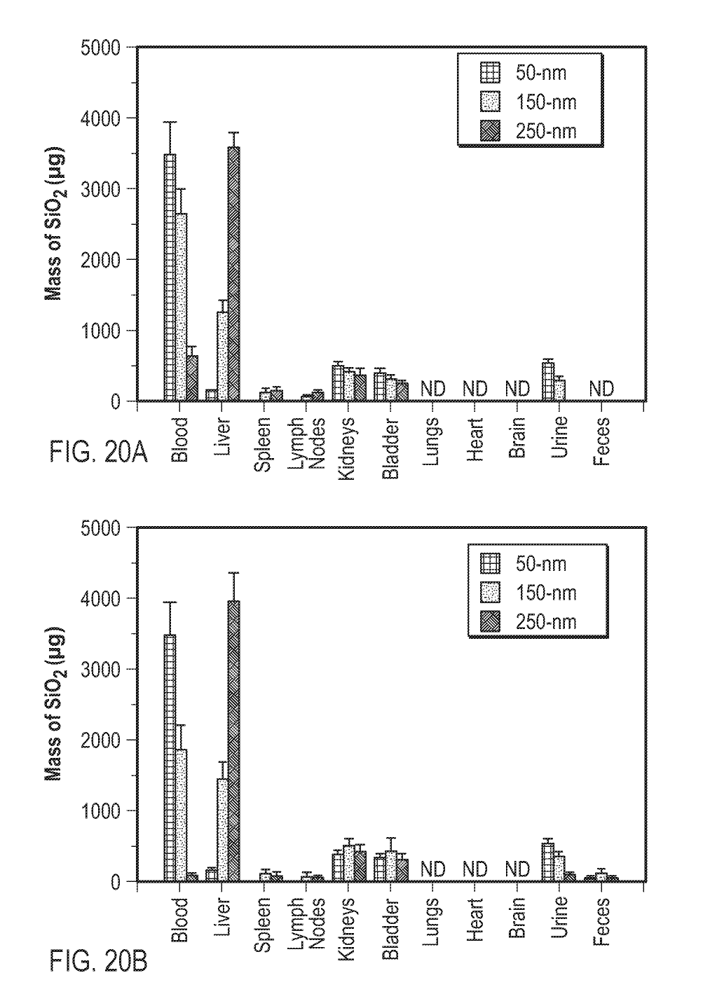

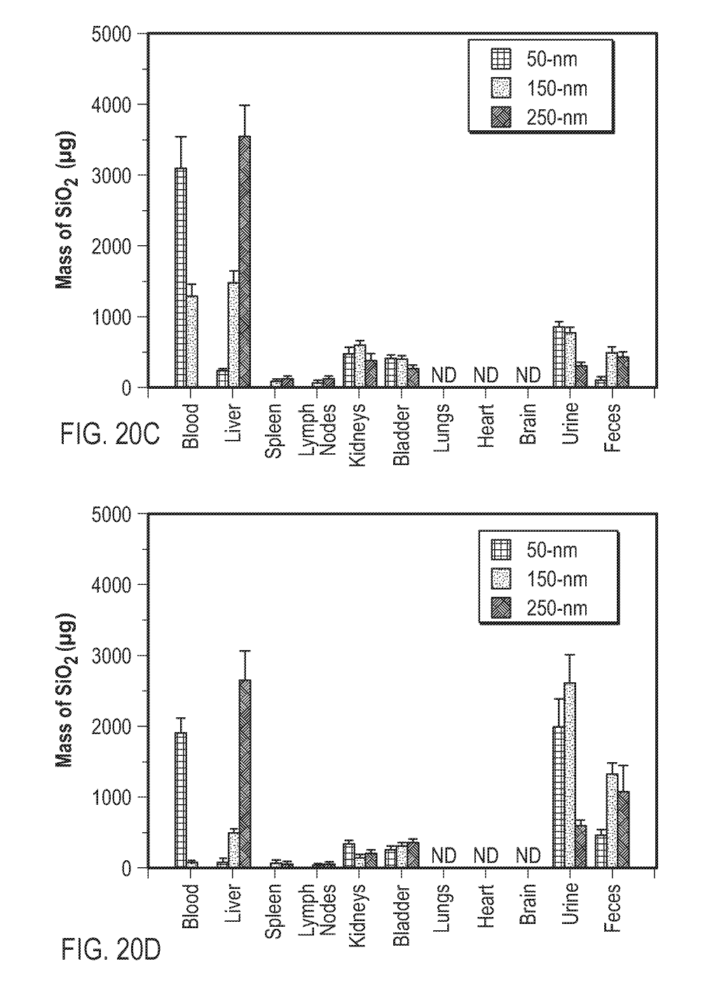

Since liposomes and multilamellar vesicles are the most similar nanoparticle delivery vehicles to protocels, every effort is made to benchmark the performance of protocells against the performance of lipid-based nanoparticles. Liposomes and multilamellar vesicles, despite being more elastic that protocels, have biodistributions that are largely governed by their overall size and size distributions (Sommerman, 1986), an observation that holds true for protocells as well. The sizes of liposomes and multilamellar vesicles are difficult to control and subject to slight variations in lipid content, buffer pH and ionic strength, and chemical properties of cargo molecules, however (Sommermon, 1986: Comiskey et al., 1990: Moon et al., 1998). In contrast, the diameter of protocells is governed by the size of the MSNP core, which, as we have previously described, is easy to precisely modulate (Ashley at al., 2011). As demonstrated by FIG. 20, the hydrodynamic size of protocells dramatically affects their bulk biodistributions: protocells 250-nm in diameter accumulate in the liver within 1 hour of injection, while protocells 50-nm in diameter remain in circulation for >1 month. Size-dependent biodistribution can be altered, however, by modifying the surface of DOPC protocells with various types of targeting ligands.

For example, modifying 150-nm protocels with CD47, a molecule expressed by erythrocytes that innate immune cells recognize as ‘self’ (Oldenborg et al., 2000), substantially enhances their circulation half-life (see FIG. 21). In contrast, modifying 150-nm protocells with an antibody against aminopeptidase P³⁷ causes them to rapidly amass in the lung (see FIG. 22). The ability to engineer protocells for both systemic circulation and targeted accumulation within specific organs indicates that a combination of sizes and targeting moieties may enable efficient delivery into the CNS.

Optimization of the BBB Penetration Potential of Protocells Using an In Vitro Transwell Assay.

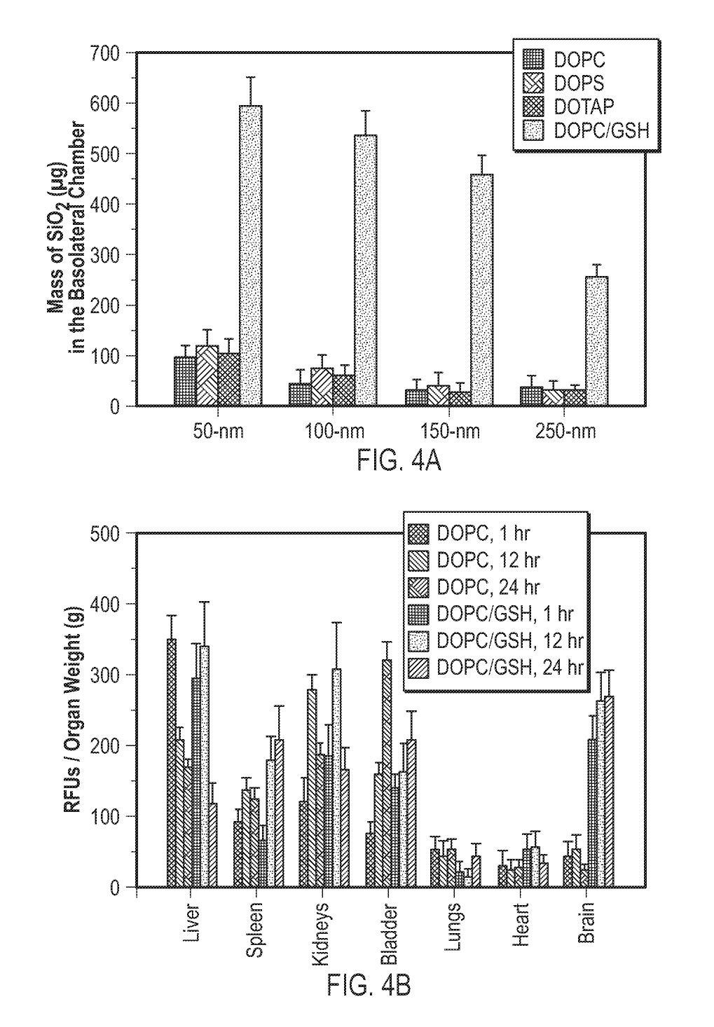

The preliminary data indicate that various parameters affect the BBB penetration potential of protocels, including their hydrodynamic size, surface charge, and surface modifications; as shown in FIG. 4, modifying DOPC protocells with 1 wt % of glutathione increased their in vitro penetration across cerebral microvessel endothelial cells by 50% and their in vivo accumulation within the brains of Balb/c mice by about 10-fold. Modify protocells with ligands (e.g., L-dihydroxyphenylalanine, or L-DOPA) that SwRI has used to enhance BBB penetration of small molecule drugs in an attempt to further increase the BBB penetration potential of antiviral-loaded protocells 50 to 150-nm in diameter.

To assess penetration and antiviral delivery efficiency, a high-throughput transwell assay was used where cerebral microvessel endothelial (hCMEC/D3) cells (Weksler et al., 2013) are grown to confluence on collagen-coated, microporous, polycarbonate membranes and inserted into 12-well Costar Transwell plates. Various concentrations of ribavirin-loaded protocells were added to the apical or basolateral chamber, incubate the wells for 2 hours at 37° C., and inductively-coupled plasma mass spectrometry (ICP-MS) and HPLC were used to measure Si and ribavirin concentrations, respectively, in both chambers. Ribavirin was used in these studies since there is a fluorescence-based HPLC method to detect it in cell culture medium. Monolayers are co-dosed with lucifer yellow to ensure that the cells are not damaged during the course of the experiment. DLS, transmission electron microscopy (TEM), and UV-visible spectroscopy are used to characterize the size, charge, and loading capacity of protocels that cross the cel monolayer. hCMEC/D3 cells are used because they are robust, easy to grow, and better replicate the human BBB than other in vitro models (Weksler et al., 2013).

TABLE 2
Table 2. The pore-templating surfactant, mean hydrodynamic diameter,
average surface charge, Brunauer-Emmett-Teller (BET) surface
area, and Barrett-Joyner-Halenda (BJH) pore size of MSNPs synthesized
using aerosol-assisted EISA. Mean diameter and average surface
charge were measured in 1X PBS, pH 7.4.

| | Mean | Average | BET | BJH |
|---|---|---|---|---|
| | Diameter | Surface Charge | Surface Area | Pore Size |
| Sufactant | (nm) | (mV) | (m²/g) | (nm) |
| CTAB | 102 ± 4 | −34 | 1080 ± 48 | 2.5 ± 0.1 |
| F68 | 105 ± 5 | −35 | 440 ± 20 | 4.4 ± 0.2 |
| F127 | 125 ± 6 | −30 | 240 ± 11 | 7.9 ± 0.4 |
| F127 + FC-4 | 480 ± 56 | −35 | 190 ± 32 | 18-25 |

TABLE 3
Table 3: mAb's to Treat Arbovirus Infections as cited in Sautto et al., (2013).

| | | | Target | | Cloning | | Neutralizing | |
|---|---|---|---|---|---|---|---|---|
| mAb | Origin | Reactivity | protein | Epitope | strategy | Format/isotype | activity | Reference |
| 4.8A | Human | DENV1-4 | E | DII | EBV | IgG | IC50(μg/mL): | [37] |
| D11C | | | | | transformation | | DENV1: 1.3 | |
| | | | | | | | DENV2: >40 | |
| | | | | | | | DENV3: 2.4 | |
| | | | | | | | DENV4: >40 | |
| | | | | | | | IC50 (μg/mL): | |
| | | | | | | | DENV1: 1.5 | |
| | | | | | | | DENV2: 1.0 | |
| | | | | | | | DENV3: 10.2 | |
| | | | | | | | DENV4: 1.6 | |
| | | | | | | | IC50(μg/mL): | |
| | | | | | | | DENV1: 1.5 | |
| | | | | | | | DENV2: 0.2 | |
| | | | | | | | DENV3: 0.5 | |
| | | | | | | | DENV4: 2.7 | |
| 2H12 | Murine | DENV1-4 | E | DIII | Hybridoma | lgG2b | IC50 nM; | [38] |
| | | | | (AB | from | | DENV1: 0.56-54 | |
| | | | | loop: | BALB/C | | | |
| | | | | aa | mouse | | DENV3: 29 | |
| | | | | | immunized | | | |
| | | | | 314-317) | with | | DENV4: 145 | |
| | | | | | DENV2 | | | |
| | | | | | E/DIII | | | |
| C9 | Murine/ | DENV2 | E | DIII | Phage | VH1/Vκ1 | PRNT 50 | [39] |
| | chimeric | | | | display of | | 850 μg/mL | |
| | | | | | a chimeric | | | |
| | | | | | murine | | | |
| | | | | | hybridoma | | | |
| | | | | | library | | | |
| 4E11 | Murine | DENV1-4 | E | DIII | Hybridoma | IgG2a/κ | IC50 | [40] |
| | | | | (strand A: | | | (μg/mL): | |
| | | | | 308, 312 | | | DENV10.16 | |
| | | | | and | | | DENV2: 0.13 | |
| | | | | strand G: | | | DENV-3: 8 | |
| | | | | 387, 389, | | | DENV-4: 15 | |
| | | | | 391) | | | | |
| 4G2 | Murine | DENV1-4 | E | DII | Hybridoma | IgG | PRNT50 | [39] |
| | | | | (fusion | from | | (μg/mL): | |
| | | | | loop) | DENV2 E | | DENV2: 15 | |
| | | | | | Immunized | | | |
| | | | | | mice | | | |
| 9F12 | Murine | DENV1-4, | E | DIII (aa | Hybridoma | IgG1k | DENV1- | [41] |
| | | WNV | | 305, 307, | from | | 4PRNT50: | |
| | | | | 310, 330: | BALB/C | | 2-10⁻⁶- | |
| | | | | A strand | mouse | | 2-10⁻⁷ M | |
| | | | | and BC | immunized | | | |
| | | | | loop) | with | | | |
| | | | | | DENV-2 | | | |
| | | | | | E/DIII | | | |
| 2A10G6 | Murine | DENV1-4, | E | DII | Hybridoma | IgG1 | PRNT50 | [36] |
| | | YFV, WNV, | | (Fusion | from | | (μg/mL): | |
| | | JEV, TBEV | | loop: aa | BALB/C | | DENV1: 2 | |
| | | | | 98-101) | mouse | | DENV2: 1.5 | |
| | | | | | immunized | | DENV3: 2.1 | |
| | | | | | with | | DENV4: 1.8 | |
| | | | | | inactivated | | YNF: 3.6 | |
| | | | | | DENV2 | | WNV: 46 | |
| mAB11 | Human | DENV1-4, | E | DII | Phage | Fusion | PRNT80 | [42, 43] |
| | | WNV, SLEV, | | (fusion | display of | protein | (μg/mL): | |
| | | YFV, JEV | | loop) | human | scFv-Fc | WNV: 1.25 | |
| | | MVEV | | | scFv | | DENV2: 6.25 | |
| E16 | Murine | WNV | E | DIII | Hybridoma | IgG2b/humanized | PRNT50: | [44, 45] |
| (MGAWN1) | | | | (LR; aa | from | | 4-18 ng | |
| | | | | 302-309) | immunized | | PRNT90: | |
| | | | | | mice with | | 53-297 ng | |
| | | | | | WNV E | | | |
| 1A1D-2 | Murine | DENV1-3 | E | DIII | Hybridoma | IgG2a | DENV2 | [46, 47] |
| | | | | (strand: aa | from | | PRNT50: | |
| | | | | 307, 310 | immunized | | 2.1 nM | |
| | | | | and 312) | mice with | | | |
| | | | | | different | | | |
| | | | | | pH-treated | | | |
| | | | | | virus | | | |
| 1F4 | Human | DENV1 | E | DI-DII | Electrofusion | IgG | DENV1 IC50 | [48] |
| 2D22 | | DENV2 | (virion) | DIII | of infected | | 0.11 μg/mL | |
| 5J7 | | DENV3 | | DI-DII | memory B | | DENV2 IC50 | |
| | | | | | cells from | | 0.08 μg/mL | |
| | | | | | DENV- | | DENV3 IC50 | |
| | | | | | immune | | 0.10 μg/mL | |
| | | | | | subjects | | | |
| | | | | | with EBV | | | |
| E105 | Murine | DENV1 | E | DIII | Hybridomas | IgG | PRNT50 | [32] |
| E106 | | | | (BC loop: | derived | | DENV-1: 0.5-59.2 | |
| E111 | | | | G328, T329 | from | | ng/mL | |
| | | | | and D330: | C57BL/6 | | PRNT50 | |
| | | | | DE loop: | IFN-α βR^{-/-} | | DENV-1: 0.6-59.2 | |
| | | | | K361 E and | mice | | ng/mL | |
| | | | | E362 K; FG | infected | | PRNT50: | |
| | | | | loop: K385) | with | | 3.8-25 | |
| | | | | DIII | DENV1 | | μg/mL | |
| | | | | | | | | [49] |
| | | | | (BC loop: | | | | |
| | | | | G328, T329 | | | | |
| | | | | and D330: | | | | |
| | | | | DE loop; | | | | |
| | | | | K361 E and | | | | |
| | | | | E362 K: FG | | | | |
| | | | | loop: K385: | | | | |
| | | | | A strand: | | | | |
| | | | | S305, K307, | | | | |
| | | | | E309, K310 | | | | |
| | | | | and E311) | | | | |
| | | | | DIII | | | | |
| | | | | (CC′ loop) | | | | |
| CF4374 | Human | WNV | E | DIII | Phage | VH2-05/VL1e | PRNT50: | [50] |
| CR4353 | | | | | display of | VH3-30/Vk3- | 0.18 μg/mL | |
| | | | | | scFv IgG | A27 | PRNT90: | |
| | | | | | library | | 0.95 μg/mL | |
| | | | | | | | PRNT50: | |
| | | | | | | | 0.026 μg/mL | |
| | | | | | | | PRNT90 | |
| | | | | | | | 36.4 μg/mL | |
| 1A5 | Chimpanzee | DENV1-4, | E | DII | Phage | Humanized | DENV1: 048 | [51] |
| | | WNV, JEV, | | (aa G106) | display of | IgG1 | DENV2: 0.95 | |
| | | LGTV | | | Fab library | | DENV3: 3.2 | |
| | | | | | from | | DENV4: 4.3 | |
| | | | | | DENV1-4 | | WNV/DEN | |
| | | | | | infected | | V4: 3.8 | |
| | | | | | chimpanzees | | JEV: 21 | |
| | | | | | | | LGTV: 28 | |
| mAb11 | Human | DENV1-4, | E | DII | scFv | scFv-Fc | PRNT80 | [42, 43] |
| | | WNV | | (fusion loop, | library | | (μg/mL): | |
| | | | | W101, G104, | | | DENV2: 6.25 | |
| | | | | G106) | | | WNV: 1.25 | |
| 3B4C-4 | Murine | SLEV | E | 1a | Hybridoma | IgG | SLEV | [52, 53] |
| 1B2C-5 | | SLEV | | 1b | | | PRNT: <1.7 | |
| 6B5A-2 | | SLEV | | 1c | | | SLEV | |
| 4A4C-4 | | SLEV | | 1d | | | PRNT: <1.7 | |
| 1B5D-1 | | SLEV, | | 2 | | | SLEV | |
| 2B5B-3 | | JEV | | 3 | | | PRNT: 4.8 | |
| 2B6B-2 | | SLEV, | | 4a | | | SLEV | |
| 6B6C-1 | | JEV, | | 4b (in | | | PRNT: 2.9 | |
| | | MVEV, | | DII) | | | SLEV | |
| | | WNV, | | | | | PRNT: <1.7 | |
| | | YFV | | | | | SLEV | |
| | | All | | | | | PRNT: 2.3 | |
| | | Flavivirus/ | | | | | SLEV | |
| | | All | | | | | PRNT: <1.7 | |
| | | Flavivirus | | | | | SLEV | |
| | | | | | | | PRNT: 2.3 | |
| A3 | Chimpanzee | JEV | E2 | DI | Phage | Humanized | PRNT50 | [54] |
| B2 | | | | aa.K179 | display | | 0.04-0.2 | |
| E3 | | | | DII | Phage | | nM | |
| | | | | aa.I126 | Display | | PRNT50 | |
| | | | | DIII | Phage | | 0.02-2 nM | |
| | | | | aa.G132 | display | | PRNT50 | |
| | | | | | | | 0.14-0.93 | |
| | | | | | | | nM | |
| FabTJE12 | Human | JEV | E | N/A | Phage | Fab | FRNT50 | [55] |
| B02 | | | | | display | | 50.2 | |
| | | | | | | | μg/mL | |
| 5F10 | Human | CHIKV | E2 | Domain | EBV | IgG1λ2 | IC50 < | [56] |
| 8B10 | Human | CHIKV | E1-E2 | B | transformation | IgG1k | 100 ng/mL | |
| | | | | E2 | EBV | | IC50 < | |
| | | | | Domain A | transformation | | 100 ng/mL | |
| CHK-152 | Murine | CHIKV | E2 | aa 59 | Hybridoma | IgG2c | IC50 1-3 | [57] |
| | | | | | | | ng/mL | |
| 11E7 | Murine | CCHFV | Gn | C-ter | Hybridoma | IgG | PRNT80 | [58] |
| | | | | | | | diluted | |
| | | | | | | | 1/2560 | |
| 4-39-CC | Murine | RVFV | G2 | Domain | Hybridoma | IgG | PRNT80 | [59] |
| | | | | IV | | | diluted | |
| | | | | | | | 1/20480- | |
| | | | | | | | 1/81920 | |
| 2C9 | Murine | YFV | E | | Hybridoma | IgG2a | PRNT90: | [60] |
| | | | | | | | i/10⁴ 17D | |
| | | | | | | | 1/10^{5.2} | |
| | | | | | | | Asibi | |
| 5A | Human | YFV | E | DI-DII | Phage | ScFv | PRNT90: | [61] |
| 7A | | | | | Display | | 0.1-0.3 | |
| R3(27) | | | | | | | μg/mL | |
| 13D6 | Murine | TBEV | E | DIII | Hybridoma | IgG/chimeric | PRNT50: | [62] |
| | | | | | | | 1.9 μg/mL | |
| | | | | | | | PRNT50: | |
| | | | | | | | 4.5 μg/mL | |
| | | | | | | | IC50 1.9- | |
| | | | | | | | 16.7 | |
| | | | | | | | μg/mL | |
| 3B4C-4 | Murine | VEEV | E2 | aa | Hybridoma | IgG/humanized | PRNT70 | [63] |
| (Hy4-26C) | | | | 182-209 | | | (μg/mL) | |
| | | | | | | | 39.4-125 | |

### Example 2

Bioinformatics to identify and analyze target bacterial genes and to design effector RNAs.

Commercially-available strains of Klebsiella pneumonia (Kpn) (ATCC No. BAA-2146) and MRSA (ATCC No. BAA-1556) can be used to identify and analyze target bacterial genes and related RNA. The β-lactamase genes as well as single-gene determinants of resistance to other antibiotics can serve as suitable targets in the Kpn genome. Similar antibiotic resistance gene analyses can be performed for the MRSA strain. Genes that contribute to virulence (e.g., ETC produced by Kpn and hemolysin produced by MRSA), as well as direct bactericidal targets, such as RNA polymerase or gyrase can also serve as suitable targets. Once target genes are selected, the most vulnerable sites are identified. Several recent discoveries have illuminated the multiple regulatory mechanisms of small RNAs in bacteria, which have been found to directly induce degradation of target mRNAs and to inhibit translation of mRNAs by targeting the ribosome-binding site (RBS) (Gottesman and Storz, 2010). Assess the secondary structure of the RBS and other potential sites in target mRNAs, since single-stranded loops or segments in mRNAs are typically the first contact points with effector RNAs. Recent studies have also shown that transformation of bacteria with plasmids that encode full-length antisense RNAs (i.e., RNAs that are antisense along the full length of the target mRNA) effectively knocks down gene activity. Explore both natural and full-length antisense models.

When designing effector RNAs, limit nuclease degradation by including nuclease-protective secondary structures and/or unnatural backbones, such as those found in peptide nucleic acids (PNAs). Maximize gene knockdown by engineering antisense RNA to: (1) promote binding by the protein, Hfq, which is known to activate a number of small bacterial regulatory RNAs or (2) drive the formation of a loop-loop kissing complex between antisense RNA and target mRNA, which will result in full pairing between effector and target. Assess potential off-target effects by screening candidate RNAs against the human (or mouse) genome.

Multiple target/effector pairs are designed for each gene or, in the case of multi-locus traits like β-lactamase resistance, for each component locus. Antisense RNAs must be amenable to being assembled into PNAs. Our approach enables delivery of multiple RNAs simultaneously, such that multiple designs for targeting a given gene can be tested as a pool. Only one member of the pool need be effective, and this member can be identified in subsequent tests. Identify surface molecules on MRSA and Kpn that are conserved across most strains (AxioMx, Branford, Conn, automated phage display screening method can be used to develop targeting ligands that bind to these surface molecules with high affinity). Resulting targeting ligands are conjugated to PNA-loaded protocells to promote concentration of protocells at the sites of MRSA or Kpn infection.

Results

Identifying target antibiotic resistance genes.

To combat antibiotic resistance gene it is first essential to identify the target genes. An emerging pathogen of the Carbapenem-Resistant Enterobacteriaceae (CRE group), Klebsiella pneumoniae ATCC 2146 (Kpn2146), was selected. This strain was resistant to all 34 antibiotics tested at ATCC, and known to carry the carbapenemase gene blaNDM-1. A partial genome sequence (Kim at al., 2013) was inadequate for evaluating the antibiotic resistance gene repertoire. The genome was completed by preparing a Pacific Biosciences sequence dataset (PacBio) and using this data to connect the contigs in the partial genome, yielding five circular replicons: a chromosome and four plasmids (Hudson et al., 2014).

ATCC has reported resistance of Kpn2146 to each of the 34 antimicrobial and antimicrobial/inhibitor combinations tested, including tests for 23 β-lactams (penicillin with or without inhibitors, cephalosporins, carbapenems and aztreonam), five fluoroquinolones, three aminoglycosides (tobramycin, amikacin and gentamicin), and four others (tetracycline, tigecydine, nitrofurantoin, and trimethoprim/sulfamethoxazole); see http://www.atcc.org/˜/media/BA6C8F7C7C4C4649B2AEF501E51 D76B8.ashx for the full list. Kpn2146 resistance genes have also been surveyed with a combination of microarray and amplicon sequencing. The genome sequence fully rationalized the resistance profile, with ample evidence for one or more mechanisms explaining each observed antibiotic-resistance, and supported the gene survey. It further identified previously untested genes (like qnrB9), allelic multiplicity (aac(6′)-Ib, sul1, bla_{SHV-11} and bla_{CTM-M-15}) and location (plasmid vs. chromosome), as well as housekeeping gene mutations (Table 4). These gene duplications can increase resistance; duplication of bla_{SHV-11} has been shown to increase amoxicillin-resistance 16-fold.

Eight genes for β-lactamases representing all four Ambler classes were identified; together these explain the broad β-lactam and inhibitor resistance of Kpn2146. We further identified specific resistance genes for tetracycline, trimethoprim, sulfonamides, macrolides, and multiple aminoglycoside resistance genes, including three aac(6′)-Ib variants, one shown to confer additional low-level resistance to quinolones in addition to the usual spectrum of aminoglycosides inactivated by AAC(6′)-Ib which includes tobramycin, amikacin, and gentamicin C1a and C2.

The complete genome also reveals certain housekeeping gene mutations that are related to drug resistances. For example, the GyrA Ser83>11e and ParC Ser80>11e combination has previously been found in K. pneumoniae isolates with high-level resistance to several fluoroquinolones. QnrB9 of Kpn2146, like other plasmid-encoded quinolone resistance enzymes, confers low-level resistance to fluoroquinolones, and may facilitate selection of mutations in gyrA and parC associated with high-level resistance. A frameshift mutation in the nitroreductase gene nfsA is likely responsible for the observed resistance to nitrofurantoin.

The above observations explain the entire known resistance profile, except the tigecycline resistance. Mechanisms previously suggested for tigecycline resistance are mutations in the gene for the ribosomal protein S10 (Kpn2146 has the wild type allele) and mutations increasing the expression of the AcrAB/TolC efflux system. One mutation class causing overexpression of this efflux system is inactivation of its repressor RamR; Kpn2146 has such a ramR disruption (insertion of ISKpn18) that can thereby explain the observed tigecydine resistance. Additional efflux systems (Table 4), such as the macrolide-specific efflux system MacAB/TolC, may contribute to the intrinsic spectrum of resistance, especially if overexpressed.

An early nonsense mutation that disrupts the porin gene ompK35 was detected, fitting with many ESBL-producing K. pneumoniae strains that lack OmpK35. The concomitant loss of OmpK36 that significantly decreases susceptibility for meropenem and several cephalosporin β-lactams was not observed: ompK36 and ompK37 appear to be intact. In a recently reported Klebsiella carbapenem resistance mode, the marR regulatory gene is inactivated and the yedS porin gene is active; this mode is unlikely to pertain here since marR is intact and yedS is lacking in Kpn2146.

One third of the antibiotic resistance enzyme genes listed in Table 4, including all three of the aac(6′)-Ib alleles, are associated with five scattered class 1 integrons or integron fragments. Four of these are on plasmids, often within recognizable fragments of transposons, and the fifth is within a genomic island on the chromosome. We discuss below a case of cassette swapping where comparative analysis suggests the swap may have been mediated by homologous recombination rather than class 1 integron integrase action.

TABLE 4
Enzymes, efflux pumps, and mutations expected to confer
resistance to antibiotics of clinical relevance^{a}

| Enzyme^{b} | Gene locations(s) | Coordinates | Resistance phenotype |
|---|---|---|---|
| NDM-1 (class | pNDM = US Tn125 | 122191-123003 | Penicillins, cephalosporins, |
| B) | | | carbapenems, inhibitor-resistant |
| SHV-11 (class | 1. pKpn2146b | 36313-37173 | Penicillins, some |
| A)^{c} | 2. Chromosome | 2612996-2613856 | cephalosporins, inhibitor- |
| | | | sensitive |
| CTX-M-15 | 1. pKpN2146b | 47130-48005 | Penicillins, some |
| (class A) | ISEcp1 | | cephalosporins, aztreonam, |
| | 2. Chromosome | 5407530-5408405 | inhibitor-sensitive |
| | ISEcp1 | | |
| TEM-1 (class | pKpn2146b Tn2 | 50827-51687 | Penicillins, some |
| A) | | | cephalosporins, inhibitor- |
| | | | sensitive |
| CMY-6 (class | pNDM-US ISEcp1 | 72203-73348 | Penicillins, some |
| C) | | | cephalosporins, inhibitor- |
| | | | resistant |
| OXA-1 (class | pKpn2146b ΔIn37 | 38798-39673 | Penicillins, inhibitor-resistant |
| D) | | | |
| AAC(3)-Ile | pKpn2146b | 41116-41976 | Gentamicin, tobramycin, |
| | | | netilmicin, sisomicin |
| ACC(6′)-Ib | pNDM-US In46 | 115114-115737 | Tobramycin, amikacin, |
| (43) | | | netilmicin, sisomicin |
| ACC(6′)-Ib (1) | pKpn2146b | 82745-83350 | Tobramycin, amikacin, |
| | ΔInTn1331 | | netilmicin, sisomicin |
| ACC(6′)-Ib-CR | PkPN2146B ΔIn37 | 38113-38712 | Tobramycin, amikacin, |
| (29) | | | netilmicin, sisomicin, quinolones |
| | | | (low-level) |
| ANT(3″)-Ia | Kpn23SapB In127 | 2297711-2298502 | Streptomycin, spectinomycin |
| APH(3″)-Ib | pKpn2146b ISCR2 | 53244-54047 | Streptomycin |
| (StrA) | | | |
| APH(6)-Id | pKpn2146b ISCR2 | 52408-53238 | Streptomycin |
| (StrB) | | | |
| Sul2 | pKpn2146b ISCR2 | 54108-54923 | Sulfonamides |
| RmtC | pNDM-US ISEcp1 | 120100-120945 | Aminoglycosides (via rRNA |
| | | | modification) |
| Sul1 | 1. Kpn23SapB In127 | 2299007-2299846 | Sulfonamides |
| | 2. pNDM-US In46 | 116245-117084 | |
| DfrA14 | pKpn2146b In191 | 8281-8754 | Trimethoprim |
| QnrB9 | pKpn2146b | 26074-26742 | Quinolones, fluoroquinolones |
| Mph(A) | pKpn2146c | 16503-17408 | Macrolides, Erythromycin |
| FosA | Chromosome | 667960-668379 | Fosfomycin |
| Efflux pump | Gene location | | Probable Substrate(s)^{d} |
| AcrAB-TolC | Chromosome | 1249681-1254043 | Aminoglycosides, β-lactams, |
| | | | tigecycline, macrolides |
| AcrEF-TolC | Chromosome | 4936203-4940465 | Minor role |
| EefABC | Chromosome | 5354323-5329922 | Chloramphenicol, tetracyclines, |
| | | | ciprofloxacin |
| MacAB-TolC | Chromosome | 1857393-1860445 | Macrolides |
| MdfA | Chromosome | 1781588-1782820 | Aminoglycosides, |
| | | | fluoroquinolones, |
| | | | chloramphenicol |
| MdtG, H, K, | Chromosome | * | Many possible substrates (MFS |
| L, M, NOP | | | superfamily pumps) |
| OqxAB | Chromosome | 4169609-4173960 | Chloramphenicol, |
| | | | fluoroquinolones, trimethoprim |
| EmrAB | Chromosome | 4218886-4221612 | Nalidixic acid, hydrophobic |
| | | | compounds |
| TetA(A) | pKpn2146c Tn1721 | 19168-20367 | Tetracyclines |
| Gene | Mutation | | Resistance phenotype |
| gyrA Gyrase | Ser83TTC→DeATC | 3763583-3766216 | Quinolone, fluoroquinolones |
| parC Topo IV | Ser80AGC→DeATC | 4689294-4691552 | Quinolone, fluoroquinolones |
| nfsA | Frameshift | 1826275-1826998 | Nitrofurantoin |
| Nitroreductase | | | |

^{a}Excluding the resistance for bleomycin, an antibiotic used clinically only as an antitumor agent
^{b}Variant number from Table 1 of Ramirez et al. is used to distinguish the AAC(6′)-Ib variants.
^{c}Two silent differences between two copies.
^{d}Probable efflux substrates identified from literature sources including ARDB; the substrates list is not comprehensive and in many cases has been deduced from organisms other than K. pneumoniae.
^{e}Mdt genes are scattered over the chromosome

Design of PNA sequences to target antibiotic resistance genes.

Twenty-one of the above-described antibiotic resistance enzyme genes (not transporter genes, which probably contribute less to resistance) from Klebsiella pneumoniae ATCC 2146 were chosen as targets. Work on gene inhibition in Salmonella (Soofi and Seleem, 2012) and Brucella (Rajasekaran et al., 2013) elucidated design principles for peptide-nucleic acid (PNA) gene-specific inhibitors, which were applied to our targets. Anti-antibiotic-resistance PNA sequences are listed in Table 5, with the six beta-lactamase genes being listed first.

TABLE 5
Enzymes, efflux pumps, and mutations expected to confer resistance to
antibiotics of clinical relevance

Metallo-beta-lactamase NDM-1; p843:239-1051
CATcaagttttc (SEQ ID NO: 28)
SHV-11 beta lactamase; csome:1155376-1154516, p850:72286-71426
CATaaccacaat (SEQ ID NO: 29)
CMY-6 AmpC-type beta-lactamse: p843:91073-92218
CATgaaatcagt (SEQ ID NO: 30)
CTX-M-15 extended spectrum beta-lactamase; csome:3948604-3949479
CATgggattcct (SEQ ID NO: 31)
TAM-1 beta-lactamase; p850:766-1626
OXA-1 beta-lactamase; p850:73911-74786
CAAttaaatgagg (SEQ ID NO: 32)
Aminoglycoside-(3)(9)-adenyltransferase AADA2; csome:838991-840016
CATtcaaaggcc (SEQ ID NO: 33)
SulI dihydropteroate synthase; csome:840521-841360
CATggcgtcggc (SEQ ID NO: 34)
Undecaprenyl-diphosphates; csome:325972-3257151
CATccaattaaa (SEQ ID NO: 35)
16S ribosomal RNA methyltransferase: csome:4799637-4798816
CATtgggtatta (SEQ ID NO: 36)
AAC (6)-Ib aminoglycoside 6-N-acetyl transferase type Ib; p843:133984-134607
CAAttaatgagg (SEQ ID NO: 37)
SulI dihydropteroate synthase; p843:135117-135956
CATggcgtcggc (SEQ ID NO: 38)
16S rRNA methyltransferase RmtC; p843:139815-138970
CATatatggtct (SEQ ID NO: 39)
Aminoglycoside 3 phosphotransferase APH(3)-1b (strA); p850:3986-3183
CAAtggaggttc (SEQ ID NO: 40)
Sul2 sulfonamide insensitive dihydropteroate synthetase; p850:4862-4047
CATggggcttc (SEQ ID NO: 41)
Streptomycin 3-O-adenyltransferase aadA ANT (3)-Ia; p850:32614-32420
CATgatgtttaa (SEQ ID NO: 42)
Dfra14 trirnethoprim-resistant dihydrofolate reductase; p850:43383-43856
CAAggttctcat (SEQ ID NO: 43)
QnrB10; p850:61200-61844
CATatttgtacc (SEQ ID NO: 44)
Aminoglycoside N(3)-acetyltransferase II (Acc(3)-II); p 50:76229-77089
CATcgcgatatc (SEQ ID NO: 45)
Tetracycline efflux protein TetA; p852:90940-92139
CACgtctggcct (SEQ ID NO: 46)
Macrolide 2-phosphotransferase mphA; p852:88273-89178
CATgattcactc (SEQ ID NO: 47)

### Example 3

Use of Quantitative PCR (qPCR) to Quantify the Decrease in Target mRNA Expression

To assess the efficiency of target gene knock down, electroporating commercially-available strains of Kpn (2146) and MRSA (1556) are used in the presence of candidate RNAs and quantitative PCR (qPCR) employed to quantify the decrease in target mRNA expression. These strains have numerous genes that contribute to antibiotic resistance via complex, interconnected, orthogonal mechanisms. Once antisense RNA cocktails using commercial strains of Kpn and MRSA are optimized, test them against clinical isolates of extended-spectrum β-lactamase (ESBL)-producing K. pneumoniae and mecA-positive S. aureus. When compared to commercial strains, clinical isolates might have different or additional genes that contribute to antibiotic resistance; therefore, re-design antisense RNA cocktails for each target pathogen is necessary. In parallel, the effects of RNA length, structure, and composition on PNA formation, stability, and size are assessed. Assess the ability of candidate PNAs to penetrate Kpn-2146 and MRSA-1556 and effectively knock down gene expression using qPCR to quantify target mRNA.

Results

Choice of E. coli ER2420/pACYC177 as Model System

Given the large number of antimicrobial resistance mechanisms present in our K. pneumoniae BAA-2146 strain the cell penetrating peptide-peptide nucleic acid (CPP-PNA) conjugate silencing approach with a simpler experimental system, with fewer overlapping resistance mechanisms was selected. Non-pathogenic laboratory strains of E. coli are routinely used for cloning or protein expression, utilizing plasmids with specific drug resistance genes for selection in the laboratory. In most cases the resistance genes are carried on small, high-copy number plasmids, whereas our K. pneumoniae (and many wild Gram-negative pathogens) carry resistance genes on large, low-copy number plasmids. As a model organism, we chose a non-pathogenic E. coli strain, ER2420, which harbors a small, low-copy plasmid with two drug resistance genes: bla_{TEM-1} (TEM-1 beta-lactamase) and aph-3′-ia (aminoglycoside-3′-phosphotransferase type Ia, or APH(3′)-Ia). The E. coli APH(3′)-Ia confers resistance to the aminoglycoside drug kanamycin. The K. pneumoniae has several other aminoglycoside-modifying enzymes, but not this specific gene.

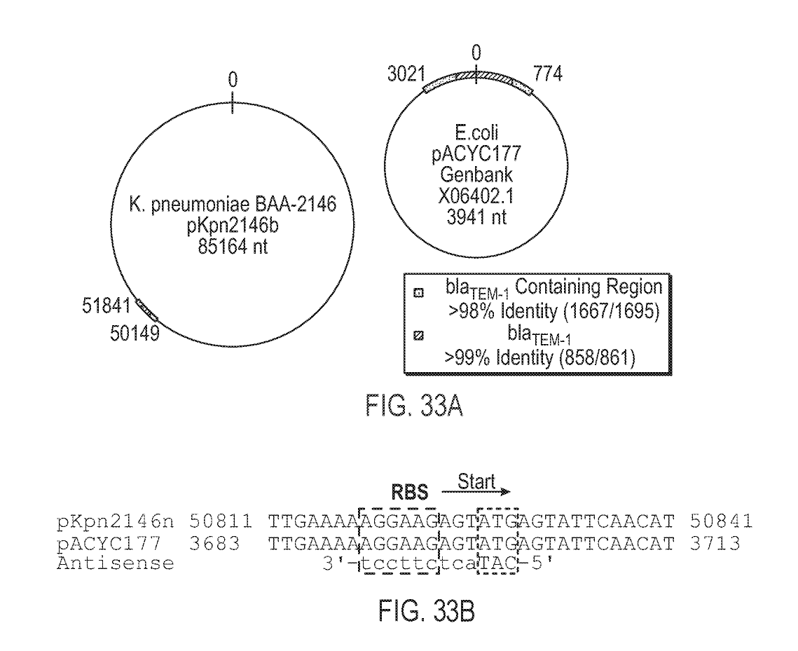

TEM-1 is one of six beta-lactamases harbored by K. pneumoniae BAA-2146, and it is a common mechanism of resistance in clinical isolates worldwide. TEM-1 confers resistance to several beta-lactam drugs including penicillin, and is susceptible to classical beta-lactamase inhibitors such as clavulanic acid (Paterson and Bonomo, 2005). The E. coli TEM-1 beta-lactamase has the identical amino acid sequence as TEM-1 in our K. pneumoniae strain, and is found in almost the same genetic environment, including the same start codon and ribosomal binding site, as shown in FIG. 33. This means that an antisense CPP-PNA conjugate designed for the E. coli gene would silence the same gene in K. pneumonia (See FIG. 33).

Rapid, microscale testing for MIC with PNA antisense compounds.

The “gold standard” test for determining the minimum inhibitory concentration (MIC) or efficacy of an antimicrobial compound is a broth dilution assay. In this assay, a standardized stock of the microbe (the inoculum) is introduced into a series of test tubes containing a series of two-fold dilutions of the antimicrobial compound in sterile growth media (usually Mueller-Hinton broth for aerobic growth, or occasionally Mueller-Hinton agar). The tubes are incubated (typically overnight), and monitored for microbial growth, indicated by onset of turbidity. The lowest concentration that prevents the growth of the microbe is called the minimum inhibitory concentration, or MIC. Traditional antibiotics are relatively inexpensive and are active at fairly low concentrations, and the broth dilution protocol can easily be carried out in volumes of 5-10 mL per concentration. More recently, the protocol has been adapted to 96-well plate, where growth is performed in volumes of 100 μL per well, and typically 8 to 12 concentrations of drug are used per test to establish MIC. This scaled-down protocol is termed a broth microdilution assay.

Testing the CPP-PNA conjugates for silencing drug resistance genes presented a unique challenge. For testing and development purposes, the CPP-PNA conjugates are custom-synthesized at small scale, for relatively high cost, on the order of $1,000/mg (100 nmol synthesis scale). To test whether the CPP-PNA conjugates effectively silence drug resistance genes, we need to demonstrate that the CPP-PNA restores susceptibility to a drug, or in other words demonstrate that the CPP-PNA lowers the minimum inhibitory concentration (MIC) of the drug to a resistant microbe. Based on prior literature, we expected effective concentrations of the CPP-PNA conjugates on the order of 5-40 μM (Soofi and Seleem, 2012; Bai et al., 2012a; Bai et al., 2012b). A 100 nmol-scale batch of CPP-PNA provides enough material to produce 2.5 mL at 40 μM concentration, or approximately 25 wells of a standard broth microdilution. Since a typical MIC test requires at least 8 wells per drug, the typical 100 nmol synthesis scale for CPP-PNA provides barely enough material for three tests (for example, one drug repeated twice, plus a no-drug control experiment). Clearly, even the small-scale broth microdilution is not small enough to enable cost-effective screening of CPP-PNA conjugates.

To address this shortcoming for CPP-PNA conjugates, microscale “test strip” compatible with either broth or agar microdilution was developed, allowing MIC to be determined with only 5-10 μL of test solution. The test strip comprises a linear or rectangular array of wells (up to 20 per strip), designed with spacing of 4.5 mm or 3 mm between wells. These spacings are one-half or one-third the 9 mm spacing of an SBS-standard microtiter plate, ensuring that our test strips are compatible with multichannel pipettes and other laboratory instrumentation designed around standard 9 mm spacing. (SBS-standard 384-well plates with 9/2=4.5 mm spacing and 1536-well plates with 9/4=2.25 mm spacing also retain compatibility with multichannel pipettes designed for 96 well plates).

The closest commercially available items identified are 8- or 12-tube strips designed for PCR. These tubes are typically conical with rounded bottoms and are typically used for volumes of 20-50 μL. The present test strips, by contrast, have a flat bottom with shallow, open chambers, which allows for efficient gas exchange for microbial growth without shaking or agitation. The shallow wells also accommodate either liquid or solid growth media (e.g., agar). 1536-well plates would accommodate similar liquid volumes as the present test strips, but in relatively deep wells which would present a greater challenge for gas exchange as well as pipetting. Typical experiments would use far less than 1536 wells, but once a sterile microwell plate is used, it is inadvisable to re-use it for subsequent experiments (e.g., simply using 1-2 rows per day until the plate was filled will likely lead to trouble with contamination).

The shallow wells present a challenge for detection of microbial growth by turbidity. To enable faster and more sensitive growth determination in shallow wells, the colorimetric and fluorogenic redox indicator resazurin (Mann and Markham, 1998; Palomino et al., 2002; Sener et al., 2011) was used. This well-known “viability indicator” undergoes a conspicuous color change from dark blue to bright pink, and non-fluorescent to fluorescent, as a consequence of microbial respiration (resazurin is irreversibly reduced to resorufin, and eventually undergoes a further reversible reduction to colorless dihydroresorufin). With this test strip, screening capability, for the standard 100 nmol scale of CPP-PNA conjugate synthesis was increased.

Fabrication of microwell test strips.

Test strips were fabricated by laser micromachining and lamination of thin plastic sheets and pressure-sensitive adhesives. Ideally, the test strips would be fabricated of materials that laser-cut easily, and can withstand autoclave sterilization at 121° C. Black Delrin was found to be a suitable material on both of these points. Black Delrin also provides high contrast and minimal autofluorescence for either colorimetric or fluorescent readout of the resazurin growth assay. A variety of materials were tested for use as a clear bottom, with 0.2 mm PMMA or 0.25 mm PET providing acceptable results. PMMA typically has a glass transition temperature (Tg) below the temperature of the autoclave sterilization, but in this case since the PMMA was tightly bonded to the overlying Delrin, with relatively small clear apertures, we did not observe noticeable sagging or melting upon autoclaving.

Laser micromachining is acceptable as a laboratory prototyping technique, but mass production of such test strips would likely be performed by injection molding, and sterilization would more likely be performed by ethylene oxide gas or gamma irradiation, and thus different materials would likely be used—polypropylene may be an acceptable choice.

Proof of concept with conventional antimicrobials.

The microwell resazurin growth assay was tested with several classes of antimicrobial compounds to understand the performance of the test strips and rapid growth test. Antimicrobial compounds that were used for testing included amoxicillin (with or without the beta-lactamase inhibitor clavulanic acid), kanamycin, ciprofloxacin, nalibdixic acid, tetracycline, and rifampicin (resistant E. coli for several of these drugs were available in the laboratory). Test strips were prepared with a liquid suspension of the drug, or with drugs dried down into the wells, or with the drugs prepared in molten agar media dispensed into the wells prior to solidification. Overnight cultures of E. coli were diluted to give an inoculum equivalent to approximately 5×10⁵ cells/mL (about 5000 cells per 10 μL well), which is typical for broth dilution. A typical final concentration of resazurin was 100 μg/mL, which has previously been demonstrated to be non-inhibitory to bacteria.

Test strips include at least one well as a no-drug control, and one well as a sterility control (no cells inoculated). Test strips were typically placed in a sterile Petri dish along with several moist tissues (Kimwipes) to provide a humid atmosphere. The Petri dish was covered, wrapped with Parafilm, and placed in a 37° C. incubator. The test strips would monitored periodically for signs of color change, which would typically become evident within 4-5 hours.

Most frequently, cells were inoculated by pipette. We did experiment with producing a comb-like inoculating device, with “pins” or teeth that would dispense a controlled volume of cell suspension to each well of the device simultaneously. In the case of test strips fabricated with solid agar pads, we also experimented with using a small “spatula” to spread cell suspension across the wells.

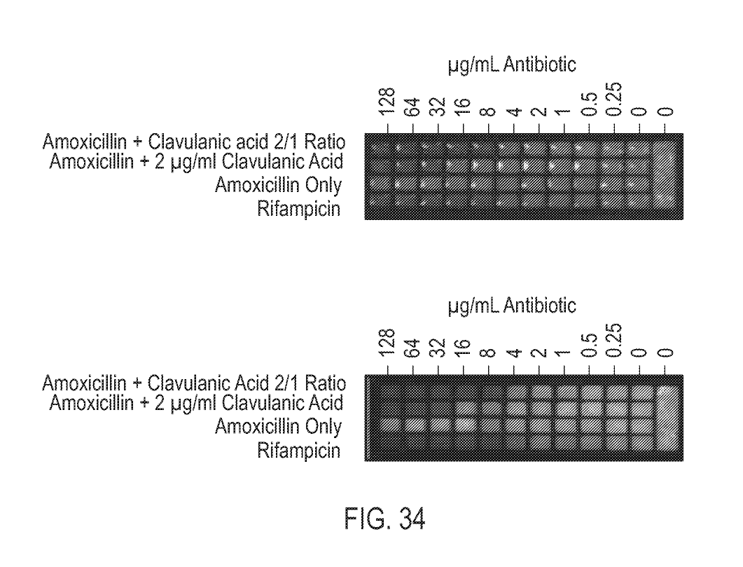

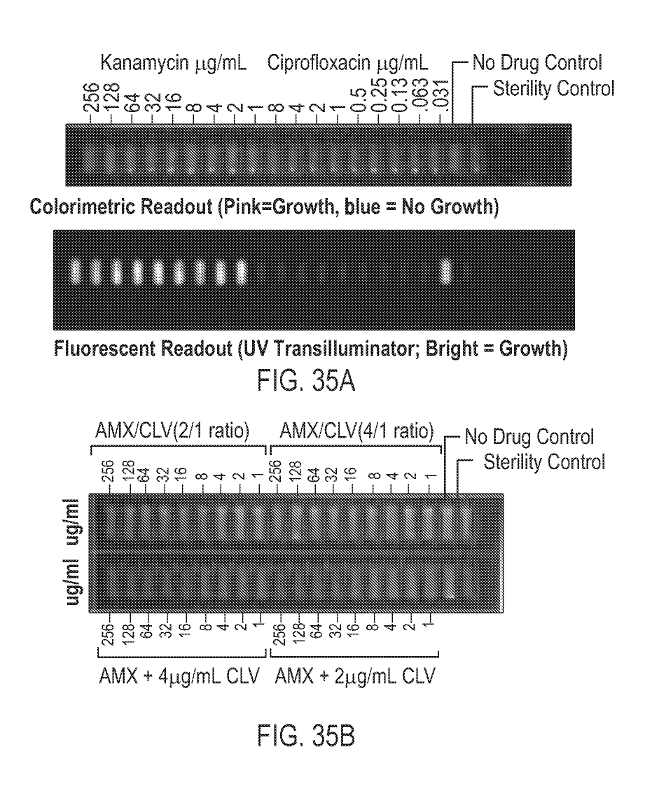

Examples of test strips used for conventional antibiotic testing are shown in FIGS. 34 and 35, which also illustrate a variety of configurations developed and tested with rapid prototyping. Note a common “test” was amoxicillin with and without the beta-lactamase inhibitor davulanic acid, because the use of an inhibitor to restore susceptibility to an antibiotic is similar in principle to the intended use of the CPP-PNA silencers. The key difference is that davulanic acid inhibits the gene product (the TEM-1 beta-lactamase) whereas the CPP-PNA silencer blocks translation of the gene product from the mRNA. In other words, the CPP-PNA targets an earlier step in the expression of the drug resistance.

Proof of concept with CPP-PNA.

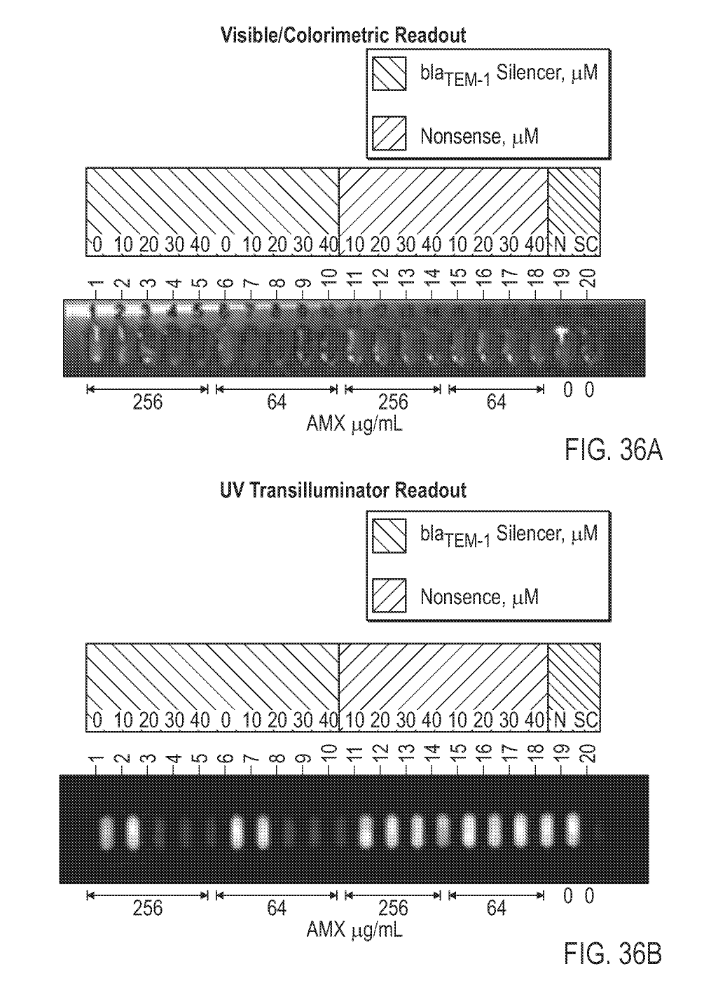

The test strip described above was used for a microvolume screening assay with a CPP-PNA conjugate designed to silence the biaTEM-1 gene. In our first trial, two concentration of amoxicillin were tested: 256 and 64 μg/mL, with CPP-PNA concentration ranging from 0-40 μM. Results are shown in FIG. 36.

This strain of E. coli is resistant to AMX, MIC>256 mg/mL (well 1). With AMX=256 or 64 μg/mL, the E. coli still grows with 10 μM of the silencer probe, but not with the silencer probe at 20-40 μM, suggesting that the silencer probe suppresses the resistance due to the bla_{TEM-1} gene. Growth is unaffected by presence of the nonsense control probe. As expected, the cells grow in the no-antibiotic control well (N), and no growth is observed in the sterility control (SC) well. Thus this initial test demonstrated the concept of lowering MIC by silencing translation of the resistance gene, and suggested there was some promise for moving forward. Subsequently, we attempted to determine the efficacy at a lower drug concentration (E. coli is considered “resistant” to amoxicillin at an MIC>8 pglmL).

However, in several subsequent trials we could not reproduce even the initial result of silencing at 20 μM CPP-PNA and 64 μg/mL AMX. We eventually suspected this was due to aggregation of the CPP-PNA upon storage in solution at −20° C. The manufacturer recommended heating immediately prior to use. Eventually we were able to reproduce the initial result, although we found that longer incubations (>8 hours) led eventually to growth of the E. coli even in wells with the silencer. This suggested that the CPP-PNA did not fully or permanently suppress expression of the TEM-1, but merely slowed the growth rate of the E. coli.

A second possible explanation for incomplete inhibition of growth is that the inoculum already contains some active TEM-1 enzyme. The overnight growth and subsequent re-growth of the culture was performed without drugs, so as to prevent inducible overexpression of the resistance enzyme. However, this particular gene may be constitutively expressed regardless of whether the drug is present. Thus, the initial inoculum may contain enough active enzyme to survive, if not grow, in the presence of drug. B-lactam drugs such as amoxicillin are relatively unstable in aqueous media (half life on the order of a few hours), and thus the E. coli with pre-existent TEM-1 may simply persist in a viable but not actively-growing state until the active concentration of drug drops low enough that the E. coli can proliferate. Additionally, β-lactamases such as TEM-1 can be secreted into growth media, or released upon cell death. Small amounts of secreted or released TEM-1 can slowly hydrolyze amoxicillin, eventually reducing it to non-inhibitory levels.

The products of gene expression, e.g., a protein or an enzyme, are generally much more stable than the corresponding mRNA. Proteins or enzymes can have half-lives of hours or days, and once expressed they tend to persist in cells. Bacterial mRNA, on the other hand, is highly unstable. Once a gene is expressed, the mRNA may persist for only a few minutes before being degraded. Bacterial mRNA (apart from highly expressed housekeeping genes) may be present at very low copy number per cell, on the order of zero, one, or a few copies present at any one time, and in many cases are transcribed in bursts (Chong et al., 2014). Protein translation from bacterial mRNA also proceeds in “bursts” (Yu, Science 311:16000-1603, 2006) with numerous copies of protein produced from a single mRNA transcript in a matter of minutes before the mRNA is degraded.

To be effective at completely suppressing gene expression, a silencing probe needs to effectively out-compete the protein translation machinery (i.e., the ribosome) for binding to the mRNA. Bacterial rRNA is present in high copy number, on the order of 10,000 copies per cell. Considering a typical E. coli has a volume of about 2 fL (about 1 μm×1 μm×2 μm), 10′ copies of rRNA translates to a concentration of 8 μM. Even if a silencing probe binds to the mRNA with 1-2 orders of magnitude higher affinity than the rRNA, the silencer must still be present within the cell at micromolar or tens of micromolar concentration at all times to effectively outcompete the rRNA for binding of every transcript produced, to prevent even a single burst of translation. This may account for previous reports of CPP-PNA silencing probes having active concentrations of tens of micromolar.

As a therapeutic approach, combine the bla_{TEM-1} CPP-PNA to silence translation of TEM-1, in combination with clavulanic acid to inhibit any TEM-1 that is expressed. The CPP-PNA conjugate should be much more stable in solution than clavulanic acid, which itself is a β-lactam with a half-life of a few hours in solution. In combination, clavulanic acid provides inhibition of already-formed TEM-1, while the CPP-PNA prevents new TEM-1 from being formed.

Besides the growth-based assay to determine MIC with and without the CPP-PNA silencer, verify that the CPP-PNA silencer prevents expression of the TEM-1 by growing E. coli in the presence or absence of the CPP-PNA silencer with no drug (so there is no selection pressure), and then test for beta-lactamase activity. This can be done easily using colorimetric or fluorogenic beta-lactamase substrates such as nitrocefin or fluorocillin. Additionally, plate or culture from wells grown with AMX+CPP-PNA silencer to determine whether the CPP-PNA restores the bactericidal effect of AMX, or simply prevents growth of a subpopulation of viable cells (bacteriostatic effect).

### Example 4

### Novel Rapid Diagnostic Assay Based on PMA-PCR

Quantitative PCR provides accurate determination of bacterial growth: each doubling of the bacteria should lead to a decrease of 1 cycle (delta-Ct=−1) for detection in real-time PCR. Thus sampling at multiple time points could give an indication of whether bacteria are growing in presence of antibiotics. However, PCR does not discriminate between cells that are alive and cells that are dead or dying. Depending on their mechanism of action, antibiotics may or may not immediately kill bacteria, and in some cases bacteria may survive through one or more rounds of DNA replication or even cell division in the presence of antibiotics.

To improve the discrimination between living and dying or dead cells at early time points, we employed propidium monoazide (PMA). PMA is a photoreadive intercalating dye that is excluded from cells with intact membranes, but penetrates into dead cells with permeabilzed membranes. Activation with blue light results in formation of a nitrene radical, which crosslinks, modifies, or otherwise damages DNA to which PMA is bound, rendering it non-amplifiable by PCR. Thus, PMA treatment biases PCR against amplification of DNA from dead cells. In qPCR, this effect manifests as a large delta-Ct between a sample treated with PMA versus an untreated control (Nocker et al., 2006).

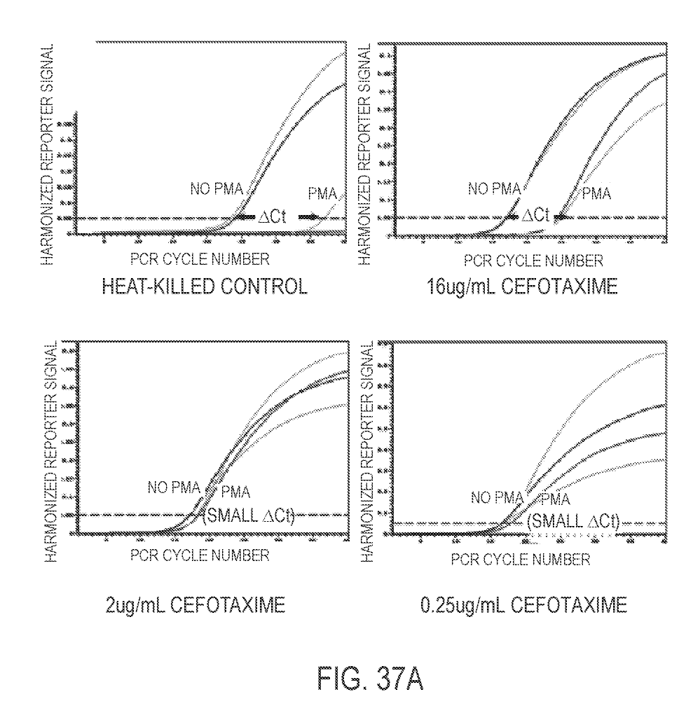

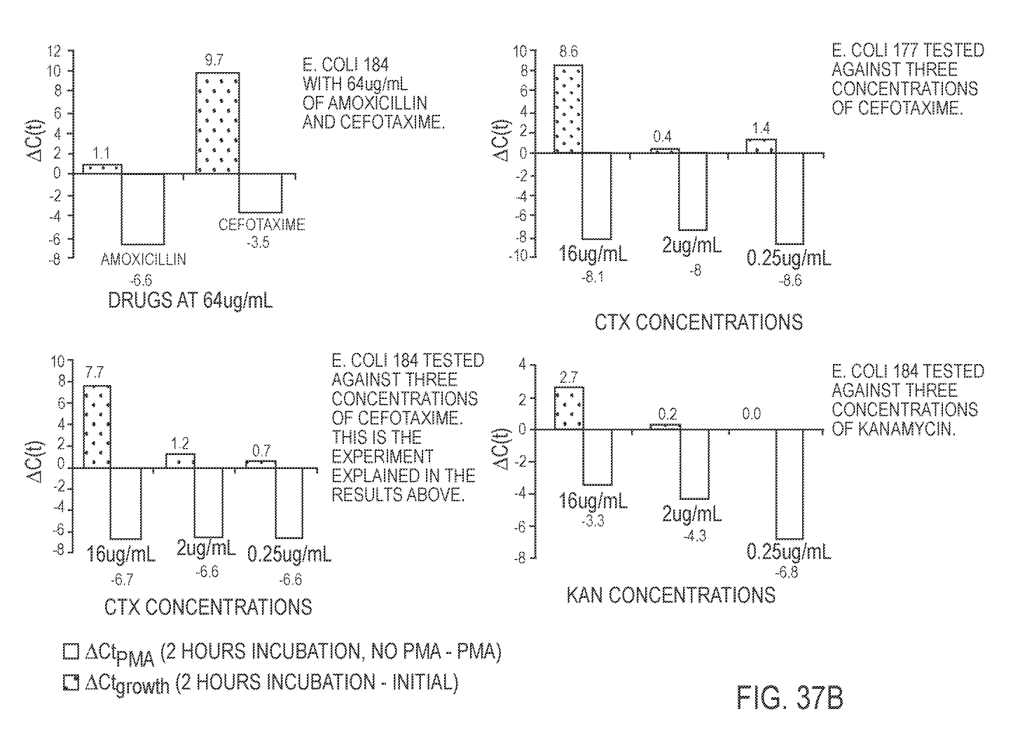

E. coli ER2420 with pACYC177 (amoxicillin/kanamycin resistance) or pACYC184 (tetracycline/chloramphenicol resistance) was measured. As early as 2 hours later, based upon the delta-Ct for PMA treatment, the killing action of amoxicillin or kanamycin in the susceptible strain (pACYC184) and not in the resistant strain (pACYC177) was detected. The killing effect of cefotaxime (a cephalosporin which TEM-1 beta-lactamase does not cleave) in both strains was also detected. However, the technique does have its limits: the killing action was most evident (large delta-Ct) within 2 hours only for concentrations of drugs that were well above the expected MIC for amoxicillin, kanamycin, or cefotaxime. Selected results are shown in FIG. 37.

Ciprofloxacin was another drug for which the PMA technique was not useful. Ciprofloxacin is rapidly bactericidal to both E. coli at low concentrations. However, the mechanism of killing (inhibition of DNA replication) does not result in immediate permeabilization of the cell membrane (Mason et al., 1995). So in the case of ciprofloxacin, PMA treatment did not result in a noticeable delta-Ct. We did observe, however, minimal delta-Ct (minimal growth) between the initial inoculum (heat-killed at the beginning of the experiment), and the inoculum after several hours of growth, indicating that qPCR alone (without PMA treatment) can provide an indication of growth in presence of ciprofloxacin. This is not entirely surprising, as qPCR detects DNA copy number as an indicator of bacterial concentration, and ciprofloxacin suppresses growth by preventing DNA replication.

Apart from the rapid growth step (2 hours incubation), the PMA-PCR experiments were performed with purely conventional techniques, including spin columns for DNA extraction, and conventional real-time PCR. Even so, the entire protocol required approximately 5 hours. Automating the DNA extraction, and using newer, rapid-cycling PCR technology could likely reduce the total protocol to under 3 hours. It is questionable whether the growth step could be reduced significantly below 2 hours, as bacteria often undergo a lag phase upon being introduced into new media (i.e., a test medium containing antibiotics). During this period, the bacteria do not actively divide, but rather adjust their metabolism to new conditions. This is the case whether a stationary-phase overnight culture is diluted to form the inoculum, or a clinical sample is placed directly into a test medium. Slow-growing or fastidious microbes such as Mycobacterium tuberculosis or Francisella tularensis which inherently divide more slowly would require longer incubations to detect a phenotypic response to drugs. The PMA-PCR technique may prove useful for determining viability or antimicrobial susceptibility for fast-growing biodefense pathogens such as Bacillus anthracis or Yersinia pestis, and literature suggests the technique also has some ability to discriminate infectious from non-infectious viruses (Parshionikar, Appl. Environ. Microbiol. 76:4318-4326, 2010). The PMA-PCR technique also presents the possibility of combining rapid phenotypic response (growth vs death) with molecular specificity. This may be useful in mixed samples (e.g., clinical samples) where total growth (as given by the color change of rifampicin, for example) would not distinguish between pathogens. In contrast, the PMA-PCR technique could be adapted to detect only certain species in a mixed culture.

### Example 5

### Optimization of Pore Chemistry

Despite recent improvements in encapsulation efficiencies and serum stabilities, state-of-the-art liposomes, multilamellar vesicles, and polymeric nanoparticles still suffer from several limitations, including complex processing techniques that are highly sensitive to pH, temperature, ionic strength, presence of organic solvents, lipid or polymer size and composition, and physicochemical properties of the cargo molecule, all of which impact the resulting nanoparticle's size, stability, entrapment efficiency, and release rate (Conley et al., 1997: Couvreur and Vauthier, 2006; Morilla and Romero, 2011; Wong at al., 2003).

In contrast, MSNPs formed via aerosol-assisted EISA have an extremely high surface area (>1200 m²/g), which enables high concentrations of various therapeutic and diagnostic agents to be adsorbed within the core by simple immersion in a solution of the cargo(s) of interest. Furthermore, since aerosol-assisted EISA yields MSNPs that are compatible with a range of post-synthesis modifications, the naturally negatively-charged pore walls can be modified with a variety of functional moieties, enabling facile encapsulation of physicochemically disparate molecules, including acidic, basic, and hydrophobic drugs, proteins, small interfering RNA, minicircle DNA vectors, plasmids, and diagnostic agents like quantum dots and iron oxide nanoparticles (Ashley et al., 2012; Ashley et al., 2011; Epler et al., 2012).

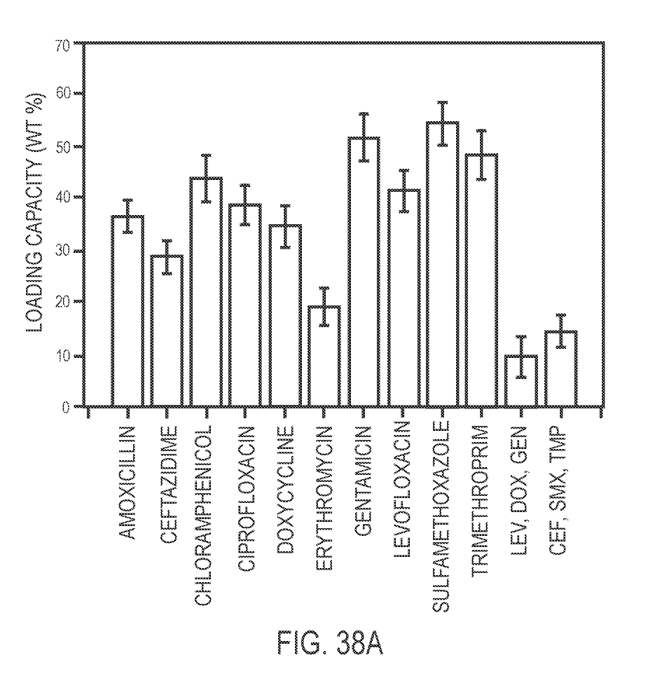

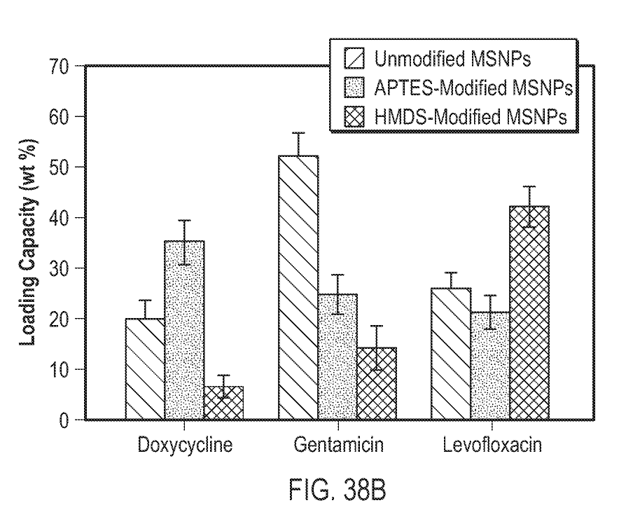

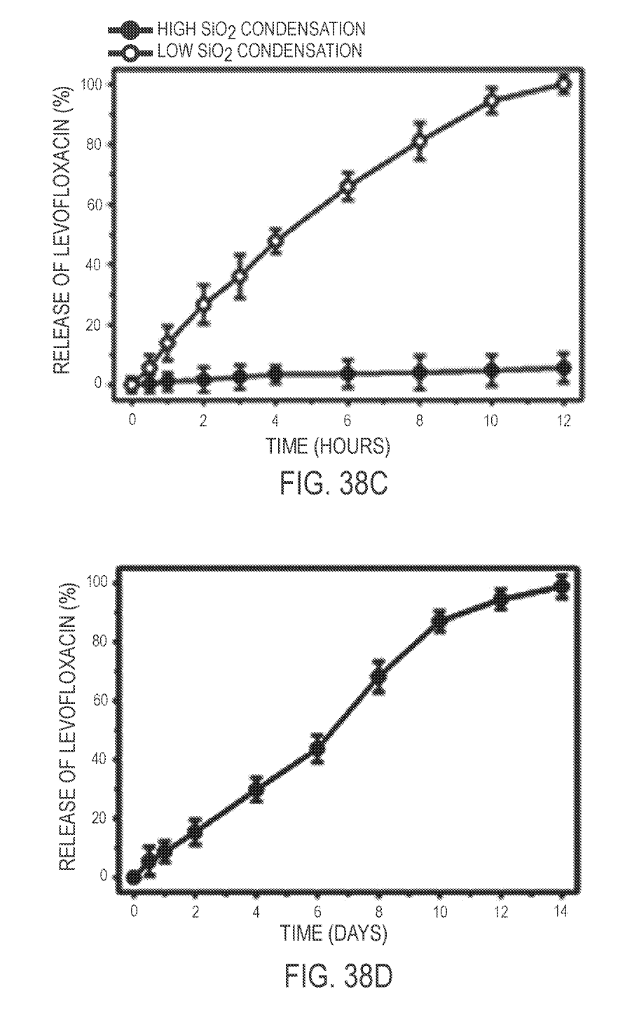

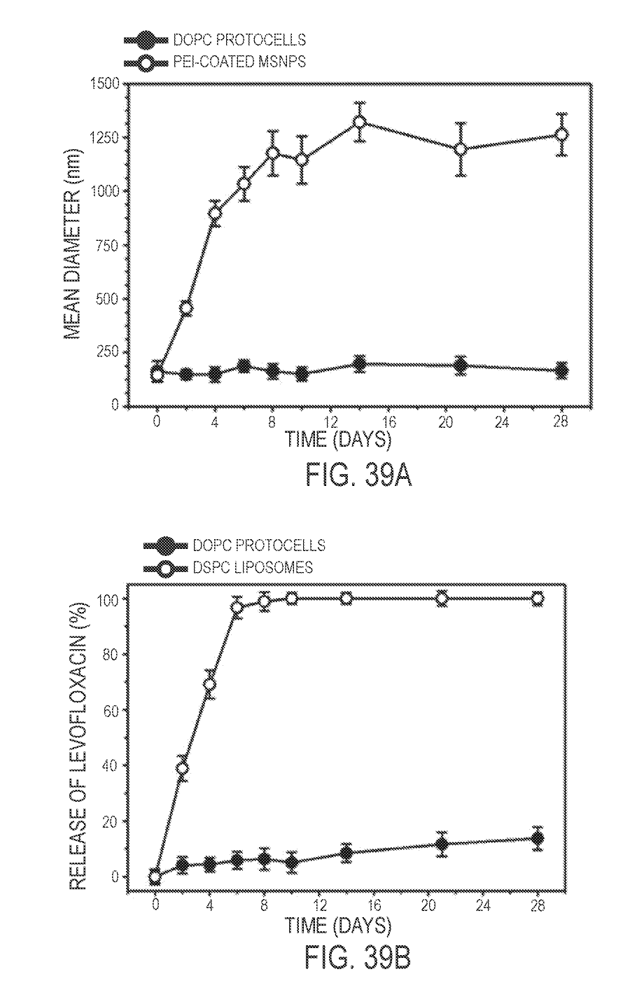

As demonstrated by FIG. 38A, MSNPs formed via aerosol-assisted EISA have loading capacities of 20-55 wt % for various individual antibiotics and 10-15 wt % for individual antibiotics in three-drug-cocktails; it is important to note that these capacities are 10-fold higher than other MSNP-based delivery vehicles (Clemens et al., 2012) and 100 to 1000-fold higher than similarly-sized liposomes and polymeric nanoparticles (Couvreur and Vauthier, 2006; Morilla and Romero, 2011; Wong et al., 2003). High loading capacities were achieved for acidic, basic, and hydrophobic drugs by modulating the pore chemistry (see FIG. 38B) and by altering the solvent used to dissolve the drug prior to loading. Unlike MSNPs formed using solution-based techniques, MSNPs formed via aerosol-assisted EISA are compatible with all aqueous and organic solvents, which ensures that the maximum concentration of drug loaded within the pore network is essentially equivalent to the drug's maximum solubility in its ideal solvent. Another unique feature of our MSNPs is that the rate at which encapsulated drug is released can be precisely modulated by varying the degree of silica framework condensation and, therefore, the rate of its dissolution via hydrolysis under physiological conditions (Ashley et al., 2011).

As shown in FIG. 38C, MSNPs with a low degree of silica condensation release 100% of encapsulated levofloxacin within 12 hours, while MSNPs with a high degree of silica condensation release encapsulated levofloxacin over a period of 2 weeks (see FIG. 38D); it is important to note that these data represent burst and sustained release and that the release rates of our MSNPs can be further tailored between these extremes. The ability to achieve high loading capacities for individual antibiotics and antibiotic cocktails enables a protocell formulation that reduces the required dose of levofloxacin compared to free drug. Furthermore, the ability to precisely tailor release rates allows for control of the pharmacokinetics of protocell-encapsulated levofloxacin over a wider range than is achievable with free antibiotics or antibiotic-loaded liposomes (Wong et al., 2003).

Fusion of liposomes to Drug-Loaded MSNPs Creates a Coherent SLB that Enhances Colloidal Stability and Enables Long-Term Cargo Retention.

Liposomes and multilamellar vesicles have poor intrinsic chemical stability, especially in the presence of serum, which decreases the effective concentration of drug that reaches target cells and increases the potential for systemic toxicity (Couvreur and Vauthier, 2006; Morilla and Romero, 2011). In contrast, lipid bilayers supported on mesoporous silica particles were shown to have a high degree of stability in neutral-pH buffers and serum-containing simulated body fluids, regardless of the charge or fluidity of lipids used to form the SLB (Ashley et al., 2011). In addition to being highly stable, the SLB provides a biocompatible interface with tailorable fluidity for display of functional molecules, such as polyethylene glycol (PEG) and targeting ligands (Ashley et al., 2011).

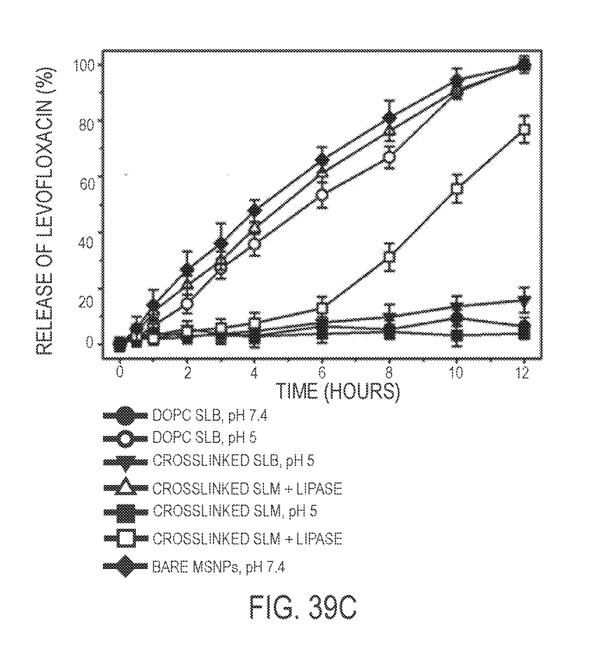

Protocells with SLBs composed of the zwitterionic, fluid lipid, 1,2-dioleoyl-sn-glycerol-3-phosphocholine (DOPC) with 30 wt % cholesterol and 10 wt % of PEG have a high degree of colloidal stability (see FIG. 39A) and stably encapsulate small molecule drugs, like levofloxacin for up to 4 weeks (see FIG. 39B) when incubated in a serum-containing simulated body fluid at 37° C. In dramatic contrast, MSNPs coated with cationic polymers, such as polyethyleneimine (PEI) rapidly aggregate in the presence of serum (see FIG. 39A), and levofloxacin-loaded liposomes rapidly leak their encapsulated drug (see FIG. 39B), even when composed of the fully-saturated lipid, 1,2-distearoyl-sn-glycero-3-phosphocholine (DSPC), which has a higher packing density than fluid lipids (e.g., DOPC) and should limit diffusion of drug across the bilayer. Although protocells are highly stable under neutral pH conditions, the SLB can be destabilized under endo/Iyso/phagosomal conditions, such as acidic pH (see FIG. 39C); SLB destabilization, as described in the next section, triggers dissolution of the MSNP core and enables intracellular delivery of encapsulated drugs (Ashley et al., 2012; Ashley et al., 2011; Epler et al., 2012). It is important to note, however, that the stability of the SLB, which influences the rate at which protocells release drug under intracellular conditions by initiating MSNP dissolution, can be tailored by optimizing the thickness and degree of crosslinking in the lipid bi/multilayer. For example, inclusion of photopolymerizable lipids, such as 1-palmitoyl-2-(10,12-tricosadiynoyl)-sn-glycero-3-phosphocholine (16:0-23:2 Diyne PC), enables formation of an intrabilayer-crosslinked SLB. If a higher degree of stability is required, supported lipid multilayers (SLMs) can be formed around protocels via liposome fusion in the presence of divalent cations (Mann and Markham, 1998). Interbilayer-crosslinked SLMs, produced through inclusion of 1,2-dioleoyl-sn-glycero-3-phosphoethanolamine-N-[4-(p-maleimidophenyl)butyramide](18:1 MPB PE) in the liposome formulation, followed by use of dithiothreitol to crosslink maleimide-containing headgroups of apposed lipid layers (Moon at al., 2011), have the highest degree of stability and can only be degraded in environments that mimic phagolysosomes (see FIG. 39C). Our ability to control the thickness of the lipid layer, as well as the approximate number of intra- and/or interbilayer bonds, will be critical to balancing the stability of orally-administered protocells with the rates of extra- and intracellular antibiotics release necessary to treat relevant diseases.

Modifying the SLB with Various Targeting Ligands Promotes Efficient Uptake of Antibiotic-Loaded Protocels by Model Host Cells and Enables Highly Efficacious Killing of Intracellular Bacteria.

In order to effectively kill intracellular bacteria, such as F. tularensis and B. pseudomallei, nanoparticle delivery vehicles must release antibiotics directly into the cytosol of host cells, which not only increases the concentration of drug in the vicinity of the pathogen but is also important since many classes of antibiotics, including β-lactams, lincosamides, and fluoroquinolones, show poor penetration or rapid efflux from mammalian cells (Pinto Alphandary et al., 2000). A number of factors govern cellular uptake and processing of nanoparticles, including their size, shape, surface charge, and degree of hydrophobicity (Peer et al., 2007). Additionally, a variety of molecules, including peptides, proteins, aptamers, and antibodies, can be employed to trigger active uptake by a plethora of specific cells.

Incorporation of targeting and endosomolytic peptides that trigger endocytosis and endosomal escape on the protocell SLB enables cell-specific delivery and cytosolic dispersion of encapsulated cargos (Ashley et al., 2011). As importantly, SLB fluidity can be tuned to enable exquisite (sub-nanomolar) specific affinities for target cells at extremely low targeting ligand densities (about 6 targeting peptides per protocel) and that SLB charge and degree of PEGylation can be modulated to reduce non-specific interactions, resulting in protocells that are internalized by target cells 10,000-times more efficiently than non-target cells (Ashley et al., 2011).

To promote uptake by model F. tularensis host cells, including innate immune cells, alveolar type II epithelial cells, and hepatocytes, levofloxacin-loaded MSNPs were encapsulated within SLBs composed of DOPC with 5 wt % of 1,2-dioleoyl-sn-glycero-3-phosphoethanolamine (DOPE), 30 wt % of cholesterol, and 10 wt % of PEG. Heterobifunctional crosslinkers were used to modify DOPE moieties with Fcγ from human IgG (binds to Fcγ receptors on macrophages and dendritic cells (Moon et al., 2012; Sandor et al., 2012; Schmidt et al., 2007), human complement C3 (binds to CR1 on macrophages and dendritic cells), ephrin B2 (binds to EphB4 receptors on alveolar type II epithelial cels (Hafner et al., 2004)), and the SP94 peptide (binds to unknown receptor(s) on hepatocyte-derived cells (Lo et al., 2008)). Additionaly, protocels targeted to the mannose receptor (a.k.a. CD206) were targeted by incorporating mannosylated cholesterol (Kawakami et al., 2000) into liposomes used to form SLBs.

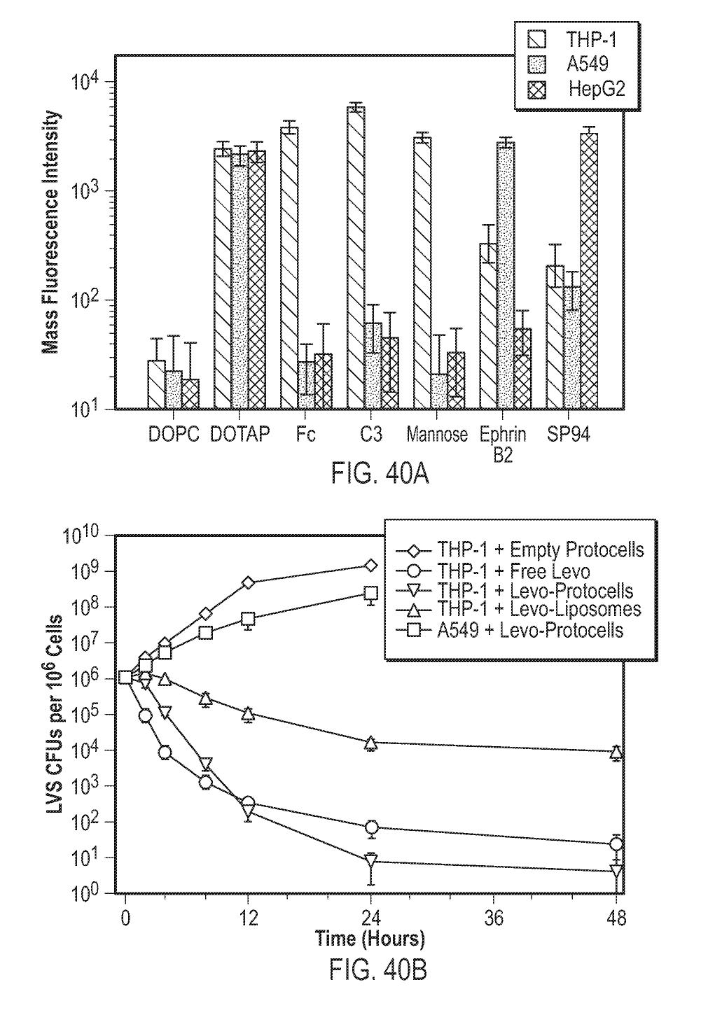

As demonstrated by FIG. 40A, all of the aforementioned targeting ligands promote efficient, cell-specific uptake of protocells by human monocyte-derived macrophages (THP-1), human alveolar type II epithelial cells (A549), and human hepatocytes (HepG2), while non-targeted protocells with a SLB composed of 60 wt % DOPC, 30 wt % cholesterol, and 10 wt % PEG showed minimal non-specific internalization. In contrast, protocells with SLBs composed of the cationic lipid, 1,2-dioleoyl-3-trimethylammonium-propane (DOTAP) were non-specifically internalized by all cell types, which demonstrates an important point: although numerous researchers coat MSNPs with cationic lipids and polymers, the resulting non-specific uptake reduces the effective drug concentration that reaches target cells and tissues (Clemens et al., 2012).

As shown by FIG. 408, cell-specific internalization of Fcγ-targeted protocells by THP-1 cells enables cell-specific delivery of levofloxacin and effective killing of intracellular F. tularensis, subspecies holarctica live vaccine strain (LVS), when MSNP cores exhibiting burst release kinetics are used and the SLB is further modified with a high density of the endo/lyso/phagosomolytic peptide, H5WYG (Moore et al., 2008), which disrupts endo/lyso/phagosomal membranes upon vesicle acidification and triggers dispersion of MSNP cores in the cytosol. Furthermore, with only 2 wt % loading of levofloxacin (about 1/20th of the protocell's maximum capacity), cytotoxicity toward intracellular LVS exceeded that of free levofloxacin and levofloxacin-loaded DSPC liposomes.

Levofloxacin-loaded protocells are not toxic to host cells, even upon burst release of 1 mg/mL (about 25,000 times the MIC90 value of levofloxacin). In summary, protocells are highly flexible and modular. In dramatic contrast to other state-of-the-art nanoparticle delivery vehicles, we can load high concentrations of physicochemically disparate molecules and specifically tailor release rates without altering the protocel's size, size distribution, stability, or synthesis strategy. Properties of the SLB and MSNP core can be modulated entirely independently, which enables us to optimize such properties as surface charge, colloidal stability, and targeting specificity independently from overall size, type of cargo(s), loading capacity, and rate of release and offers us several options for engineering and tailoring triggered cargo release.

### Example 6

### Synthesis and Characterization of CPP-PNA-Loaded Protocols

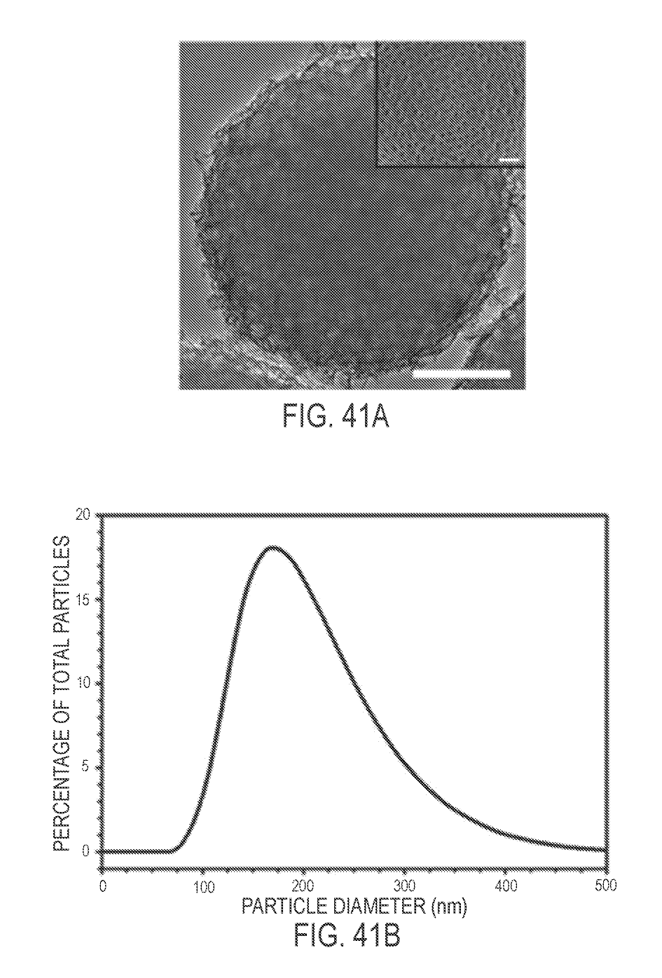

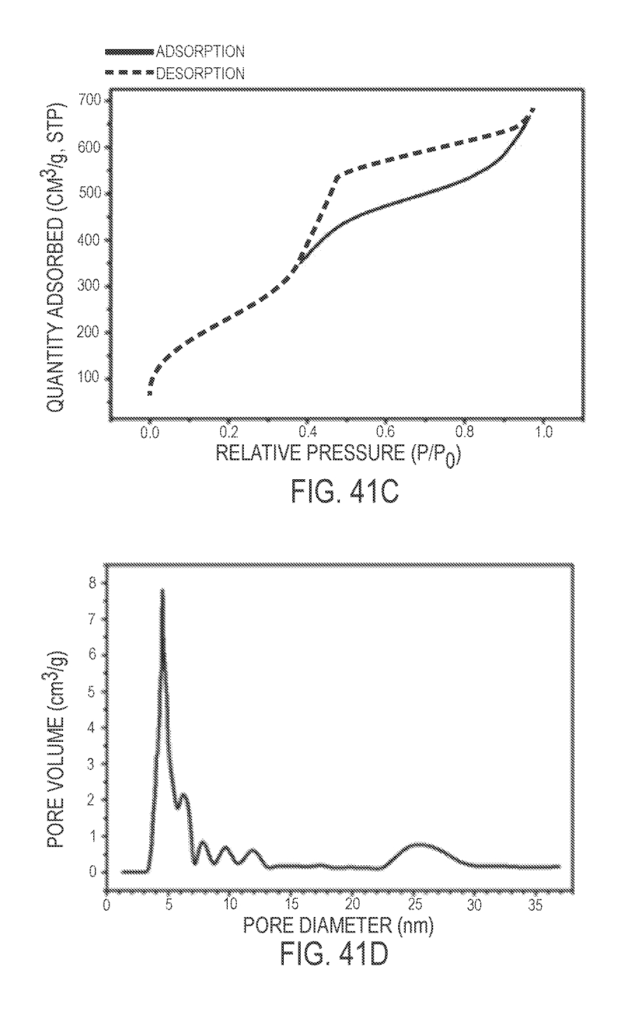

Mesoporous silica nanoparticles were prepared using the emulsion processing technique described by Carroll et al. (2009) and were characterized by a Brunauer-Emmett-Teler (BET) surface area of 850 m²/g, a pore volume fraction of about 65%, and a multimodal pore morphology composed of large (23-30 nm), surface-accessible pores interconnected by 3-13 nm pores (see FIGS. 41A and 41D). Silica nanoparticles were size-separated before being loaded with CPP-PNA as described in the Methods section, resulting in particles with an average diameter of 165-nm (see FIG. 41B). PEGylated liposomes were then fused to CPP-PNA-loaded cores, and the resulting supported lipid bilayer was chemically conjugated with a targeting peptide and an endosomolytic peptide.

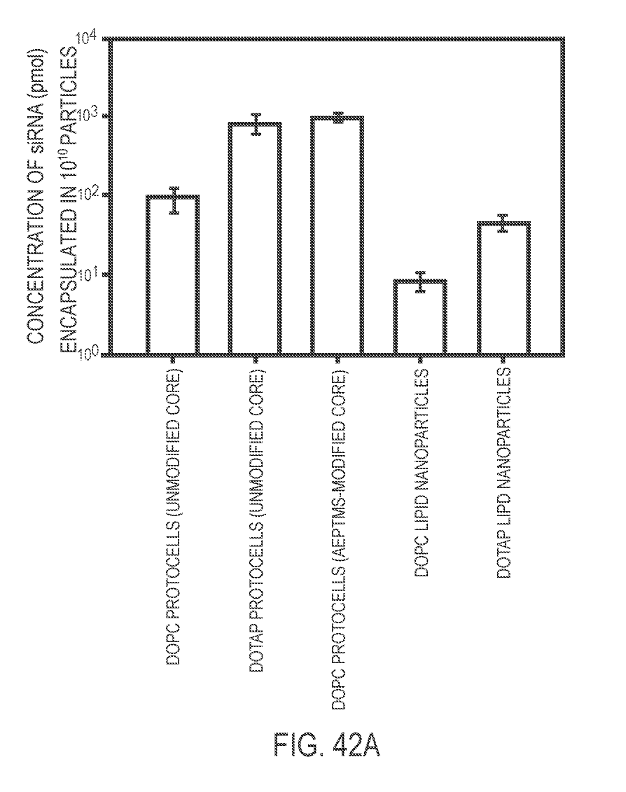

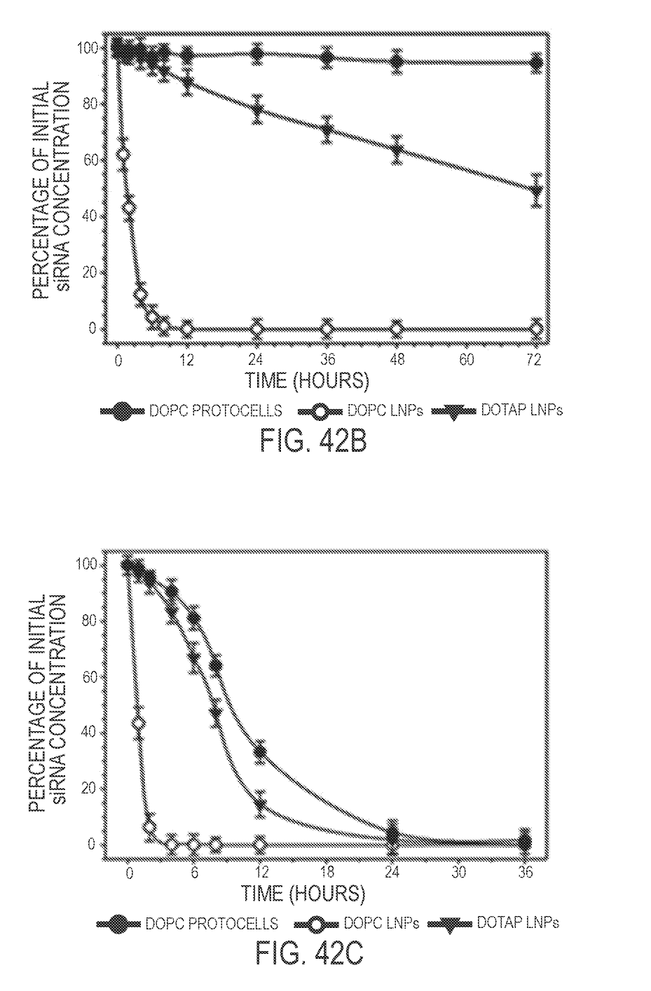

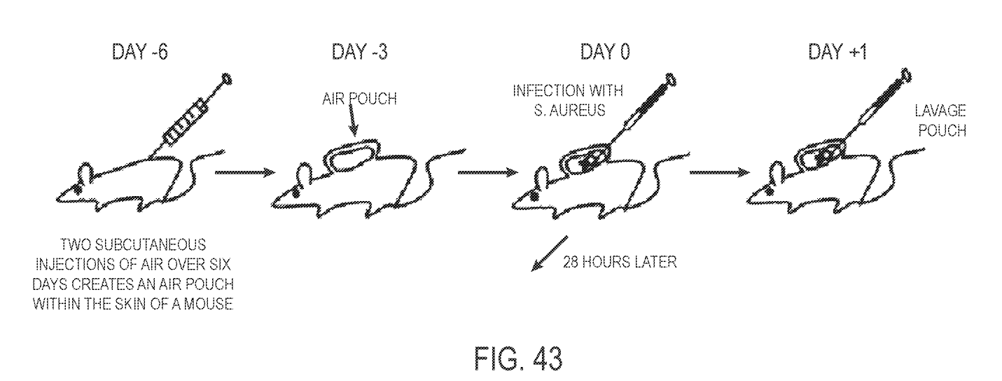

The CPP-PNA loading capacity of protocells is compared to that of zwitterionic and cationic lipid nanoparticles (LNPs) in FIG. 42A. Cationic lipids and polymers form the basis of most commercially-available transfection reagents and non-viral CPP-PNA delivery vehicles (Zhang et al., 2007), making LNPs, also referred to as lipoplexes and liposomes, the most appropriate system by which to judge the performance of protocells. LNPs composed of the zwitterionic phospholipid, DOPC, encapsulated about 10 pmol of CPP-PNA per 10¹⁰ particles. Construction of LNPs composed of the cationic lipid, DOTAP, resulted in a 5-fold increase in the CPP-PNA cargo, presumably due to attractive electrostatic interactions between the negatively-charged oligonucleotide and the positively-charged lipid components. Protocels containing a negatively-charged silica core with a zwitterionic (DOPC) lipid bilayer had a capacity roughly equivalent to the cationic LNP.

Modification of the silica core with the amine-containing silane, 3-[2-(2-aminoethylamino)ethylamino]propyltrimethoxysilane (AEPTMS), increased the zeta potential (ζ) from −32 mV to +12 mV and resulted in a CPP-PNA capacity of about 1 nmol per 10¹⁰ particles. Use of DOTAP liposomes to synergistically load CPP-PNA into negatively-charged cores (OBrien et al., 2007) resulted in protocells with a similar capacity, more than 100-fold higher than that of the zwitterionic LNPs that are commonly utilized in particle-based therapeutic applications. DOPC protocells with AEPTMS-modified cores were selected for further studies due to their high capacity for CPP-PNA and their low intrinsic cytotoxicity.

It should be noted that CPP-PNA-loaded protocells were slightly larger (178±24.3 nm) than CPP-PNA-loaded DOPC LNPs (135±19.1 nm) and DOTAP LNPs (144±14.8 nm), resulting in a about 2-fold increase in particle volume. When the capacities shown in FIG. 42A are normalized against particle volume, however, DOPC protocells with AEPTMS-modified cores still encapsulate 50 and 10-fold more CPP-PNA than DOPC and DOTAP LNPs, respectively, which demonstrates that the high-surface-area nanoporous silica core confers a higher intrinsic loading capacity than that expected based on volumetric differences alone. Furthermore, since the positively-charged core promotes electrostatic-driven loading of CPP-PNA, zwitterionic lipids can be used to form the protocell's supported lipid bilayer, thereby eliminating cytotoxicity associated with delivery vehicles that employ cationic lipids to complex CPP-PNA.

FIGS. 42B and 42C compare the CPP-PNA release profiles of DOPC protocells with AEPTMS-modified cores to those of DOPC and DOTAP LNPs upon dispersion in either a surrogate biological fluid at pH 7.4 (FIG. 42B) or a pH 5.0 (FIG. 42C) buffer that mimics endosomal conditions. DOPC LNPs rapidly released their encapsulated CPP-PNA under both neutral and mildly acidic pH conditions, resulting in a complete loss of the nucleotide content within 4-12 hours. Although DOTAP LNPs were more stable than DOPC LNPs under neutral pH conditions, approximately 50% of their CPP-PNA content was lost over a 72-hour period. In marked contrast to both LNPs, DOPC protocells with AEPTMS-modified cores retained 95% of their encapsulated RNA when exposed to the simulated body fluid for 72 hours.

Under mildly acidic conditions comparable to those in the endosome/lysosome pathway, the reduced electrostatic and dipolar interactions between the CPP-PNA-loaded, AEPTMS-modified core and the PE and PC headgroups of the supported lipid bilayer caused membrane destabilization and exposure of the core to the acidic medium. After membrane destabilization, the combined rates of cargo diffusion and core dissolution resulted in the release profile seen in FIG. 42C. Thus, in terms of CPP-PNA loading capacity, particle stability, and release characteristics, protocells represent a dramatic improvement over corresponding LNPs prepared using state-of-the-art techniques.

### Example 7

### Efficacy of Novel Protocells in Mouse Model of Invasive MRSA Infection

The efficacy of lead protocel formulations was tested in a mouse model of invasive MRSA infection. To assess the biodistribution of designed, synthesized, and optimized protocels, we injected Balb/c mice via the tail vein with 200 mg/kg of protocells labeled with the near-infrared fluorophore, DyLight 680 and used an IVIS Lumina II to monitor whole animal fluorescence as a function of time after injection. If necessary, the size of the MSNP core could be modified to achieve a broad systemic distribution, which will be required to treat invasive MRSA infections. A dermanecrosis or air pouch Balb/c mouse model of invasive MRSA infection was used to assess the efficacy of protocells targeted to MRSA and co-loaded with CPP-PNAs specific for antibiotic resistance genes (e.g., mecA) and appropriate antibiotics (e.g., cephalosporins like cefotaxime or cefazolin). Various concentrations of protocells were administered to infected mice via the tail vein; at 1, 12, 24, and 48 hours after injection, and assigned each mouse an overall morbidity score and attempted to quantify bacterial burden in the spleen and either the abscess site (dermanecrosis model) or air pouch. Free antibiotics, free PNAs, and antibiotic-loaded protocells are used as controls in all experiments.

Results

A well-established mouse air-pouch model of infection (Fleming at al., 2013) was used to assess the biodistribution of fluorescently labeled (Dylight-633) protocells during methicilin-resistant Staphylococcus aureus (MRSA) skin and soft tissue infections (SSTI). The air-pouch model is based on subcutaneous infection of air over the course of six days on C57B/6 mice with 7×10⁷ colony forming units (CFU) of the MRSA USA300 isolate LAC, and co-injected 2.5 mg of DyLight-633 stained protocells. At four and 24 hours post-infection, mice were sacrificed, kidney, spleen and liver removed and the pouch contents recovered by lavage.

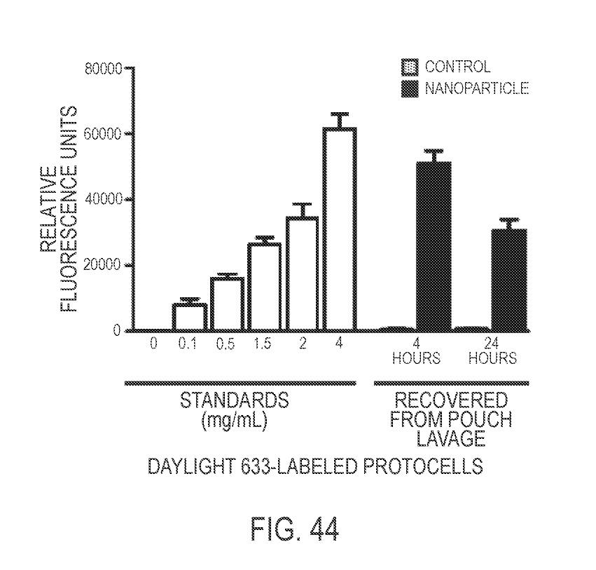

The post-infection biodistribution of DyLight-633 stained protocels was determined using an IVIS in vive imaging system (PerkinElmer, Waltham, Mass.) and flow cytometry. To determine the relative concentrations of protocells remaining in the air-pouch at four and 24 hours post-infection, flow cytometry was used to compare the amount of DyLight-633 fluorescence recovered from pouch lavage to in vitro standards. As shown in FIG. 44, the protocells were recoverable and stable within the pouch at four hours post-infection, with fluorescence present but decreasing at 24 hours compared to the four hour time-point.

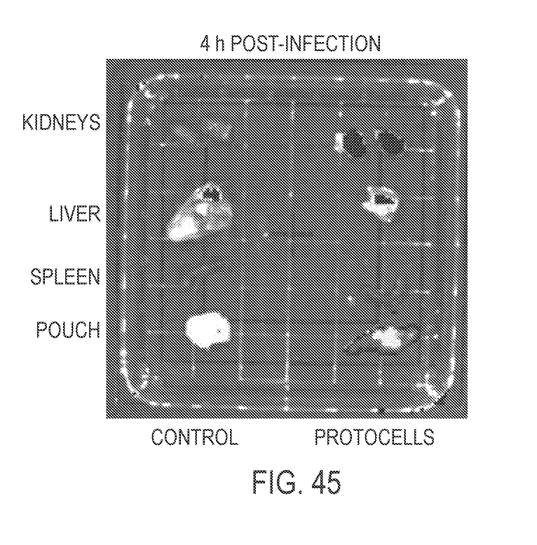

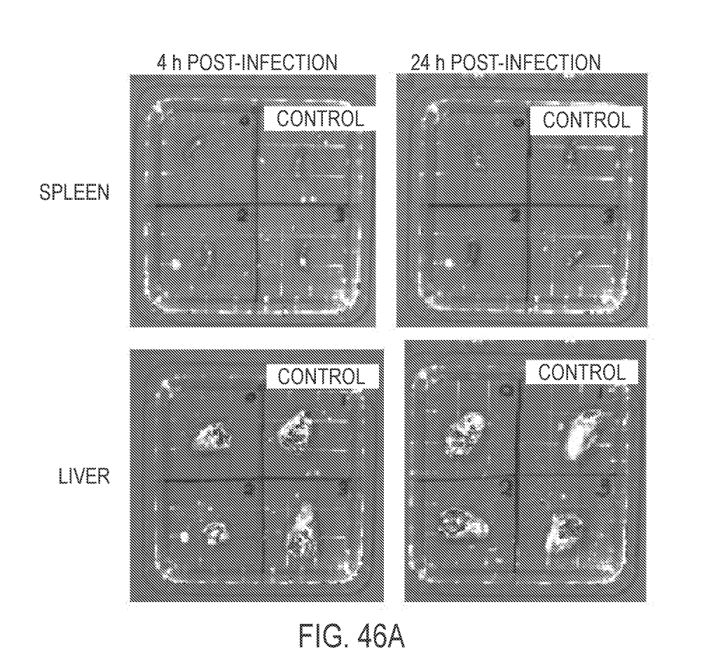

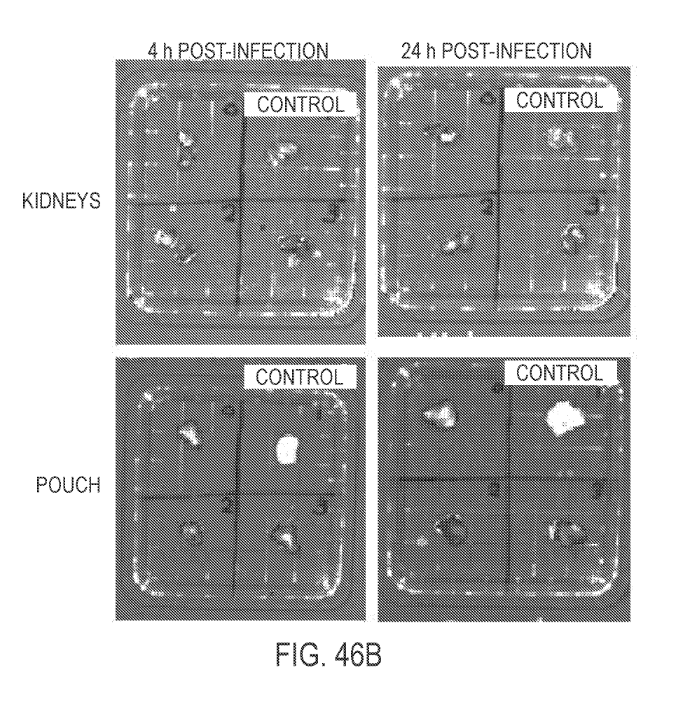

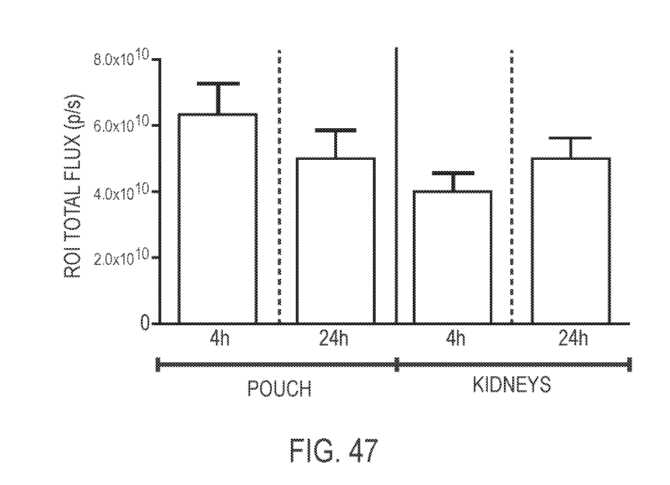

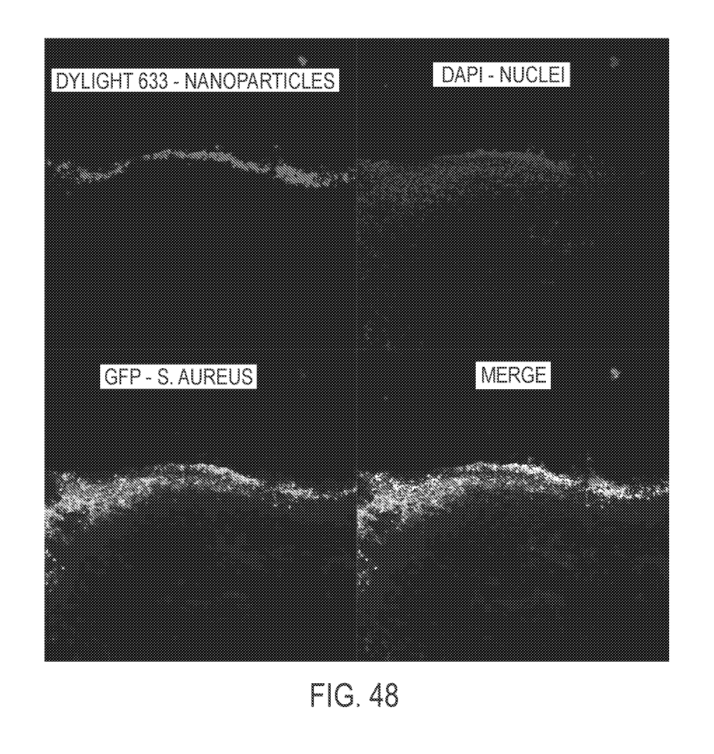

Next fluorescence imaging (IVIS) was used to visualize the protocells within lavaged and extracted air-pouches and dissemination of protocells to mouse kidneys and liver, at four and 24 hours post-infection (FIG. 45). Protocells were absent from the spleens and in large part from the livers of infected mice at both time points (FIG. 46). In contrast, high fluorescence in the extracted air-pouches and kidneys indicated that the protocells were physically associated with the air-pouch epithelium and dissemination to the kidney was the primary clearance pathway (FIGS. 47-48).

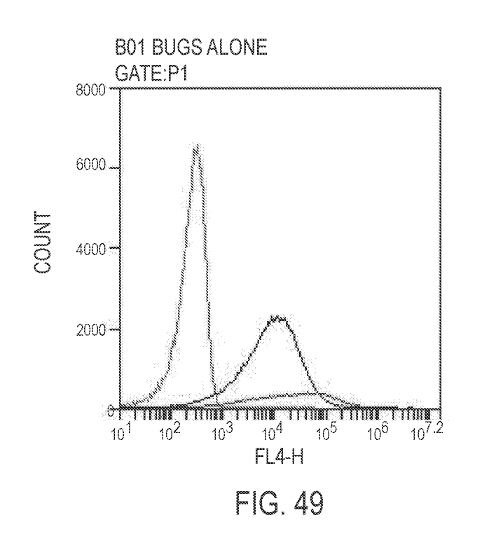

Next, it was determined whether the protocells co-localized with MRSA during infection. To address this, confocal microscopy was employed to demonstrate that fluorescently labeled protocells overlapped with GFP-expressing MRSA in the epidermis and dermis of the air-pouch during SSTI (FIG. 48). This is supported by in vitro flow cytometry data showing that the protocells are able to bind to MRSA in solution (FIG. 49).

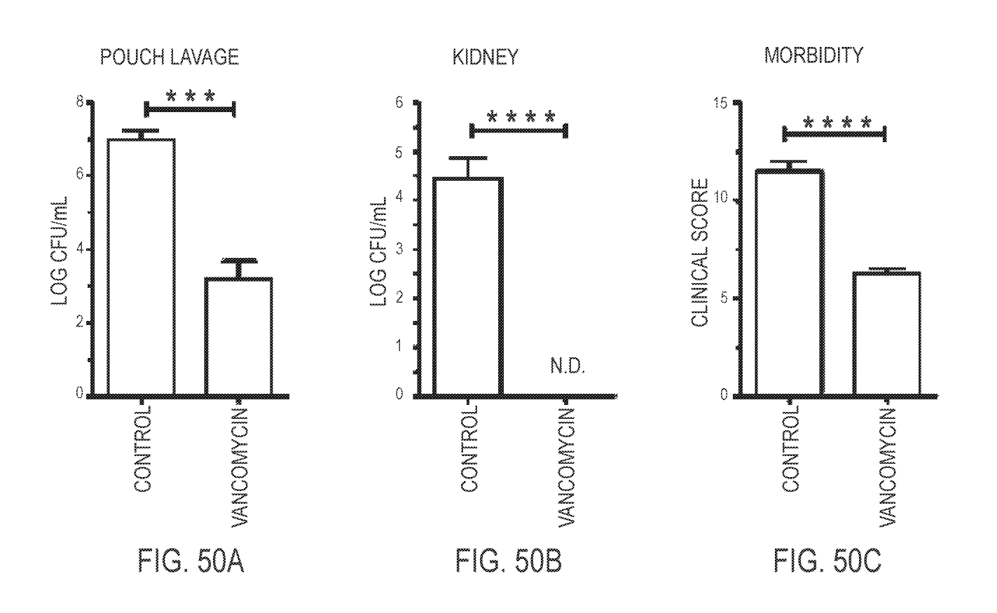

Finally, it was determined whether protocells loaded with the anti-MRSA antibiotic vancomycin, and injected into the infected air-pouch, could mediate bacterial clearance. As shown in FIGS. 50A-B, protocels loaded with vancomycin significantly decreased bacterial burden in the air-pouch lavage and prevented bacterial dissemination to the spleen at 24 hours post-infection, compared to empty protocell controls. In addition, based on the overall clinical scores, vancomycin-protocells significantly limited MRSA pathogenesis in this mouse model of SSTI (FIG. 50C).

The aforementioned data proved proof-of-principle that antibiotic-loaded protocells can bind to MRSA and mediate bacterial clearance at the site of infection.

### Example 8

### Versatile MSNPs and Protocells

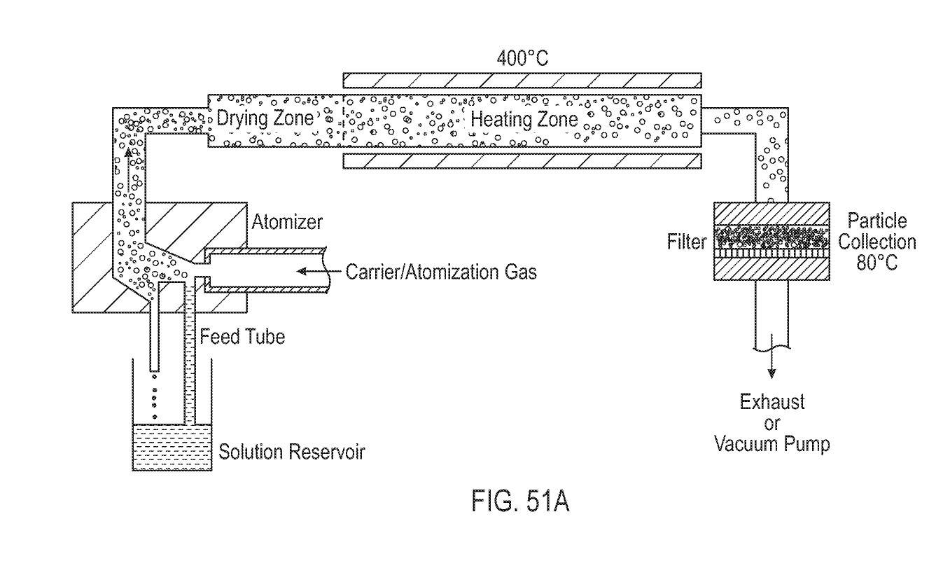

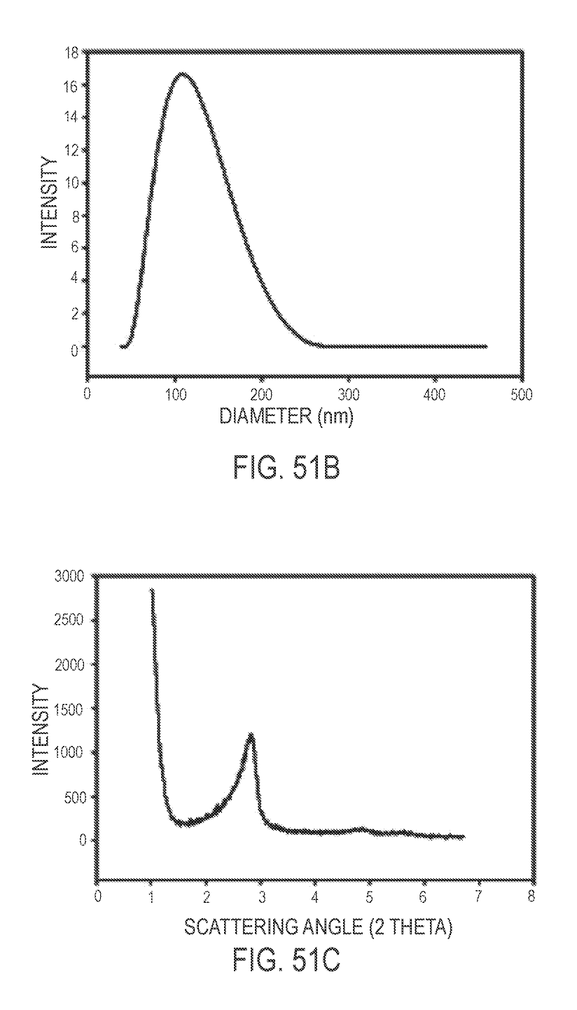

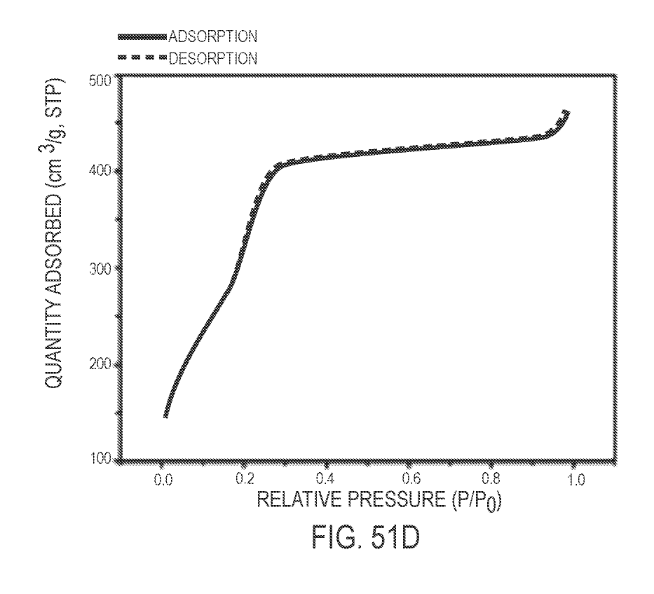

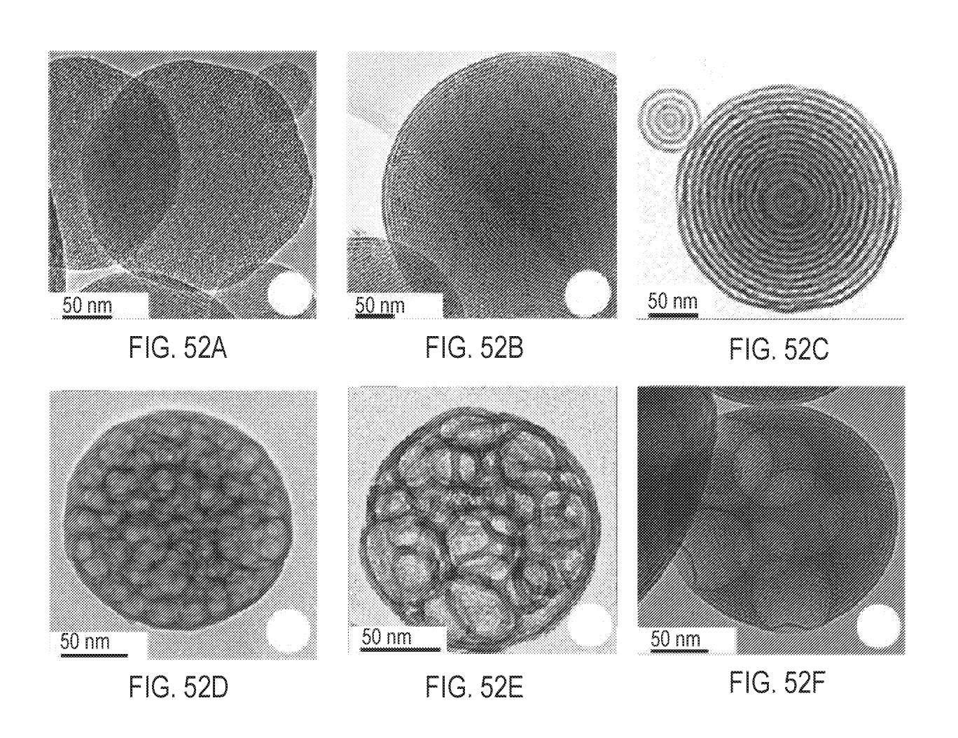

As illustrated in FIG. 51, silica nanoparticles can be generated using an EISA process in which the precursor solution is prepared by combining the surfactant, TEOS, ethanol, and water well below the surfactant's critical micelle concentration. The sol is atomized and the droplet is carried into a drying zone where solvent evaporation begins, increasing the effective surfactant concentration, facilitating self-assembly. The droplet enters the heating zone, which evaporates the remaining solvent and drives silica condensation to form solid particles. This robust process allows for tunable pore size, controllable particle diameter, and dissolution kinetics that can be modulated.

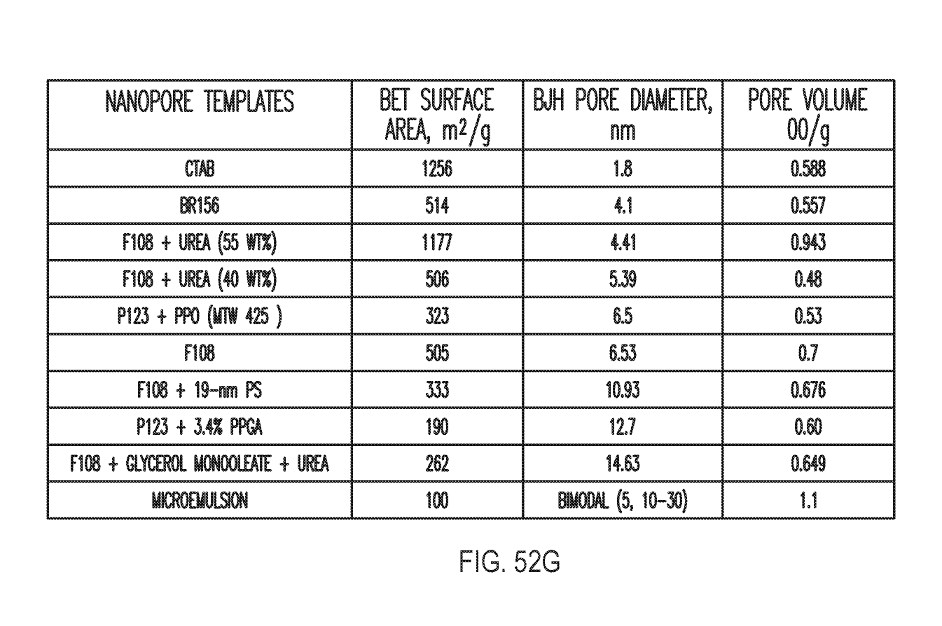

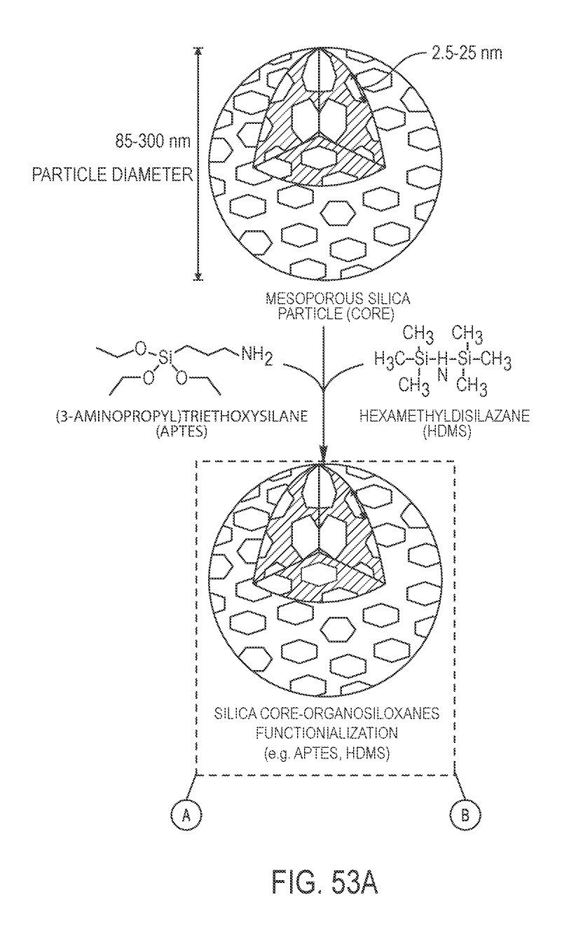

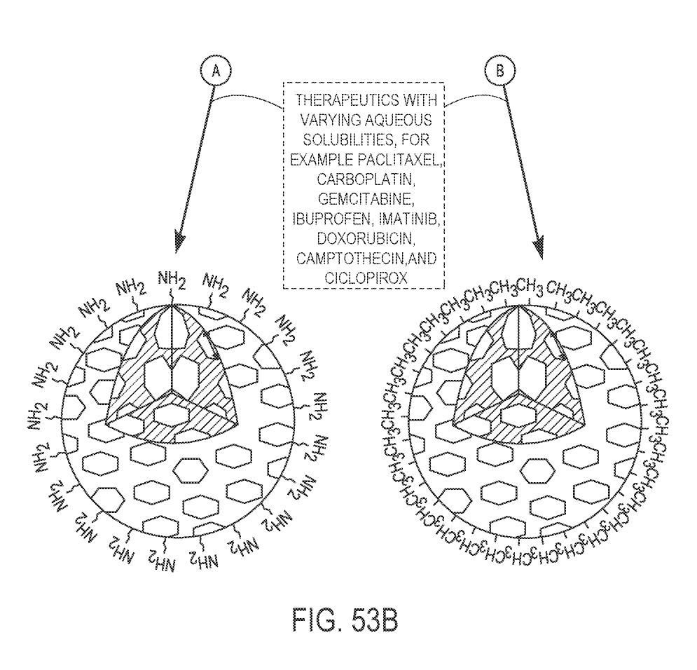

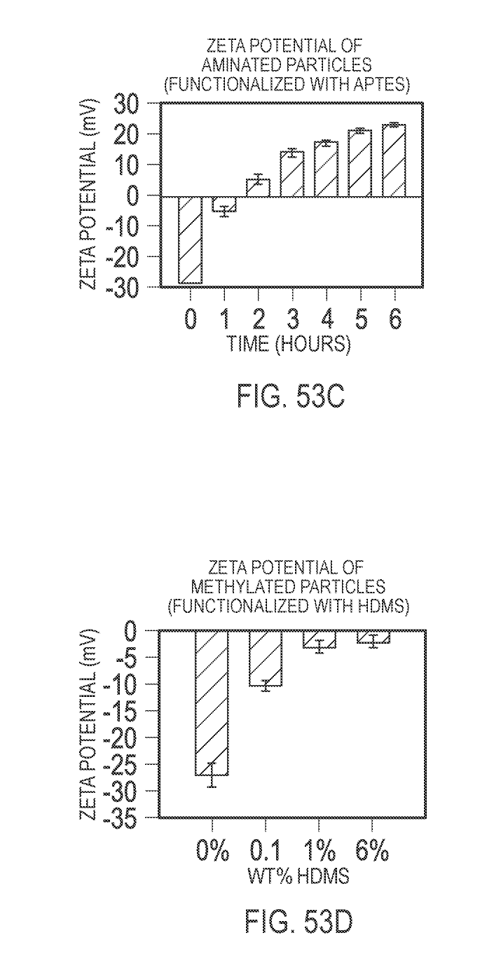

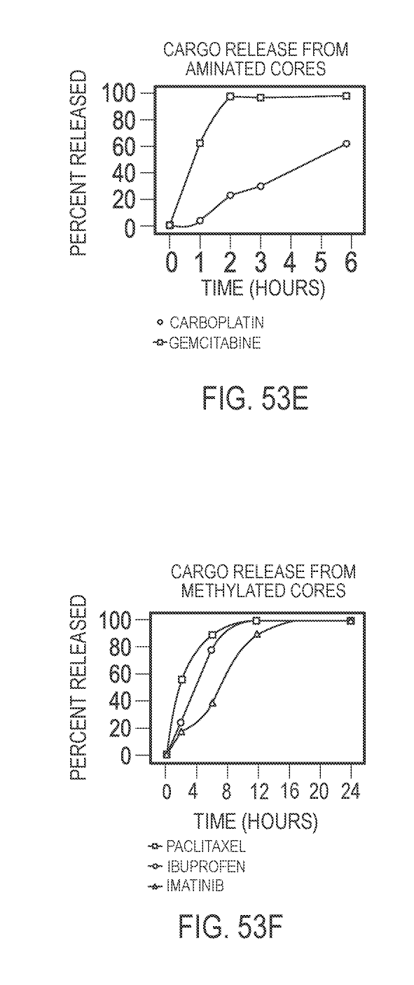

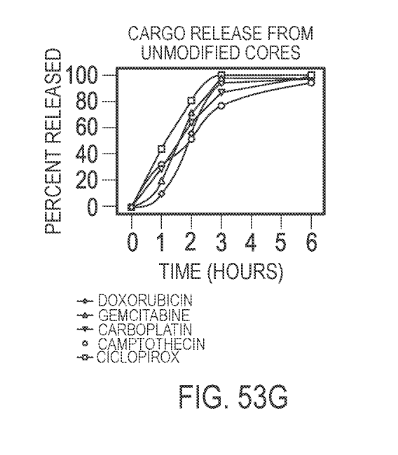

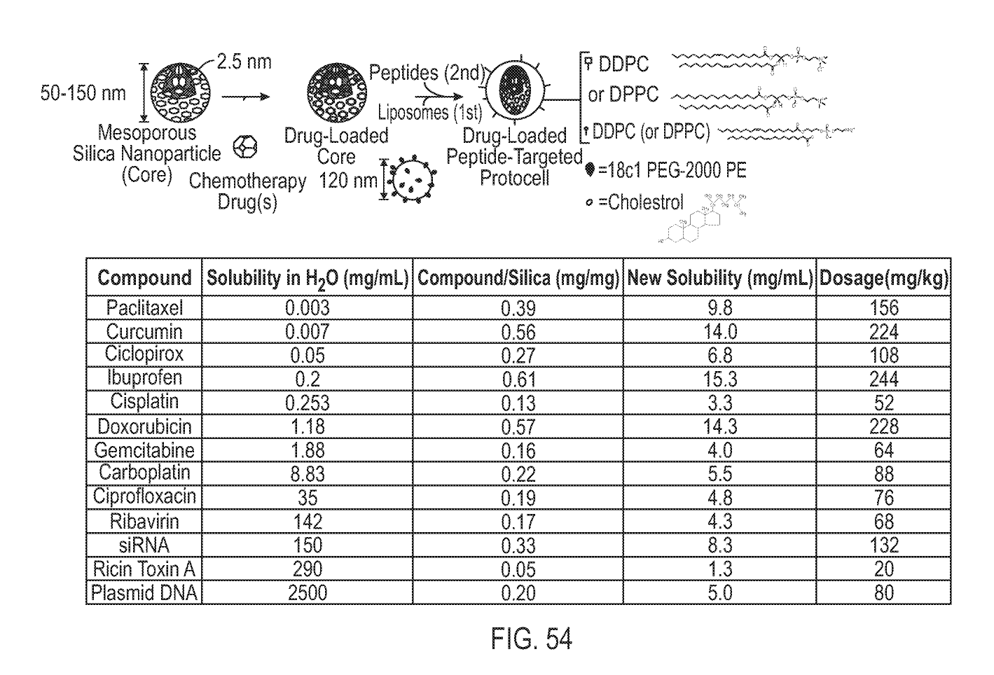

Using EISA as described herein, MSNP's were prepared that have a nominal BET surface area of 1,200 m²/g, zeta potentials that range from −30 mV to +30 mV, and a highly ordered pore structure that enables high-capacity loading of disparate types of cargos. See FIGS. 52-53. Our MSNPs enable control over stability and release of protocell cargo, which is useful for drug delivery applications. At a pH of about 7 (physiological pH), the MSNP's lipid bilayer proved stable and the cargo remained encapsulated within the particles. At a pH of about 5 (endosomal pH), the lipid bilayer destabilized and core/cargo was exposed to water, allowing for the release of the encapsulated cargo. In addition to loading disparate cargo types, silica core functionalization as described herein also allows for control over the cargo release rates. See FIG. 53.

Further, together with silica core functionalization, the supported-lipid bi- or multilayer (SLB) can be formulated with lipids that are zwitterionic, positively charged, or negatively charged. This allows for synergistic-cargo loading upon fusion with the core particle, an additional method for controlling the type and amount of cargo that is loaded. See FIGS. 52-53.

### Example 9

### Pore Size and Chemistry Enables High Capacity Loading of Physicochemically Disparate Antivirals, while Optimization of Silica Framework Condensation Results in Tailorable Release Rates

Using aerosol-assisted EISA, MSNPs were prepared that have an extremely high surface area (>1200 m²/g), which enables high concentrations of various therapeutic and diagnostic agents to be adsorbed within the core by simple immersion in a solution of the cargo(s) of interest. Furthermore, since aerosol-assisted EISA yields MSNPs that are compatible with a range of post-synthesis modifications, the naturally negatively-charged pore walls can be modified with a variety of functional moieties, enabling facile encapsulation of physicochemically disparate molecules, including acidic, basic, and hydrophobic drugs, proteins, small interfering RNA (siRNA), minicircle DNA vectors that encode small hairpin RNA (shRNA), plasmids, and diagnostic/contrast agents like quantum dots, iron oxide nanoparticles, gadolinium, and indium-111 (Ashley et al., 2012; Ashley et al., 2011). Table (supra) lists the MSNP and SLB properties we can precisely control and how these properties can be used to tailor the in vitro and in vivo functionality of protocells.

Aerosol-assisted evaporation-induced self-assembly (EISA) (Lu et al., 1999) is a robust, scalable process that we pioneered over a decade ago to synthesize spherical, well-ordered oxide nano- and microparticles with a variety of pore geometries and sizes (see FIG. 52). In the aerosol-assisted EISA process, a dilute solution of a metal salt or metal alkoxide is dissolved in an alcohol/water solvent along with an amphiphilic structure-directing surfactant or block co-polymer; the resulting solution is then aerosolized with a carrier gas and introduced into a laminar flow reactor (see FIG. 51). Solvent evaporation drives a radially-directed self-assembly process to form particles with systematically variable pores sizes (2 to 50-nm), pore geometries (hexagonal, cubic, lamellar, cellular, etc.), and surface areas (100 to >1200 m²/g).

Aerosol-assisted EISA, additionally, produces particles compatible with a variety of post-synthesis processing procedures, enabling the hydrodynamic size to be varied from 20-nm to >10-μm and the pore walls to be modified with a wide range of functional moieties that facilitate high capacity loading of physicochemically disparate diagnostic and/or therapeutic molecules. Importantly, aerosol-assisted EISA produces MSNPs that can be easily dispersed in a variety of aqueous and organic solvents without any appreciable aggregation, which enables us to load drugs that have high and low solubility in water. Our MSNPs are also easily encapsulated within anionic, cationic, and electrically-neutral SLBs via simple liposome fusion. In contrast, MSNPs generated using solution-based techniques tend to aggregate when the pH or ionic strength of their suspension media changes (Liong et al., 2009), typically require complex strategies involving toxic solvents to form SLBs, and have maximum loading capacities of 1-5 wt %,²⁰ which, our MSNPs exceed by an order of magnitude.

As demonstrated by FIG. 1, MSNPs formed via aerosol-assisted EISA can be loaded with up to 70 wt % of small-molecule antivirals like ribavirin (>3 million molecules/MSNP), 32 wt % of siRNA-based antivirals (about 30,000 molecules/MSNP), 7.2 wt % of a 2000-base-pair minicircle DNA vector that encodes shRNA-based antivirals (about 60 vectors/MSNP), and 8.9-12 wt % of various antibody-based antivirals (about 700-5400 molecules/MSNP), including single-chain variable fragments (scFvs), F(ab′)₂ fragments, and whole IgGs. It is important to note that these capacities are 10-fold higher than other MSNP-based delivery vehicles (Clemens et al., 2012) and 100 to 1000-fold higher than similarly-sized liposomes and polymeric nanoparticles (Couvreur et al., 2006; Morilla et al., 2011; Wong et al., 2003).

It is also important to note that the present MSNPs can be loaded with complex combinations of physicochemically disparate anticancer agents, antibacterial agents and antiviral agents (e.g., three small molecule drugs in combination with five separate siRNAs), a capability other nanoparticle delivery vehicles typically do not possess. We are able to achieve high loading capacities for acidic, basic, and hydrophobic drugs, as well as small molecules and macromolecules by altering the solvent used to dissolve the drug prior to loading and by modulating the pore size and chemistry of the MSNP (see FIG. 1). Unlike MSNPs formed using solution-based techniques, MSNPs formed via aerosol-assisted EISA are compatible with all aqueous and organic solvents, which ensures that the maximum concentration of drug loaded within the pore network is essentially equivalent to the drug's maximum solubility in its ideal solvent.

Furthermore, since MSNPs formed via aerosol-assisted EISA remain stable upon post-synthesis processing, the pore chemistry can be precisely altered by, e.g., soaking naturally negatively-charged MSNPs in amine-containing silanes (e.g., (3-aminopropyl)triethoxysilane, or APTES), in order to maximize electrostatic interactions between pore walls and cargo molecules. Non-polar compositions may also be used such as methylating agents (e.g., hexamethyldisilazane (HDMS), sodium bis(tnmethylsilyl)amide (NaHDMS) or potassium bis(trimethylsilyl)amide (KHDMS) to enhance MSNP core loading with relatively water-insoluble active ingredients.

Another unique feature of the present MSNPs is that the rate at which encapsulated drug is released can be precisely modulated by varying the degree of silica framework condensation and, therefore, the rate of its dissolution via hydrolysis under physiological conditions (Ashley et al., 2011). As shown in FIG. 8, silica (SiO₂) forms via condensation and dissolves via hydrolysis. Therefore, MSNPs with a low degree of silica condensation have fewer Si—O—Si bonds, hydrolyze more rapidly at physiological pH, and release 100% of encapsulated ribavirin within 12 hours. In contrast, MSNPs with a high degree of silica condensation hydrolyze slowly at physiological pH and can, therefore, release ˜2% of encapsulated ribavirin (about 60,000 molecules/MSNP) per day for 2 months. The degree of silica condensation between these extremes can be tailored by employing different methods to remove structure-directing surfactants from pores (e.g., thermal calcination, which maximizes the number of Si—O—Si bonds vs. extraction via acidified ethanol, which favors the formation of Si—OH bonds over Si—O—Si bonds) and by adding various concentrations of amine-containing silanes to the precursor solution in order to replace a controllable fraction of Si—O—Si bonds with Si—R—NH₂ bonds, where R=hydrocarbons of various lengths.

### Example 10

### Fusion of Liposomes to Antiviral-Loaded MSNPs Creates a Coherent SLB that Enhances Colloidal Stability and Enables DH-Triggered Release

Liposomes and multilamellar vesicles have poor intrinsic chemical stability, especially in the presence of serum, which decreases the effective concentration of drug that reaches target cells and increases the potential for systemic toxicity (Couvreur et al., 2006; Morilla et al., 2011). In contrast, lipid bilayers supported on MSNPs have a high degree of stability in neutral-pH buffers, serum-containing simulated body fluids, and whole blood, regardless of the melting temperature (Tₘ, which controls whether lipids are in a fluid or non-fluid state at physiological temperature) of lipids used to form the SLB. Specifically, we have demonstrated that protocells with SLBs composed of the zwitterionic, fluid lipid, 1,2-dioleoyl-sn-glycerol-3-phosphocholine (DOPC) have a high degree of colloidal stability (see FIG. 9) and retain small molecule drugs, such as ribavirin, for up to 4 weeks (see FIG. 10) when incubated in whole blood or a serum-containing simulated body fluid at 37° C.; it is important to note that surface-modification with polyethylene glycol (PEG) is not necessary to stabilize DOPC protocels, which is significant given the FDA's increasing concerns about hypersensitivity reactions induced by PEGylated nanoparticles and therapeutic molecules. In dramatic contrast to the behavior of DOPC protocells, serum proteins rapidly adsorb to bare MSNPs and MSNPs coated with cationic polymers, such as polyethyleneimine (PEI), upon dispersion in whole blood or serum-containing simulated body fluids (see FIG. 9), and ribavirin-loaded liposomes rapidly leak their encapsulated drug (see FIGS. 6 and 10), even when composed of the fully-saturated lipid, 1,2-distearoyl-sn-glycero-3-phosphocholine (DSPC), which has a higher packing density than fluid lipids (e.g., DOPC) and should limit diffusion of drug across the bilayer (Ashley et al., 2011).

Although protocells are highly stable under neutral pH conditions, the SLB can be selectively destabilized under conditions that simulate the interior volume of intracellular vesicles (e.g., endosomes, lysosomes, macropinosomes), which become acidified via the action of proton pumps. Specifically, DOPC SLBs are destabilized at pH 5.0, which exposes the MSNP core and stimulates its dissolution at a rate dictated by core's degree of silica condensation; DOPC protocells with MSNPs cores that have a low degree of condensation are, therefore, ribavirin was retained at pH 7.4 but rapidly release it at pH 5.0 (see FIG. 2A). pH-dependent release rates can be further tuned by controlling the thickness of the protocell's lipid shell. Fusing liposomes to MSNPs in the presence of divalent cations²⁵ results in protocells with supported lipid multilayers (SLMs), the thickness of which can be used to control release rates under acidic conditions (see FIG. 11). Protocells with SLMs are also able to withstand iterative exposure to neutral and acidic pH conditions (see FIG. 2B), which indicates that protocels will be able to retain encapsulated antivirals while traversing cellular barriers, such as the nasal epithelium and the blood brain barrier (BBB), and release encapsulated antivirals within target CNS cells.

### Example 11

### PTX Protocell

Paclitaxel (PTX) is a complex diterpenoid natural product, which has become a first-line treatment for NSCLC. However, because of its poor water solubility, paclitaxel has been used in an encapsulated form with the organic co-solvents ethanol and polyethoxylated castor oi (a formulation marketed as Taxol; Bristol-Myers Squibb, New York, N.Y., USA) for clinical trials, but has been shown to cause toxic effects, including life-threatening anaphylaxis. Liposomal paclitaxel (Lipusu; Luye Pharma Group Ltd., Nanjing, China) is a new formulation of paclitaxel and phosphatidylcholine liposomes. Pharmacokinetic studies in animal models have shown that, compared with the current pacditaxel formulation, liposomal paclitaxel has a significantly prolonged elimination half-life and mean retention time, and an apparent larger volume of distribution. Even more unexpected and exciting is that the concentration of liposomal paclitaxel in tissues is dramatically higher than that of paclitaxel, especially in the reticuloendothelial system, including the lymph nodes, liver, and spleen. Several clinical studies recently indicated that the efficacy of liposomal paditaxel equaled or slightly exceeded that of the current paclitaxel (Taxol®) formulation, while having a superior safety profile. Hu, et al., Trials 2013, 14:45 doi:10.1186/1745-6215-14-45 (citations omitted).

PTX MSNPs are made according to EISA techniques described herein. PTX nanoparticles comprise PTX loaded into MSNPs whose 20 nm-50 nm pores. MSNPs are methylated with hexamethyldisilazane (HDMS); the EISA surfactant is hexadecyltrimethylammonium bromide (Cis; CTAB). Nanoparticles have a differential pore volume of between about 1.0 cm³/g and a BET surface area of about 1,000 m²/g. The MSNP weight ratio of PTX to silica is about 0.75 and the MSNPs have a Zeta (ζ) potential of about −20 mV. The MSNPs are coated with a lipid bilayer comprising DOPC or DPPC in combination with DOPE, cholesterol and PEG-2000 PE (18:1). Other small-molecule water-insoluble cargos can be used in the protocell in place of PTX.

In some examples, the lipid bilayer comprises a c-MET binding peptide having sequence selected from SEQ ID Nos. 1-4 below:

SEQ ID NO: 48:
Ala Ser Val His Phe Pro Pro
SEQ ID NO: 49:
Thr Ala Thr Phe Trp Phe Gln
SEQ ID NO: 50:
Thr Ser Pro Val Ala Leu Leu
SEQ ID NO: 51:
Ile Pro Leu Lys Val His Pro

The drug loading efficiency is determined in triplicate by HPLC (Agilent 1100 series. Agilent Technologies, Diegem. BE). The mobile phase consists in acetonitrile/water (70:30 vv). The reverse phase column is a CC125/4 Nucleod UR100-5 C18. The column temperature is maintained at 30° C. The flow rate is set at 1.0 ml/min and the detection wavelength is 227 nm. Sample solution is injected at a volume of 50 μl. The HPLC is calibrated with standard solutions of 5 to 100 μg/ml of PTX dissolved in acetonitrile (correlation coefficient of R²=0.9965). The limit of quantification is 0.6 ng/ml. The coefficients of variation (CV) are all within 4.3%. Nanoparticles are dissolved in acetonitrile and vigorously vortexed to get a clear solution. The encapsulation efficiency is defined by the ratio of measured and initial amount of PTX encapsulated in nanoparticles, the recovery corresponds to the ratio of the amount of PTX in the supernatant and in the pellets to the initial amount of PTX. About 1.4 mg of the 2 mg PTX starting material is encapsulated.

In vitro measurements determine that at a pH of about 5, the protocells release between about 30 wt % to about 100 wt % of PTX at about three hours after delivery, and releases about 60 wt % to about 100 wt % of PTX at about six hours after delivery. At a pH of about 7, less than about 10 wt % of PTX is released from the protocells after about eight weeks.

### Example 12

### Protocells are Biocompatible, Biodegradable, and Non-Immunogenic

The biocompatibility, biodegradability, and immunogenicity of protocells were evaluated after repeat intraperitoneal (IP) or subcutaneous (SC) injections in Balb/c and C57B6 mice. Balb/c mice injected IP with 200 mg/kg doses of DOPC protocels three times each week for 4 weeks showed no signs of gross or histopathological toxicity (see FIG. 17). Furthermore, it was demonstrated that intact and partially-degraded MSNPs, as well as silicic acid and other byproducts of silica hydrolysis are excreted in the urine and feces of mice at rates that are determined by the dose, route of administration, and biodistribution (see FIGS. 18 and 20-22). Finally, protocells loaded with a therapeutic protein and modified with a high density (about 10 wt % or about 5000 peptides/protocell) of a targeting peptide induced neither IgG nor IgM responses upon SC immunization of C57Bl/6 mice at a total dose of 1000 mg/kg (see FIG. 19).

### Example 13

### The Biodistribution of Protocells can be Controlled by Tuning their Hydrodynamic Size and Surface Modification with Targeting Ligands

Since liposomes and multilamellar vesicles are the most similar nanoparticle delivery vehicles to protocels, every effort is made to benchmark the performance of protocells against the performance of lipid-based nanoparticles. Liposomes and multilamellar vesicles have biodistributions that are largely governed by their overall size and size distributions. This holds true for protocells as well. However, the sizes of liposomes and multilamellar vesicles are difficult to control and subject to slight variations in lipid content, buffer pH and ionic strength, and chemical properties of cargo molecules. In contrast, the diameter of protocells is generally governed by the size of the core, which can be modulated by the methods described herein. As demonstrated by FIG. 20, the hydrodynamic size of protocells dramatically affects their bulk biodistributions: protocells 250-nm in diameter accumulate in the liver within 1 hour of injection, while protocels 50-nm in diameter remain in circulation for >1 month. Size-dependent biodistribution can be altered, however, by modifying the surface of DOPC protocells with various types of targeting ligands.

For example, modifying 150-nm protocels with CD47, a molecule expressed by erythrocytes that innate immune cells recognize as ‘self’, substantially enhances their circulation half-life (see FIG. 21). In contrast, modifying 150-nm protocels with an antibody against aminopeptidase P causes them to rapidly amass in the lung (see FIG. 22).

### Example 14

### Optimization of the BBB Penetration Potential of Protocells Using an In Vitro Transwell Assay

The preliminary data indicate that various parameters affect the BBB penetration potential of protocels, including their hydrodynamic size, surface charge, and surface modifications; as shown in FIG. 4, modifying DOPC protocells with 1 wt % of glutathione increased their in vitro penetration across cerebral microvessel endothelial cells by 50% and their in vivo accumulation within the brains of Balb/c mice by about 10-fold. Protocells can be modified with other ligands (e.g., L-dihydroxyphenylalanine, or L-DOPA) to enhance BBB penetration of small molecule drugs in an attempt to further increase the BBB penetration potential of antiviral-loaded protocells 50 to 150-nm in diameter.

To assess penetration and cargo delivery efficiency, use a high-throughput transwell assay where cerebral microvessel endothelial (hCMEC/D3) cells are grown to confluence on collagen-coated, microporous, polycarbonate membranes and inserted into 12-well Costar Transwell plates. Various concentrations of ribavirin-loaded protocells are added to the apical or basolateral chamber, the wells incubate for 2 hours at 37° C., and inductively-coupled plasma mass spectrometry (ICP-MS) and HPLC used to measure Si and ribavirin concentrations, respectively, in both chambers. Ribavirin is used since a fluorescence-based HPLC method is developed to detect it in cell culture medium. Monolayers are co-dosed with lucifer yellow to ensure that the cells are not damaged during the course of the experiment. Use DLS, transmission electron microscopy (TEM), and UV-visible spectroscopy to characterize the size, charge, and loading capacity of protocells that cross the cell monolayer, hCMEC/D3 cells will be used because they are robust, easy to grow, and better replicate the human BBB than other in vitro models.

### Example 15

### Exemplary Protocells

In one embodiment, a mesoporous silica or metal oxide nanoparticle is provided having a pore size ranging from about 0.001 to about 100 nm, e.g., from about 0.01 to about 50 nm, from about 0.1 to about 100 nm, from about 0.1 nm to about 35 nm, or from about 2 nm to about 25 nm, and a diameter ranging from about 25 nm to about 500 nm, e.g., from about 100 nm to about 300 nm, said nanoparticle being functionalized with either a polar group for loading of hydrophilic cargo or a non-polar group for loading of hydrophobic cargo. In one embodiment, the mesoporous metal oxide nanoparticle of embodiment 1, wherein the polar group is an amino group and the non-polar group is a methyl or a phenyl group. In one embodiment, the mesoporous metal oxide nanoparticle of embodiment 2, wherein the nanoparticle is aminated or methylated with an organosiloxane. In one embodiment, the mesoporous metal oxide nanoparticle of embodiments 1-3, wherein the nanoparticle is aminated with aminopropyltriethoxysilane (APTES) or 3-[2-(2-aminoethylamino)ethylamino]propyttrimethoxysilane (AEPTMS) or is methylated with hexamethyldisilazane (HDMS), sodium bis(trimethylsilyl)amide (NaHDMS) or potassium bis(trimethylsilyl)amide (KHDMS), or is functionalized with phenyltriethoxysilane (PTS). In one embodiment, the mesoporous metal oxide nanoparticle of embodiments 1-4, wherein the nanoparticle is a silica nanoparticle (MSNP) or an aluminum oxide (Al₂O₃) nanoparticle. In one embodiment, the mesoporous metal oxide nanoparticle has a differential pore volume of between about 0.25 cm³/g to about 10 cm³/g, e.g., from about 0.25 cm³/g to about 1.5 cm³/g. In one embodiment, the mesoporous metal oxide nanoparticle, wherein the nanoparticle has a nominal BET surface area of between about 50 m²/g to about 1,500 m²/g, more for example from about 100 m²/g to about 1,300 m²/g. In one embodiment, the mesoporous metal oxide nanoparticle of embodiments 1-7, wherein either: (a) the nanoparticle is methylated and is loaded with a cargo having a water solubility of between about less than 0.001 mg/mL to about 0.5 mg/mL; or (b) the nanoparticle is aminated and is loaded with a cargo having a water solubility of between about 0.2 mg/mL to greater than about 3,000 mg/mL In one embodiment, the mesoporous metal oxide nanoparticle of embodiments 1-10, wherein the nanoparticle is a silica nanoparticle (MSNP). In one embodiment, the mesoporous metal oxide nanoparticle, wherein the nanoparticle is methylated and is loaded with one or more small molecules which have a water solubility of between about less than 0.001 mg/mL to about 0.50 mg/mL. In one embodiment, the mesoporous metal oxide nanoparticle, wherein the small molecule is selected from the group consisting of paclitaxel, imatinib, curcumin, ciclopirox and ibuprofen.

In one embodiment, the mesoporous metal oxide nanoparticle is aminated and is loaded with a cargo having a water solubility of between about 0.2 mg/mL to greater than about 3,000 mg/mL, said cargo being selected from the group consisting of a small molecule, a mRNA, a siRNA, a shRNA, a micro RNA, a protein, a protein toxin (e.g., ricin toxin A-chain or diphtheria toxin A-chain) and/or DNA (including double stranded or linear DNA, minicircle DNA, plasmid DNA which may be supercoiled and/or packaged (e.g., with histones) and which may be optionally modified with a nuclear localization sequence). In one embodiment, the small molecule is selected from the group consisting of cisplatin, doxorubicin, gemcitabine, carboplatin, ciprofloxacin and ribavirin. In one embodiment, the nanoparticle is a silica nanoparticle (MSNP) which is loaded with cargo, and wherein the weight ratio of cargo to silica ranges from about 0.10 to about 0.75. In one embodiment, the nanoparticle is made by evaporation-induced self-assembly (EISA) or emulsion processing. In one embodiment, the mesoporous metal oxide nanoparticle of embodiments 1-15, wherein the nanoparticle is aminated or methylated and wherein the Zeta (ζ) potential of an aminated nanoparticle is between about 0 mV to about +40 mV, and wherein the Zeta (ζ) potential of a methylated nanoparticle is between about −40 mV to about 0 mV. In one embodiment, the nanoparticle is a silica nanoparticle (MSNP) made by an evaporation-induced self-assembly (EISA) process which includes the steps of: (a) preparing a precursor solution comprising (1) a surfactant (2) tetraethyl orthosilicate (TEOS), tetramethyl orthosilicate (TMOS) or a mixture thereof (3) a C₁₋₄ alcohol (for example ethanol), and (4) water, wherein said surfactant, tetraethyl orthosilicate (TEOS), tetramethyl orthosilicate (TMOS) or a mixture thereof, C₁₋₄ alcohol, and water are combined at a temperature below the surfactant's critical micelle concentration; (b) atomizing the precursor solution to generate droplets; (c) drying the droplets, thereby evaporating solvent and increasing effective surfactant concentration and inducing nanoparticle self-assembly; and (d) heating dried droplets, thereby evaporating residual solvent, inducing silica condensation and forming solid nanoparticles, wherein the degree of silica condensation is increased by thermal calcination to maximize the number of Si—O—Si bonds and reduced by using acidified ethanol to extract structure-directing surfactants.

In one embodiment, the surfactant is a cationic surfactant selected from the group consisting of a dodecylsulfate salt (e.g., sodium dodecylsulfate or lithium dodecylsulfate (SDS)), a tetradecyl-trimethyl-ammonium salt (e.g., tetradecyl-trimethyl-ammonium bromide (C₁₄ TAB) or tetradecyl-trimethyl-ammonium chloride), a hexadecyltrimethylammonium salt (e.g., hexadecyltrimethylammonium bromide (C₁₆; CTAB)), an octadecyltrimethylammonium salt (e.g., odadecyltrimethylammonium bromide (C₁₈; OTAB)), a dodecylethyldimethylammonium salt (e.g., dodecylethyldimethylammonium bromide), a cetylpyridinium salt (e.g., cetylpyridinium chloride (CPC)), polyethoxylated tallow amine (POEA), hexadecyltrimethylammonium p-toluenesulfonate, a benzalkonium salt (e.g., benzalkonium chloride (BAC)), or a benzethonium salt (e.g., benzethonium chloride (BZT)) and mixtures thereof. In one embodiment, the nanoparticle has a nominal BET surface area of about 1,200 m²/g and the surfactant is hexadecyltrimethylammonium bromide (C₁₆; CTAB). In one embodiment, the MSNP is further modified with SiOH. In one embodiment, the MSNP is further modified with PEG. In one embodiment, the nanoparticle is loaded with two or more different cargos. In one embodiment, the two or more different cargos are of different kinds. In one embodiment, the nanoparticle further comprises a targeting ligand. In one embodiment, the nanoparticle is loaded with one or more cargo components, said cargo being loaded either exclusively onto the nanoparticle surface or is loaded through pore and/or surface loading. In one embodiment, (a) the MSNP is methylated with hexamethyldisilazane (HDMS) and is loaded with a cargo having a water solubility of between about less than 0.001 mg/mL to about 0.5 mg/mL; or (b) the MSNP is aminated with aminopropyltriethoxysilane (APTES) and is loaded with a cargo having a water solubility of between about 0.2 mg/ml to greater than about 3,000 mg/ml; and (c) the surfactant is hexadecyltrimethylammonium bromide (C₁₆; CTAB). In one embodiment, the precursor solution is dispersed within an oil phase to form a multiphase emulsion, and wherein: (a) the precursor solution comprises (1) tetraethyl orthosilicate (TEOS), tetramethyl orthosilicate (TMOS) or a mixture thereof, and (2) at least one cationic surfactant; and wherein (b) the oil phase comprises a C₁₂-C₂₀ alkane and a non-ionic emulsifier soluble in the oil phase. In one embodiment. (a) the emulsion is an oil-in-water emulsion; (b) the precursor solution comprises one or more components selected from the group consisting of: (1) hexadecyltrimethylammonium bromide (C₁₆; CTAB), (2) a Brij® surfactant (for example Brij®56), (3) a block copolymer based on ethylene oxide and propylene oxide (for example Pluronic® F108), optionally in combination with urea and/or polystyrene (PS) or glycerol monooleate, (4) a difunctional block copolymer surfactant terminating in a primary hydroxyl group (for example Pluronic® P123), optionally in combination with (i) a triblock copolymer of poly(ethylene oxide) (PEO) or poly(propylene oxide) (PPO), and/or (ii) polypropylene glycol acrylate (PPGA); and wherein (c) the volumetric ratio of the precursor solution:oil phase is between about 1:2 to 1:4.

In one embodiment, the nanoparticle is a mesoporous silica nanoparticle (MSNP) which is self-assembled using a templating surfactant system comprised of at least one cationic surfactant. In one embodiment, protocell is also provided comprising a nanoparticle coated with a lipid bi- or multilayer. In one embodiment, the lipid bi- or multilayer is comprised of lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures thereof. In one embodiment, the lipid bi- or multilayer comprises DOPC in combination with DOPE. In one embodiment, the lipid bi- or multilayer comprises DOTAP, DOPG, DOPC or mixtures thereof. In one embodiment, the lipid bi- or multilayer comprises DOPG and DOPC. In one embodiment, the lipid bi- or multilayer further comprises cholesterol. In one embodiment, the lipid bi- or multilayer further comprises a cell targeting species. In one embodiment, the targeting species is a targeting peptide. In one embodiment, (a) the lipid bi- or multilayer comprises DOPC or DPPC in combination with DOPE, cholesterol and PEG-2000 PE (18:1); and (b) the targeting peptide targets cancer cells. In one embodiment, wherein at a pH of about 5, the protocel releases approximately 30% to approximately 100% of its cargo over a time period of about six hours after delivery. In one embodiment, (a) the nanoparticle is an aminated silica nanoparticle (MSNP); (b) the lipid bilayer comprises DOPC or DPPC in combination with DOPE, cholesterol and PEG-2000 PE (18:1); (c) the targeting peptide targets cancer cells; (d) the cargo comprises one or more hydrophilic anticancer active ingredients; and (e) at a pH of about 5, the protocell releases between about 30% to about 100% of its cargo at about three hours after delivery, and releases about 60% to about 100% of its cargo at about six hours after delivery. In one embodiment, (a) the nanoparticle is a methylated silica nanoparticle (MSNP) or a MSNP with functionalized with a phenyl group; (b) the lipid bilayer comprises DOPC or DPPC in combination with DOPE, cholesterol and PEG-2000 PE (18:1); (c) the targeting peptide targets cancer cells; (d) the cargo comprises one or more hydrophobic anticancer active ingredients; and (e) at a pH of about 5, the protocell releases between about 40% to about 90% of its cargo at about three hours after delivery, and releases about 90% to about 100% of its cargo at about twelve hours after delivery. In one embodiment, upon delivery, the protocell releases substantially all of its cargo through burst release over a period of about twelve hours. In one embodiment, upon delivery, the protocell releases its cargo through sustained release at a rate of about 10% weight cargo per day over a period of about ten days. In one embodiment, the targeting peptide is selected from the group consisting of a S94 peptide, a MET binding peptide or mixtures thereof. A pharmaceutical composition comprising: (a) a therapeutically effective amount of nanoparticles, said nanoparticles being loaded with one or more active ingredients; and (b) optionally, one or more pharmaceutically acceptable excipients. In one embodiment, the composition may be administered intranasally, intradermally, intramuscularly, intraosseously, intraperitoneally, intravenously, subcutaneously or intrathecally. Embodiment 47. A pharmaceutical composition comprising: (a) a therapeutically effective amount of protocells; and (b) optionally, one or more pharmaceutically acceptable excipients. In one embodiment, the composition may be administered intranasally, intradermally, intramuscularly, intraosseously, intraperitoneally, intravenously, subcutaneously or intrathecally.

In one embodiment, a method of treating cancer is provided, the method comprising administering a therapeutically effective amount of a pharmaceutical composition to a subject in need thereof, wherein the compositions comprises nanoparticles that are loaded with one or more anticancer agents. In one embodiment, the cancer is hepatocellular carcinoma. In one embodiment, an evaporation-induced self-assembly (EISA) process for making a mesoporous silica nanoparticle (MSNP) is provided. The process including the steps of: (a) preparing a precursor solution comprising (1) a surfactant (2) tetraethyl orthosilicate (TEOS), tetramethyl orthosilicate (TMOS) or a mixture thereof (3) a C₁₋₄ alcohol (for example, ethanol), and (4) water, wherein said surfactant, tetraethyl orthosilicate (TEOS), tetramethyl orthosilicate (TMOS) or a mixture thereof, C₁₋₄ alcohol, and water are combined at a temperature below the surfactant's critical micelle concentration; (b) adding either (1) an amine-containing silane to the precursor solution to replace a controllable fraction of Si—O—Si bonds with Si—R—NH₂ bonds, where R is a C₁₋₁₂ hydrocarbon, or (2) a methyl or phenyl-containing organosiloxane to replace a controllable fraction of Si—O—Si bonds with Si—R—CH₃ or Si—R-Ph bonds, where R is a C₁₋₁₂ hydrocarbon; (c) atomizing the precursor solution to generate droplets; (d) drying the droplets, thereby evaporating solvent and increasing effective surfactant concentration and inducing nanoparticle self-assembly; and (e) heating dried droplets, thereby evaporating residual solvent, inducing silica condensation and forming solid nanoparticles, wherein the degree of silica condensation is increased by thermal calcination to maximize the number of Si—O—Si bonds and is reduced by using acidified ethanol to extract structure-directing surfactants. In one embodiment, the precursor solution is dispersed within an oil phase to form a multiphase emulsion, and wherein: (a) the precursor solution comprises (1) tetraethyl orthosilicate (TEOS), tetramethyl orthosilicate (TMOS) or a mixture thereof, and (2) at least one cationic surfactant; and wherein (b) the oil phase comprises a Ci₂-C₂₀ alkane and a non-ionic emulsifier soluble in the oil phase. In one embodiment, the precursor solution is functionalized with an organosiloxane selected from the group consisting of aminopropyltriethoxysilane (APTES), 3-[2-(2-aminoethylamino)ethylamino]propyltrimethoxysilane (AEPTMS), hexamethyldisilazane (HDMS), sodium bis(trimethylsilyl)amide (NaHDMS), potassium bis(trimethylsilyl)amide (KHDMS) and phenyltriethoxysilane (PTS). In one embodiment, a method of diagnosing and/or treating a cancer, a bacterial infection or a viral infection is provided, the method comprising administering to a subject in need thereof a population of nanoparticles, wherein said nanoparticles are optionally coated with a lipid bi- or multilayer. In one embodiment, a method of diagnosing and/or treating a cancer, a bacterial infection or a viral infection, the method comprising administering to a subject in need thereof a population of nanoparticles, wherein said nanoparticles are coated with a lipid b- or multilayer which comprises a reporter. In one embodiment, a kit comprising a population of nanoparticles and/or a population of protocells and, optionally, instructions for the use of the protocells in the diagnosis and treatment of a cancer or a bacterial or viral infection is provided. In one embodiment, (a) the nanoparticle is loaded with the naked siRNA TD101 and the protocell is useful in the treatment of Pachyonychia Congenita; or (b) the nanoparticle is loaded with the naked siRNA 15NP and the protocell is useful in the treatment of delayed graft function associated with kidney transplant; or (c) the nanoparticle is loaded with the naked siRNA SYL040012 and the protocel is useful in the treatment of glaucoma and/or ocular hypertension; or (d) the nanoparticle is loaded with the naked siRNA SYL1001 and the protocell is useful in the treatment of dry eye syndrome; or (e) the nanoparticle is loaded with the naked siRNA Bevasiranib and the protocell is useful in the treatment of Wet AMD or Diabetic AMD; or (f) the nanoparticle is loaded with the naked siRNA QPI-1007 and the protocel is useful in the treatment of chronic optic nerve atrophy; or (g) the nanoparticle is loaded with the naked siRNA Sima-027/AGN211745 and the protocell is useful in the treatment of AMD and CNV; or (h) the nanoparticle is loaded with the naked siRNA PF-655 and the protocell comprising is useful in the treatment of AMD/DME.

In one embodiment, the nanoparticle is loaded with the siRNA siG12D and the protocell is useful in the treatment of pancreatic cancer. In one embodiment, the nanoparticle is loaded with the siRNA TKM-PLK1 (PLK1 SNALP, TKM-080301) and the protocell is useful in the treatment of a solid tumor and primary and secondary liver cancer. In one embodiment, the nanoparticle is loaded with the siRNA siRNA-EphA2-DOPC and the protocell is useful in the treatment of a solid tumor. In one embodiment, (a) the nanoparticle is loaded with the aptamer C2 (2′F RNA) and the protocell is useful in the treatment of leukemia cancer or a skin cancer, or (b) the nanoparticle is loaded with the aptamer EpDT3 (2′F RNA) and the protocell is useful in the treatment of colon cancer or breast cancer, or (c) the nanoparticle is loaded with the aptamer PSM-A10 (2′F RNA) and the protocell is useful in the treatment of prostate cancer; or (d) the nanoparticle is loaded with the aptamer S6 (2′F RNA) and the protocell is useful in the treatment of breast cancer; or (e) the nanoparticle is loaded with the aptamer C1 (2′F RNA) and the protocell is useful in the treatment of breast cancer; or (f) the nanoparticle is loaded with the aptamer CL4 (2′F RNA) and the protocel is useful in the treatment of breast cancer; or (g) the nanoparticle is loaded with the aptamer YJ1 (2′F RNA) and the protocell is useful in the treatment of metastatic colon cancer; or (h) the nanoparticle is loaded with the aptamer Aptamer 14 (2′F RNA) and the protocel is useful in the treatment of leukemia; or (i) the nanoparticle is loaded with the aptamer C10 (DNA) and the protocell is useful in the treatment of Burkitt like lymphoma; or (j) the nanoparticle is loaded with the aptamer Sgc8 (DNA) and the protocell is useful in the treatment of acute lymphoblastic leukemia; or (k) the nanoparticle is loaded with the aptamer TA6 (DNA) and the protocell is useful in the treatment of breast cancer, lymphoma and melanoma.

### REFERENCES

2007. Target Product Profile—A Strategic Development Process Tool, Food and Drug Administration
Arias et al., Nat. Rev. Microbiol., 10:266 (2012).
Al et al., Curr. Pharm. Des., j1:1644 (2010).
Aloush et al., Antimicrob. Agents Chem., 50:43 (2006).
Ashley et al., ACS Nano, 6:2174 (2012).
Ashley et al., Nat. Mater., 10:389 (2011).
Bai et al., Biomaterials, U:659 (2012b).
Bai et al., PLOS ONE, 7:e29886 (2012a).
Berge et al., Am. J. Hyg 73: 209-18 (1961).
Brinker at al., Advanced Materials. 11:579 (1999).
Calhoun et al., Clin. Orthoo. Related Res., 466:1356 (2008).
Carroll et al., Lanamuir. 25:13540 (2009).
Cauda et al., Nano Letters, 10:2484 (2010).
Chen et al., Accounts of Chemical Research, 44:841 (2011).
Chong et al., Cell, 158:314 (2014).
Chrastina, A, et al. J Vasc Res. 47: 531-43. 2010.
Clemens et al., Antimicrobial Agents and Chemotherapy, 56:2535 (2012).
Comiskey et al., Biochemistry, 22:3626 (1990).
Conley et al., Antimicrobial Agents and Chemotherapy, 41:1288 (1997).
Couvreur and Vauthier, Pharmaceutical Research, 21:1417 (2006).
Dengler et al., Journal of Controlled Release, 168:209 (2013).
des Rieux et al., Journal of Controlled Release, 116:1 (2006).
Diamond, Antiviral Res., 83:214 (2009).
Dietz et al., J. Virol., 87:6172 (2013).
Edwards, Immunodiagnostic: A Practical Approach. Oxford University Press, Oxford; England, (1999)
El-Sayed et al., J. Biol. Chem., 283:23450 (2008).
Epler at al., Advanced Healthcare Materials, 1:348 (2012).
Fleming et al., J. Trauma Acute Care Surg. 14:1067 (2013).
Gao et al., J. Phys. Chem. B., 11:1796 (2009).
Gavrilov et al., Yale J. Biol. Med., 855:187 (2012).
Georges-Courbot at al., Antimicrobial Agents and Chemotherapy, 50:1768 (2006).
Giri at al., Nanomedicine, 2:99 (2007).
Goodchild et al., Antiviral Res., 90:1 (2011).
Gottesman and Storz, Cold Spring Harbor Perspectives in Biology.
Grabrucker et al., PLoS ONE. 6:e17851 (2011).
Grassin-Delyle et al., Pharmacology & Therapeutics, 134:366 (2012).
Hafner et al., Clinical Chemistry, 50:490 (2004).
Hanson et al., J. Vis. Exp. (2013).
Harlow and Lane, Antibodies: A Laboratory Manual, Cold Spring Harbor Laboratory Press (1999).
Harmon at al., J. Virol., 86:12954 (2012).
He et al., Small, 5:2722 (2009).
Hosoya et al., Antimicrobial Agents and Chemotherapy, 48:4631 (2004).
Hudson et al., PLOS ONE. 9:e99209 (2014).
Huggins et al., Antimicrobial Agents and Chemotherapy, 26:476 (1984).
Hujer et al., Antimicrobial Agents and Chemotherapy. 50:4114 (2006).
Jackson et al., Vet. Pathol., 28:410 (1991).
Jain et al., N. Engl. J. Med., 364:1419 (2011).
Kawakami et al., Gene therapy, 7:292 (2000).
Kim et al., PLOS ONE, 8:e68988 (2013).
LaCasse at al., Nucl. Acids Res., 23:1647 (1995).
Lin et al., Journal of the American Chemical Society, 132:4834 (2010).
Liong et al., Journal of Materials Chemistry, 19:6251 (2009).
Liu et al., Chem. Comm., ___:5100 (2009).
Liu et al., J. Amer. Chem. Soc., 11:7567 (2009).
Liu, J W, et al., Journal of the American Chemical Society, 131:1354-5. 2009.
Lo et al., Molecular Cancer Therapeutics, 1:579 (2008).
Lu et al., Nature, 28:223 (1999).
Lu et al., Small, 6:1794 (2010).
Mann and Markham, Journal of Applied Microbiology, 84:538 (1998).
Mason et al., Antimicrobial Agents and Chemotherapy, 39:2752 (1995).
Meng et al., ACS Nano. 4:4539 (2010).
Meng et al., ACS Nano. 5:4434 (2011).
Merkus et al., Drugs in R & D, 8:133 (2007).
Meyer et al., Fresenius' Journal of Analytical Chemistry, 363:174 (___).
Mofaie at al., Asian Pac J Cancer Prev., 14:7045 (___).
Moon et al., Advanced Materials, 24:3724 (2012).
Moon et al., Journal of Chromatography A. 813:91 (1998).
Moon et al., Nat Mater, 12:243 (2011).
Moore et al., The Journal of Gene Medicine, 10:1134 (2008).
Moreau et al., Chem. Mater., 25:2394 (2013).
Morilla et al., Intracellular Delivery, ed. A Prokop, pp. 745-811: Springer Netherlands
Murat Cokol et al., at the website ubic.bioc.columbia.edu/papers/2000 nls/paper.html#tab2.
Murray at al., Bacteiology of War Wounds at the Time of Injury, Military Medicine, 171:826 (2006).
Musa at al., J. Applied Sci., 11: 3650 (2011).
Musa et al., J. Applied Sci. 11:3850 (2011).
Nasser et al., J. Virol., 1.2:10169 (2010).
National Institute of Standards and Technology (NIST) (Particle Size Characterization, Special Publication 960-1, January 2001).
Nielsen et al., Molec. Biol., 1:89 (1999).
Nocker et al., Journal of Microbiological Methods. 67:310 (2006).
Nordmann et al., Lancet Infect. Dis., 9:228 (2009).
O'Brien et al., Virology, 426:100 (2012).
O'Brien. Antiviral Res., 75:20 (2007).
Oldenborg et al., Science, 288:2051 (2000).
Paessler et al., J. Infect. Dis., 18:2072 (2004).
Palomino et al., Antimicrobial Agents and Chemotherapy, 46:2720 (2002).
Pascal et al. ACS Nano. 7, 11174 (2013).
Paterson and Bonomo, Clinical Microbiology Reviews, 18:657(2005).
Peer et al., Nat. Nano., 2:751 (2007).
Pinto-Alphandary et al., International journal of antimicrobial agents. 13:155 (2000).
Rajasekaran et al., Int J Antimicrob Agents, 41:358 (2013).
Reed et al., J. Infect. Dis., 189:1013 (2004).
Reed et al., Vaccine, 3:3139 (2005).
Rockx et al., J. Virol., 84:9831 (2010).
Rosi et al., Science, 12:1027 (2006).
Rulker et al., PLoS One, 1:e37242 (2012).
Rungta et al., Molecular Therapy Nucleic Acids, 2:e136 (2013).
Salazar et al., Zoonoses Public Health, 59:278 (2012).
Sambrook et al., Sambrook et al., 2001; Ausubel, ed., 1994, Current Protocols in Molecular Biology Vol. I-III; Cells, ed. (2001).
Sambrook et al., Animal Cell Culture: IRL (1986).
Sambrook et al., Cell Biology: A Laboratory Handbook. Vol. I-III; Coligan, ed. (1994),
Sambrook et al., 1994, Current Protocols in Immunology, Vol. I-III; Gait ed. (1994).
Sambrook et al., 1985, Nucleic Acid Hybridization: Hames & Higgins, eds., (1985).
Sambrook et al., 1984, Oligonucleotide Synthesis; Hames & Higgins eds. (1984).
Sambrook et al., ranscription And Translation; Freshney, ed., (1984).
Sandor et al., Immunobiology (2006).
Sautto et al., Biomed Research International, 2013: ___(2013).
Schloßbauer et al., Advanced Healthcare Materials, 1:316 (2012).
Schmidt C S, Morrow W J W, Sheikh N A. 2007. Smart adjuvants. Expert Review of Vaccines 6: 391-400
Sener et al., Journal of Clinical Microbiology, 4:1124 (2011),
Sindac, J, et al. J. Med. Chem. 55: 3535-45 (2012).
Snell, Expert Opin Pharmacother., 2:1317 (2001).
Sommerman, Factors Influencing The Biodistribution of Liposomal Systems. The University of British Columbia. 163 pp.
Soofi and Seleem. Antimicrob Agents Chemother., 56.6407 (2012).
Souris et al., Biomaterials. 1:5564 (2010).
Steele et al., Vet Pathol., 47:790 (2010).
Stephen et al., ICN Pharmaceuticals Scientific Symposium, (1979).
Takeuchi et al., Advanced Materials, 17:1067 (2005).
Tam et al., Accounts of Chemical Research, 46:792 (2013).
Townson et al., Journal of the American Chemical Society. 35:16030 (2013).
Walters et al., ASN NEURO, 4:art:e00099 (2012).
Weaver et al., Am J. Trop. Med. Hyg., 60:441 (1999).
Weaver et al., Lancet VEE Study Group, 436:40 (1996).
Weis, TIBS, 2:185 (1998).
Weis, Trends Biochem. Sci., 1998 23:235 (1998).
Weksler et al., Fluids and Barriers, 102013.
White et al., Am. J. Trop. Med. Hyg., 84:709 (2011).
Wong et al., Journal of Controlled Release, 92:265 (2003).
Woodford and Wareham, Journal of Antimicrobial Chemotherapy, 3:225 (2009).
Zanta et al., Proc. Natl. Acad. Sci. USA, 96:91 (1999).
Zhang et al., J. Controlled Release, 123:1 (2007).
Zhang et al., Journal of the American Chemical Society, 134:15790 (2012).
Zhao et al., ACS Nano, 5:1366 (2011).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles.

1. An antimicrobial protocell comprising a mesoporous silica or metal oxide nanoparticle which is loaded with an anti-viral or anti-bacterial cargo and which is coated with a lipid bi- or multilayer, wherein: (a) the mesoporous metal oxide nanoparticle has a pore size which ranges from about 0.001 to about 100 nm, and a diameter ranging from about 25 nm to about 500 nm; and (b) the lipid bi- or multilayer comprises at least one targeting moiety that targets a virally-infected or a bacterially-infected host cell.

2. The protocell of claim 1, wherein the targeting moiety is a peptide or single chain variable fragment (scFv), or wherein the targeting moiety targets ephrin B2 and/or ephrin B3 or is a peptide or single chain variable fragment (scFv) that targets ephrin B2 and/or ephrin B3, or wherein the targeting moiety comprises one or more amino acid sequences selected from the groups consisting of TGAILHP, QGAINHP, QHIRKPP, QHRIKPP and QHILNPP, or wherein the targeting moiety is a peptide or single chain variable fragment (scFv) and optionally wherein the targeting moiety is a Fcγ from human IgG, human complement C3, ephrin B2 or mannosylated cholesterol.

3. The protocell of claim 1, further comprising an endosomolytic moiety, which optionally is a peptide and optionally ruptures acidic intracellular vesicles of the virally-infected cell, and optionally is a peptide selected from the group consisting of octaarginine (RS), H5WYG, Penetratin-HA2, modified HA2-TAT, 43E and Histidine 10, or further comprising an endosomolytic moiety wherein the endosomolytic moiety optionally ruptures a bacterially-infected cell membrane ruptures acidic intracellular vesicles of the bacterially-infected cell.

4. The protocell of claim 1, wherein the antiviral cargo is selected from the group consisting of a small molecule, a mRNA, a siRNA, a shRNA, a micro RNA, a PNA, a PNA comprised of RNA's, an antibody, a protein, a protein toxin (e.g., ricin toxin A-chain or diphtheria toxin A-chain) and/or DNA (including double stranded or linear DNA, minicircle DNA, plasmid DNA which may be supercoiled and/or packaged (e.g., with histones) and which may be optionally modified with a nuclear localization sequence), ribavirin or a nucleic acid or wherein the antiviral cargo is a siRNA or microRNA which targets conserved regions of EEEV or VEEV RNA-dependent RNA polymerase (RdRp) or nsp1 and E1 glycoprotein genes, an antibody fragment, or an IgG molecule or a fragment thereof.

5-6. (canceled)

7. The protocell of claim 1, wherein the nanoparticle is an aminated mesoporous silica nanoparticle (MSNP) optionally the nanoparticle is aminated with aminopropyltriethoxysilane (APTES) or 3-[2-(2 aminoethylamino)ethylamino]propyltrimethoxy silane (AEPTMS).

8. (canceled)

9. The protocell of claim 1, wherein the nanoparticle has a differential pore volume of between about 0.25 cm³/g to about 10 cm³/g, from about 0.3 cm³/g to about 3 cm³/g or from about 0.25 cm³/g to about 1.5 cm³/g, or has a nominal BET surface area of between about 50 m²/g to about 1,500 m²/g, or from about 100 m²/g to about 1,300 m²/g.

10. The protocell of claim 1, wherein the nanoparticle is a mesoporous silica nanoparticle (MSNP) and wherein the weight ratio of antiviral cargo to silica ranges from about 0.10 to about 0.75.

11. The protocell of claim 1, wherein the nanoparticle has a pore size ranges from about 0.001 to about 100 nm, from about 0.01 nm to about 50 nm, from about 0.1 to about 100 nm, or from about 2 nm to about 25 nm.

12. (canceled)

13. The protocell of claim 1, wherein the antibacterial cargo is effective in the treatment of an infection caused by a bacterium selected from the group consisting of multidrug-resistant (MDR) Klebsiella pneumoniae (Kpn), methicillin-resistant Staphylococcus aureus (MRSA), F, tularensis and B, pseudomallei pr wherein the antibacterial cargo is a nucleic acid molecule capable of inhibiting the translation of a mRNA selected from the group consisting of a TEM beta-lactamase (class A) mRNA, a SHV beta-lactamase (class A) mRNA, a CTX-M beta-lactamase (class A) mRNA, an OXA beta-lactamase (class D) mRNA, a PER mRNA, a VEB mRNA, a GES mRNA, an IBC beta-lactamase mRNA, an AmpC type β-lactamase mRNA, and a carbapenemase mRNA (including but not limited to KPC (K, pneumoniae carbapenemase) (Class A) mRNA), and the mammalian and non-mammalian orthologs thereof or wherein the antibacterial cargo comprises a nucleic acid molecule capable of inhibiting the translation of a mRNA selected from the group consisting of Metallo-beta-lactamase NDM-1 mRNA, SHV and TEM beta-lactamase mRNA, CMY-6 AmpC-type beta-lactamase mRNA, CTX-M-15 extended spectrum beta-lactamase mRNA: TEM-1 beta-lactamase mRNA: OXA-1 beta-lactamase mRNA: Aminoglycoside-(3)(9)-adenyltransferase AADA2 mRNA: Sul1 dihydropteroate synthase mRNA: Undecaprenyl-diphosphatase mRNA: 16S ribosomal RNA methyltransferase mRNA; AAC(6)-Ib aminoglycoside 6-N-acetyl transferase type Ib mRNA; Sul1 dihvdropteroate synthase mRNA: 16S rRNA methyltransferase RmtC mRNA; Aminoglycoside 3 phosphotransferase APH(3)-Ib (strA) mRNA; Sul2 mRNA, sulfonamide insensitive dihvdropteroate svnthetase mRNA: Streptomycin 3-O-adenylyltransferase aadA ANT(3)-Ia mRNA: Dfra14 trimethoprim-resistant dihydrofolate reductase mRNA: QnrB10 mRNA: Aminoglycoside N(3)-acetyltransferase II (ACC(3)-II)mRNA; Tetracycline efflux protein TetA mRNA; and Macrolide 2-phosphotransferase mphA mRNA, and the mammalian and non-mammalian orthologs thereof, and optionally, wherein the nucleic acid molecule is selected from the group comprising siRNA, miRNA, shRNA and/or asRNA, or wherein the antibacterial cargo is a peptide nucleic acid (PNA) comprising nucleic acid molecules which inhibit the translation of a mRNA selected from the group consisting of a TEM beta-lactamase (class A) mRNA, a SHV beta-lactamase (class A) mRNA, a CTX-M beta-lactamase (class A) mRNA, an OXA beta-lactamase (class D) mRNA, a PER mRNA, a VEB mRNA, a GES mRNA, an IBC beta-lactamase mRNA, an AmpC type β-lactamase mRNA, and a carbapenemase mRNA (including but not limited to KPC (K, pneumoniae carbapenemase) (Class A) mRNA), and the mammalian and non-mammalian orthologs thereof or wherein the antibacterial cargo comprises a peptide nucleic acid (PNA) comprising nucleic acid molecules that inhibit the translation of a mRNA selected from the group consisting of Metallo-beta-lactamase NDM-1 mRNA, SHV and TEM beta-lactamase mRNA, CMY-6 AmpC-type beta-lactamase mRNA, CTX-M-15 extended spectrum beta-lactamase mRNA: TEM-1 beta-lactamase mRNA; OXA-1 beta-lactamase mRNA; Aminoglycoside-(3)(9)-adenyltransferase AADA2 mRNA; Sul1 dihydropteroate synthase mRNA; Undecaprenyl-diphosphatase mRNA: 16S ribosomal RNA methyltransferase mRNA: AAC(6)-Ib aminoglycoside 6-N-acetyl transferase type Ib mRNA; Sul1 dihydropteroate synthase mRNA; 16S rRNA methyltransferase RmtC mRNA: Aminoglycoside 3 phosphotransferase APH(3)-Ib (strA) mRNA: Sul2 mRNA, sulfonamide insensitive dihydropteroate synthetase mRNA: Streptomycin 3-O-adenylyltransferase aadA ANT(3)-Ia mRNA: Dfra14 trimethoprim-resistant dihydrofolate reductase mRNA; QnrB10 mRNA; Aminoglycoside N(3)-acetyltransferase II (ACC(3)-II)mRNA: Tetracycline efflux protein TetA mRNA: Macrolide 2-phosphotransferase mphA mRNA, and the mammalian and non-mammalian orthologs thereof, asRNA molecules which comprise one or more nucleotide sequences selected from the group consisting of caagttttc, gaaatcagt, gaaatcagt, gggattcct, actcttcct, ttaatgagg, tcaaaggcc, eggctcggc, ccaattaaa, tgggtatta, ttaatgagg, ggcgtcagc, atatggtct, agaggttc, aggggcttc, gatgttaa, attctcat, atttgtacc, cgcgatatc, gtctggcct and gattcactc and equivalents and fragments thereof, a peptide nucleic acid (PNA) which binds to a ribosomal binding site of one or more genes selected from the group consisting of qnrB9, aac(6′)-Ib, sul1, bla_{SHV-11}, bla_{CTX-M-15}, blaNDM-1, the bla gene encoding TEM-1 and equivalents thereof, clavulanic acid, Gentamicin, Kanamycin, Neomycin, Netilmicin, Tobramycin, Paromomycin, Spectinomycin, Geldanamycin, Herbimycin, Rifaximin, Streptomycin, Ertapenem, Doripenem, Imipenem/Cilastatin, Meropenem, Cefadroxil, Cefazolin, Ceohalothin, Cephalexin, Cefaclor, Cefamandole, Cefoxitin, Cefprozil, Cefuroxime, Cefixime, Cefdinir, Cefditoren, Cefoperazone Cefotaxime, Cefpodoxime, Ceftazidime, Ceftibuten, Ceftizoxime Ceftriaxone, Cefeoime, Ceftaroline fosamil, Ceftobiorole, Teicoplanin, Vancomycin, Telavancin, Daptomycin, Oritavancin, WAP-8294A, Azithromycin, Clarithromycin, Dirithromycin, Erythromycin, Roxithromycin, Telithromycin, Spiramycin, Clindamycin, Lincomycin, Aztreonam, Furazolidone, Nitrofurantoin, Oxazolidinones, Linezolid, Posizolid, Radezolid, Torezolid, Amoxicillin, Ampicillin, Azlocillin, Carbenicillin, Cloxacillin Dicloxacillin, Flucloxacillin, Mezlocillin, Methicillin, Nafcillin, Oxacillin, Penicillin G, Penicillin V, Piperacillin, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, Mafenide, Sulfacetamide, Sulfadiazine, Sulfadimethoxine, Sulfamethizole, Sulfamethoxazole, Sulfasalazine, Sulfisoxazole, Trimethoprim-Sulfamethoxazole, Sulfonamidochrvsoidine, Demeclocycline, Doxycycline, Vibramycin Minocycline, Tigecycline, Oxytetracycline, Tetracycline, Clofazimine, Capreomycin, Cycloserine, Ethambutol, Rifampicin, Rifabutin, Rifapentine, Arsphenamine, Chloramphenicol, Fosfomycin, Fusidic acid, Metronidazole, Mupirocin, Platensimycin, Quinupristin/Dalfopristin, Thiamphenicol, Tigecycline or Tinidazole, and combinations thereof.

14. The protocell of claim 1, wherein the nanoparticle is a silica nanoparticle (MSNP) which is coated with a lipid bi- or multilayer and wherein: (a) at a pH of about 7 and a period of about 12 days after delivery, the protocell will release no more than about 10 wt % of its antiviral cargo; and (b) at a pH of about 5 and a period of about one day after delivery, the protocell will release no less than about 90 wt % of its antiviral cargo, or wherein the nanoparticle is a mesoporous silica nanoparticle (MSNP) which is coated with a lipid multilayer and wherein: (a) at a pH of about 7 and a period of about 12 days after delivery, the protocell will release no more than about 5 wt % of its antiviral cargo; and (b) at a pH of about 5 and a period of about ten days after delivery, the protocell will release no less than about 10 wt % to about 60 wt % of its antiviral cargo.

15. (canceled)

16. The protocell of claim 1, wherein the nanoparticle is loaded with: (a) an anti-HIV agent selected from the group consisting of 3TC (Lamivudine), AZT (Zidovudine), (−)-FTC, ddI (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), T20, fuseon, and mixtures thereof; or (b) an anti-HBV agent selected from the group consisting of hepsera (adefovir dipivoxil), lamivudine, entecavir, telbivudine, tenofovir, emtricitabine, clevudine, valtorcitabine, amdoxovir, pradefovir, racivir, BAM 205, nitazoxanide, UT 231-B, Bay 41-4109, EHT899, zadaxin (thymosin alpha-1), and mixtures thereof; or (c) an anti-HCV agent selected from the group consisting of interferon, pegylated interferon, ribavirin, NM 283, VX-950 (telaprevir), SCH 50304, TMC435, VX-500, BX-813, SCH503034, R1626, ITMN-191 (R7227), R7128, PF-868554, IT033, CGH-759, GI 5005, MK-7009, SIRNA-034, MK-0608, A-837093, GS 9190, ACH-1095, GSK625433, TG4040 (MVA-HCV), A-831, F351, NS5A, NS4B, ANA598, A-689, GNI-104, IDX102, ADXI84, GL59728, GL60667, PSI-7851, TLR9 Agonist, PHX1766, SP-30, and mixtures thereof.

17-20. (canceled)

21. The protocell of claim 1, wherein the nanoparticle has a differential pore volume of between about 0.25 cm³/g to about 10 cm³/g, optionally from about from about 0.3 cm³/g to about 3 cm³/g or from about 0.25 cm³/g to about 1.5 cm³/g, or wherein the nanoparticle has a nominal BET surface area of between about 50 m²/g to about 1,500 m²/g, optionally from about 100 m²/g to about 1,300 m²/g.

22-28. (canceled)

29. A nanoparticle comprising silica or metal oxide, the nanoparticle functionalized with a hydrophobic group and loaded with a water-insoluble cargo.

30. The nanoparticle of claim 29, wherein the nanoparticle is porous and wherein the pores optionally have a diameter of about 0.01 nm to about 50 nm.

31. The nanoparticle of claim 29, wherein the hydrophobic group is a methyl group or a phenyl group.

32. The nanoparticle of claim 29, wherein the nanoparticle is functionalized with a hydrophobic organosiloxane which hydrophobic organosiloxane optionally is hexamethyldisilazane (HDMS), sodium bis(trimethylsilyl)amide (NaHDMS), potassium bis(trimethylsilyl)amide (KHDMS), or phenyltriethoxysilane (PTS).

33-39. (canceled)

40. An evaporation-induced self-assembly (EISA) process for making functionalized silica nanoparticles loaded with a water-insoluble cargo comprising:
(a) atomizing a precursor solution to generate droplets; wherein the precursor solution comprises (1) a surfactant, (2) tetraethyl orthosilicate (TEOS) or tetramethyl orthosilicate (TMOS), (3) a C₁₋₄ alcohol, (4) a hydrophobic organosiloxane, and (5) water;
(b) drying and heating the droplets, thereby evaporating solvent and increasing effective surfactant concentration; and
(c) loading the nanoparticles with a water-insoluble cargo or
(i) combining an aqueous phase precursor solution and an oil phase precursor solution, thereby forming an emulsion, whereinthe aqueous phase precursor solution comprises a hydrophobic organosiloxane, a first surfactant, tetraethyl orthosilicate (TEOS) or tetramethyl orthosilicate (TMOS), an acid, and water, and the oil phase precursor solution comprises a second surfactant and an oil;
(ii) heating the emulsion, thereby generating nanoparticles;
(iii) separating the nanoparticles from the remaining emulsion;
(iv) loading the nanoparticles with a water-insoluble cargo.

41. The evaporation-induced self-assembly (EISA) process of claim 40, wherein the surfactant is below the critical micelle concentration of the surfactant.

42. The evaporation-induced self-assembly (EISA) process of claim 40, wherein the surfactant comprises a cationic surfactant or wherein the surfactant is selected from the group consisting of a dodecylsulfate salt, a tetradecyl-trimethyl-ammonium salt, a hexadecyltrimethylammonium salt, an octadecyltrimethylammonium salt, a dodecylethyldimethylammonium salt, a cetylpyridinium salt, polyethoxylated tallow amine (POEA), hexadecyltrimethylammonium p-toluenesulfonate, a benzalkonium salt, a Brij® surfactant, a poloxamer, and a benzethonium salt or wherein the surfactant is selected from the group consisting of benzethonium chloride, benzalkonium chloride, cetylpyridinium chloride, dodecylethyldimethylammonium bromide, octadecyltrimethylammonium bromide, hexadecyltrimethylammonium bromide, tetradecyl-trimethyl-ammonium bromide, tetradecyl-trimethyl-ammonium chloride, sodium dodecylsulfate, lithium dodecylsulfate, Brij®-56, Pluronic® F108, and Pluronic® P123.

43-56. (canceled)


SEQUENCE LISTING (61 sequences)

SEQ ID NO: 1 - DNA, 21 bp, Artificial Sequence
  A synthetic siRNA sequence
gcccccgauugggggcgactt

SEQ ID NO: 2 - DNA, 21 bp, Artificial Sequence
  A synthetic siRNA sequence
gcgucuagccauggcguuatt

SEQ ID NO: 3 - DNA, 21 bp, Artificial Sequence
  A synthetic siRNA sequence
ggacgaccggguccuuucutt

SEQ ID NO: 4 - DNA, 21 bp, Artificial Sequence
  A synthetic siRNA sequence
ggccuugugguacugccugtt

SEQ ID NO: 5 - DNA, 21 bp, Artificial Sequence
  A synthetic siRNA sequence
ggccuugugguacugccugtt

SEQ ID NO: 6 - DNA, 21 bp, Artificial Sequence
  A synthetic siRNA sequence
ggucucguagaccgugcactt

SEQ ID NO: 7 - DNA, 21 bp, Artificial Sequence
  A synthetic siRNA sequence
caggcaguaccacaaggcctt

SEQ ID NO: 8 - RNA, 27 nt, Artificial Sequence
  A synthetic siRNA sequence
cugcucugccuuuagcagauccuccuu

SEQ ID NO: 9 - RNA, 25 nt, Artificial Sequence
  A synthetic siRNA sequence
ggaggaucugcuaaaggcagagcag

SEQ ID NO: 10 - RNA, 27 nt, Artificial Sequence
  A synthetic siRNA sequence
gcugguacaaauauccuuaucuugguu

SEQ ID NO: 11 - RNA, 25 nt, Artificial Sequence
  A synthetic siRNA sequence
ccaagauaaggauauuuguaccagc

SEQ ID NO: 12 - RNA, 27 nt, Artificial Sequence
  A synthetic siRNA sequence
gaauccugccauaccaguuuccucgac

SEQ ID NO: 13 - RNA, 25 nt, Artificial Sequence
  A synthetic siRNA sequence
cgaggaaacugguauggcaggautc

SEQ ID NO: 14 - RNA, 27 nt, Artificial Sequence
  A synthetic siRNA sequence
cuugaguucuguugcugauugcuggau

SEQ ID NO: 15 - RNA, 25 nt, Artificial Sequence
  A synthetic siRNA sequence
ccagcaaucagcaacagaacucaag

SEQ ID NO: 16 - RNA, 27 nt, Artificial Sequence
  A synthetic siRNA sequence
aaauauucucagagcuugaugcuuguc

SEQ ID NO: 17 - DNA, 25 bp, Artificial Sequence
  A synthetic siRNA sequence
caagcaucaagcucugagaauautt

SEQ ID NO: 18 - RNA, 27 nt, Artificial Sequence
  A synthetic siRNA sequence
ccagaaucauugagcuuugugauacug

SEQ ID NO: 19 - RNA, 25 nt, Artificial Sequence
  A synthetic siRNA sequence
guaucacaaagcucaaugauucugg

SEQ ID NO: 20 - RNA, 27 nt, Artificial Sequence
  A synthetic siRNA sequence
aucuucuugcguuucccugucucuggg

SEQ ID NO: 21 - DNA, 25 bp, Artificial Sequence
  A synthetic siRNA sequence
cagagacagggaaacgcaagaagat

SEQ ID NO: 22 - RNA, 27 nt, Artificial Sequence
  A synthetic siRNA sequence
accacuagucaguacuuucuuccacgg

SEQ ID NO: 23 - DNA, 25 bp, Artificial Sequence
  A synthetic siRNA sequence
guggaagaaaguacugacuaguggt

SEQ ID NO: 24 - protein, 42 aa, Artificial Sequence
  A synthetic nuclear localization amino acid sequence
GNQSSNFGPMKGGNFGGRSSGPYGGGGQYFAKPRNQGGYGGC

SEQ ID NO: 25 - protein, 7 aa, Artificial Sequence
  A synthetic nuclear localization amino acid sequence
RRMKWKK

SEQ ID NO: 26 - protein, 7 aa, Artificial Sequence
  A synthetic nuclear localization amino acid sequence
PKKKRKV

SEQ ID NO: 27 - protein, 16 aa, Artificial Sequence
  A synthetic nuclear localization amino acid sequence
KRPAATKKAGQAKKKK

SEQ ID NO: 28 - DNA, 12 bp, Artificial Sequence
  A synthetic oligonucleotide
catcaagttttc

SEQ ID NO: 29 - DNA, 12 bp, Artificial Sequence
  A synthetic oligonucleotide
cataaccacaat

SEQ ID NO: 30 - DNA, 12 bp, Artificial Sequence
  A synthetic oligonucleotide
catgaaatcagt

SEQ ID NO: 31 - DNA, 12 bp, Artificial Sequence
  A synthetic oligonucleotide
catgggattcct

SEQ ID NO: 32 - DNA, 13 bp, Artificial Sequence
  A synthetic oligonucleotide
caattaaatgagg

SEQ ID NO: 33 - DNA, 12 bp, Artificial Sequence
  A synthetic oligonucleotide
cattcaaaggcc

SEQ ID NO: 34 - DNA, 12 bp, Artificial Sequence
  A synthetic oligonucleotide
catggcgtcggc

SEQ ID NO: 35 - DNA, 12 bp, Artificial Sequence
  A synthetic oligonucleotide
catccaattaaa

SEQ ID NO: 36 - DNA, 12 bp, Artificial Sequence
  A synthetic oligonucleotide
cattgggtatta

SEQ ID NO: 37 - DNA, 12 bp, Artificial Sequence
  A synthetic oligonucleotide
caattaatgagg

SEQ ID NO: 38 - DNA, 12 bp, Artificial Sequence
  A synthetic oligonucleotide
catggcgtcggc

SEQ ID NO: 39 - DNA, 12 bp, Artificial Sequence
  A synthetic oligonucleotide
catatatggtct

SEQ ID NO: 40 - DNA, 12 bp, Artificial Sequence
  A synthetic oligonucleotide
caatggaggttc

SEQ ID NO: 41 - DNA, 11 bp, Artificial Sequence
  A synthetic oligonucleotide
catggggcttc

SEQ ID NO: 42 - DNA, 12 bp, Artificial Sequence
  A synthetic oligonucleotide
catgatgtttaa

SEQ ID NO: 43 - DNA, 12 bp, Artificial Sequence
  A synthetic oligonucleotide
caaggttctcat

SEQ ID NO: 44 - DNA, 12 bp, Artificial Sequence
  A synthetic oligonucleotide
catatttgtacc

SEQ ID NO: 45 - DNA, 12 bp, Artificial Sequence
  A synthetic oligonucleotide
catcgcgatatc

SEQ ID NO: 46 - DNA, 12 bp, Artificial Sequence
  A synthetic oligonucleotide
cacgtctggcct

SEQ ID NO: 47 - DNA, 12 bp, Artificial Sequence
  A synthetic oligonucleotide
catgattcactc

SEQ ID NO: 48 - protein, 7 aa, Artificial Sequence
  A synthetic peptide
ASVHFPP

SEQ ID NO: 49 - protein, 7 aa, Artificial Sequence
  A synthetic peptide
TATFWFQ

SEQ ID NO: 50 - protein, 7 aa, Artificial Sequence
  A synthetic peptide
TSPVALL

SEQ ID NO: 51 - protein, 7 aa, Artificial Sequence
  A synthetic peptide
IPLKVHP

SEQ ID NO: 52 - protein, 7 aa, Artificial Sequence
  A synthetic amino acid sequence
TGAILHP

SEQ ID NO: 53 - protein, 7 aa, Artificial Sequence
  A synthetic amino acid sequence
QGAINHP

SEQ ID NO: 54 - protein, 7 aa, Artificial Sequence
  A synthetic amino acid sequence
QHIPKPP

SEQ ID NO: 55 - protein, 7 aa, Artificial Sequence
  A synthetic amino acid sequence
QHIRKPP

SEQ ID NO: 56 - protein, 7 aa, Artificial Sequence
  A synthetic amino acid sequence
QHRIKPP

SEQ ID NO: 57 - protein, 7 aa, Artificial Sequence
  A synthetic amino acid sequence
QHILNPP

SEQ ID NO: 58 - protein, 7 aa, Artificial Sequence
  A synthetic amino acid sequence
SILPYPY

SEQ ID NO: 59 - protein, 7 aa, Artificial Sequence
  A synthetic amino acid sequence
ASYSGTA

SEQ ID NO: 60 - DNA, 31 bp, Artificial Sequence
  A synthetic nucleotide sequence
ttgaaaaaggaagagtatgagtattcaacat

SEQ ID NO: 61 - DNA, 12 bp, Artificial Sequence
  A synthetic nucleotide sequence
catactcttcct